SEQUENCE LISTING (339 sequences)

SEQ ID NO: 1 - DNA, 80001 bp, Homo sapiens
  source 1..80001: mol_type = genomic DNA
gctaccatcaactctttctcctagactgttccagggccttgcaactagccttgtgctgtagttttgtttcatcacgtccagttctccactctacacctgcaacatagatcagacagctcctggctcaaaatcctctgagggcttctcatcttagaataaactctcggttctggccgggtgcggtggcttacgcctgtagtcccagcactttgggagtccgaggcgggcggatcacttgaactcaggagtttgagaccagcctgggcaacatggtgaactcccatctctatcaaaaatacaaaaacttagccaggcgtggtggttcgcatctgtggtcccagctacttaggacgctgaggagggaggatcgcttgagctcagggtggacgttgcagtgagccaagattgcgccactgcactgcagcctgggtgacagaatgagaccccatccccaccccccccaaaaaagaatgaactcccagttctcatagtggccccagctgcctttccaatcacattccctaccactctccagcaacactgacttcctcgttagtccccaacatgccaggcatagtctctcctcatgtcctttgaacttgcctggaatgttctttccccagatattcatatgagggagtaaaatgagggtgaaaaccagcagatatctaaatagcacccccttcacttagtttatctttctcaaagcccttatcactatgtgaaatgatatattatacttatttgtatgctagtatgaatcttcccggcaagaatgttagtttgctgtctgttcagtaccgtgcatccagagcctggaagagtgcctggcacatagcaggtagtcaataaatgaatgggggcaagcagccaaatcagaatcaggttttcttgctaagcatagaactaacagaaggatcattgaatggattggataatgactggcatcagggtaaggtccccttaacaaacactcctgtcctgaacacctggttagctaacagttttctcatactcttattttcccaaaacacaattgctggatctcagctccaaatcaactcttctaggaaagtgaaaaattgctggatctcagctcccaaatcatatcttccaggcagagctaacattgccccttattcacacctccaccaaaccatctgatccaacagtgacaggtgtcacgaggccttggcatgcactctcttcccccgccagagttctgcgaaagccagggttgcgatttgttgtcagtttattccccgcctctatgagagtgtgagcactgggcaggctcggatgaaataatgcattgagtaggcctctgaaaccaaggcccctcagctggggcaacgtcaggctccagggtgggcaactttgctgcttctgccgaagatagtgatattgagaaaatgtgggtgcaatgaaacgcttattgcagcgcactcggtgcatctgtggacagagggtcaatcgcccctcagagcagcgcaaacaggcgtcccaagcctaggccttcacttgccccagcatccgcaagggtccattaatccttaacattcaaattccgcccactaaaccagtccctccgcgcccaagccgcctcttttccccaaacgacggccgaaagcagccaatcaacagctggaggggtccgcccccttttccctggccgaaatggacaggaaatctcgccaattgacggcatcgccgctgagacctcccccctcccccgtcctccccgtcccagcccggccatcacagccaatgacgggcgggctcgcagcggcgccgagggcggggcgcgggcgcgcaggtgcagcagcgcgcgggccggccaagagggcggggcgcgacgtcggccgtgcggggtcccggcgtcggcggcgcgcgcgctccctcctctcggagagagggctgtggtaaaagccgtccggaaaatggccgccgccgccgccgccgcgccgagcggaggaggaggaggaggcgaggaggagagactgtgagtgggaccgccgtggccgcgggcggggacccttgccggggggcgggggtcaggggcgggacgtggcgcgggaggggcccgcggggtcggacgacacggctggcggatggcgtccctcctctctaccctccccctcccgccgccgccggtggcgactctcccctcggcccgtcacccgtgctcgcgggtgaccgtcctcggcgcggcctccctggagccgccttcgcctgacgcccctcttcctcccgccctcgacgcgcatcccggcccccggccccgcgggcgcccctgtcgccggggttcgcctgtcggggctgcgcgcgctcctgcccttctcggggctttgggccgcggcgccgtcgcgcgcccgcggccccggcctctccctggatcgcgctctccccctccctccctcgcgcgccccctctcccgttactcggcccccccaccggcgcgcgtgcgcacttcgcctcccgtcgggagagtgcgccacaagggctcctgagctctcacccccatctctgggctttgcctccccctccttctcgcccattccatgaatttctgccccccgctacccccccgcgagcgagtaggtccaccggctcctttccccatctagcaggaacaagtaggtggggattattatccacaaaagggactagacattgtgttctgggtcccacaactcatcataaagaggtggttatagttcccatcaggagccgtgggtaggggactgtgcgtccagcagcacccgaggctcttcggcgccagaggctctaaggtgcgagcgtgtccccagggtgctcagaggttctttggagtgctgtggcctcggaatgtgagcaccctcccatcctaccctccccttcgccggcgatcttccagttacataagtggagtgggacatagtaggtaacgggctctcatcgccctggagcgctcgggatcaccggtaccagatggggcaagttcatcgcgacgctgtggctcctgttaattgtgcctgaaaggttatcctctgttcagtttggtcatgaccgaatcacccaaactgaaactcagatgacttttatatggcagtggcaatgtttctgtggttttgcactaaagacttaaggtcattatgaagagtgtaaaaggattgctgtcgcaggggagaccgtttatgacattgctaagtatggaccccgagggggaaaaagctcattctggaattgctctgtggcggttgcttctgttgcgagtgtgttcgggtgtgggttggtcacagcagaaatgggctccaccacaaaattgatattcctgactcatggtcaggcagatgtgtgcttctggttatttttccaggaacaggaatgggctctgccgagcctttcacacgttgtgtctctgctgtgcgggatcagtaccccagactgtgagataacaggatcgaattcaggggttggttggtgatccaggaaccatacctacttggatgtgatactgagtggcctagacgttgtaggactctcataagttttagacattttgttatactgtaaaaatgaagcatatacggcctcagtagtaacattgcactaataggtaattattactctagagaattttgggttccatggctggaatgttcatcagtatctcagcaatttcagttaataaccagattaagctgacctcttgagtcttttgaaatctgctgggtccctataataagacagggtctggattttttaatcttttgggaatttccaccaaggatttagggaaataggcttttatgggcttttcaggatttctatttcaaatcccaatgaggaagtctaggaaacacttgcttttattttcttgcctgaatatgtcagaaccttaaatcacccaatcaatctagaagtactacagttgattaaagtaagtcaccacaggtcacccattcgtggcaggtcagctaggtggctgatcactggaatacagtcatatccccaggcatacacacgagcctcacttgacgatcccaaagtttgtacatctgagcttgagacgtgacaagttgaggtctctgctttgttcttcatagttggcagtttgtctcctcgggtgcccctccggttagctgtttatcatacgtctagccctatcaacagtgcaggcagcttccagagtatagttttctctgcaaagtgcctggtggaggggtacctccttttccattgtcctaatggctccactgggcttccataccagccctttccagtgtcttaacagctcaaagaaacccatctgtggctttcttgttagcactggcctatcacaccttcctattgcctgttaactaaaattttgcctggaatgctttaagcttagagtactgggtctttgtttcacccgattgataggagagtgtcatgcagtcagccgaactccgacttctgtgctgtttaaatccttaggtaggtctggggcaaggcacaaaccactgtgtattcaaagggttatggtgcttgcattctgtgtgtatatgtttgtaatttccaaccagagtcgtttcccgtgcctgccctgcctctgtgcaatctagtctagtagggtagggaggcaagctgtggagtttgaaaaaaaaaaaaaaagctagatgagtttaataggctgttaccccctccagaacattcctcccatgtggagatgaaacgagaagttcatttctttaacctaaaataaagaacaacagagataaaattgttagttccttattggagcctggtttccattgctaatgtacctgcttgggactctgcatgataaatgacgtgtcctggaacctgaaatcttttataaaggttaatatcagcagaaatcttttaattagctaagagcagcttgttctcaagctgcccgagttatcttggaggagagaatcacaggaaatgtttgatatttctagtaaccagtgttttcagtgcgtggggtaaagcacactcctagactggccagtcccaaagagtgtggcaggacacctgcttgttttccctgccctctgctatggtgacatccttgcagaccagtcttgcgtggctggcagacgcaatctttttttcttgggtagagcattatcaccgctgcgatggaaggaccttgtcctcggtgcctccataccagccctttccagtgaaactactgctgtcccggcaagcccctgtgcgtgtgcatcattccgggtaggacaagatgtgaacaggtccctcttctttgggcttaagtagaagttgtgctcttctgcttttacctgtgtgctcttctgcttttacctgttctcttctgcctttctcttgcaagaaacctgtcgaaagcttgctttgcatgccattgcttaaatactttgatggtatgtatggtatgtgggataactgagtgggagccggaattggggggtgggcagggcatccccacccccacccccacatcaaagggggaatgaccatttcgttagagaataaagcctgagtcttataacttcttcaagcacatgtatgctgggtctcctggcgtgtgaatgtgttcccgctgtgctgtgtggctgttttgcagttactgtagacactgtagtctgggctctcattattgcctctgaagttgacaggaccaagccttagtaaggatgcacttgtcttcctagcccaagagctaggggtgttttatatatatattaactttcttttttagatttcataaactgctctcattttctcttttctttctttctttttttttttttttttttgagacagtctcactctgtcacccatgctggagtgcagtggcacgatctcagctcactgcaacctctgcctcccgggctcaagcagttctctgcctcagcctcccaagtagctgggattacaggcacctgccaccacgcctggctaatttttgtattttttttttttttttttttttttttttagtcgagacggggtttcaccatcttggccacgctggtcttgaactcctgacctcgtgatccacccgcttcagcctcccaaagtgcagggattacaggcgtgagccaccgcacccagcctctggctcttttctttttgacatgaaatcttaagttaattatcttaaactttaagaactagaaggattcttagtccagcaccatcagtttacaaacaaaaaaaacttgtgtccctgagaagttgtgacttttccagggcctcctagccaggataccagtttggtttttattatatagctagccgagtaatactattaacttgacttctgggatttcaaagtattctatgacttgactgtttaagggaattatgaggcctcactgaacctcaaaagataattttaggtaccatcctggtagctgtagagcagcaacagaccagtaaaagacttggttggtgttgcccttcttctgggtttgattacatgagtaattgtgtgaatagtctctaagttgtatgctctgagctttcgtttttagcttataaaagtgctactcttgggccagcatagtatcagaaattagctaattcatatggggcagtttaggctttaactaggaaatgatggttatcttaatgacaaaaaaggtactgacaaaagtccttttttgaacatgtgttcaaaagaaaaagagaaactatcaaactaataactgagtttgttgaacatcgagattgactgatctgagaggcttaaaactgattgccctgaaatagacttgcatgttttggatgatggctttgggtctcccagagcacttggtttcccttggtgctgattttttctctcaggattaaagcccctttatgtgatgttgttacagcagcttattgcaacttcattcagctgcttgaaaaagaagggagactgcctctaggttccatgtgttctttcagggatggtattttgatgttgcgtattctcttgaacatgtattcttccctgagaatttctgtgtgccgtggtagaagaaatacttgccagaaatcgccactcatggtatgcttttgtagtgtcgaagtgtcccctagaggtgacaaggcttgtgatagtgttgattctaacaagcatgaatctttcctttattttagcactgtgtgttacgtgccagtaatttgcagcttatcctttgtttctagctaggtaagctgggaaatagcctagtactttgtctatgtgtttatcttcaaaatgtcccaaatagccctgggaaaaaggtcgtgcagctcaatgggggctttcaacttacaattttctttgttttaggctccataaaaatacagactcaccagttcctgctttgatgtgacatgtgactccccagaatacaccttgcttctgtagaccagctccaacaggattccatggtagctgggatgttagggctcaggtaagtaaccttcctttttttttttttagtatatgtcctggtttggccatctgttttttttttttttaaaaaaaaaaaaaaaaggaaaagaggaaaaaaatatactactcttggacagtataaaagtaccccaaagactaaagacataactgtgccaaactgtgccatataataaaaaaaagtcacttccctgagccctgaaaggtcagtgtgtgtagggttacttggtcgccacagcgtgatctgggggcgggcgtcagattagagccggaactggtgatctgcaacttcagttcaccttgaagcaggtcagctgagctgagagcgcttgcactgagcccttttgcgctgctgctgttgccttagcggtcttcagcatgtgtttgctttggtttggggtaaatggcttagtggtcaacattagtgagcagtggtaatgcattttcagatatgggaactggtatgtggttggttccctagaaggacaccctcctgaaagctgtctcagaacaccgggggccatggctaatgtcatgtgcttgctacactcctcccatggtaactaaagagagtacccagatatacatctggttcttgggactctagaggaggatgagtactttgtaaaaacctgggggccccagtcattctaggtctgacactcaggtgctgtatcagctgcagtttgaactacttggcaccattgtgtggactttagttttgtaaaaatgaatggttccttaatttctcaaccttcaggctcaacatctcaagctgtttgttgttgttgttgttgttgttgttttgttttgtctttttgattagtggtcactccgcatttgatgttttgacatgtggatttcagaacttctgtgtggtaaagcagaatgttccaattgaattttcccattttttttcccctaagcaaaaatgtgagttttcacttatgcggccattgtgattccgactgagaccctaagtcgttctttgttgtctctctagtggtttctgacacctcagtgtgaagctgttgtagacatccataagaaatagcctgcttagggattatgtgagggcaaggtttggctgaaggagaatagaaggtgtggaaggaagcggaagaccagaagagcatcacactgcctcaagtcccaaatttgattctgctcctgatcctgtgactcaaattgtcattctcttttactgctgtggggtgtgctctgggcccagctgacaggacattctttgtactccacgtatttatgctggtgggagttgtatggctagtgctttgtgctttgtctcagaaattgtgtggactaaatgtaatatgggcagtgaatgggtttcttctggaagatgttaggcctggagggtggtttgggtttttacgagtctttttctcagattacagattacgaatggatgttataaaacaggcagtgttgacaccatacagtttctcctaagaaatgtacagagagttcaacctgaggaagctaattgaaaataaacttttaaaaagaaatgtagaaagaggaaatttaaaaagtatcttatgaatgggaaagatgtagccatttgagcttcaaagaacttttgagggaattctcaaaggacagtacccgtctttagtagataaatgcttgatatctatttttcttcttttactttttaaagaattactaaaataatacatggatttatagaaaaatcaaaccacttaaaaattgatcaggtggctgggcaaggtggctcatgcctgtaatcacagcgctttgggaggcctaggcgggtggatcacctgaagtcaggagttcgagaccagcctggctaacacggcgaaaccccatctctattaaaaatacaaaaattagctggccatggtggcttgagcgtgtagtcccagctactcggaaggctgaggcaggagaatcacttgaacctgggaggcggaggttgcagtgagccaagatcacaccactgcactcctgcctgggtgacagagtgagactcggtctcaaaaaaaaaaaaaaaaaaaagtatcaggtaaaaaaatgaaagctccccgcttaatctctgctcccactacccagaataactgctgttaatagcttaatgtagatccttctaagtcttctaagactaaactgtgtactttttgtgttgtaattaacaggagggtttttgttttgttttgttttttctttttttgagatggagtcttgctctgtcatccaggctggagtacagtggcacaatcttggctcactgcaacctccacctcctgggttcaagtgattctcctgcctcagcctcctgagtagctgggattacaggcgcctgccaccacacccagctaatttttgtatttttagtagagatgggtttcaccatgttggccaggctggtgtcgaactcctgacctcaggcgatccacctgccttggcctcccaaagtgctgggattacaggcgtgagccaccgtgcctggcctcaggatggtgttatatatacatattctgcatttttccatctcatatattcatctctgcctcatttcttttaaatagcagtgtagtccagggctgacttaccgtcatgcatttcgttgtctctctatgttattaaaaatgctacaggattgaaattcctcatgtgccagtcattttgaagaatggctgcctagacttggaatttctggtggataccaccacatcactgtccaaaatggctatcccagttcccacaacagtctgagaaagtgccattttcccatatcgtcccagaattggatatacatcttttaaattggtgccgcttggattattactgaggctaaacctcaataatcattgtcatagctcttaatgtattaaagatgttaactctttgtcatgcagatattttcctgttttgtcttacgtcttgattcttgtctttctgatatataaaatttatgttatgatttattcttttgctttcatacttaggaaatccttttttactctttttttttttttttttttttttttttgagacggagtctcgctctgtcgcccaggttggagtgcaatggcgtgatcttggctcactgcaagctccatctcccgggttcattccattctcctgcctcagcctcccgagtagctgggactacaggcgcccgccaccacgcctggctaatttttttgtatttttagtagagacagggtttcaccatgttagccaggctggtctcgatctcctgaccttgtgatccgcccgtctctgcctcccaaagtgctgggattacaggcgtgagccaccgcgccccgccaggaaatcctttcctactcttaacaacagatcaatagtcatctatattttcttcctaaaaaacctgatgatgtattatattcatacatacgtatttaatacatacaaaagggccagatgcggtggctcatgcctgtaatcccagcactttgggaggctgaggcgggtggatcacctgaggtcaagagttcaagaccagcctggccaagatggtgaaatcccgtctctactaaaaatacaaaaattagccaggcgtggtggcgggcacctgtaatctcaactactggggtgactgaggcaggagaatcgcttgaacccaggaggcagaagtttcggtgagccaagatcacgccactgcactcctgcctgggcgatagagtgaggctctgtctcaaaaataaaaaataaaaataataattttatagttaagttgccctgtgtttttcctgtgattaaaaaaaaaaatacctgttttgtatttaactttggggtgggataaggtatagcccacttagtcagttgtgtctaagccacttgatgaaccagtcaatccagtcttcacctttaacaaatgctgatttcttcaagtgagcaaaatatctcttaaactttttcctattttctctataaagttgcttttttctgaattttttctttatgaggcttgatgggcagccctatcagcagacaatcattcattcacccaacaaatatttgagtatctgcttcaagccaggggcagtgctaagccctggaagtggtatcttctccttatccgggagtggggctacagtgttcattccagaagactcaagaaaatagcagatcatcacaaaagtaggttttacataaatgaggaggaatccacagggttgtgtatatgtgtgtgtattttaaattctgacataagttggagctttagggtagactgtgacataagagtagtttgttttccaactcatgtaattgccggctgatttttgtcttgaattatgataatggtaacttctgttgaaacaattctgtattcatcttagcttggttgggatgcaataaaagttttgtactggtaaaaatgataattttcttccatgatgattttgaagcttttctgtaacacctgataaaaatagaggggccaggcatggtggctcatgcctgtagtcccagctactcgggaggatgaggtgagaggatcccatgagcccaggagttttaggctacagtgagccatcatcacaccactgtactcctgtactctagcctgggcagcctcgttctttagagtaagacactgtctctttaaaaaaaaaaaaaaagaaaaaagaaaaaaagaaaaggcttttagtgaatatatttttagggtagcaagtgtaatttttataaattgcattatttgaatctaataaataccctcaaagctgaagtcgaattctgattttgattgaaaatattttttacttaaaggaagagggatagaataaatgacacgattaaggaaaagacttcttgtataaggccggggttttttaaatgattttttgaatagttagatggacaatacggaattaaaaaggtggtgtaaaaagttatacggggctgggcacagtgactcacgcctgcaatcccagtactttgggaggccgaggtgggtggatcacctgaggtcaggagttcgagaccagcctagccaacatgacgaaacatcatttctactaaaaatacaaaaattagctgggcttggtggtatgcacctgtaatccagctactcgggaggctgaggcagaagaatcgtttgaacccaggaggcagaggttgcagtgagccaagatcgtgccattgcactccagcctgggcgacagagtgagactccatctcagaaaaataataatagaaaaaagttatatggggctgggcatggtggctcatgcctgtaatcccagcagtttgggaggctgaggcagcttgatcacttgaggtcaggaattcgagactagcctggccaacatggtgaaactccgtctccactaaaaatacaaaaattagccaagcgtggtggtgcgtgcctgtagtcccagctacttgggaagctgaggcaggagaatcgcctgaacctgggaggcagaggttgcagtgagccaagatcatgccgttgcactccagcctggacgacaagagcggaactccgtctcaaaaaaaaaaaaaaaagagttatattgtaaagtatctctgctaccctgccccaaacattcatttctttccctggaggcagccactattacctgtttctttaaaaaaaaaaaaaaattgtggccgggcacggtggctcacgcctgtaatcctagcactttgggaggtggaggtgggtggatcatgaggtcaagagatcgagaccatcttggccaacatggtaaaaccccatctctactaaaaatacaaaaaattagccgggcatggtggcgggcacctgtagtcccagctactcgggaggctgaggcaggagaatcgcttgaacctgggaggcagaggttgcagtgaactgagatcacgccgctgcactccagcctggcaacagaatgagactccatctcaaaaaaaaaagcgtgtgtgtacccgtgtgttaaatttttattataaaatatatataacaggctgggcgtggtggctcacgcctgtaatcccagcactttgggaggccgaggcaggctgatcacgaggtcaggagatcgagaccatcctggcgagcacggtgaaacctcgtctttactaaaaatacaaaaaattagccgggcatggtggcgggcgcctgtagtcccagctactctgagagcctgaggcaggagaatggcgtgaacccaggaggcggagcttgcagtgagccgagatcgcgtcactgcactccagcctgggcgacagaccgagactccgtctcaaaaaatatatatcgatatatataacaatttacagttttaatcatcttaactgtacagttcagtggcactgagtacattcacattgttgtacagccattactaccatccatctctacaacctttcgtcttgcaaaactgaaatttcatacctattaaacactaactcccacttctcccctttcccccaccccaccctctggcaactactattctactctttgtctctattggacatatttttgtgtgtttaggtttgtgtatatattttacatgtgtgtataaatacacaagggtatgtgggcgttgtgtttttacacatactataaaatattgtgcacatttgtttattttaatgtatcctgtagattatttagaatcggttcatacagagccgtatcattccttttaagggttgggtttaaatagtctgttatggatgtaccacaatttatttaaccagtttttaaattttttacttattttttttaatgtttttttagagacaggatcccactccatcacccagactggagtgcaacagcatgatcatagcttactgcagccttgaactccggggctgatgtgatcctcccacctcaggctctcttgtagctgggactacagggatgtgccgccatgcctggctaaattttttatttttatttttatttttatttttggtagacacggggtttcactgtgttgcccaggctggtcttgaactccttgggcttgagtaatcctcctgccttggcctcccaaagtgccgagattacaaccatgagccatggagcccgacaagagtagccttttatttgcagaccattagagaaaaagttgcgtgtaagtagataagcatacgaatttatcactgcaatactgtttgtagagatcacagagctgttaggctaggaagaagtttttagcagttagcactagaaagcaaaactttggaaacttccatttcatcaaaagtgacctcatctgagtagggcatgtgagggaaggacctttttactcgacaaggggaacttgccagttcttgtttgtgaaccttgaagaaagtggagcatttttattttgggaccattctaggttgtggtagttccttggtactcggggttggagaatctccctctgctgctggaccttctgcctcctcatgctgtaatatgcataagaggtgtcctagaagtattcaggaacaggctgtggttgctttgttgttctgtgactgttgaactctttatatagcagtacttttttaagctgtactaattgagcatttattatgtgtcagttactgtgctagctgctggaaactatcctaccctcagcagtcttctggtctaatgggaggtacagacaggcaaatgggctatgactttacaagaggctaacttctgtgatggagagaagttccagccactaagggagcacataaaaatcagaggagatcctaccaagaattgggaggtaggtcagggaggagacttctttgaaaaatggcatctaagacctgaacagcaaggaaacattagctaggtcaaggacaaagcatcagtggggagagatgcatgagtcttttggaaggaccagcatgtgtagaggctctgacaggagagaatctctcaggcccattcagagagtccactgtgtgaagacgaggcatgaacaaccagccaggacatgtggagaggagcatttttctttttttcttttttctttcttttttttttttttttttttttgagacgtctttctctgtcacccaggctggagtgcaatggcatgatctcggctcactgcaacccccacttccagggttcaagcaattcttctgccttagcctcccaagcagctaggattacaggcacccgccaccatgcccggttaatttttgtatttttagtagagacggagtttcaccatgttggcctcaaactcctgaccttaggtgatccacccgtctcggcctcccagagtgctgggattacaggtgagagcccccacgcccggccaggctcggctttctttccaggccccgtcacactcttagatgttccctgggccccagaaggcagcgtgaggtgctctgcccatctatgctgtccttttccaccccaccctatccactgggcccctggggctcccactccactccaggtctcttcatggacccttctttcccttcacttctgtcccaggagacctgggagactcatgggaacaggcatcatcttctccagcacagttcattgtctaatgcacgaaagtgctgggctgtgagaaggtcaggagcttacatggaatgggagtcagctgtggtacaaagtacgctgcttagttctactgattttttattttttatttttacataagactttgatggtaaaggaagcagtgtgtgagcaagctgactcctgtcttgtcaagtaccaggtccactctgatgagcccctgtggacagcactgctgtaactccccaagcctgggctcaggaggtgggccctaagcgctgttcccagctcttcctccatgctgtcagctcttccaccattcttcctggcgtcattctaaggtcctgtcttcccccgccctcttccttcctggcttctacgctcagctcaagtagtagctgtttgggtacttttcacccagacttaggaaatgttatcagttggtcgtattcctgtcagccctcttactatggaactcaaataggacagattcagctgaattcccagccctgtggttgcactctttgtctgccctgcttccccaaggtagcctgctccctaaaggtcaccgagatactccccatgcaaggtttttgttgtattacacatgacaacatggaataaagtgttttgcaaatttactggatgatatcctactatgtggaacatcctacaacttactcttttcactcaacatttgacttttgaacctggtctgtattgctccgtgcagaccaatgttcattcatttgatatgtctccttgtgccatagaaatagttttacaatagaagcctgtccagagccatgagggagaaggggattgtagagaaacaaggattggcatctggtttatgtttacatcctttagtcctgttaaccgagaagaagatatttttatttccatgtttgcgaaagctttccacaaaaaaaagcatccattttctagacttattaatagggcagtccaaaggttttatttttagatttgaaagaaagcaaagtaggcttctgagatatgcacatttccctgtgtccctgtctcttttgcaacattccacattgctgctaaaacttgaacatgagcactttcaagcatgtgcttactttctatgtatgactcattttttaaaacattaaagcattttcggccgggtgtggtggctcacgcctatattcctagcactttagaaggccgaggcgggtagatcacttgaggtcaggagttcaagaccagcctagccaatatagtgaaaccccatctccactaaaaatacaaaaattagccgggcatggtggcgggcgcctgtaatcccagctactcgggaggctgaggcaggagaattgcttgaacccaggaggtggaggttgcagtgagcagagatcgagccactgcactctagcctaggtgacagagcgagactctgtctcaaaaaaataaaaataaagcattaaagcactttgtctggtggtatgtaagttttggcattacttagataagttgtattttaagtctttctacacacaataacttatttttaaaatacaagtttctgtgtacttccgggttttgtgtttttgtaaacagcagaaggtattagtccatattaagtactaatttgttctaatattttcagctgtttttggaattacacttggcaaaaaaatagacaaatgtctgcagttgtgacagtttgtttttttgttctttttaactccagaataaataattctagtgagtttttttcttggcttgtaagaacatgggaatgattagagtcatcataacacttaagtagttatagaaggttgaacatctgtagtcagttgtataaaatttcctagtcatccttccaggttgtttaagacaaacaaatacatagtatttattaagctcttataaattttaattgtaaaacatcattgtacaaaactttgaaaatgcgtataggtacaaagataaaaaattaggccaggcatgccatggctcacgcctgtaatcccagcactttgggaggccaaggtgggaggatcttttgagcccaggagttcaagaccagcctgagcaacatattgagaccctatctctacaaaaatatcataaaacaaaatagaatcactctggcgattccactatccaaaaaaaaaaaaaactgctgttaatatttggtatgtatctttctagaccaaggcctccaactttggttgagaattcagccgggtctgtgaccttggatgggaaaaacattcactctttattttcactaatgtctctgaaaacccagatttttagctgggcatggtggctcgtgcctgtaatcccagtgctttggaagcccgtggagggcagatcacttgagcccaggagtttgagaccagcctgggcaacatagcaagaccctgtcttttaaaaaaaaaaaaaatccagccgggtgtggtggctcatgcctgtaatcccagcactttgagaggctgaggcaggcagatcacctgaggtcaggagtttgagaccagcctgaccaacatggtgaaatcctgtctctactaaaaatacaaaaatcagccgggcatggtggtgggcgcctgtaatcccagctactccggaggctgaggcaggagaatcacttgaacccggaaggtagaggttgcagtgagccaagatcacaccattgcactccagcctgggcgacagagcgagactctgtctcaaaaaaaaaaaaaaaaatccacattttcttcaatgacaattgtaagcaacagatcatggtagtattagaatagcagtgactgtcaccagtagagatcaaagatactttcatatcatattatagttgttacagatattttgaaatatatatgtccatcactgcttcaaacttaaggtaattattagacccatctctagatctggttatttaatgatttactgaagaagcacatattatttgagaacgaactcttgaaatatgttgttaatttctgtgcattttacacatttcaaaacattcttctgaggaggggtccagaaggccaaaagggtccatggcagaaaaaggttaagaacacctgttctgtactttgtaccatgtggctacaaatataaaaacagacgttaagccgggtatggtgatgcacacctgtagttccacctacttgggaggctgaggtgggaaaatcacttgagcccaggagtttgaggccagcctgggcaacagagcaagaccccatctcttaaaaaccagtcagcacttgactagtcagttagtgggggaaaacacaggtacagtaaaaggagtcaaaattacacttaaacttaggtcatcttattatatcaaacatttattaacatgtttagtgttttgtgtatattatgcaaagtgtaatgcaactaaaactgtttaacacttctcgatatcacataaatttgttggtgcataatagcctaaaagttacttttgctgttggcattttattttattacttacttatttatttatttatttatttattttgagacagagtcacactgtcgccaggctggagtgcagtggtgcgatctcagctcactgcaacctctgcctccctggttcaagcgattctcccgcctcagcctcctgactagctgggactacaggtgcgcgccaccatgcccagctaatttttgtatttttagtagagatagggtttcgccatgttggccaggatggtcttcatctcttgaccttgtgatctgcccacctcggcctcccaaagtgctaggattacaggcatgagccaccacgctgggcctgctgttggcattttaaacatccttctagagtctttcttatttgcctgtaagcacgtacacatgttgttgttgttttgagacaaggtctgtcgcccaagctggagcacagtggtgcaatcacagctcactacagcttcgacctcttgggtgcaagtgatcctcccaatcagcctcccaagtaggtgggactacaggtgtccgctaccatgcccagctaatttttgtattttttttgtagagacggagtttcaccatgttgcccagactggtctcaaactcctttgctcaaacgatccttccgcctcggcctcccaaagtgccgggattacaagtgtgcacctctgtgcctggcccacatattgttttagatgaatgagatcatatatgtacttttgtaacttgctacctttcctcaacaaaatgttgtaaatatccatgtccataaaaatagacgtatatcttcacttttccctaaaatgaaaataacttaacttgcattttcttttttgtttttgtttttgttttgagacaggctcttgctctgttgctcaggctggagtgcagtgatgtcacagctcactacagcctccacctccagtttcaagtattccacctacctcagcctccaagatagctaagactacaggcacatgtcacgacgcctggctaatttttaaaatattttttgcagaaatgggatttcactatgttgcccaggctggcctcaaactgctggcctcaagtgatcttcctgccttggcctcccaaagtgctgggattacagtcgtgagccactgtgcccagcctgcattttcttattataaaagtaatacatgttcaatggacaaaaaattttcagaaaatatgcaaagatgaaaagtaaaaattgtccataaatctgtcatctacagataaagataacttctggataatgtttttctaccatcatttttagtaatcacataacatttcgttgtatgtctatgccttcatttaattaagaggcatttccattatttctgcatgttcatgactctgaatgatgatatgtctgcctgctgatggctacaaccctgtttctgcatttcaaacctctctcttgagctccagatttgatggcctcgtcagcatttacttgagttgctcataagtgtctcaaatttaacaagtcccagtcttgatttttttcccccttcaaacctattcctcatgttgtctctatttcagtaaacaatatcaacatccacccagttactcagtccaaaattctaggagtcatccttgattctgctctttctttttttttttttttttgagacggagtctcgctctgtcgcccaggccggactgcggactgcagtggcgcgatctcggctcactgcaagctccgcttcccgggttcacgccattctcctgcctcagcctcccgagtagctgggactacaggcgcccgccaccgcgcccggctaattttttgtatttttagtagagacggggtttcaccttgttagccaggatggtctcgatctcctgacctcatgatccacccgcctcggcctcccaaagtgctgggattacaggcgtgagccaccgcgcccggcctcttttttttttttgatacggagtctcattctcgtcatccaggctggagtgcagtggtgcaatcttggctcactgcaacctccgcctcccgggttcaagcaattcttctgccccaacctcctgagtagctgggcctggctaattttttgtatttttagtagagatggggtttcaccatgttggccaggctggtcctgaactcctgaccttgtgatccgcccacctcagcctcccaaagtgctgggattacaggcgtgagccactgcacccgccctgattctgctctttctctcatcatccctgatctgtcagcaagtcctgttaggccaaccacttctcagcatctgcactgccagtatgctagtccaagccacaaaacacctctcagaggtacgcaacaggactttgttgttgttgtgtgtgtgtgtgttttttttttttttacggtagaattattttataacttgaagtgtagggatgaccttccttttaaaattttatttattttcttaattttttattcccatagattttaggggaacaggtggtatttggtgacatgagtaagttctttaatggtgatttctgagattttggtgcacccatcacccgagcagtgtacactgaacccaatttgtagtcttttatccctcaccccctcccaccctttccctcaagtccccaaagtccattgtatcattctgatgcctttgcatcctcatagcttagctcccacttatgagtgagaacatacggtgtttggttttccattcctgagttacttcacttagaataatagtctctagttccatccaggttgctgtgaatgccattaagtcattcctttttatggctgagtagtattcctttgtatatatatgccacaatttctctattcactttctgattgatgggcatttgggctggttctatatttttgtaattgtgaattgtgctgctataagcatgcgtgtgcaagtatcattatgcaacgggacttcggatgacatattgaacaacttcctcactgatatccttgtgaaacgctacatatttcatgggtctgtttgttataccaacactcaattctgaatgatggaattatttcacatttagtctctttttcatctagccattatagtttagtttaaacaccaagttccattcctgctgcatgctgaaggatttgcagtacatcaggctatagtccatcaattcattccacaattgagtaagtaaggtatctatagtacctcaggccctgtgttaggcagggatggggtaacctagacagatttggtacctgtcctcatggagcttgactttagcggcagagacagatcttagataattgtaccagtacttaagtgttcagaaggacaagtatggaggtgggagccactccagtcctctacagtcacgtgctgcacaacaacgtttcaaccgacaacagagcacacatacagtggtgttcccataagattagaatgccatatttttactgtaccttttctatgtttagatatacaaatacttaccattgtgttccagtgatctacagtattcagtatagtaccatgctgtacagttttatagcccaggagcaataggctatataccgtatagcctaggtgtgtagtatgctacaccatctagatttgtgtaagaacactctctgatgttagcacaaataaatcgcccaatgacacatttctcagaacatatccctgttgttgagcgactcatgactgtacaatgggggaacagctcagcctgccttccccaaggaggcaacgccgatgcagggaaccgagggttgaacagaagtgggcaaggtgcagagaggaaaaaggctttccagatggagaagagcatgtgtgaaggccctcagtctggcaagagcttagtcctgtgtggcaggagctgaacgaggggttaggggagagtggcaaagggtccgggccatcagagaggtcaacaggggctggatcatgcaggattttgaatgttaacatcagagcgatagaaaacatgaagggtaaggtggtttgggttttgaatttttatcaaaatattgcaactactcggcaacaagttagatataacaaaagaggtcatcgtaaaaacagcagtctaccccttccccttcccttccctgactcagacatgacagcttttaactgtttctgattttagttcctttgatggttacctccaaaattaaatcatatgcttatacatctttcttgatttgccaactttagggaactgtgttaactccctgaaatgaaagagaatgacttagcttacgttacctccccatgttgaaattcatattcacatttctctgtaattttttttttgagatggagtctcactctgtcgcccaggctggagtgcagtgatgtgatctcggctcactgaaacctctgcctcccgggttcaagcaattctcgtgcctcagcctcctgagtagctgggactacaggcgcacaccagtgcactgggctaatttttgtatttttagtagaggcggggtttcaccatgttggccaggctggtcttaaactcctgacctcaggtgatctgcctgccttggcctcccaaagtgctgggattacaggtgtgagctaccatgccaagcccacatttctctgtaaattttaaacagtattttagtccatatgtcagttcccctctttttaagataaggatattaataaccctacttttccttccactttccctccttaacttctgcgtcttaaaagctgtacttgaccatacccattcttgcatttgttaacatgtgtgttctgccctgcaaccacagccaagttgtagcttaattctaaagtcgaaaataacattatttacattattacacatgtgagtattgttcttggcgagaccaaataatgaatgtgcccaggattacatttccttctttacagatctggaccactcaaaggagaatgaccaatcaaggtcaaatggattcccttttcttagactctgtcacctgcttaaaatccttgctaggatttaatctacttcatattcaaaccttggcttgcttatatagctctttgttttgcccaccatttctagttgcctttttattttgtttactattcctccttgattatgttgtaaaatgctattagcagtgctgtttatttcgtacgattcggctttttccccactgaagacctttctcttggaaggcctctgcccttctgcaatctgggcaagtatctgctctttgatttaaggttgcatatacagctgtacctggaccttctcctcactgggctcttgggttggagcccgtgtcttaaaacccatatcttcctttcacatggtttactccctcgttttctggactgcatccttaaataatgtgctcagatagagtgcatggggagtaaactgtagtggataccgttgtcacctaccccatatccatttcttctttgcccattcctaacagaatcctgatattattcacctttctaccttattcccaacttcagaaaggatccttaagtcattccacacattgcattcctttggcaactgttactggtcaaggatggacatacgatctaagttggttcactcagactgaagggaaggacttccatggcacgttcagggaagactttctctttttctctcactggagtaagtgagaaaggtgttgctgggggctcttctgtaacctcagactgagggacaaagcctgaggataaagcctgctcaccaaggaaggcggagcagagaaacgggaagaacctgggtcttgatgccgttgcttagctggtgattgcactgtggctggggcctgtcctacctgagctagggactcagataggatggactttcttactgaatgagccagcttcagttttcttttctgttacttgtaaccaaaagcattgtaacagataggtcttctgagtcactgcatgcctgaaatacctttcttctactcttacacttgattggctgagtatagggttccaagttgaaaatcattttttctcctaactttggaggcatctcttctagtatgcagagtaacaaatgcaacaaatgcctgatgccaatcttattatggttcttttgtaggtgcccttcttttctcttccttccctggaagtttttaggatttcgtctccttatttttagtgtcctgaaatctcatgagaatatgtctagagttgtgtcttgttcactcgttgtactgtggtcattcaatctgaatacttttgtcaactcttttttttaaaaaaaaattactaatttacatgctttcattttctctatggggactactgtttaactttggacctcttatattggtcttctgtcttctctcatatttttcatttattgggcggtggggggtgctctgaattttggaagatctcctgcatttattttctttttccttttttctttttttttgaggcagagtcttgctctgtcacccaggctggagtgcagtagcctgatctcggctcactgcaacctctgccttccaggttcaagcaattctgcctctgcctcccacgtagctgggattacagacctgcaccaccacgcccggctaatttttgtatttttaatagagatggggtttcaccatgttgccaggctggtctcgaactcctgacctcaagtgatccgcccgcctcagcctcccaaagtggtgtgagccaccgcacccagcctcctgactttattttctatcccttctgttaaattctttatttcagccaaacctttttttttttttttttaacttactgtgaagcctgtgtgcctactgcctagattagacaattgttaacattttggcgtattttcctggtctgtatagatagggtgcatttttatacctttttgctcaccatttaaaaggaactcacagatgtcacgacatttcacccctaagtacttcagtatacatctcctaaaaataaggacattttccaatataagcacaataccccgatcacatctttaaaagttaacaataattctctaatgttatcaggcacccactttaaattcagatttcctccaaatgtctcttatcgctgttgaggggctccaaagcaattaagatttatacattgcatttgaatatgcttcctttgtctcttatttatttatttattttattttgagacagagtctcgctgtctcccaggctggagtgcagtggtgtgatctcagctcactgcaacctccgccttccaggttcaagcgattctcctgcctcagcctcccaaatagctgggattacaggtacgcaccaccacgcccagctaacttttttgtatttttagtagagacagggtttctactaaaccatgttggccaggctggttttgaactcctgacctcaagtgatctgcccgcctcggcctcccaaagttctaggattataggcgtgagccaccatgcccagcccctttgtctcttttaacaagaacagcctacccacctctccttcacttgtgatactgactttttgaagcagtcagcccaattgtcatctagaagggcccacattttaagatacatatttttagagatggcgtgtcactatgttgcctaggctggccttgaactcctggcctcaagtaatcctcctgcctcagcctcccaggtagctgggactacaggcttgtgccaccacgactagctggcccacattttagatgtctctcttttcttgtgatataatttaacttgtttgtccatatatattacttgtagacaagaagttaggtctacaggcatgttcaggttaaacattttgggcaagaatatttcataggcagtactgtgtacttcatatagcatcagctcaccaggcacttaatggttggttaccctgtggtttgtgatgctacgttttaacacttgattaaagaaggaggtgactgccaaatctccccattgtagacatacagttttcctgtggtaattgatatataatctgtgatgtgatgctttggcaccatacaaatatcctattgcccaacagccatttgcataattattttagcattcattgatccttgcctgaaaccattattacattgggggttgcaaaatgatagtttttttaattgtcacctcttctacatttagtactgtcattcctctgtaatgaattttccctcatcaaataaggaggcattatatttcctcctaaaaagacagtatagatgcttaatctttgcctttattacatttcagagtaaggagttggtagaataatcacctcaaatggtggcaaaatatttgctgttgtgtttccttttctctttcatgtcaatatggactcatagggatttatttattaaatgttttacaattcaaattgggtcaacaagagtcccttcatgttggttcctatattctcctggcataccctcattaattagtctttgagcaatttcttgttttctggcacaagatattccatcccaatatgtgcttttcgtgtcctagaactgattccaaggagctctggttccttatagtggcaacggtatttagaatccaacatgtgggtactaaatgtgctgattcttactggggtaacattatatctaggcactttcagaggacatcgctagaaaatatattcagtttttattttttatttttaatttttttgtagagacaaggtctcactatgttgcccaagctggtttcgaactgagctcaagcgatcctcccacctcagcctcccaaagtgctgggattacatctgtcagggcaaactgctggtaaatattttcacaaaagaaaaataaaaaaaccaaagtgctgggattacagatgtgagccactgtgccctccattttttttttaagtcataaatacataatgatgtcttctatggcaatttaacattacagggttcttccttaacttttttttttttttttcttttttttgaggcagggtcttgctctattgcccaggctggagtggtgcagtggtacaatcctaattcactgcagcttcaaatgcctaggcttcaaacaatcctcctccctcagcctcccgagtagctaagactacaggtgcatatcaccatgcccagctaatttttaaaaattttttgtagagatggggggtctcgttgcgttgcccaggctggtctcgaactcctggcctcaagtgatcctcccatctcgatttcccaaagtgctgggattacaggtgtgagccaccatgcctggccaacttttatttgatctgtatatatcttttccttacactgaaaatgttgggtcttactaatgttaacatagttacttatttgctttatcttgtaatagacataaaattacaaaattataataccaatattacgataaatagctgggcatggcggcgctcgcctgtagtcccagctactcaggaagctgaggtggagggactgcttgaacctaagagtttgaagctgcagtgagctgtggttgtgccactgtactcctacccggacaacagagtgagtggggggaaaaaaaagaaataatggaagatccagagaaagccccaaatatttaaaaattaaacaatacactttgcagcttcctcacctctctcagccttcatagaattgaagagagttagggccttgcactagattaggctttgccttaaggaaatgttgtgtttgatttgatcttttatccagaccattaaaacttccttaacgtttattagtaataaggctatttcactttcatatcatttgtgtgttcactggagtagcacttttagtttccttcaagaactttgattcacaacttggctaactttggtacaagaggcctagcttttggcttctcggctttcaacatgcctctcactaagcctgatcatttggtttttggtttaaagagaaagacatatgactcttccttttacttgaatgcttagaggccattgtagggttattaattggcctaatttcagtactgtcttgtccaagggaatagggaggcccaaggagagagagagagatgggggaacagctgattggtggagtagtcagaatatacacatttatcacttaagttttgccatcttttctttttctttttcttttttttttttgagacagtcttgctctgtcacccaggctggcatgcagtggcatgatctcagttcactgcaacctctgcctcctgggctcaagcaattctcctgcctcaacctcccgagtagctggtattacaagcgtgcgccaccatacccagctaatttttatattttttagtagagacagggtttgccatgttggccaggctggtctcgaattcctgacctcaagtgatccacccgccttggcctcccaaagtgcggggattacaggcgtaagccactgccgcctggccaagtttgccaccttttttattttttttattttttgagatggagtctcactccatcacccaggctggagtgcagtgatgcaatctcggctcactgcagtctctgcctcctggattcaagcgattctcctgcctcagcctcccgagtagctgggactacaggcatgtgccagcacgcccagcttgttttttgtatttttagtagagacggggtttcaccatgttggccaggctggtctccaactcctgacctcaggtgatctgcccggctcagcctcccaaagtgctgggattacaggcgtgagccaccccgcccggccaagtttgccatcttttatgggtgcagtttgtggtgccgcaaaatgatgatagtagtaacatcagagagcattgatcacagatcaccataacaaatataataatgagaaatttgaaacattgtgaaaattaccaaaatgtgacagagacatgaagtaagcacatgctattggaaaaatggcactggtaaacttgcttgatgcagggttgccacacaccttcaatttgttaaaaactcagtatctgcgaagtacagtaaagcaaagcacaatcaaatgaggtgcgcctgcatattacaaccaagaggcttttcgcagaaacacgagtgtgcttcaacattcaaaaatcagttggtgtaattcaccacattaagataataaaggaggccgggtgcggtggctcacacctgtaatcccagcactttcagaagccgaggtggacagatcacttgaggccaggagtttgagagcagcctggccaacatagtgaaatcctgtctctactaaaaatagaaaaattagccgggcagggtggcacgcacctttagtcccagctattcgggaggctgaggcaggtgaatccttgaaaccaagaggcggaggttgcagtaagctgagatcacgccaatgcattctagcctgggcaacagtgagacccagtatccaaaaaaaaaaaaaaaaaaaaaaaagataataaaggagaaaaaccatatagtctattcaataaatacagaaaacgcatctgacaaaattcagtgccctttcatgataaaaattcagcagattagaaatagaggaagtgcatgtatgaaagagctacagatagattgtacttaattgtgaaatattgaacatgttaccccgaaaatcaggaaaaaggcaaagatgtccactctaaccacttctattcaacattatactagaggtcatagccagtataatgaggcaagaaaaaacataagaggcattaatatttaaaggaagtaaaactgtttctatttgtaggtaatgtcgaaaatgtgaaagaaactatcagaacctgctagaactaaaaagtgaattcagcaaggtctcagggtacgagatcattcaaaaactaatcagaaatgaaatttcaaatgcaatatcgttaggaataatttaataagaaatgtacaagatttacacgctgaaactgaaaatgttgctgaaagaaattaaagacctaaataaatggaatgttgtaccatgttcatcagctgaaagactaagtgtctttacagtgtcagttctcccttcattgaactgtagattcataaccccagtcataactgcagggttttttttgtagaaattgattgtaaaatgtatatggaagctgggcacggtggcttatgcctgtaaaccagcactttgggaggctgaggcagacagatgacttgaggtcaggaatttgagaccagcctggctaacatgacgaaaccccgtctctacaaaaaatacaaaaattagccagacatggtggcacacacctgtaatcccagctacttgggaggctgaggcatgagaatcacttgaacccggaggtggaggttgcagtgagccaagatcatgccactgcacgcccgcctgggcaacagagtgagactttgcctcaaataaataaataaataaaacgtatatggaaatgcaaaggacctagaacatccaaagtaaacttgaaagagaacaaaattaaggggcttatgtgatgtgactttatagtcatttgatttttaacagaggcaccagaacagtccggtgcgggaaaggaaagtcttttagcacgtgccggatgatggcaaatgacatccataccagacaaaaatgaaccttgaccttaccctaaatattagtttcttagagttgccacaaactaggtggcttaaaaccacagaaatttggctgggtgcggtggctgacgcctgtaatcccggcctttgggaggccgagtcgggcagatcacctgaggtcaggagtgcaagactagcctggccaacgtggtgaaactttgtctctactaaaaatacaaaaaacttagtcaggtgtagtggtgcacacatataatcccagctagccaggaggctgaggcaggagaattgcatgaacctgggaggttgcactgagccaagattgcaccactgcactccagcctgggtgacagagtgagactccgtctcaaaacaaaaccacagaaatttgctgtctcataattccagagtctagaagtccaaaatcaactgtcatcagggccatggtctctgtgaaacctgtaggggagtccttccttgcctcttcctagcttccagtggttggcaacctttggcattctttgatttgcagctgcattacttcaatatctgtcttcctcatcacacagcattctccatgtatgtctctgtctttacatggcctccttcttgtaagaacaacagtaatattggattggggcctgccctactccagtgtgacctcaccttaactaattacacctgcaacaacgctgtttccaactaaggtctcagtgtgaggtactgagggttaggacttcaacatatcttttttgggggacatagtccaacccatgacactacttcacaccatacataaaaattaattcatgagagattatagacctaaatgttttaaaagctaaaaatataaagcttctgcaagaaaacataggagaatatctgtgcagtccaaagaataaggttttttttaaacacaatttcactctgttgcccaggctggagtgcagtggtgcgatctcagctcactgcaacctctgcctcccgagttcaagcgattcccatgcctcagccttccaagtagctgggattacaggtgtgcgccaccatgcctggctaatttttgtatttttagtagaggcagggttttgccatgttggccaggctggtctcaaactcctgacctcaagtgatctgcctgcctcggcctcccaaagtgctgggattacaggtatgagccaccgtgccaggccaaaaaaagatttattaggatacatgaagcaatagctattaaaagaaaaaataaattggacttcattaaaatttaaaacttttggtcctcaaaagataccattaagaaaatgaaaagacagagctgggcgtggtgaggcttgcctgtagtcactactgggtacttgagaggctgaggcaggaggatcacttgagcccaggagttctaggccaacataggcaatatagtgagacagtgtctcttaaaaaaaaaaagaaaaagaaaaaacaaaagaaaataggctgagagctgtggctcatgcttgtaatcccagcactttgggagtacaaggcagatggattgcttgagctcaggagttcaagaccagcctgggcaacagtgaaatcccatctctacaaaagatacaaaaaattagccaaccatggtggcgtgcacccgtagtccatagtcccagctactcgggaagcagaggcaggaggattgcttgatcccaaggaggtcaaggctgcagtgagctgaggtcgcgccaccgcaccccagcctgggtgactgagtgagaccctgtctcaaagaaaatgaaaagacaagtccaccaagaaaacagttgagaccagtcatagaaaaaatagaaagtcatatctgacaaaggatttgtattcagaagaattcctataactcggtaataaaaagacaacctgatttttaaagggtgaaaatattttaatggatacttttttttttttttttgagacagagtctcactgtgtcacctaggctggagtgcagtggtgccatcatacatagctcactgcagactcgaactcctgggctcaagtggtcttcctgcctcagcctcctgcgtagctaggactacaagcacgtgccactatgcctggctaatttttaaattttttgtagacatgggctggtgtgtgtctgtgttgtctaggctggtcttgaactcctgggctcaagtgatcctcccacctctgccccccaaagtgctgggccaccacgcctggtgtggtgtcaccagagatgagccaccatgccctgcctgatacttttcttttatcatctgtttatttcttgtttatgggtgtttctgttctgattttatgactattctgtattctaagatttctctgagaatattatttgtttcctctaaggacagacttggtctctcttccatggggctggttcttgagccgccagggatggggggaggacctgggtagctgctgcgctgccccttctctgctgtggtggagtggcccgttgcctgcggggtctgtctctgaagggtggtttggagaagttctgtggaggcgaggcaggggtcagctcttgccccatggaccctcagagactgggaaaaggtaggcatcacctgggctgctgccagccacatcaagagcctttggggtgggtggggtgggggccggggaagggaaggttctagctgttgatttagaaaagacccattgatttcttgcctgagggaagaagtggcaccttgccactccaagtttgctctgctcaccagccccaacagcatcagcctcacctgggagcttattgaaggtgccctgccccaaagcttgtgggtcgggatctggcagtcagcagcccccaggtgttgcacacatgacagcactgcttgcccctcctctcgtctggtctggtctggtctggtctggtctggtctgagagcttctttggggctctgacaggtatacaggctctcatctgctctacagctctcttgtagtacattcctctctgttccattagtcaacatgttcttgcctacttccagcatctagaaatttgctgagtgtatcttatccgttcagaggtcctctcctgctattgcctctgttagaaatttcttccctttcatatgtctgtacttcggtttctttgaggtttgaagggtcaagtggcaattacatgtaggcagttggccatcctctggtgaaggctttaaagcccaggaataacaagatccattatgtctaaaactagcaaaaaccctacctgtcacatggtgaactgaattagaggtgatggggacagaagccagattggagtgagtggaaggtgagggagtggagattgggtatagacaactctctttaagaagttttgttgtgatggtgaccagaaaaataggagtggggtagagggggatgagggtgtttttcacgtgggtgaatatgagagcatgtttacatccttctgggaatggcccaggagagaggggagcgtgagaaggaggaaatggggacctggaggagcagactccttgtgtggaggggggaagcggcatctagagctgttgcaggaggtgggctttgagtaaatgcttcctctttggtaaagacggggaacgggaacggtagaagttgcacggaccctttgttaaagctcagaagctgagcgagaattagagcttaagtttatggagtagtcagaataaagtacccctaactgtattcttgcacagtgcaggaagccaggggtctgaaatcaagatgttgcagggctccattccccccagagcctctagcatggatccttccttgcctcttccagcttttggtggctgcctggcagtcctagactttccttggcttgcagttgcattgctccattctctgcttgtgtcttcacatggccttctctgtgtccccgtattcttctctggatgcttgtcactgtattaggcccaccctaaatccaggatgatctcatatcaagatccttcacttaattacagaagcaaagaccttccaaataaggtcagagccacagggtctgggggttaagacatacacctaactattcggggctaccatcgaacccctgtagaagcctgtccatttccaggatagcagagcccaaggaagggccagaagtccccccaaaacagttgtttgcattcaccagattctaagctataagcagatgggcagtggcagtcggtcctaatccatataccattggcaacaatagtttagttcactgtagacataatgagatgcttatcttctgctaagtagtcctcatggtaacagccatattactcctggctttgagtgacagcgctgtgctcttgtctggtgcccatatacttcagcagctggaaacaagacagtgctcatgattcaccggaaagtttctgtagttaaaatcaagtgatcccttgagtctattctaatatttgtctgtaccatgttctgtgacgagacatggaaacaaaacattaagaagtgaaagtatctttgattatgctcttgaagacaactttttgtttgtttgttttgtttgagacggagtttcactcttgttgcccaggctggagtgcaatggtgtgatctcggctcactgcaacctccgcattcccgggttcaagcgattctcctgcctcagcctcccaagtagccaggattacaggcatgcaccaccacgcctggctaattttttttattttagtagagacggggtttcaccatgctagtcagactggtctcaaactcctgacctcaggtgatccgcctgcctctgcctcccaaagtgccgggattacaggtgtgagccaccgtgcctggccgaagacaactttttaaagatgcatatgctctgggctgggtgtggtggctcacacctgtaatcccagcactttgggaggccaaggcgggcagatcacctgaggtcaggggttcaagaccagcctggccaacatggtgaaacaccgtctctactaaaagtataaaaattagccgggtgtggtggcgggtgcctgtaatcacagctactcaggaggctgaggcaggagaatcgcttgaaccccagaggcggaggttgcagtgaattgagactgtgccactgcactccagcctgggcgacagagtgagacttggtctcaataaataaaataaaataaagatgcatatatgctctgaaggctggctacattttattttttctcaaacaacttttacagtggatagtgctgtaaaagttgtttagctctggtcattttatcttttttgtgatctgcaaaaggagatccttttccttttcactgcctctaatacccaactccaatactcctttgacctcacaggcacttagaatccatcagtggctccatcttttctcagacttcacctccttcgtatccttattcctctcctttgtagcttcagtgccagtcagacgtcgttactccctggcacactacctcagcccttcggcactttcttgctttgttatattcagtcgacaaagccccagcctcatgaaattcagtcatgtctgccccatgcctgctccgtcctgaagctgaatgtgactccttcattgattccccctttaagttcatgtctaccccaggtaaacttcttgccgccaagcccacctgcatactgcagttccccggtctctcctagcccctcccaccctcctcatcagctgtcacacctccttgcagctgatatcttagttcccagagagcacagaagcaagctcaagagagtgtctacaggccagcgtctactccttcgctctgcctccctcatgctgtcacggagcgcctgtcccaggccctcactcctgttctaaggcatctctccagtcctctactgcactctcccccatcagagattcctccctcgcctgccctagaaacttgttgctacttctcccaccttgacaaaagctgctcccaaccccccacctgtctagcatctctgttttcctttaccagcaagactccagtccctgtctccacaccctcttcaaccttctccagttgttcctttgctgccacagctctgccaagctgctccatgaggtctccagtgatgaccttcatgttgttaaatctgggagctgcttgtcacttcttttcccagagctgtcgtcgtcagttaaccccaatacccatacgagacaatcctttccctcccttgagtctctgcactgatgtgcaccttctcagtgcaaaccacagcttttgtctctttctagctgaatacatgactgactgtgcttatcttagctgtcacccctgctggagcagaagctccacaggacaggattgtctccttgattcacagatgtagctcaagctcctagaacagtgcctggtgtctgtgggtcctcaggaagtcctaggtgagtgtgtgaatgtctgtcctcctcggtcttttctgtttcgcatgtgatgttggaaggcctcaggttcaggcttttcttctccaccctcactcccgaagtgattccgtccagcctcatggctcccaaatttatgtctccaggctgactgggtcacgggaactccttatcctccctagaccttctctttccctggtcttctcctttccagtaaacagcaactccaactcctgctcctcaggccagaaacctggcgctggttacctcatttgttcacaaatactgttactccacctgcaagatggatacaaaatctcatgagttcccctgcctccatggcccccaccctggtccaggtgtccagcttccctgcctctgcccttgccccctggttctttgtttagtcctgcaatattgtgctcttctgttcagaaccctccagtgggtccctgcttgctcagagtcacagcccaacaggatcacctctcttgccactctcccaccactcatgcactgcggcctcactggcctcctggctcctccctggaccctccaagtatgcccctgcctcaaggccttgacctctcctctgccctctgtcccaggtatcacacatgtctgctcaagtgtcaccttcccaatgtggccttccccgagcacacccatgtctgtaagtcaggcagcctgcctctgccccaccagttccccctgcctagcataggacttgtcaccatctgccacaagctgtgttactgcttggttttgtttgtgctctcccactcccttcccctccctccctctccccacccctatagaatctgagtcccatgaggacagagatttttttttttgcctcttcctcaatatatccccagtgcctagaatcatgcatattttaatttattgagtgaattaacaaataaattctgcagtgggacattgctctaaggttttgtcttataagagggacagaagaacatgcagcctgctctgctgttggctccttagcttctccatggctaacaggaacaatattgtttatgagataatcaaagccatagtcatgagttagaaggtacatataaaggcaaatatgtggagattgagttactttggcgggggcggaggggtgtgtagttatctgaaaacatggaacaaatcaatgagagtctaaatagcacccacatatttgcccctcttttaaaacatacctcctttatgagtgtagaatttaacagcacagtttcccagtgcagctttgcgaaacaagagaaatgaattttaaaggctgtcaggtgcctttgcaatctgtggcttagtatttgtgtaacaacatttgtgttttgccaacagatgtactgcttaaagtacccatgagaatggcctctgtggttttctactttctcggatggaaccttaggaactgattattgagaagaggctgatactgcccacctcagctcccaggcaggcttcagggccttggttactactcctttgggccctcactgacccttgcagacgtcttttgcaacctctgtagccagaagagacaactgctttctggcatgttgctctttccctaatggctttatgagcttcttgaacaccaaagcatgtgtgacttcctcacgccttcccctgtatagtatagcacccctaactgtatgtgtaaggagaaggagtgagcaattgatggcgtccccctggatctccaagggaatgggaaggggcagggtttctggattcagtcatgtggaatctggtcgttggtgtctaaatgtgcctgttacttgtgcccaatgtccaggttcaccaggaggcttagcctgctcatttccctgctagtagctatattttgggagagcattttttatgctctaattgaactttgtgggtctcagcaatttgaacaatgttttgtttttcttctggtcaggttacaaaggaaagaaaaggaatctggtgttgtaggaggtggtgtgttgtgtaattacttgttttttttttttaagtttttttatatttttgcaacaaggcaagttttctttgaataaataatgctgggagcttatcacatagaaggcatatgtgttgtactcagtacattctgaattatctgggtttatttttatctcttgaattcacttataaacaattcaaaggctgcttttttagctaacagaaataggaagagaatctcagaatctgttttgtcattttttcaatgggttgagttatagtgattatgctaactcggtggaaaagcagtgtagtgagtaaaccccacatacggcatatcatgaacattttgttgcttttttggcttctgaccatcttcatacctttttttgtgttaaaagtctccccattgtgtatatcttagtaagaggcaggtggaccctagtttctacctaagaaaacctgaagatggggttgggggcctgtggtgtacttccttctcatgctgtccatgaccactgcagtggctttgtaaggatccaggacccagggcccctcagtgggtgtcttccatttctcaagattgcctcttggttgtgagatgacggttgctgctctagtcattgcatccactcctccaggtaggaagaaagaggtcataggggaaaggcaaaatggcacttgacatcttaacttctaggcccattcaatatttccatttccatcttcacacccatcatcttgtcctagggaacaaacagcaccagcagctcagtagctataacagcaattattatttctcacacatgggtctgggtcagctgcaagatgggattcagatctgttctcaagcctgtcgttccaggacccaggagggagaagcagctgccaggggaagtctcttcgtaggcggaggtcaggagtccaagaggagtgagcagagtcacagaagcctcttaaagcctcttcttcccccatcccatcaacacgtgagcaagcccagagtcagtgggtgggaggtgtgctccgcccatagtcagtagaacaagagcaagcttgaaagagaacgatgaacaggagtgcactcggccactgtctccttagccataacgtagtcccatggtctgctgggaagcggggaataactctccttagctgagcgcagggtctcattagtaaggcagatggagaacatggatgttgggggacaactggcagtgcctgccacaggggaatggatatattccctccccttgctctctggaaactaggggatggcgatgaagcctaggtttggccaaacccattctgccttcagggactttgactcttgagggacagtgagggattagtcatggtggtggcagttgagtcacaaaaccagcagtggtggctaatgtccagtgatgacatgcaacagtgcccagggtgggtgtcccaaccagcaggtccctgcagtataaccttggctgtgtttgcagtggtgcagcctctattcctgctgcttttctgagcctagttctctagttctctctccatttccagatctgctgcccaagaactcctttgtgattgaatttaaccagagttgatttctgttgcttgaaactcaggaccttgatgcagtgtgcagggtctgggcaaggtaaaaacacactttcatgatttcgcctcaagtatagtagatgagcttaggctttttgagccaggtagccttgggattaattctcagctcttgcacctagcaggtgacctcagcaatcaagtcacttgccctccttgagcctcagtgttgccctcagttaaacattgctctcgtgtgttgctgagcattcagtgagattgtatataaagtgatcattgtgaaggtattgggacataatttgggaaagcgtcctggtctaagcttttgattggctctccatcatttgggtaatgggatagtgatttaggagtatcatagcagctcaaggggcccttctggaaagggtatgaggaaggtcagggacaagtctgaaatgaatcatcctaggcttaatgcctgtgactgggcagtgggtacaattatctcaggctagtcagtagtcatttattgtgagaatacatgggcagattttttaatttccttctcagtctagacttgaatgcaaacagttcttattgctaacttaccactagtcaccactaatgaacaaagactatgaacaggaaattcataaaagaagagatacagatggccaatgaagataggaaaagagttctgcctgctggtaatcaaagagatgcaaacgagaacaaaaatgatgccttttcacctaccaaatttgtcaagattaaaaagaaagcaaagagccagcgtcagctgatgttcatacctgcacctgctcggtagcttgctaatgttctgcctgctccacacgccaggccagcctccacggcgcagccaggtgaggcctttccttgtatacaggcacgtttggattcactgttgcataagtgagacatgtgcatgcttcacaattcaggaagtgcaaactccagagtgatgcaaggtctgagtgtccctgctgccctggtctcccagctgcctaggcctctcttcagggaacggctgcatccagggtcttgtctgtgtcttttgaggtgttctatgtacacgcagacacatctaaacctatttttattttccctgcacctgcttgggctactgtaagctgttaaatttttttttctaggaatttgtccattttgtctgactagcataaagttgttcatgttatagtttatacatttgcaacatctgtaattatgtctgctttttcattcctagtgtttgttgtttacttgtaccttctcccgtttttccttgtcttggtaaaagctcattaggttcattggtcttacaagaagcagacatgtaggcattattgccctgtattactctgtgtctcttttctagttcattaaattctgcttttgcttttatctttattattttttctatggtcttagagtacattctctgtgttttgttgttgttgttgttgttgtttgttttttgttttgagagagagagtcttgctctgtcgcccaggctggagtgcagtagtgcaatctcagctcactgcaacctccacctcctgggttcaagtgattctcgtgcctcaacctcccgagtagctgagatttacaggcatttgccccacacccggcaaattttttgtatttttagtagagatggggttttggcatgttgaccaggcgggtctcgaactgctgacgccaggtgctccacccgccttggcctcccaaagtgctgggattacaggcatgagccaccacgcctggccctaacttcttaagttataaatttggttcattgatttttctacccttttttcaaatgtaaacatttaaggctaaccctctggaactacttcaactgtatctagcaagttctgatagtattgtcactttaagttagcccaaagatattttaaattttctttgtttcttcctttgactcacatattattttaaaatgtgtttttcaatttccaagcacatggctttgggtttgttttttttttttcctagttgctctagaaaaaaaaattgactgcatgaggtggctcatgcctgtaatcccagcactttgggaggccgaggcgggcagatcacctgaggtcaggagttcgagaccagcctggccaacatgccaaaacctcgtctctactaaaaatacaaaattagccgggcatggtggcacacacctgtagttccagctactcgggaggctgaggcaggagaatcacttgaaccgggaggcagtggtcacagtgagccaagattgtgccagtgtactccagcctgggcaacaagagcaatactccgtctcaaaaaaaaaaaagaaaaaaagaaaaaatcaacttctaactaaatggcactgtgatcaaagaacttggtctgtgtgacaccagttcttgcagttggttgagaagtgctccatgacacattatccacaaccagtggacagaatatgtcaccaccaagcatgggtgcaggattctacatatattcattacctctgtcttatccgtttgttgtgaaagtattttctcttcttactgtcattttgtttacttgatttatcaactgatggaatacgtgttaaaacctactgtggtgatggacacatcagtttctccacagagtttggtcattttcattttattatttattttgaggccacattttcagttttagatacagtttcacattcctggtgtattgaaccctttggtattatgaagcgaccatactaatatgttatttttttttaaattattatttattttatttatttttctttcttttttttttttttgagacagggtctccctctgtcacccagtctggagtgcagtggtgcaatctcagctcactgcaacctccgcctcctgggctcaagtggtcctcccacctcagcctcctgagtagctgggattacaggcttgcaccagcgcgcctggctaagtttttgtatttttagtagagacagggttttgccatgttgcccagactggtctcaaactcctgggctcaagcagttcaccagccttggcctctaaaagtgttgggattacaggtgtgagccaccacgcccggccatcccagttttttattttcctcctttctgtgtctttatgcatcagtgaggtggagcttttgaaaatgtcagatagctagattttttttttttgagacggagttttgctcttgttgcccaggctggagtgcaatggcgtgatctcggctcactgcaacctccacctcccaggttcaaaacaattctcctgccgcagcctcctgagtagctgggactacaggcacgtgccaccacacccggctaattttgtatttttagtagagatgtggtttcaccatgttggccaggctggtctcaaactcctcacctcagctgatcacccgcctcagcctcccaaagtgttggaattacagacgtgagccaccatgcccagctgatagctagattttttttttttttttttttttgagacggagtcttgctctgtcgcccaggctggagtgcggtggcgcgatctcggctcactgcaagctccgccccccgggttcatgccattctcctgcttcagcctccagagtagctgggactacaggtgcccgccaccacgcgtggctgattttttttttttttttttttttgtatttttagtagagacggggtttcaccgtgtcagccaggatggtctcgatctcctgacctcgtgatccgcccgcctcagcctcccaaagtgctgggattacaggcgtgagccaccgcacccggcctcgagattttttaaaaaatccattgtctgtttcgtcctttaacatctatttgatttctgatgtatttagattcatttatagcatatttgtgcattctatttttccatattttctgttcttttaaattttccttctcctttcttgccttctcttagacttttaaaaaaattgtagtaaaatacacatcacctaaaatttaccatttttaaatgtacagttcagtggcattaaatacaaattggaggccaggcatggtggctcatgcctggaatcccagcactttgagaggctgaggtgggaggattgcttgaacctggaggtcgaggttgcagtgaggtgtgatcacactactgctatagcccaggcaacagagtgagaacctatctaaaaaaacaaacaaacaaacaaacaaacagattggcatcacacagtgtgtgtgctgctgtgctgtgctgttatcatttaacattaaaacataaacatcagcactgttatggactgaatgtttgtgtcccccaaaattccagtgttgaagctcaaacctccaatgtgatgttatttggagatggggtatttgagaggtggttaggtttagatgaaatcaggagggagggtgggtttcgcatgatgagattagtgtccttataacaagagacagcagagagcttgctttctccaccatgtgagtatgcaatgagaaggcagccatctccaagtcagaaagagagccctcaccaggggctgaatgtgctgacaccttgatcttggacttctagcctccagaactgtgagaaatttcagttgaataggccccccagtctgtactattttgttatagcttgagcagactaatacacccatgttatgaaaagctataaacatccctttaaaggccacctaacgcgtcgtattacctcatgcatcttctctgctgagctttccaaaagagtggcttaggctgggcctggtggctcatgcctgtaatcccagcactttgggaggccaaggcaggtggatcacctgaggtcaggagttcaagaccagcctgaccaacatggagaaatcccgtccctactaaaaatacaaaattagctgggcgtggtggcgcatgcctgtaatcccagctacttgggaggctgagccaggagaatcacttgaacctgggagatggagggtgcagtgagccgagatcgtgccattgcattccagcccggccaacaagagcgaaactccatctcaaaaaaaaagagtggctggccgggcgcagtggctcacgcctgtaatccccgcactgtgggaggccgaggtgggtggatcacgaggtcaggagttcaaaaccagcctggccgagatggtgaaaccccgtctctactaaaaatagaaaaactagccaggcgcagtggcaggcgcctgtaatcccagctacttgggtggctgaggcaggagaatcgcttgaacctggggggcggaggttgcagtgagccaagatggtgccactgcactccagcctgggtgacacagtgagaccccgtctcaaaaaaaaaaaaaaaaaaaaaaaaagtggcgtatgttcccggtctccacctctcattcgccagcccctgtgaccaaacccctgggattagatctccatcgggtccctcccctcaccctaacctcacatgcagtggttcctgagaagctgctgtttatctgatcacgggatccagcaggatctgagggctcattcactgaatctgtactacccaacaaggcaggcttgtccttacccacacttttcaggtgaaaacttaatcagattcaagcgtttatcttagtggcctcttcgtggtgtgtgatttccttcttgcagctctttactggctaaaactcttcactgggagttaaaccaggtgtggtccttgaccctcatcattttgtcccatctcctatgctgggctctgtatccttgtctccaagcagtctcgtgtgacagggaagttacctattattgtatggtcccctcttcttgtcacctctggtgtggcacttgttccatttcttgttcaatgaatgtgatttgtgaatgtgccacatctgtgggaggaaggcagttcgcagcaagagttgtagttcccttgctttgccctgagggccaggactttacactagatttttttgttgcattcctgcactcataaagtacacgtaaacttagaggtggtgtgttaaatgctagaacttaaaactgagttcagctaggctcaagtgccccacaatttttgtcccaccagacaatttcatgaatatctacagattgttccctcaatatgcctcaatattgaggcatattgatttggggagcacagacaagtcaaggttgatgtgcatccgctgaacatgatcttaagttgttgaagtaggtattattaatgaacgaacgtctattttagctccaagagcccctggtcttccagaagagactcccaatgatcttccagaagcagctgggcttcttttggtcctcccccacccaggtgcagtcccgttctccctgcctgggtgagtttgattcctctgccgtggaataggctaggctgctacctcttgagcctcttccaccaggccatccattaggtggcgacagagagcactaaaaggaactactatggaaataaaacttgttttgcttcttggggggaaaaaaaaagaaaaaggcttgtggggcgtgtgtgcattttagtcagattttactgtgcaaaacatttgagagatttctgccctctttctcccttccattcttctcaacccactgggcgccctaccacccctgtctccttcaagataaggtagatcagaagaccaaatagacaaatgccatgtccactgttttctgtcacagttgatagccataccagtcacccaagctggaaacccaacaggcccctgcccttcactgcccacatccaagggagccaccaagtcccacaaattgttcctttaactgtttgtccctcttttgtcttctgtcatcccttctaccgctgtcctggttcaatccctcatcttgctctgggactcccatgataacttatgatctttcctccacctctctatcttccacacaatccctatcatcaatctcattcccctgctttaaacccaagagtggcaccctgtgtttgagtgacatcagggcccctcatgtggctcctgcttctctttgtggccccttctgttgctgctgcttttcttatggacatagccctcagcagtccaggactgcttgtagttcctcccaggtaccatgtttcttgcactcctggcctttgtcattggctcccctgcccaagtccccttctcttctcctttccttcccctgtggccttgccttgtcgtgcttatccttgaagaccctgtcaggtatctcttccccaaagagccctgcctaaccaccgcccccctcccccccgcgacacacacacctgggttaggccctgcctctccacgcctgtgcctcctgccaagtcctcagaggtctctgatcccatgtgcctccaggcatggcatagggggactgtgacacagcctctcggcccacagacccctgattgtgggcttgctcactgccccagtcagtcctcttattgaggattccgtgccagttcctgactccagcacacacacgcatgtaggccgccctcctagtgcctggctccttccctgtctcttctgccactcattcccatggtcgtatcctggagttggtcatctctggaactccacccacggagttctaacttagctctgcctatgatgaggacttactgtcttcccagcttgcagtgtcaaccactgtttgatgcccctgggactccattggtcttccctcctcttgtgaactgtgcccatcaccagacgaacttgttttagaatttaccatcgtaactgagctcccttcagcgcatacctccttggctcctgctcagtctctgtttctccttttagccaaactctctaagagttctctccactcatgggccctcttccttgcctgctgctccttgctcagcttgctccatgaacacttgcttttccacattccatgaaataatcttaacagtcatcagtggctgtcactccttctcagccctttccttgtgtctcttctggtacctcatactctccaggtggccatctcacctcacatcttgaaccctctgggtttgggagaattctcacctcacgagttgatgggaggcctggcgacctctgattgtagtcactgcaaatgccagatgcttatttgcctagcttccctttcggtcagggcagcagttggcaaacggtatctcaagggccaagtccggcccactgcctgttttcgtaaatgaagctttaccaggacccagttccaccccgtcctttgtgcattgtccaagactgctctcctgtacaacagcaggtgggctggttgtgacagatactgtctggcccacaaggtggaaaatactatctggccccttacagaaagaaattacctgagttctacacgagggtgtaggtatgtaccctggcctctgccagtcagactcacatgctaggaggagcagggccaggcaggagagccgtgtctcctggcattggggctgcaggaaagacgagctcctaggccacagtgccagtggccagtgctgctgccttgcagggtcgcacaggcagtgctggctgtttggactggcttggcagaaggatttgaagtattgttcctagccaactggactcaaacctgttcctccaaccctctagggattctcttatcacccagtagccttttccccctagttttaaaaattgcagtaaaatacacaaaagataaaatgtactgccttaaccatttttaagtgtacaattctgtggtattgagcacatttatattgtgcaaccatcaccacatgcatcttcaatagcttttcatcagtccttgtctgcataaatcagctagagttggtttctattgattgccaccaataatcttgacagatatgtctgatcttccttctctgcactgactcctctttctctgtctcctctgctgattctttctcctcatcacaactccaaatagcagggggagggttgtttacacttatattcagcacgccctcacgcactcgcctggctcttcagacctctcccctgaactccagattcatacatccagctgccctttaggcctctagaaagctaaatacctcagtatcaaaaacatgagaaacggaagccagagtcctggatctctcgccctttcgctgtcccggtgcccatgcccttcagggatggcacctagaccagctccctgcatccctgcctagactctgcagtagtctcacgtggcagacccggaggtcactctcctctctggatgccctgcggacactccggttagcctcagctgcaagagcctcctcacccaaggtcacgccatttccaggacagcaccctggtgactgagcgaggtaagggtacaaggcccttatagggccttctctttgacttcttcccacctccctttatggatgtcagttcctgatgaacatcttacacctcaaaccgtgacccaacatctgcttctgcagagcccccctgtggcatcttgtgccctcctgccccactccctctactctatatcttcccatagttagaatgggctttttttttttttttttgagataaggtctccctttgtcacccaggctggaatgcaatggcacagtcacgactcactgcagcctcaacctcctgggctcaagcagtcctcccacctcagcctcccaagtagctgagactacaggtatgcaccaccacacctggctaatttttatattttttgtagagactggggtctccctatgttgcccgggctggtctcgaactcccaggctcaagcaatcctcccgccttggcctcccaaaatgcaggattacaggcgtgtaccagtgtgcccagcctcagatgggctctttgaaaaatgtttattagatcaggttactcccttgagaaaaaccctctcagaataaaacccgaagtccattacttgaaagcagagccagcttcatgggcctttgtaactgcccatgggcttgctggaacccaggcttggtttaatactctactctcaccatctcaaaattcttaatttttgaacaagaggccctgcattttcattttgcaccaggctccacaaattgtgtaactgggcctgcctcagcagccacatggaacatgatccctccttgcctatttgagctcattctctaccattctctcccttgctcacctggctcccactgctcttgctcttcctggaacttgccataatgttgctagagtcagagccttggctcttgctgctcctcattggctggacccctctttacttgacctgctccctcaccactcacttccttcagatctgtgttcagatttcgtcttctcagagaggcttttggcccctgtccatctctctgaatttacctctgacctctcccccaccaccactgcgctaagctgcctcagaactttgtagacattctgtctggtcttctgatgtttcccccttggaagaatcccaaggtgcctgaagaatgctttctttatccttgaagttgagtaggttgactagagtctggcttgctattgagcattctttatcaaattgtcctgggaacatggtgtattctttcagtctgcagatgtagtcgttgctgtttcaggtcagctctcttatggatctttgacagcatcttctgttccatttgttgagttctgtacttcagggacacaaattcctcatgttggattgtctttgtctctcttccaatgctattagctttgccgtaattggtttagcttttgtctttttcatctgcattcactttgtctaatttgattttcagctatgtatattctgtttctggctgtttttcaatgtatttattagtttcataatgatgtgttttggtctgcagtttgtttctctaggttggaaatttgtcttttcatcttattctgttttatcatcccatctttgaactcttttattgggaacatgttcttatgaagttgtggggaatttttttcccttccttgtgtattctcttcttggtgggagactttgcctttctcgtgccatctccctccctgggcctcttttttttcttctggccataatatgtttgcctagttaccatgtcacttcttttcgtcttggctcaggcttggatggctctgcatagtcgttctgtttgctttgagacagtggaggaattcttggctctctcttcccagtttcttggcatcttctcttgctgttttcccctctgagctatcgcgtgcaggcttgttatcttgtatccggagagaatttgcacgctggagggagctgcagccatgtagtcttcagccctatctggattcttctctttgttctaagaactgtgttggatgtcttactaaggctcactctagctgcacgagggatgtgtgtgccatttccatggggatagggggcacctcagtctctgggtggttccataatctgtgtatacctaagagcagttgcttcccacagagctgggctggctcactgggcactttgccatttctcctgcacctcccagctggagtttctgggtctaaaaggaaaaagtgaaggactcccacttggttgcttctctccagcttactgactgcaaattcccagggcgttgcccgctccctagggtggtttctgggggacggggcaggagcctggctttgctgctgctttgtcctctggagtcttttctcagactgctttgaattacacccctttcctttgtgtgctgaaatcttcccttcacactctccttccctgcctttcttttgtgtcttatcttactaggactggaagagggcagttgtgcaggaggtctgcatctaattccctaatccatgtgagagtgctcctgttgtgtgctttgtgacatgatggtgagaaatactccctagagcagtggctaccagaaggaagccatccccccccctgcacacacacacacacacacacacacacacacacacacacacacacactccttctggctgtcctcagatgcctattctaggtaaatgtcagattccaaagaaaatccagttgagatcttttttctttttttgagacgagagtcttgctgtgttgcccaggctggagtgcagtggcacaatctcggctcactgtaacctccgcctcctgggttcaagcgattctcctgcctcagcctcccaagtagctgggactacaggcgcgcaccaccacaccaggctaaaatttttttgtatttttagtagagacagggtttcactgtgttggccaggctggtctcgaactcctgacctcgtgttctgcccgcctcagcctcccagagtgctgggattacaggcttgagccatcgcgcccggcctccagttgggatcttgactggaatgactggtgttcaatcattatagtttccacctaatttgtatttgtacacaggacagttactaatttgttggtacttgtttgatccccagtccctagagttgtttatggggtggagcttcagtccctgctgcttcccctgtggcagcagctggagtcagggtggggacccagggtgctgctggcagattcttgagacaggtagaatctcctctatattggtgtctctctctgtcccagcagtgcccaggaaaacctggccagcctgtcactgacctctccacctagggcactggtggttcaggtccttctatttgccaccggcaaaccgtacttctgccagtctggctcttaggcccagtttctctgatcttgcagattttcttgggctctgctacggaatcctcatacctccggcaggtccctcctttgcccatgtgtttaactgtggtggaatcgtgtgagagctgcttctctcgcatggatcccagccacaccacattctacagcggttcctctgaaggcattgatagagatatttcctcctgttttgcatttcgttggtcatttcagtagaatcagggtgaaataaacatgggggctcagatgtcagcattacgaaccaagtacgtcaggcaggctgatgtggactgacctacactagtgagacgcaagatgacgaaaacaagggcactcactccaagttactgatgagatgtttggatcaaatgagccagtccttaagcagagttctctagtaaaagagatctcctttctgccctttcttgttccccaaaatgtgttgccttcatggtgaaaatttattttggcagattttctcttctttgataaaagcagccaacactttgttaaaagtctgtgaaacttatttacatgaagtatgtaaaggtaagaaaaaaacattatcaacaagaaatggagaaagccagcagctgaggacagaaaagtcatgcacagtgtcagtgtctatggaaacaggccacttggaccttgaggactaggtatttggaattggaggtgagcttggcctggtgagtctctaaccacttgtgtgtaggatcagtgtgagaaccctgctagaatatagtggcagagatgcaaggggaaatcattggagaagttaccagggaatgatgagctaatctgaaaaaaatacatgtttctaagttgggcgtggtagctcatctgtagtcccagctacttgagagcctgaggcaggaggatcgcttgagcccagcctgggcagcacagcgagaccctatctccctaaaaaaaactttttcgttgtttagttttgggatttttttttttcctggtcttttttccccctttttgtgaataacgggatctcactatgttgcccaggcagatctcgaactcctgggcccaagcaatcctcctgcctctgccttcctaagattacaggtatgagccactgtgttaagcaaaaaaacttttttaaatgaaaatcattttttaaaagacaggctttccaggggagggtattattccacttatatgaagtgtcaacagtaggcagatttgtggagacaaatagattagtggttaccaggggctgagaggagtgggagtggggagcaactgcttaatgggtaaagggttgtctttggaactagagagtagtgatggtcgcatgacattgtgaatgtactaaatgctattaatgataaattttatgttatgtgtattttaccacaattaaaaaaaaaagatcaaatgtcctcagaatagccaacaaccttccacttggctaaatgcctactcattaaacttcttgaactaaattcctttctgattgtcatggttattgtgtcctgggcttcagagtttcacattcaggttggcttggtccagtctgtcatgtatcactataggtccccacattggcctcttcctcagacggacagcccatctatctgccggggctctgtgccacagccagatagacttgctctgagacagctgtgtgggctctgagcactggccaggcatcacaaaacctatctttatgatttagaataattggtggtcagctgctgttttaatgttgttgttttttttaatttagatataattcacataccatgaaatttactcatttaaagtgtacaattcattcttcagtataattcataggctcacagaaaaaattgtttaaaaataaaatgtgcaattcagtgtcttttagtacattcacagagttgtgcaaccatcgcctctgtgtcattccagaacactttcagcacccaaaagaaaccccagacacaggagcagtcacctcttattacccgcagcccctggcaacaactcatccacttcctgtctctatggatttgcctattctggacatttcctataaatggaattatgcactatttggccttttgtgtctggcctctttcactgagcgtaatgtcctcaaggttcatctgcattgtagcatgtgtcagaatttctttcctttttgaggctgaatgatattatatcctatagataatgaggttttgattatccacccatcccttgggaatgcatatttgggttgcccccaccatttggctgttgtaaactgtgctgccatgaacactggtgtacggatatctgtttggttactggttttggttttttgtttgtttgttttggttttttgagacaaggtcttgctctgtcgcccaggctggattacagtggcacgatctctgctcactgcaacctccacctcccaggttcaagcaattctcctgcctcagtctcctgagtagctgggactacaggtagcactcaccaccatgcccggctaaatttttttgtatttttagtagagacaaggtttcgccatattggccaggctggtctcaaactcctgacctcaggtgatccacccacctcagcctcccaaagtgctgggattacagacgtgagccaccgcacccggcatagttgtggctttttgagagtgtatggctaggagtaaaattgccaggtcatatggtaactccatgtttaacatttgagaaactgccaaactgttctccacagcaggaattttttaacctgtatgtggtgggcttgtgtttcggttttcattttacacatctataaagatgagatttgctgtatggcactggttgcctgtatttggggagggttctgcttttggttggcaagaactgcattttatttaagcttagcaaaacataactggtttctcgcatcttctcaaaagtggaggattaagaaatggactgcgaattcagagcagggcagctgaacctcaggctccacccttgtagccttcaagctgaacctcattctctcttgctccctggagaccactgagacactctgcctgtgccagtttgatttctcacatttttaaagggccaaagcttgtgtctcaaagtgctatagcctttattgattcatgcagagaagcctccttgattccgtaattctgcagctaatactggaagtagaaagaattggaaacaccatctggatgacactttagggtggaagcagccagtacaagggggggctcattatttcctctggtcccagactgttcacctggagctgtagccaccaccctgcccttaggttaactgcctcgagtggtagtttagctctttgtgctgtgccgagggataactggaagtgaaaggtgctgagaaatgccatctcctgaaagtggcgagcatgagtgaatttacgaaaggttgggatattgctggggctctggaagtttctctggagctcactccaggggacagggagggggctggattccaattcaagtgaaaaatacctttcatctgccttgttcacctggcttttttgcctttttgtaaaatctgaaaacctcagggattgagtagtctttccttaactgcagttgcctgtctggccacacctgccagctgttgcttgtacccctgtaatttgcacggccttccgggcctttctcacaagatcactgcaggtcacattcatgaggaaaatgcaggcagttcctgccatcagacccctcaggatgtcatggtttggcctgaaaacaagattcctgcaactctaattttcctttgctagatcaaatgaaggatttgatctaatgtttgcattctagcagcaaaatcattgaatattttatttcttaagagccttacttcatattttgtaggtattttaagattttgtaaaggcctttctgcttcaacgtgtgatgtgtgcattcttagaaaaagatcttgtgttctgtaaatcacacaataaaaacatgagttcgtgcaggaaaaactggggcggggtggatcactccaaacttgtgtggtgtggtaactggagctcactgatgaaaccatgaacagttctggctgaaagaaccccacagtacactgaggtctgttggcatcgccgccagcaccgccccggtccctttgtgcgcgccaccacacctggctaattttttatttttagtagagagggggtttcaccatgttggtcaggatggcctcgaactgacctcgtgatccgcccacctcagcctcccaaagtgctgggattacaggtgtgagcctccgtgcccagccttcagctttttttcttaatgtctttgtgtaatatgaaggcattctctttaattgttaaaaagcttgcctaccactgcttcaaaatattactgtcagttggcatagcttctgattatattgatgtcatctcccttcttttaaacatgttaccatattggcactgtatttcccctagcccattgatcacttgagagttagtcatagtcctcgtgctgtttcactcctaaacgtttaagcatgcctttcctaagagcagacagtacagttaagacactcaggaagtttagcaatgagctaacacagaaccttacatttctccacataccccataaatgccttttttagagcctttgagcctggaatgcagagtccaggactgtgtgttgtattcgattgtgtcatcccttcagtctcctttatcagaaatgttcccccacccccttgttttctctcatcatcttgagtccagaccgtggttgcatggcacgccctctctggactcttcctgctgtctcctcacggtaagcttcagtctcctactcgtgatgctggttcatctcagcagcactgaggggcctgagctcagtttggtcgtgtgttaaggtggtgcctgccagatttctccacagaaaagggccccgtacttttatttgctcttcagcctgtgtattccttcctttcttgccagcttgtgggtggcttctctagcagcttctgtaagatactcagtttggcagttgtagttgtttcagcaggagaggtgtctgcatacctgaccaccacatggctagaagtcgatccatcctctggcgtaaccatcctccatgctactgtccctggggcactgaggccctcctcagtgacttcctccacctcatcttccgccatctccaagccaccagtgtccaacggactactgatatgtcccaaactcatcatttgtttattcaccaattcagagcccacctttttttcagcactgagctagcctctccttgctagaagcttacggtcgaaggtctccagccatcagaagaagcacgtggagcgctgcgtccgtgttgtggttattcatccagcatgtgttgagtaagggttgcacctgtgcctggcattatgcattgagcggggagatgggggttggcacgcacagtggggtgttctaagtacactgagggctcgggtgccctggctcatagagcagggagggaggcaggagcagggaaggtgtctcagaagtgccatctttttttttttttttttgggaagtggaatcttgttctatcgcctggtctggagtgcaatggcactatctcaactcactgcagcctccgcctcccaggttcaagcgattctcatgcctcagtccccgagtagctgggaccacaggcgcacaccaccacacccggcaaatattttgtatttttagtagaggcggagttttgccatgttggccaggctggtcttaaactcctggcctcaagtgatctgctcacctcggcctcctaaggtgttgggattacaggcatgagccactgcgcccggtctagaagtgccatcctaactgaacctgaaagatgaaagttctccagatgaacctgaaagttctccaaatgaaaaggtgggagggggtgacagggttaggcccagagcctctggtgacacagggtgggcatcattggtcactttttcctcgagggaggggcgtcacacgggtgatagggtgggagctataaccatgttgatagtgccgcctctgcccatctggcctggcatgccctgagccctctgtcccacctgtggaactcataagccctgacagcccactcactcctgattcattatccacacccctgtgcttccgctgtgcctggagcaagctttcttcagggggaagggaggctggaactatgttgtagttacctatttgtcctcccttccaaactgtgagttcttggaggtggaaggatgctgcaggatctggctcaggacgaaggcagttggtgaacagacacgtgtgtttttgactcacggtgatctcagacaagttcctctgtctagtcgaacttctttttttccatctgtaacactcaggagttgaataggtggtttttctgaggatacttcaactgtaaaatgtatgaacttgtgaactagctatttagttctcctcataatcaagattgtgtgtgtgtggggggttctgattagagggaggatgaagagaggtgtatggggttttttttgttttgttttctgtttgtttgtttgtttttgagatggagtctcactctgtcacccaggctggagtgcagtggcacgatttcggttcactgcaacctctgcctcctgggttcaagtgattctcctgcctcagcctcctgagtaactgggattacaggcatgcaccaccatgcccagctaatttttgtatttttaatagagatggggttttcgccatgttggccaggctggtctcgaactcctgacctcaggtgatccacctgcctcggcctcccaaagtgctgggattataggcatgagccaccacacctggcaggtttctttgaaaaagtttgtgtttcggcaaacaccataaaccccctgggggacagccttggggagtcacctggcaccctagcccagcctccctcccttgggtcctgcagtgaaggcttagtgagggtgtgcaaatgcccaggtcaccctgggactgggcaggccctctgggctaagggtaaactcatttggaatacctgttttctatcattgttttttatttgttaaatttaaagggtacaagtgcagttttgttgcgtggatatattgtatagtagtgaagtctgagctttcagtgtaaccatcacctgaatagtggacattgtacccgttaagtaatgtctcatccctcaccccctcccacccttcccagtctctccagtgtctgccattcctcactctgtccatgtgcacatgctattcagctcctgcttctaagtgagaacgtacggtatttgactttctgtgtctgagctgtgtcactgaagacaatggactccagctccatccacgttttttatcatttttacctgcactccacacccagcacaatccaggcttctttgtgggttttttgaaatttgtctttaattataaaagtagcagccagcaaattaacaaacacccatgtgcctttcattgcacagaattgaaaatcatcatactatatttgcttcaagtaatttccattagaaagaactagaatattacagtagagttaaagaccctttatttcccatcttcagtgctcttaaaagttcatttagggccaggcatggtggctcacacctataatcccagcactttgagaggcccaggtgggtggatcacctgaggtcaggagttcaagaccagcctggccaacatggcgaaaccccatctctactaaaaatacaaaaattagctgggcgtggtggtgcgcatctgtaatctcagctactcgggaggctgaggcaggagaatcgcttgaactcaggagggaggcagagcctgtatgcagtaagccgagattgcgccaccacaccccagcatgggtgacagagcaagactccgtctcaaaaaaaaaaaaaaaaaagttcattcattgtacacttagaaatagttaaaaaggtaaactttgttttgtgtgtgtgtgtatatatatatatatatatatatatatatatatatatatatatgtatttttttttgagacatatatgtgtctggtttgttcgcccaggcaggagtgcagtggcatgatcaacggctcactgcagcctcaacttcctaggctcaagtgatcctcctgcctcagcctcccgagtagctaggattacaggcacacaccaccatgcccagctaattttttttttttttttttttttttgtagagacagggttttgctatgttgcccaggctggtctcaaactcctgagctcaagcgatccacctgcctcacctcccaaagtgctgggattacaagtgtaagccaccacacctgacctgttttgtatattttaccacaataaaaagcctttaaaaccccaagcagacagttcattttcattcaggcccaactcagaatctgatcacagcggggtttcccccctttctagcgagtagctgaagaactgttttctctccttgatggtataactgtctctgtgggtgttgctcccctgccgctccagtggttttgtttttgtttttgtttttttgagacggagtcttgcgatctcagctcactgcgacctctgcctcctgggttcaggcgattcttctccctcagccttccatgtagctgggacttacaggcacctgccaccacgcccggctcatttttgtatttttagtagagacggagtttcaccatgttggccaggttgggctcgaactccagacctcaggtgatccacctgcctcagcctcccacagtgctgggattacaggtgagagccactgcacccagccggcccctcagtcttttccttctcaatcagtggcaccaccatcttcccaggctttggacatggtccctgactcacccttgcccctcacccccacactaatccacctgcgagctctgttgcttcaccacctagaccagccccaaatcctcaactgcccccaccctgggccacacctggaccactgctagaggcctctcatgggccctccctgtttttctcttgcactccccgggctttctggcacaagatgccccagaagcagaatcacatatctctcctgggagccaatctagtgtgtttactgcccctggagtgttattgttggccttagaatatgtcccactacaggtttgcagagcactgtagtcaaaagtcatttgaaataaatcttttctctgtggtatattgtcaatttgatatagaattaaatttgtttcttttcttttttcttttctttttttttttttttaagagacagggtcttggccaggcgtggtgtctcatgcctgtaatcccggcactttgggaagccaaggtgggtggatcgcctgaggtcaggaattcaagaccaacctggccagcatggtgaaaccccgtctctgctaaaaatacaaaaattagccgggcgtgatggcaggtacctgtaatcccagctactcgggaagctgaggcaggagaaggcttgaactcaggagtcggaggttgcagtgagccaagatcacgccatggcactctagcaagactctgtctcaaaaaaaaaaaaagagagagagagagacagggtcttgctctgtcactcaggctggagtgcagtgatgtagtcatggctcactgcagcctcatactcctgacctcaggtgatcgacccgcctcggcctcccaaagtgctgggatcgtaggctgaagccaccatgcctggcccgaactcatttgtttttatttgcattaagtgtaataaggttttgttacttttagtttgaattttattttgggtaatataaacatttacatgattcagaagtcagaattacactgaggcatgttcagctaggcctcactcctgtgcctgtacccttacctttttccccctaccccatgcagggaatcaatttcattagttcctggtgtgtccttccttaatacccccccttctttctttggtagaatgcagtagatatgtttgcgctttgctccctttgcttcacagtggatcctggaaatgactccatcgcagttcttagagctcttgtttagtccctttggatctgcacagtactccagtgtgtgggcgcaccataagtttattcagcaagtgccctggtgatagggatcgggggtattggaagcctttggctggtaaaaataatgttgtagcaaataacatcatgcatatgttctttgagatttctggaggtgtctctttagcatagatttctagaaggcattccttacgtcacgtttaggtttatagtttgacctcatggtgattaaggtatttgggaaatgcaaatgagagtttcgaagaagccagatctttcccattagttcactgtttttctctgacattaggaccgtccgggttctagggagcagccctggcgttaggcagtgccatgatggattgtgtagaagtagcgattcccatctgtctgctttcttggcccactctgctggtggctccccttccctccctcctttatggggagctggggagctgcctaggggtccattctcaaaggctgatctctggtgggcaacaggccacacctagctttccagggttcttcgtcatttcccattgagagctgtaagactcagagacatgaaaaggaagctctggctgggcacggtggctcacgcctgtaatcccagcactttgggaggctgaggcgggtcgatcacctgaggtcaggagttcgagaccagcctgcccaacatggtgaaaccccgtctccactaaacatacaaaaattagcctggcgtggtagcacacgcctgtgatcccagctactccggaggctgaggcaggagaataacttgaacccggcaggcggaggttgcgatgagccgatattatgccatcggcaacagagcgagactccatctcaaaaaaaaaacaaaaaacaaaacaaggcgagctctgtgctgggacagattagggacccctctttacagcaagaaagactgctctgtgggttgtaggatgcctttgtgtatgcagtggctctaggtgactctggcagccacactctgggccctaaacttctggaggaagatacaggatagggaggaactcaggggtgagtcatggtggggacaagacattccctcactctaagaccttgtcactagattggaacatctcttgcctccctacacctgacctgatggttctggagagatacgtccttgcagcttctgagtcccagcactaagcagcctttggtaacttccctacatcatttgagttctggtttcctaaggatgcttgccagtgagtgccatggtgccctcattgcacagtctgtgcagtgtagacaagaggggaagtctcttggggtagaccagccgcaaggcggtgactagcactgatgtgaaccacatgggacaggggagttgtggggctgagaacacggagggtgggagtagtcatgctcttttccagaatgaactgctaacgaagggactcgcaggtggctgctgcttctttccaagctgcccctgttgttgcagaggctctggagtcctaggaggtttcacggtggcatactcgacagagtactagagcatcatggccagatagtgatgctggggtgtggggcctcacggtggccatttctgacgagaccccaccgggccaaagtgatgtgtagagaaggagctgcttcggtcaccagaaaagaacggggaagcctcccacactagaataggtagggtgctcttctccaccggcaggaaggggatgtatggctctgcctggaccacacctttttccttgctcttccctccgcatctgctgtggccgaggccattcctcatcagggaacatgtgttagaggctcacgccacctgggaccacttgtcttatcacccccaggaccctaggcggtagtttcctgtggcctgagttagctgatatttatatagtgccattgtgccttttctcctgtgatgctcacagtaaggatgcctagatggggttacctgttgtcaagataaggaaactgaagcacagaatgctgaggtcatttgctgggttcatgtttggaaagcggcaaaggatttcagtgcaggttggctggctccaaacctgtgtgtgctttccatgacactgtactgtgtgcctcattgagcctcattctagaaaaccaaaaacacacccaaggcccggccttcacaaaggagacccctcccccatttggctccctttccagcagtcgacggcctcttgtcagccatcgagcccagagtcccttgaagtgcgactcatgctggggtggtatgctcaggagccgcagtgtttccgctcagaggaaagggctctgattctcctgcagtgctaggagacttgtgggtggccacagtgcaggtcaggcacaccggccagcaccacccacagcccaaattcctaaagaaatatttgggtcccagcttggcccgagtctctgttgtcctggggaaggacatcaagatctgagtgtatgatggcctggggccttgcatgtggtgggggtccaagcctgcctctgctcacttgttctgcagactggcatgttctctgtgatacttacatacttgtttaacacttcagggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaagtaagtaagagcaactcctatctctacagggcagggagggcagggacaaggatccctcatggagcaggaaaatgtatgtgcccagggtggggtcggggggaacataaacaatgaacactgagaccaggtgtgcttgaaatgaccgtgtacagaggtcgctgccctgagtgggaagttctcaaggtagcaggccctctatcctctccacacctcaagtctttatctggggatggaatagctgcggaagcagaggaacttgcagagctaggggttcagaggggtgaagaagcatgtttcagttctgccttttaaatgatcccaaaaaggttagcagttttcaaatgacatttgcagacagcctcatttaattccatgagaagggtgagcaaaggattatcttgttgaaactgattcctggagagactgagcaccgtacctgagttcaaacttgggaatgttctagatggtgactcaggcccaggcaccaaccagcagaatgggcctcagcctgacaacccttctgtaccaggcctgactctttggttgctgaactttggagaggcctgggggggtcagcggcaggcagacgagtgagtggctttggtgacaggtcctcaggggcagccaggcagtgtgactctcgttcaatagtaacgtttgtcagagcgttgtcaccaccatccgctctgccctatctctgacattgctatggagagcctctaattgttccttgtgtctttctgtttgtccccacagtccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgactttacacggagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcagggtgggagtagttggagcattggggatgtttttcttaccgacaagcacagtcaggttgaagacctaaccagggccagaagtagctttgcacttttctaaactaggctccttcaacaaggcttgctgcagatactactgaccagacaagctgttgaccaggcacctcccctcccgcccaaacctttcccccatgtggtcgttagagacagagcgacagagcagttgagaggacactcccgttttcggtgccatcagtgccccgtctacagctcccccagctccccccacctcccccactcccaaccacgttgggacagggaggtgtgaggcaggagagacagttggattctttagagaagatggatatgaccagtggctatggcctgtgcgatcccacccgtggtggctcaagtctggccccacaccagccccaatccaaaactggcaaggacgcttcacaggacaggaaagtggcacctgtctgctccagctctggcatggctaggaggggggagtcccttgaactactgggtgtagactggcctgaaccacaggagaggatggcccagggtgaggtggcatggtccattctcaagggacgtcctccaacgggtggcgctagaggccatggaggcagtaggacaaggtgcaggcaggctggcctggggtcaggccgggcagagcacagcggggtgagagggattcctaatcactcagagcagtctgtgacttagtggacaggggagggggcaaagggggaggagaagaaaatgttcttccagttactttccaattctcctttagggacagcttagaattatttgcactattgagtcttcatgttcccacttcaaaacaaacagatgctctgagagcaaactggcttgaattggtgacatttagtccctcaagccaccagatgtgacagtgttgagaactacctggatttgtatatatacctgcgcttgttttaaagtgggctcagcacatagggttcccacgaagctccgaaactctaagtgtttgctgcaattttataaggacttcctgattggtttctcttctccccttccatttctgccttttgttcatttcatcctttcacttctttcccttcctccatcctcctccttcctagttcatcccttctcttccaggcagccgcggtgcccaaccacacttgtcggctccagtccccagaactctgcctgccctttgtcctcctgctgccagtaccagccccaccctgttttgagccctgaggaggccttgggctctgctgagtccgacctggcctgtctgtgaagagcaagagagcagcaaggtcttgctctcctaggtagccccctcttccctggtaagaaaaagcaaaaggcatttcccaccctgaacaacgagccttttcacccttctactctagagaagtggactggaggagctgggcccgatttggtagttgaggaaagcacagaggcctcctgtggcctgccagtcatcgagtggcccaacaggggctccatgccagccgaccttgacctcactcagaagtccagagtctagcgtagtgcagcagggcagtagcggtaccaatgcagaactcccaagacccgagctgggaccagtacctgggtccccagcccttcctctgctcccccttttccctcggagttcttcttgaatggcaatgttttgcttttgctcgatgcagacagggggccagaacaccacacatttcactgtctgtctggtccatagctgtggtgtaggggcttagaggcatgggcttgctgtgggtttttaattgatcagttttcatgtgggatcccatctttttaacctctgttcaggaagtccttatctagctgcatatcttcatcatattggtatatccttttctgtgtttacagagatgtctcttatatctaaatctgtccaactgagaagtaccttatcaaagtagcaaatgagacagcagtcttatgcttccagaaacacccacaggcatgtcccatgtgagctgctgccatgaactgtcaagtgtgtgttgtcttgtgtatttcagttattgtccctggcttccttactatggtgtaatcatgaaggagtgaaacatcatagaaactgtctagcacttccttgccagtctttagtgatcaggaaccatagttgacagttccaatcagtagcttaagaaaaaaccgtgtttgtctcttctggaatggttagaagtgagggagtttgccccgttctgtttgtagagtctcatagttggactttctagcatatatgtgtccatttccttatgctgtaaaagcaagtcctgcaaccaaactcccatcagcccaatccctgatccctgatcccttccacctgctctgctgatgacccccccagcttcacttctgactcttccccaggaagggaaggggggtcagaagagagggtgagtcctccagaactcttcctccaaggacagaaggctcctgcccccatagtggcctcgaactcctggcactaccaaaggacacttatccacgagagcgcagcatccgaccaggttgtcactgagaagatgtttattttggtcagttgggtttttatgtattatacttagtcaaatgtaatgtggcttctggaatcattgtccagagctgcttccccgtcacctgggcgtcatctggtcctggtaagaggagtgcgtggcccaccaggcccccctgtcacccatgacagttcattcagggccgatggggcagtcgtggttgggaacacagcatttcaagcgtcactttatttcattcgggccccacctgcagctccctcaaagaggcagttgcccagcctctttcccttccagtttattccagagctgccagtggggcctgaggctccttagggttttctctctatttccccctttcttcctcattccctcgtctttcccaaaggcatcacgagtcagtcgcctttcagcaggcagccttggcggtttatcgccctggcaggcaggggccctgcagctctcatgctgcccctgccttggggtcaggttgacaggaggttggagggaaagccttaagctgcaggattctcaccagctgtgtccggcccagttttggggtgtgacctcaatttcaattttgtctgtacttgaacattatgaagatgggggcctctttcagtgaatttgtgaacagcagaattgaccgacagctttccagtacccatggggctaggtcattaaggccacatccacagtctcccccacccttgttccagttgttagttactacctcctctcctgacaatactgtatgtcgtcgagctccccccaggtctacccctcccggccctgcctgctggtgggcttgtcatagccagtgggattgccggtcttgacagctcagtgagctggagatacttggtcacagccaggcgctagcacagctcccttctgttgatgctgtattcccatatcaaaagacacaggggacacccagaaacgccacatcccccaatccatcagtgccaaactagccaacggccccagcttctcagctcgctggatggcggaagctgctactcgtgagcgccagtgcgggtgcagacaatcttctgttgggtggcatcattccaggcccgaagcatgaacagtgcacctgggacagggagcagccccaaattgtcacctgcttctctgcccagcttttcattgctgtgacagtgatggcgaaagagggtaataaccagacacaaactgccaagttgggtggagaaaggagtttctttagctgacagaatctctgaattttaaatcacttagtaagcggctcaagcccaggagggagcagagggatacgagcggagtcccctgcgcgggaccatctggaattggtttagcccaagtggagcctgacagccagaactctgtgtcccccgtctaaccacagctccttttccagagcattccagtcaggctctctgggctgactgggccaggggaggttacaggtaccagttctttaagaagatctttgggcatatacatttttagcctgtgtcattgccccaaatggattcctgtttcaagttcacacctgcagattctaggacctgtgtcctagacttcagggagtcagctgtttctagagttcctaccatggagtgggtctggaggacctgcccggtgggggggcagagccctgctccctccgggtcttcctactcttctctctgctctgacgggatttgttgattctctccattttggtgtctttctcttttagatattgtatcaatctttagaaaaggcatagtctacttgttataaatcgttaggatactgcctcccccagggtctaaaattacatattagaggggaaaagctgaacactgaagtcagttctcaacaatttagaaggaaaacctagaaaacatttggcagaaaattacatttcgatgtttttgaatgaatacgagcaagcttttacaacagtgctgatctaaaaatacttagcacttggcctgagatgcctggtgagcattacaggcaaggggaatctggaggtagccgacctgaggacatggcttctgaacctgtcttttgggagtggtatggaaggtggagcgttcaccagtgacctggaaggcccagcaccaccctccttcccactcttctcatcttgacagagcctgccccagcgctgacgtgtcaggaaaacacccagggaactaggaaggcacttctgcctgaggggcagcctgccttgcccactcctgctctgctcgcctcggatcagctgagccttctgagctggcctctcactgcctccccaaggccccctgcctgccctgtcaggaggcagaaggaagcaggtgtgagggcagtgcaaggagggagcacaacccccagctcccgctccgggctccgacttgtgcacaggcagagcccagaccctggaggaaatcctacctttgaattcaagaacatttggggaatttggaaatctctttgcccccaaacccccattctgtcctacctttaatcaggtcctgctcagcagtgagagcagatgaggtgaaaaggccaagaggtttggctcctgcccactgatagcccctctccccgcagtgtttgtgtgtcaagtggcaaagctgttcttcctggtgaccctgattatatccagtaacacatagactgtgcgcataggcctgctttgtctcctctatcctgggcttttgttttgctttttagttttgcttttagtttttctgtcccttttatttaacgcaccgactagacacacaaagcagttgaatttttatatatatatctgtatattgcacaattataaactcattttgcttgtggctccacacacacaaaaaaagacctgttaaaattatacctgttgcttaattacaatatttctgataaccatagcataggacaagggaaaataaaaaaagaaaaaaaagaaaaaaaaacgacaaatctgtctgctggtcacttcttctgtccaagcagattcgtggtcttttcctcgcttctttcaagggctttcctgtgccaggtgaaggaggctccaggcagcacccaggttttgcactcttgtttctcccgtgcttgtgaaagaggtcccaaggttctgggtgcaggagcgctcccttgacctgctgaagtccggaacgtagtcggcacagcctggtcgccttccacctctgggagctggagtccactggggtggcctgactcccccagtccccttcccgtgacctggtcagggtgagcccatgtggagtcagcctcgcaggcctccctgccagtagggtccgagtgtgtttcatccttcccactctgtcgagcctgggggctggagcggagacgggaggcctggcctgtctcggaacctgtgagctgcaccaggtagaacgccagggaccccagaatcatgtgcgtcagtccaaggggtcccctccaggagtagtgaagactccagaaatgtccctttcttctcccccatcctacgagtaattgcatttgcttttgtaattcttaatgagcaatatctgctagagagtttagctgtaacagttctttttgatcatctttttttaataattagaaacaccaaaaaaatccagaaacttgttcttccaaagcagagagcattataatcaccagggccaaaagcttccctccctgctgtcattgcttcttctgaggcctgaatccaaaagaaaaacagccataggccctttcagtggccgggctacccgtgagcccttcggaggaccagggctggggcagcctctgggcccacatccggggccagctccggcgtgtgttcagtgttagcagtgggtcatgatgctctttcccacccagcctgggataggggcagaggaggcgaggaggccgttgccgctgatgtttggccgtgaacaggtgggtgtctgcgtgcgtccacgtgcgtgttttctgactgacatgaaatcgacgcccgagttagcctcacccggtgacctctagccctgcccggatggagcggggcccacccggttcagtgtttctggggagctggacagtggagtgcaaaaggcttgcagaacttgaagcctgctccttcccttgctaccacggcctcctttccgtttgatttgtcactgcttcaatcaataacagccgctccagagtcagtagtcaatgaatatatgaccaaatatcaccaggactgttactcaatgtgtgccgagcccttgcccatgctgggctcccgtgtatctggacactgtaacgtgtgctgtgtttgctccccttccccttccttctttgccctttacttgtctttctggggtttttctgtttgggtttggtttggtttttatttctccttttgtgttccaaacatgaggttctctctactggtcctcttaactgtggtgttgaggcttatatttgtgtaatttttggtgggtgaaaggaattttgctaagtaaatctcttctgtgtttgaactgaagtctgtattgtaactatgtttaaagtaattgttccagagacaaatatttctagacactttttctttacaaacaaaagcattcggagggagggggatggtgactgagatgagaggggagagctgaacagatgacccctgcccagatcagccagaagccacccaaagcagtggagcccaggagtcccactccaagccagcaagccgaatagctgatgtgttgccactttccaagtcactgcaaaaccaggttttgttccgcccagtggattcttgttttgcttcccctccccccgagattattaccaccatcccgtgcttttaaggaaaggcaagattgatgtttccttgaggggagccaggaggggatgtgtgtgtgcagagctgaagagctggggagaatggggctgggcccacccaagcaggaggctgggacgctctgctgtgggcacaggtcaggctaatgttggcagatgcagctcttcctggacaggccaggtggtgggcattctctctccaaggtgtgccccgtgggcattactgtttaagacacttccgtcacatcccaccccatcctccagggctcaacactgtgacatctctattccccaccctccccttcccagggcaataaaatgaccatggagggggcttgcactctcttggctgtcacccgatcgccagcaaaacttagatgtgagaaaaccccttcccattccatggcgaaaacatctccttagaaaagccattaccctcattaggcatggttttgggctcccaaaacacctgacagcccctccctcctctgagaggcggagagtgctgactgtagtgaccattgcatgccgggtgcagcatctggaagagctaggcagggtgtctgccccctcctgagttgaagtcatgctcccctgtgccagcccagaggccgagagctatggacagcattgccagtaacacaggccaccctgtgcagaagggagctggctccagcctggaaacctgtctgaggttgggagaggtgcacttggggcacagggagaggccgggacacacttagctggagatgtctctaaaagccctgtatcgtattcaccttcagtttttgtgttttgggacaattactttagaaaataagtaggtcgttttaaaaacaaaaattattgattgcttttttgtagtgttcagaaaaaaggttctttgtgtatagccaaatgactgaaagcactgatatatttaaaaacaaaaggcaatttattaaggaaatttgtaccatttcagtaaacctgtctgaatgtacctgtatacgtttcaaaaacaccccccccccactgaatccctgtaacctatttattatataaagagtttgccttataaatttacataaaaatgtccgtttgtgtcttttgttgtaaaaatcaagtgattttttcataaggttcttttactattggaaaagatgggcagcacgcagttttattttatttttgtaagttttttaatacatgtgaaagcaaagaatactcagcatgcctttctaagtgacgcgtttgcaccttttgttgggaagtactgtatcctgtgctgttagcattctcgataaatctctctgtgaaagtgactcaaggtctgggctttcattataagacagaagtccccctccagctcacatgacagcatggtgctgcgtttcctcattggatctggctgtccctggacacaggtagctgccttcaggcctgccacgagcggccaagggaagcctcctccatatgctggcctcgctggcccctcagcttcttccaagccagtgctctccaggcacactgctccagcgtgtgacgggaagggcctggcatgagtcagcctgcagcacaacctccctgctccagacccgtatggtaggggcaccccctaggtctggatgtgctgtggtgcttttggacacccccacccccgcaggctgtggctcctcctgtgtctcattctggccaggaccctcacgtgccctctgttgactgctaacgtggttctctgaccaggcaagggcaggctgaggggtttgcccaaagggggcccccttgttactggcttccttggctctcaggagcagcctcaccaggttggtaaggggctggaggagacaactgctcaaaggagtccagcttcacatgcacatgctagaaggtaccctcggaaggcctggccttcaaaggtagatcccagggttgaaaagtcaacttgtatgcattgagcatctcgtatgccagccctgttccgtgagctgatgggcctttgtgtgtaagtaggaccaagtgcccccgtggaggttagcatgggtgtgcagtcatttcagatacttgagttggtacatctcagtaaagtctgtcccgtgagaagccatgggtttcatggtatggttggcatcttccttgggagtggccacagtggtggtggcttcaggaaagagactccaacaggggccagctgtgggccttgggcacttctcgtttctaggaaaagtcctaagtctgtagggctaggggtggggaaccccttcgctgtcaggatcaagagggcaaggggaactgtcgctggaggagacatccagctggagaaacaaaagagtaagtctgcgttgctgcttgtggggtcttccccatctcagggcggggaccgggggtggcggtccagacaagtaatcaaggacgatgcccaggaggggacaggtacggggtggcaggagctctgccggcgggctcaggaagccttcaccacagctgcctgagctcacccttgccaaatgagggctggggcagcagcaacgcatacactcacggctgtggcgggcagcgttctcggcatatttcaggacacctaaggagactgaatggctcaaggctgctgccgtgtgcagggggctagacgtggggcgggcaggcagggctcctggtaacagccctgcaggccgcagtggagagcagggttccggcagggccgcccaggagctttcggaaggcccggccccggcccctttccgagcagcccgggcctccgccctgccctctgtccccaacgccgggagccgccgttcgtcctccagagccccgcccgggcgagcccgggaggccgatcgccgctcgcggaacccgccgggacccgggccctccccggcgcggggcgcccccgtgtgacccagcgcgcggccgcggcgcgcaagatggcggcgggcccgggcaccgccccttccgccccgccgggcgtcgcacgaggccggctcgaaggggaagtgagtcagtgtccgcggacccggccggcccaggcccgcgcccgccgcggccctgagaggccccggcaggtcccggcccggcggcggcagccatggccggggggccgggcccgggggagcccgcagcccccggcgcccagcacttcttgtacgaggtgccgccctgggtcatgtgccgcttctacaaagtgatggacgccctggagcccgccgactggtgccagttcggtgggtggcggcgggctgccggggggcgggaggcgcgcgggctcctggcgccgacgcctgacgccc

SEQ ID NO: 2 - RNA, 10241 nt, Homo sapiens
  source 1..10241: mol_type = mRNA
ccggcgtcggcggcgcgcgcgctccctcctctcggagagagggctgtggtaaaagccgtccggaaaatggccgccgccgccgccgccgcgccgagcggaggaggaggaggaggcgaggaggagagactgctccataaaaatacagactcaccagttcctgctttgatgtgacatgtgactccccagaatacaccttgcttctgtagaccagctccaacaggattccatggtagctgggatgttagggctcagggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgactttacacggagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcagggtgggagtagttggagcattggggatgtttttcttaccgacaagcacagtcaggttgaagacctaaccagggccagaagtagctttgcacttttctaaactaggctccttcaacaaggcttgctgcagatactactgaccagacaagctgttgaccaggcacctcccctcccgcccaaacctttcccccatgtggtcgttagagacagagcgacagagcagttgagaggacactcccgttttcggtgccatcagtgccccgtctacagctcccccagctccccccacctcccccactcccaaccacgttgggacagggaggtgtgaggcaggagagacagttggattctttagagaagatggatatgaccagtggctatggcctgtgcgatcccacccgtggtggctcaagtctggccccacaccagccccaatccaaaactggcaaggacgcttcacaggacaggaaagtggcacctgtctgctccagctctggcatggctaggaggggggagtcccttgaactactgggtgtagactggcctgaaccacaggagaggatggcccagggtgaggtggcatggtccattctcaagggacgtcctccaacgggtggcgctagaggccatggaggcagtaggacaaggtgcaggcaggctggcctggggtcaggccgggcagagcacagcggggtgagagggattcctaatcactcagagcagtctgtgacttagtggacaggggagggggcaaagggggaggagaagaaaatgttcttccagttactttccaattctcctttagggacagcttagaattatttgcactattgagtcttcatgttcccacttcaaaacaaacagatgctctgagagcaaactggcttgaattggtgacatttagtccctcaagccaccagatgtgacagtgttgagaactacctggatttgtatatatacctgcgcttgttttaaagtgggctcagcacatagggttcccacgaagctccgaaactctaagtgtttgctgcaattttataaggacttcctgattggtttctcttctccccttccatttctgccttttgttcatttcatcctttcacttctttcccttcctccgtcctcctccttcctagttcatcccttctcttccaggcagccgcggtgcccaaccacacttgtcggctccagtccccagaactctgcctgccctttgtcctcctgctgccagtaccagccccaccctgttttgagccctgaggaggccttgggctctgctgagtccgacctggcctgtctgtgaagagcaagagagcagcaaggtcttgctctcctaggtagccccctcttccctggtaagaaaaagcaaaaggcatttcccaccctgaacaacgagccttttcacccttctactctagagaagtggactggaggagctgggcccgatttggtagttgaggaaagcacagaggcctcctgtggcctgccagtcatcgagtggcccaacaggggctccatgccagccgaccttgacctcactcagaagtccagagtctagcgtagtgcagcagggcagtagcggtaccaatgcagaactcccaagacccgagctgggaccagtacctgggtccccagcccttcctctgctcccccttttccctcggagttcttcttgaatggcaatgttttgcttttgctcgatgcagacagggggccagaacaccacacatttcactgtctgtctggtccatagctgtggtgtaggggcttagaggcatgggcttgctgtgggtttttaattgatcagttttcatgtgggatcccatctttttaacctctgttcaggaagtccttatctagctgcatatcttcatcatattggtatatccttttctgtgtttacagagatgtctcttatatctaaatctgtccaactgagaagtaccttatcaaagtagcaaatgagacagcagtcttatgcttccagaaacacccacaggcatgtcccatgtgagctgctgccatgaactgtcaagtgtgtgttgtcttgtgtatttcagttattgtccctggcttccttactatggtgtaatcatgaaggagtgaaacatcatagaaactgtctagcacttccttgccagtctttagtgatcaggaaccatagttgacagttccaatcagtagcttaagaaaaaaccgtgtttgtctcttctggaatggttagaagtgagggagtttgccccgttctgtttgtagagtctcatagttggactttctagcatatatgtgtccatttccttatgctgtaaaagcaagtcctgcaaccaaactcccatcagcccaatccctgatccctgatcccttccacctgctctgctgatgacccccccagcttcacttctgactcttccccaggaagggaaggggggtcagaagagagggtgagtcctccagaactcttcctccaaggacagaaggctcctgcccccatagtggcctcgaactcctggcactaccaaaggacacttatccacgagagcgcagcatccgaccaggttgtcactgagaagatgtttattttggtcagttgggtttttatgtattatacttagtcaaatgtaatgtggcttctggaatcattgtccagagctgcttccccgtcacctgggcgtcatctggtcctggtaagaggagtgcgtggcccaccaggcccccctgtcacccatgacagttcattcagggccgatggggcagtcgtggttgggaacacagcatttcaagcgtcactttatttcattcgggccccacctgcagctccctcaaagaggcagttgcccagcctctttcccttccagtttattccagagctgccagtggggcctgaggctccttagggttttctctctatttccccctttcttcctcattccctcgtctttcccaaaggcatcacgagtcagtcgcctttcagcaggcagccttggcggtttatcgccctggcaggcaggggccctgcagctctcatgctgcccctgccttggggtcaggttgacaggaggttggagggaaagccttaagctgcaggattctcaccagctgtgtccggcccagttttggggtgtgacctcaatttcaattttgtctgtacttgaacattatgaagatgggggcctctttcagtgaatttgtgaacagcagaattgaccgacagctttccagtacccatggggctaggtcattaaggccacatccacagtctcccccacccttgttccagttgttagttactacctcctctcctgacaatactgtatgtcgtcgagctccccccaggtctacccctcccggccctgcctgctggtgggcttgtcatagccagtgggattgccggtcttgacagctcagtgagctggagatacttggtcacagccaggcgctagcacagctcccttctgttgatgctgtattcccatatcaaaagacacaggggacacccagaaacgccacatcccccaatccatcagtgccaaactagccaacggccccagcttctcagctcgctggatggcggaagctgctactcgtgagcgccagtgcgggtgcagacaatcttctgttgggtggcatcattccaggcccgaagcatgaacagtgcacctgggacagggagcagccccaaattgtcacctgcttctctgcccagcttttcattgctgtgacagtgatggcgaaagagggtaataaccagacacaaactgccaagttgggtggagaaaggagtttctttagctgacagaatctctgaattttaaatcacttagtaagcggctcaagcccaggagggagcagagggatacgagcggagtcccctgcgcgggaccatctggaattggtttagcccaagtggagcctgacagccagaactctgtgtcccccgtctaaccacagctccttttccagagcattccagtcaggctctctgggctgactgggccaggggaggttacaggtaccagttctttaagaagatctttgggcatatacatttttagcctgtgtcattgccccaaatggattcctgtttcaagttcacacctgcagattctaggacctgtgtcctagacttcagggagtcagctgtttctagagttcctaccatggagtgggtctggaggacctgcccggtgggggggcagagccctgctccctccgggtcttcctactcttctctctgctctgacgggatttgttgattctctccattttggtgtctttctcttttagatattgtatcaatctttagaaaaggcatagtctacttgttataaatcgttaggatactgcctcccccagggtctaaaattacatattagaggggaaaagctgaacactgaagtcagttctcaacaatttagaaggaaaacctagaaaacatttggcagaaaattacatttcgatgtttttgaatgaatacgagcaagcttttacaacagtgctgatctaaaaatacttagcacttggcctgagatgcctggtgagcattacaggcaaggggaatctggaggtagccgacctgaggacatggcttctgaacctgtcttttgggagtggtatggaaggtggagcgttcaccagtgacctggaaggcccagcaccaccctccttcccactcttctcatcttgacagagcctgccccagcgctgacgtgtcaggaaaacacccagggaactaggaaggcacttctgcctgaggggcagcctgccttgcccactcctgctctgctcgcctcggatcagctgagccttctgagctggcctctcactgcctccccaaggccccctgcctgccctgtcaggaggcagaaggaagcaggtgtgagggcagtgcaaggagggagcacaacccccagctcccgctccgggctccgacttgtgcacaggcagagcccagaccctggaggaaatcctacctttgaattcaagaacatttggggaatttggaaatctctttgcccccaaacccccattctgtcctacctttaatcaggtcctgctcagcagtgagagcagatgaggtgaaaaggccaagaggtttggctcctgcccactgatagcccctctccccgcagtgtttgtgtgtcaagtggcaaagctgttcttcctggtgaccctgattatatccagtaacacatagactgtgcgcataggcctgctttgtctcctctatcctgggcttttgttttgctttttagttttgcttttagtttttctgtcccttttatttaacgcaccgactagacacacaaagcagttgaatttttatatatatatctgtatattgcacaattataaactcattttgcttgtggctccacacacacaaaaaaagacctgttaaaattatacctgttgcttaattacaatatttctgataaccatagcataggacaagggaaaataaaaaaagaaaaaaaagaaaaaaaaacgacaaatctgtctgctggtcacttcttctgtccaagcagattcgtggtcttttcctcgcttctttcaagggctttcctgtgccaggtgaaggaggctccaggcagcacccaggttttgcactcttgtttctcccgtgcttgtgaaagaggtcccaaggttctgggtgcaggagcgctcccttgacctgctgaagtccggaacgtagtcggcacagcctggtcgccttccacctctgggagctggagtccactggggtggcctgactcccccagtccccttcccgtgacctggtcagggtgagcccatgtggagtcagcctcgcaggcctccctgccagtagggtccgagtgtgtttcatccttcccactctgtcgagcctgggggctggagcggagacgggaggcctggcctgtctcggaacctgtgagctgcaccaggtagaacgccagggaccccagaatcatgtgcgtcagtccaaggggtcccctccaggagtagtgaagactccagaaatgtccctttcttctcccccatcctacgagtaattgcatttgcttttgtaattcttaatgagcaatatctgctagagagtttagctgtaacagttctttttgatcatctttttttaataattagaaacaccaaaaaaatccagaaacttgttcttccaaagcagagagcattataatcaccagggccaaaagcttccctccctgctgtcattgcttcttctgaggcctgaatccaaaagaaaaacagccataggccctttcagtggccgggctacccgtgagcccttcggaggaccagggctggggcagcctctgggcccacatccggggccagctccggcgtgtgttcagtgttagcagtgggtcatgatgctctttcccacccagcctgggataggggcagaggaggcgaggaggccgttgccgctgatgtttggccgtgaacaggtgggtgtctgcgtgcgtccacgtgcgtgttttctgactgacatgaaatcgacgcccgagttagcctcacccggtgacctctagccctgcccggatggagcggggcccacccggttcagtgtttctggggagctggacagtggagtgcaaaaggcttgcagaacttgaagcctgctccttcccttgctaccacggcctcctttccgtttgatttgtcactgcttcaatcaataacagccgctccagagtcagtagtcaatgaatatatgaccaaatatcaccaggactgttactcaatgtgtgccgagcccttgcccatgctgggctcccgtgtatctggacactgtaacgtgtgctgtgtttgctccccttccccttccttctttgccctttacttgtctttctggggtttttctgtttgggtttggtttggtttttatttctccttttgtgttccaaacatgaggttctctctactggtcctcttaactgtggtgttgaggcttatatttgtgtaatttttggtgggtgaaaggaattttgctaagtaaatctcttctgtgtttgaactgaagtctgtattgtaactatgtttaaagtaattgttccagagacaaatatttctagacactttttctttacaaacaaaagcattcggagggagggggatggtgactgagatgagaggggagagctgaacagatgacccctgcccagatcagccagaagccacccaaagcagtggagcccaggagtcccactccaagccagcaagccgaatagctgatgtgttgccactttccaagtcactgcaaaaccaggttttgttccgcccagtggattcttgttttgcttcccctccccccgagattattaccaccatcccgtgcttttaaggaaaggcaagattgatgtttccttgaggggagccaggaggggatgtgtgtgtgcagagctgaagagctggggagaatggggctgggcccacccaagcaggaggctgggacgctctgctgtgggcacaggtcaggctaatgttggcagatgcagctcttcctggacaggccaggtggtgggcattctctctccaaggtgtgccccgtgggcattactgtttaagacacttccgtcacatcccaccccatcctccagggctcaacactgtgacatctctattccccaccctccccttcccagggcaataaaatgaccatggagggggcttgcactctcttggctgtcacccgatcgccagcaaaacttagatgtgagaaaaccccttcccattccatggcgaaaacatctccttagaaaagccattaccctcattaggcatggttttgggctcccaaaacacctgacagcccctccctcctctgagaggcggagagtgctgactgtagtgaccattgcatgccgggtgcagcatctggaagagctaggcagggtgtctgccccctcctgagttgaagtcatgctcccctgtgccagcccagaggccgagagctatggacagcattgccagtaacacaggccaccctgtgcagaagggagctggctccagcctggaaacctgtctgaggttgggagaggtgcacttggggcacagggagaggccgggacacacttagctggagatgtctctaaaagccctgtatcgtattcaccttcagtttttgtgttttgggacaattactttagaaaataagtaggtcgttttaaaaacaaaaattattgattgcttttttgtagtgttcagaaaaaaggttctttgtgtatagccaaatgactgaaagcactgatatatttaaaaacaaaaggcaatttattaaggaaatttgtaccatttcagtaaacctgtctgaatgtacctgtatacgtttcaaaaacaccccccccccactgaatccctgtaacctatttattatataaagagtttgccttataaattt

SEQ ID NO: 3 - DNA, 20 bp, synthetic construct
  source 1..20: mol_type = other DNA
tatttgatcaatccccaggg

SEQ ID NO: 4 - DNA, 20 bp, synthetic construct
  source 1..20: mol_type = other DNA
ctccctctcccagttaccgt

SEQ ID NO: 5 - DNA, 22 bp, synthetic construct
  source 1..22: mol_type = other DNA
aggagagactggaagaaaagtc

SEQ ID NO: 6 - DNA, 20 bp, synthetic construct
  source 1..20: mol_type = other DNA
cttgaggggtttgtccttga

SEQ ID NO: 7 - DNA, 23 bp, synthetic construct
  source 1..23: mol_type = other DNA
ctcaccagttcctgctttgatgt

SEQ ID NO: 8 - DNA, 22 bp, synthetic construct
  source 1..22: mol_type = other DNA
aggagagactggaggaaaagtc

SEQ ID NO: 9 - DNA, 25 bp, synthetic construct
  source 1..25: mol_type = other DNA
cttaaacttcagtggcttgtctctg

SEQ ID NO: 10 - DNA, 21 bp, synthetic construct
  source 1..21: mol_type = other DNA
cgggggacataaaagttattg

SEQ ID NO: 11 - DNA, 22 bp, synthetic construct
  source 1..22: mol_type = other DNA
tgcattgttttaccagtgtcaa

SEQ ID NO: 12 - DNA, 25 bp, synthetic construct
  source 1..25: mol_type = other DNA
tgaaggagtcttctatccgatctgt

SEQ ID NO: 13 - DNA, 21 bp, synthetic construct
  source 1..21: mol_type = other DNA
cacttccttgacctcgatgct

SEQ ID NO: 14 - DNA, 26 bp, synthetic construct
  source 1..26: mol_type = other DNA
agaccgtactccccatcaagaagcgc

SEQ ID NO: 15 - DNA, 20 bp, synthetic construct
  source 1..20: mol_type = other DNA
gtgcgcgcgagcccgaaatc

SEQ ID NO: 16 - DNA, 20 bp, synthetic construct
  source 1..20: mol_type = other DNA
ctctccgagaggagggagcg

SEQ ID NO: 17 - DNA, 20 bp, synthetic construct
  source 1..20: mol_type = other DNA
gccattttccggacggcttt

SEQ ID NO: 18 - DNA, 20 bp, synthetic construct
  source 1..20: mol_type = other DNA
tctctcctcctcgcctcctc

SEQ ID NO: 19 - DNA, 20 bp, synthetic construct
  source 1..20: mol_type = other DNA
acccccgccccccggcaagg

SEQ ID NO: 20 - DNA, 20 bp, synthetic construct
  source 1..20: mol_type = other DNA
agagacctcaacttgtcacg

SEQ ID NO: 21 - DNA, 20 bp, synthetic construct
  source 1..20: mol_type = other DNA
cattaagataaccatcattt

SEQ ID NO: 22 - DNA, 20 bp, synthetic construct
  source 1..20: mol_type = other DNA
ggaactggtgagtctgtatt

SEQ ID NO: 23 - DNA, 20 bp, synthetic construct
  source 1..20: mol_type = other DNA
gaagcaaggtgtattctggg

SEQ ID NO: 24 - DNA, 20 bp, synthetic construct
  source 1..20: mol_type = other DNA
ctaccatggaatcctgttgg

SEQ ID NO: 25 - DNA, 20 bp, synthetic construct
  source 1..20: mol_type = other DNA
ttttctataaatccatgtat

SEQ ID NO: 26 - DNA, 20 bp, synthetic construct
  source 1..20: mol_type = other DNA
tagccccactcccggataag

SEQ ID NO: 27 - DNA, 20 bp, synthetic construct
  source 1..20: mol_type = other DNA
actcaagcccaaggagttca

SEQ ID NO: 28 - DNA, 20 bp, synthetic construct
  source 1..20: mol_type = other DNA
gctttaatgctttattttta

SEQ ID NO: 29 - DNA, 20 bp, synthetic construct
  source 1..20: mol_type = other DNA
tgccaacagcaggcccagcg

SEQ ID NO: 30 - DNA, 20 bp, synthetic construct
  source 1..20: mol_type = other DNA
gatatcagtgaggaagttgt

SEQ ID NO: 31 - DNA, 20 bp, synthetic construct
  source 1..20: mol_type = other DNA
cgtgccatggaagtccttcc

SEQ ID NO: 32 - DNA, 20 bp, synthetic construct
  source 1..20: mol_type = other DNA
ggtgagctgatgctatatga

SEQ ID NO: 33 - DNA, 20 bp, synthetic construct
  source 1..20: mol_type = other DNA
aggcggcagtggcttacgcc

SEQ ID NO: 34 - DNA, 20 bp, synthetic construct
  source 1..20: mol_type = other DNA
agccccttaattttgttctc

SEQ ID NO: 35 - DNA, 20 bp, synthetic construct
  source 1..20: mol_type = other DNA
tggcggctcaagaaccagcc

SEQ ID NO: 36 - DNA, 20 bp, synthetic construct
  source 1..20: mol_type = other DNA
caaatattagaatagactca

SEQ ID NO: 37 - DNA, 20 bp, synthetic construct
  source 1..20: mol_type = other DNA
tgggactcagattctatagg

SEQ ID NO: 38 - DNA, 20 bp, synthetic construct
  source 1..20: mol_type = other DNA
gtcctggaacgacaggcttg

SEQ ID NO: 39 - DNA, 20 bp, synthetic construct
  source 1..20: mol_type = other DNA
ccaaatttataacttaagaa

SEQ ID NO: 40 - DNA, 20 bp, synthetic construct
  source 1..20: mol_type = other DNA
ggtgatgtgtattttactac

SEQ ID NO: 41 - DNA, 20 bp, synthetic construct
  source 1..20: mol_type = other DNA
tggtgggacaaaaattgtgg

SEQ ID NO: 42 - DNA, 20 bp, synthetic construct
  source 1..20: mol_type = other DNA
aaataagcatctggcatttg

SEQ ID NO: 43 - DNA, 20 bp, synthetic construct
  source 1..20: mol_type = other DNA
tacattgaaaaacagccaga

SEQ ID NO: 44 - DNA, 20 bp, synthetic construct
  source 1..20: mol_type = other DNA
ggatccatgcgagagaagca

SEQ ID NO: 45 - DNA, 20 bp, synthetic construct
  source 1..20: mol_type = other DNA
tataatatcattcagcctca

SEQ ID NO: 46 - DNA, 20 bp, synthetic construct
  source 1..20: mol_type = other DNA
cagcaggaagagtccagaga

SEQ ID NO: 47 - DNA, 20 bp, synthetic construct
  source 1..20: mol_type = other DNA
agaaacctgccaggtgtggt

SEQ ID NO: 48 - DNA, 20 bp, synthetic construct
  source 1..20: mol_type = other DNA
ccaggtgtggcccagggtgg

SEQ ID NO: 49 - DNA, 20 bp, synthetic construct
  source 1..20: mol_type = other DNA
ggcatcctacaacccacaga

SEQ ID NO: 50 - DNA, 20 bp, synthetic construct
  source 1..20: mol_type = other DNA
tgacttttcttccctgagcc

SEQ ID NO: 51 - DNA, 20 bp, synthetic construct
  source 1..20: mol_type = other DNA
gggtttgtccttgaggccct

SEQ ID NO: 52 - DNA, 20 bp, synthetic construct
  source 1..20: mol_type = other DNA
ctcttctttcttatctttct

SEQ ID NO: 53 - DNA, 20 bp, synthetic construct
  source 1..20: mol_type = other DNA
cttgccctctttctcttctt

SEQ ID NO: 54 - DNA, 20 bp, synthetic construct
  source 1..20: mol_type = other DNA
tggctgcacgggctcatgct

SEQ ID NO: 55 - DNA, 20 bp, synthetic construct
  source 1..20: mol_type = other DNA
gtggtgggctgatggctgca

SEQ ID NO: 56 - DNA, 20 bp, synthetic construct
  source 1..20: mol_type = other DNA
ctgctttgcctgcctctgcg

SEQ ID NO: 57 - DNA, 20 bp, synthetic construct
  source 1..20: mol_type = other DNA
aagcttccggcacagccggg

SEQ ID NO: 58 - DNA, 20 bp, synthetic construct
  source 1..20: mol_type = other DNA
ggtcacggatgatggagcgc

SEQ ID NO: 59 - DNA, 20 bp, synthetic construct
  source 1..20: mol_type = other DNA
ttccgtgtccagccttcagg

SEQ ID NO: 60 - DNA, 20 bp, synthetic construct
  source 1..20: mol_type = other DNA
cagcagagcggccagatttc

SEQ ID NO: 61 - DNA, 20 bp, synthetic construct
  source 1..20: mol_type = other DNA
tgtccctgccctccctgccc

SEQ ID NO: 62 - DNA, 20 bp, synthetic construct
  source 1..20: mol_type = other DNA
gcgaaaggcttttccctggg

SEQ ID NO: 63 - DNA, 20 bp, synthetic construct
  source 1..20: mol_type = other DNA
gaagtacgcaatcaactcca

SEQ ID NO: 64 - DNA, 20 bp, synthetic construct
  source 1..20: mol_type = other DNA
gggatgtgtcgcctaccttt

SEQ ID NO: 65 - DNA, 20 bp, synthetic construct
  source 1..20: mol_type = other DNA
cgtgaagtcaaaatcattag

SEQ ID NO: 66 - DNA, 20 bp, synthetic construct
  source 1..20: mol_type = other DNA
ttaggtggtttctgctctcg

SEQ ID NO: 67 - DNA, 20 bp, synthetic construct
  source 1..20: mol_type = other DNA
cggcctctgccagttcctgg

SEQ ID NO: 68 - DNA, 20 bp, synthetic construct
  source 1..20: mol_type = other DNA
acccttttcacctgcacacc

SEQ ID NO: 69 - DNA, 20 bp, synthetic construct
  source 1..20: mol_type = other DNA
aaggagcttcccaggacttt

SEQ ID NO: 70 - DNA, 20 bp, synthetic construct
  source 1..20: mol_type = other DNA
tggcgaagtttgaaaaggca

SEQ ID NO: 71 - DNA, 20 bp, synthetic construct
  source 1..20: mol_type = other DNA
agccttgccccctggcgaag

SEQ ID NO: 72 - DNA, 20 bp, synthetic construct
  source 1..20: mol_type = other DNA
tggatgtggtggccccaccc

SEQ ID NO: 73 - DNA, 20 bp, synthetic construct
  source 1..20: mol_type = other DNA
gcttttcgcttcctgccggg

SEQ ID NO: 74 - DNA, 20 bp, synthetic construct
  source 1..20: mol_type = other DNA
gtttcttgggaatggcctga

SEQ ID NO: 75 - DNA, 20 bp, synthetic construct
  source 1..20: mol_type = other DNA
ggctgccaccacactccccg

SEQ ID NO: 76 - DNA, 20 bp, synthetic construct
  source 1..20: mol_type = other DNA
ttcacggctttctttttggc

SEQ ID NO: 77 - DNA, 20 bp, synthetic construct
  source 1..20: mol_type = other DNA
ctcctgcacagatcggatag

SEQ ID NO: 78 - DNA, 20 bp, synthetic construct
  source 1..20: mol_type = other DNA
tctcccgggtcttgcgcttc

SEQ ID NO: 79 - DNA, 20 bp, synthetic construct
  source 1..20: mol_type = other DNA
cttcaccacttccttgacct

SEQ ID NO: 80 - DNA, 20 bp, synthetic construct
  source 1..20: mol_type = other DNA
tcagtcctttcccgctcttc

SEQ ID NO: 81 - DNA, 20 bp, synthetic construct
  source 1..20: mol_type = other DNA
cttgcttttccgcccagggc

SEQ ID NO: 82 - DNA, 20 bp, synthetic construct
  source 1..20: mol_type = other DNA
ggtgatggtggtggtgctcc

SEQ ID NO: 83 - DNA, 20 bp, synthetic construct
  source 1..20: mol_type = other DNA
gctgctgctcaagtcctggg

SEQ ID NO: 84 - DNA, 20 bp, synthetic construct
  source 1..20: mol_type = other DNA
ctccagtgagcctcctctgg

SEQ ID NO: 85 - DNA, 20 bp, synthetic construct
  source 1..20: mol_type = other DNA
aaccgcgggctgagtcttag

SEQ ID NO: 86 - DNA, 20 bp, synthetic construct
  source 1..20: mol_type = other DNA
tggcggcggtggcaaccgcg

SEQ ID NO: 87 - DNA, 20 bp, synthetic construct
  source 1..20: mol_type = other DNA
ttttctgcggccgtggcggc

SEQ ID NO: 88 - DNA, 20 bp, synthetic construct
  source 1..20: mol_type = other DNA
ctctccctcccctcggtgtt

SEQ ID NO: 89 - DNA, 20 bp, synthetic construct
  source 1..20: mol_type = other DNA
cttggcatggaggatgaaac

SEQ ID NO: 90 - DNA, 20 bp, synthetic construct
  source 1..20: mol_type = other DNA
tccggctgtccacaggctcc

SEQ ID NO: 91 - DNA, 20 bp, synthetic construct
  source 1..20: mol_type = other DNA
aatccgctccgtgtaaagtc

SEQ ID NO: 92 - DNA, 20 bp, synthetic construct
  source 1..20: mol_type = other DNA
cagctgcctttattcttgtt

SEQ ID NO: 93 - DNA, 20 bp, synthetic construct
  source 1..20: mol_type = other DNA
gtcagagccctacccataag

SEQ ID NO: 94 - DNA, 20 bp, synthetic construct
  source 1..20: mol_type = other DNA
agcgcgcgcgccgccgacgc

SEQ ID NO: 95 - DNA, 20 bp, synthetic construct
  source 1..20: mol_type = other DNA
cttttaccacagccctctct

SEQ ID NO: 96 - DNA, 20 bp, synthetic construct
  source 1..20: mol_type = other DNA
ccgctcggcgcggcggcggc

SEQ ID NO: 97 - DNA, 20 bp, synthetic construct
  source 1..20: mol_type = other DNA
tcagtttgggtgattcggtc

SEQ ID NO: 98 - DNA, 20 bp, synthetic construct
  source 1..20: mol_type = other DNA
cagcacagcgggaacacatt

SEQ ID NO: 99 - DNA, 20 bp, synthetic construct
  source 1..20: mol_type = other DNA
tatttttatggagcagtctc

SEQ ID NO: 100 - DNA, 20 bp, synthetic construct
  source 1..20: mol_type = other DNA
atgtcacatcaaagcaggaa

SEQ ID NO: 101 - DNA, 20 bp, synthetic construct
  source 1..20: mol_type = other DNA
ttggagctggtctacagaag

SEQ ID NO: 102 - DNA, 20 bp, synthetic construct
  source 1..20: mol_type = other DNA
agccctaacatcccagctac

SEQ ID NO: 103 - DNA, 20 bp, synthetic construct
  source 1..20: mol_type = other DNA
cacactgacctttcagggct

SEQ ID NO: 104 - DNA, 20 bp, synthetic construct
  source 1..20: mol_type = other DNA
taaaaaaggatttcctaagt

SEQ ID NO: 105 - DNA, 20 bp, synthetic construct
  source 1..20: mol_type = other DNA
gtacacacacgctttttttt

SEQ ID NO: 106 - DNA, 20 bp, synthetic construct
  source 1..20: mol_type = other DNA
gaaagccgagcctggccggg

SEQ ID NO: 107 - DNA, 20 bp, synthetic construct
  source 1..20: mol_type = other DNA
gaagaaaatgtggatttttt

SEQ ID NO: 108 - DNA, 20 bp, synthetic construct
  source 1..20: mol_type = other DNA
cgagaatgagactccgtatc

SEQ ID NO: 109 - DNA, 20 bp, synthetic construct
  source 1..20: mol_type = other DNA
aaacccaaaccaccttaccc

SEQ ID NO: 110 - DNA, 20 bp, synthetic construct
  source 1..20: mol_type = other DNA
aaaataaagtcaggaggctg

SEQ ID NO: 111 - DNA, 20 bp, synthetic construct
  source 1..20: mol_type = other DNA
aaaaatggagggcacagtgg

SEQ ID NO: 112 - DNA, 20 bp, synthetic construct
  source 1..20: mol_type = other DNA
ggtttttctcctttattatc

SEQ ID NO: 113 - DNA, 20 bp, synthetic construct
  source 1..20: mol_type = other DNA
tatgttggcctagaactcct

SEQ ID NO: 114 - DNA, 20 bp, synthetic construct
  source 1..20: mol_type = other DNA
tgctctcatattcacccacg

SEQ ID NO: 115 - DNA, 20 bp, synthetic construct
  source 1..20: mol_type = other DNA
gtgcagagactcaagggagg

SEQ ID NO: 116 - DNA, 20 bp, synthetic construct
  source 1..20: mol_type = other DNA
gctaagcctcctggtgaacc

SEQ ID NO: 117 - DNA, 20 bp, synthetic construct
  source 1..20: mol_type = other DNA
gtatgaacatcagctgacgc

SEQ ID NO: 118 - DNA, 20 bp, synthetic construct
  source 1..20: mol_type = other DNA
aggcgcgctggtgcaagcct

SEQ ID NO: 119 - DNA, 20 bp, synthetic construct
  source 1..20: mol_type = other DNA
cagccactctttttttttga

SEQ ID NO: 120 - DNA, 20 bp, synthetic construct
  source 1..20: mol_type = other DNA
gtacctgggaggaactacaa

SEQ ID NO: 121 - DNA, 20 bp, synthetic construct
  source 1..20: mol_type = other DNA
agggcgagagatccaggact

SEQ ID NO: 122 - DNA, 20 bp, synthetic construct
  source 1..20: mol_type = other DNA
ggattagggaattagatgca

SEQ ID NO: 123 - DNA, 20 bp, synthetic construct
  source 1..20: mol_type = other DNA
ggaaagcctgtcttttaaaa

SEQ ID NO: 124 - DNA, 20 bp, synthetic construct
  source 1..20: mol_type = other DNA
ccagatggtgtttccaattc

SEQ ID NO: 125 - DNA, 20 bp, synthetic construct
  source 1..20: mol_type = other DNA
acttctagaccgggcgcagt

SEQ ID NO: 126 - DNA, 20 bp, synthetic construct
  source 1..20: mol_type = other DNA
gtacaatgaatgaacttttt

SEQ ID NO: 127 - DNA, 20 bp, synthetic construct
  source 1..20: mol_type = other DNA
caaacatatctactgcattc

SEQ ID NO: 128 - DNA, 20 bp, synthetic construct
  source 1..20: mol_type = other DNA
acaggtaaccccatctaggc

SEQ ID NO: 129 - DNA, 20 bp, synthetic construct
  source 1..20: mol_type = other DNA
ggaggtcctggtcttctgac

SEQ ID NO: 130 - DNA, 20 bp, synthetic construct
  source 1..20: mol_type = other DNA
ttatctttcttcaccttttt

SEQ ID NO: 131 - DNA, 20 bp, synthetic construct
  source 1..20: mol_type = other DNA
ctctttctcttctttcttat

SEQ ID NO: 132 - DNA, 20 bp, synthetic construct
  source 1..20: mol_type = other DNA
cacgggctcatgcttgccct

SEQ ID NO: 133 - DNA, 20 bp, synthetic construct
  source 1..20: mol_type = other DNA
ggctgatggctgcacgggct

SEQ ID NO: 134 - DNA, 20 bp, synthetic construct
  source 1..20: mol_type = other DNA
tgcgggctcagcagagtggt

SEQ ID NO: 135 - DNA, 20 bp, synthetic construct
  source 1..20: mol_type = other DNA
gacccttctgatgtctctgc

SEQ ID NO: 136 - DNA, 20 bp, synthetic construct
  source 1..20: mol_type = other DNA
aggcagaagcttccggcaca

SEQ ID NO: 137 - DNA, 20 bp, synthetic construct
  source 1..20: mol_type = other DNA
tcatacatgggtccccggtc

SEQ ID NO: 138 - DNA, 20 bp, synthetic construct
  source 1..20: mol_type = other DNA
tttcctttgcttaagcttcc

SEQ ID NO: 139 - DNA, 20 bp, synthetic construct
  source 1..20: mol_type = other DNA
tacacatcatacttcccagc

SEQ ID NO: 140 - DNA, 20 bp, synthetic construct
  source 1..20: mol_type = other DNA
tcaactccactttagagcga

SEQ ID NO: 141 - DNA, 20 bp, synthetic construct
  source 1..20: mol_type = other DNA
gcctaccttttcgaagtacg

SEQ ID NO: 142 - DNA, 20 bp, synthetic construct
  source 1..20: mol_type = other DNA
ggtccagggatgtgtcgcct

SEQ ID NO: 143 - DNA, 20 bp, synthetic construct
  source 1..20: mol_type = other DNA
tccctctcccagttaccgtg

SEQ ID NO: 144 - DNA, 20 bp, synthetic construct
  source 1..20: mol_type = other DNA
tggagctttgggagatttgg

SEQ ID NO: 145 - DNA, 20 bp, synthetic construct
  source 1..20: mol_type = other DNA
cgtggccgccttgggtctcg

SEQ ID NO: 146 - DNA, 20 bp, synthetic construct
  source 1..20: mol_type = other DNA
aggacttttctccaggaccc

SEQ ID NO: 147 - DNA, 20 bp, synthetic construct
  source 1..20: mol_type = other DNA
aggcatcttgacaaggagct

SEQ ID NO: 148 - DNA, 20 bp, synthetic construct
  source 1..20: mol_type = other DNA
gccccctggcgaagtttgaa

SEQ ID NO: 149 - DNA, 20 bp, synthetic construct
  source 1..20: mol_type = other DNA
ccccacccccctcagccttg

SEQ ID NO: 150 - DNA, 20 bp, synthetic construct
  source 1..20: mol_type = other DNA
ccatgacctgggtggatgtg

SEQ ID NO: 151 - DNA, 20 bp, synthetic construct
  source 1..20: mol_type = other DNA
ctgagggtcggcctcagctt

SEQ ID NO: 152 - DNA, 20 bp, synthetic construct
  source 1..20: mol_type = other DNA
cccggctttcggccccgttt

SEQ ID NO: 153 - DNA, 20 bp, synthetic construct
  source 1..20: mol_type = other DNA
tggcctcggcggcagcggct

SEQ ID NO: 154 - DNA, 20 bp, synthetic construct
  source 1..20: mol_type = other DNA
tcggatagaagactccttca

SEQ ID NO: 155 - DNA, 20 bp, synthetic construct
  source 1..20: mol_type = other DNA
tacggtctcctgcacagatc

SEQ ID NO: 156 - DNA, 20 bp, synthetic construct
  source 1..20: mol_type = other DNA
acctcgatgctgaccgtctc

SEQ ID NO: 157 - DNA, 20 bp, synthetic construct
  source 1..20: mol_type = other DNA
cttctcaccgagggtggaca

SEQ ID NO: 158 - DNA, 20 bp, synthetic construct
  source 1..20: mol_type = other DNA
gggctcttacaggtcttcag

SEQ ID NO: 159 - DNA, 20 bp, synthetic construct
  source 1..20: mol_type = other DNA
ctgctgctgcgccccttggg

SEQ ID NO: 160 - DNA, 20 bp, synthetic construct
  source 1..20: mol_type = other DNA
tcgggctcaggtggaggtgg

SEQ ID NO: 161 - DNA, 20 bp, synthetic construct
  source 1..20: mol_type = other DNA
ctgggcatcttctcctcttt

SEQ ID NO: 162 - DNA, 20 bp, synthetic construct
  source 1..20: mol_type = other DNA
gtcttagctggctccttggg

SEQ ID NO: 163 - DNA, 20 bp, synthetic construct
  source 1..20: mol_type = other DNA
ggtggcaaccgcgggctgag

SEQ ID NO: 164 - DNA, 20 bp, synthetic construct
  source 1..20: mol_type = other DNA
cggccgtggcggcggtggca

SEQ ID NO: 165 - DNA, 20 bp, synthetic construct
  source 1..20: mol_type = other DNA
tgtttgtacttttctgcggc

SEQ ID NO: 166 - DNA, 20 bp, synthetic construct
  source 1..20: mol_type = other DNA
aaacaatgtctttgcgctct

SEQ ID NO: 167 - DNA, 20 bp, synthetic construct
  source 1..20: mol_type = other DNA
ctcctctctgtttggccttg

SEQ ID NO: 168 - DNA, 20 bp, synthetic construct
  source 1..20: mol_type = other DNA
agtcagctaactctctcggt

SEQ ID NO: 169 - DNA, 20 bp, synthetic construct
  source 1..20: mol_type = other DNA
tgttggtttgctttgcaatc

SEQ ID NO: 170 - DNA, 20 bp, synthetic construct
  source 1..20: mol_type = other DNA
taaggagaagagacaacagc

SEQ ID NO: 171 - DNA, 20 bp, synthetic construct
  source 1..20: mol_type = other DNA
ttaatcgggaagctttgtca

SEQ ID NO: 172 - DNA, 20 bp, synthetic construct
  source 1..20: mol_type = other DNA
gaggagggagcgcgcgcgcc

SEQ ID NO: 173 - DNA, 20 bp, synthetic construct
  source 1..20: mol_type = other DNA
ccggacggcttttaccacag

SEQ ID NO: 174 - DNA, 20 bp, synthetic construct
  source 1..20: mol_type = other DNA
ctcctcctccgctcggcgcg

SEQ ID NO: 175 - DNA, 20 bp, synthetic construct
  source 1..20: mol_type = other DNA
atgcttcatttttacagtat

SEQ ID NO: 176 - DNA, 20 bp, synthetic construct
  source 1..20: mol_type = other DNA
gagccagaggctgggtgcgg

SEQ ID NO: 177 - DNA, 20 bp, synthetic construct
  source 1..20: mol_type = other DNA
tgagtctgtatttttatgga

SEQ ID NO: 178 - DNA, 20 bp, synthetic construct
  source 1..20: mol_type = other DNA
ggagtcacatgtcacatcaa

SEQ ID NO: 179 - DNA, 20 bp, synthetic construct
  source 1..20: mol_type = other DNA
gaatcctgttggagctggtc

SEQ ID NO: 180 - DNA, 20 bp, synthetic construct
  source 1..20: mol_type = other DNA
cttccctgagccctaacatc

SEQ ID NO: 181 - DNA, 20 bp, synthetic construct
  source 1..20: mol_type = other DNA
cccacagcagtaaaagagaa

SEQ ID NO: 182 - DNA, 20 bp, synthetic construct
  source 1..20: mol_type = other DNA
accccagtagttgagattac

SEQ ID NO: 183 - DNA, 20 bp, synthetic construct
  source 1..20: mol_type = other DNA
atagtagttgccagagggtg

SEQ ID NO: 184 - DNA, 20 bp, synthetic construct
  source 1..20: mol_type = other DNA
ggcttctattgtaaaactat

SEQ ID NO: 185 - DNA, 20 bp, synthetic construct
  source 1..20: mol_type = other DNA
actggtttttaagagatggg

SEQ ID NO: 186 - DNA, 20 bp, synthetic construct
  source 1..20: mol_type = other DNA
taaaatctatgggaataaaa

SEQ ID NO: 187 - DNA, 20 bp, synthetic construct
  source 1..20: mol_type = other DNA
gaaatgtgggcttggcatgg

SEQ ID NO: 188 - DNA, 20 bp, synthetic construct
  source 1..20: mol_type = other DNA
aacatggtttagtagaaacc

SEQ ID NO: 189 - DNA, 20 bp, synthetic construct
  source 1..20: mol_type = other DNA
ggtattataattttgtaatt

SEQ ID NO: 190 - DNA, 20 bp, synthetic construct
  source 1..20: mol_type = other DNA
caacattccatttatttagg

SEQ ID NO: 191 - DNA, 20 bp, synthetic construct
  source 1..20: mol_type = other DNA
attttcaccctttaaaaatc

SEQ ID NO: 192 - DNA, 20 bp, synthetic construct
  source 1..20: mol_type = other DNA
taatacagtgacaagcatcc

SEQ ID NO: 193 - DNA, 20 bp, synthetic construct
  source 1..20: mol_type = other DNA
tccatcttgcaggtggagta

SEQ ID NO: 194 - DNA, 20 bp, synthetic construct
  source 1..20: mol_type = other DNA
gaagccaaaaaagcaacaaa

SEQ ID NO: 195 - DNA, 20 bp, synthetic construct
  source 1..20: mol_type = other DNA
ccaagacaaggaaaaacggg

SEQ ID NO: 196 - DNA, 20 bp, synthetic construct
  source 1..20: mol_type = other DNA
ctagctatcagctgggcatg

SEQ ID NO: 197 - DNA, 20 bp, synthetic construct
  source 1..20: mol_type = other DNA
tgccttgttgggtagtacag

SEQ ID NO: 198 - DNA, 20 bp, synthetic construct
  source 1..20: mol_type = other DNA
gctaagttagaactccgtgg

SEQ ID NO: 199 - DNA, 20 bp, synthetic construct
  source 1..20: mol_type = other DNA
acacgcctgtaatcctgcat

SEQ ID NO: 200 - DNA, 20 bp, synthetic construct
  source 1..20: mol_type = other DNA
caactggaggccgggcgcga

SEQ ID NO: 201 - DNA, 20 bp, synthetic construct
  source 1..20: mol_type = other DNA
agcccacacagctgtctcag

SEQ ID NO: 202 - DNA, 20 bp, synthetic construct
  source 1..20: mol_type = other DNA
ttcctcatgaatgtgacctg

SEQ ID NO: 203 - DNA, 20 bp, synthetic construct
  source 1..20: mol_type = other DNA
gaggaacttgtctgagatca

SEQ ID NO: 204 - DNA, 20 bp, synthetic construct
  source 1..20: mol_type = other DNA
cagctactcgctagaaaggg

SEQ ID NO: 205 - DNA, 20 bp, synthetic construct
  source 1..20: mol_type = other DNA
ctccccataaaggagggagg

SEQ ID NO: 206 - DNA, 20 bp, synthetic construct
  source 1..20: mol_type = other DNA
ccatcatacactcagatctt

SEQ ID NO: 207 - DNA, 20 bp, synthetic construct
  source 1..20: mol_type = other DNA
ttgaggccctggaggtcctg

SEQ ID NO: 208 - DNA, 20 bp, synthetic construct
  source 1..20: mol_type = other DNA
ttctttcttatctttcttca

SEQ ID NO: 209 - DNA, 20 bp, synthetic construct
  source 1..20: mol_type = other DNA
gccctctttctcttctttct

SEQ ID NO: 210 - DNA, 20 bp, synthetic construct
  source 1..20: mol_type = other DNA
ctgcacgggctcatgcttgc

SEQ ID NO: 211 - DNA, 20 bp, synthetic construct
  source 1..20: mol_type = other DNA
gtgggctgatggctgcacgg

SEQ ID NO: 212 - DNA, 20 bp, synthetic construct
  source 1..20: mol_type = other DNA
cctgcctctgcgggctcagc

SEQ ID NO: 213 - DNA, 20 bp, synthetic construct
  source 1..20: mol_type = other DNA
cggagcctgacccttctgat

SEQ ID NO: 214 - DNA, 20 bp, synthetic construct
  source 1..20: mol_type = other DNA
gggaggcagaagcttccggc

SEQ ID NO: 215 - DNA, 20 bp, synthetic construct
  source 1..20: mol_type = other DNA
ccagccttcaggcagggtgg

SEQ ID NO: 216 - DNA, 20 bp, synthetic construct
  source 1..20: mol_type = other DNA
cggccagatttcctttgctt

SEQ ID NO: 217 - DNA, 20 bp, synthetic construct
  source 1..20: mol_type = other DNA
tgatcaaatacacatcatac

SEQ ID NO: 218 - DNA, 20 bp, synthetic construct
  source 1..20: mol_type = other DNA
gtacgcaatcaactccactt

SEQ ID NO: 219 - DNA, 20 bp, synthetic construct
  source 1..20: mol_type = other DNA
tgtgtcgcctaccttttcga

SEQ ID NO: 220 - DNA, 20 bp, synthetic construct
  source 1..20: mol_type = other DNA
caaaatcattagggtccagg

SEQ ID NO: 221 - DNA, 20 bp, synthetic construct
  source 1..20: mol_type = other DNA
tttctgctctcgccgggagg

SEQ ID NO: 222 - DNA, 20 bp, synthetic construct
  source 1..20: mol_type = other DNA
ccagttcctggagctttggg

SEQ ID NO: 223 - DNA, 20 bp, synthetic construct
  source 1..20: mol_type = other DNA
cacctgcacaccctctgacg

SEQ ID NO: 224 - DNA, 20 bp, synthetic construct
  source 1..20: mol_type = other DNA
gagcttcccaggacttttct

SEQ ID NO: 225 - DNA, 20 bp, synthetic construct
  source 1..20: mol_type = other DNA
gtttgaaaaggcatcttgac

SEQ ID NO: 226 - DNA, 20 bp, synthetic construct
  source 1..20: mol_type = other DNA
cttgccccctggcgaagttt

SEQ ID NO: 227 - DNA, 20 bp, synthetic construct
  source 1..20: mol_type = other DNA
tgtggtggccccacccccct

SEQ ID NO: 228 - DNA, 20 bp, synthetic construct
  source 1..20: mol_type = other DNA
tttgatcaccatgacctggg

SEQ ID NO: 229 - DNA, 20 bp, synthetic construct
  source 1..20: mol_type = other DNA
ggaatggcctgagggtcggc

SEQ ID NO: 230 - DNA, 20 bp, synthetic construct
  source 1..20: mol_type = other DNA
ccacactccccggctttcgg

SEQ ID NO: 231 - DNA, 20 bp, synthetic construct
  source 1..20: mol_type = other DNA
tttctttttggcctcggcgg

SEQ ID NO: 232 - DNA, 20 bp, synthetic construct
  source 1..20: mol_type = other DNA
ctgcacagatcggatagaag

SEQ ID NO: 233 - DNA, 20 bp, synthetic construct
  source 1..20: mol_type = other DNA
gtcttgcgcttcttgatggg

SEQ ID NO: 234 - DNA, 20 bp, synthetic construct
  source 1..20: mol_type = other DNA
cttccttgacctcgatgctg

SEQ ID NO: 235 - DNA, 20 bp, synthetic construct
  source 1..20: mol_type = other DNA
ttcccgctcttctcaccgag

SEQ ID NO: 236 - DNA, 20 bp, synthetic construct
  source 1..20: mol_type = other DNA
tccgcccagggctcttacag

SEQ ID NO: 237 - DNA, 20 bp, synthetic construct
  source 1..20: mol_type = other DNA
tggtggtgctccttcttggg

SEQ ID NO: 238 - DNA, 20 bp, synthetic construct
  source 1..20: mol_type = other DNA
cggagctctcgggctcaggt

SEQ ID NO: 239 - DNA, 20 bp, synthetic construct
  source 1..20: mol_type = other DNA
agcctcctctgggcatcttc

SEQ ID NO: 240 - DNA, 20 bp, synthetic construct
  source 1..20: mol_type = other DNA
cgggctgagtcttagctggc

SEQ ID NO: 241 - DNA, 20 bp, synthetic construct
  source 1..20: mol_type = other DNA
ggcggtggcaaccgcgggct

SEQ ID NO: 242 - DNA, 20 bp, synthetic construct
  source 1..20: mol_type = other DNA
tctgcggccgtggcggcggt

SEQ ID NO: 243 - DNA, 20 bp, synthetic construct
  source 1..20: mol_type = other DNA
cccctcggtgtttgtacttt

SEQ ID NO: 244 - DNA, 20 bp, synthetic construct
  source 1..20: mol_type = other DNA
ggaggatgaaacaatgtctt

SEQ ID NO: 245 - DNA, 20 bp, synthetic construct
  source 1..20: mol_type = other DNA
tccacaggctcctctctgtt

SEQ ID NO: 246 - DNA, 20 bp, synthetic construct
  source 1..20: mol_type = other DNA
ccgtgtaaagtcagctaact

SEQ ID NO: 247 - DNA, 20 bp, synthetic construct
  source 1..20: mol_type = other DNA
tttattcttgttggtttgct

SEQ ID NO: 248 - DNA, 20 bp, synthetic construct
  source 1..20: mol_type = other DNA
cctacccataaggagaagag

SEQ ID NO: 249 - DNA, 20 bp, synthetic construct
  source 1..20: mol_type = other DNA
tatttcagttaatcgggaag

SEQ ID NO: 250 - DNA, 20 bp, synthetic construct
  source 1..20: mol_type = other DNA
acagccctctctccgagagg

SEQ ID NO: 251 - DNA, 20 bp, synthetic construct
  source 1..20: mol_type = other DNA
cggcggcggccattttccgg

SEQ ID NO: 252 - DNA, 20 bp, synthetic construct
  source 1..20: mol_type = other DNA
tggagcagtctctcctcctc

SEQ ID NO: 253 - DNA, 20 bp, synthetic construct
  source 1..20: mol_type = other DNA
ttcatggaatgggcgagaag

SEQ ID NO: 254 - DNA, 20 bp, synthetic construct
  source 1..20: mol_type = other DNA
acagaggcagggcaggcacg

SEQ ID NO: 255 - DNA, 20 bp, synthetic construct
  source 1..20: mol_type = other DNA
aagattcatgcttgttagaa

SEQ ID NO: 256 - DNA, 20 bp, synthetic construct
  source 1..20: mol_type = other DNA
tcaaagcaggaactggtgag

SEQ ID NO: 257 - DNA, 20 bp, synthetic construct
  source 1..20: mol_type = other DNA
ggtctacagaagcaaggtgt

SEQ ID NO: 258 - DNA, 20 bp, synthetic construct
  source 1..20: mol_type = other DNA
catcccagctaccatggaat

SEQ ID NO: 259 - DNA, 20 bp, synthetic construct
  source 1..20: mol_type = other DNA
caccatcctgaggccaggca

SEQ ID NO: 260 - DNA, 20 bp, synthetic construct
  source 1..20: mol_type = other DNA
taacttttttctattattat

SEQ ID NO: 261 - DNA, 20 bp, synthetic construct
  source 1..20: mol_type = other DNA
acagtcacagaacaacaaag

SEQ ID NO: 262 - DNA, 20 bp, synthetic construct
  source 1..20: mol_type = other DNA
ggcctaattttttatctttg

SEQ ID NO: 263 - DNA, 20 bp, synthetic construct
  source 1..20: mol_type = other DNA
acagggttgtagccatcagc

SEQ ID NO: 264 - DNA, 20 bp, synthetic construct
  source 1..20: mol_type = other DNA
gatcactggaacacaatggt

SEQ ID NO: 265 - DNA, 20 bp, synthetic construct
  source 1..20: mol_type = other DNA
ggaagagaaaagaagggcac

SEQ ID NO: 266 - DNA, 20 bp, synthetic construct
  source 1..20: mol_type = other DNA
catttaataaataaatccct

SEQ ID NO: 267 - DNA, 20 bp, synthetic construct
  source 1..20: mol_type = other DNA
tttaccagtgccatttttcc

SEQ ID NO: 268 - DNA, 20 bp, synthetic construct
  source 1..20: mol_type = other DNA
cagcaaatttctgtggtttt

SEQ ID NO: 269 - DNA, 20 bp, synthetic construct
  source 1..20: mol_type = other DNA
gctctcagaccagaccagac

SEQ ID NO: 270 - DNA, 20 bp, synthetic construct
  source 1..20: mol_type = other DNA
acagctgatgaggagggtgg

SEQ ID NO: 271 - DNA, 20 bp, synthetic construct
  source 1..20: mol_type = other DNA
tacacaaatactaagccaca

SEQ ID NO: 272 - DNA, 20 bp, synthetic construct
  source 1..20: mol_type = other DNA
actgccaccaccatgactaa

SEQ ID NO: 273 - DNA, 20 bp, synthetic construct
  source 1..20: mol_type = other DNA
gttagaagttgattttttct

SEQ ID NO: 274 - DNA, 20 bp, synthetic construct
  source 1..20: mol_type = other DNA
atactcacatggtggagaaa

SEQ ID NO: 275 - DNA, 20 bp, synthetic construct
  source 1..20: mol_type = other DNA
gagaagaatggaagggagaa

SEQ ID NO: 276 - DNA, 20 bp, synthetic construct
  source 1..20: mol_type = other DNA
tagagggttggaggaacagg

SEQ ID NO: 277 - DNA, 20 bp, synthetic construct
  source 1..20: mol_type = other DNA
cttagaacaaagagaagaat

SEQ ID NO: 278 - DNA, 20 bp, synthetic construct
  source 1..20: mol_type = other DNA
gacactgacactgtgcatga

SEQ ID NO: 279 - DNA, 20 bp, synthetic construct
  source 1..20: mol_type = other DNA
ggagttaccatatgacctgg

SEQ ID NO: 280 - DNA, 20 bp, synthetic construct
  source 1..20: mol_type = other DNA
cgtaagcttctagcaaggag

SEQ ID NO: 281 - DNA, 20 bp, synthetic construct
  source 1..20: mol_type = other DNA
ggtaaaaatgataaaaaacg

SEQ ID NO: 282 - DNA, 20 bp, synthetic construct
  source 1..20: mol_type = other DNA
agccttctcctgcctcagct

SEQ ID NO: 283 - DNA, 20 bp, synthetic construct
  source 1..20: mol_type = other DNA
agaagcagcagccacctgcg

SEQ ID NO: 284 - DNA, 20 bp, synthetic construct
  source 1..20: mol_type = other DNA
tggtcttctgacttttcttc

SEQ ID NO: 285 - DNA, 20 bp, synthetic construct
  source 1..20: mol_type = other DNA
cacctttttaaacttgaggg

SEQ ID NO: 286 - DNA, 20 bp, synthetic construct
  source 1..20: mol_type = other DNA
tttctcttctttcttatctt

SEQ ID NO: 287 - DNA, 20 bp, synthetic construct
  source 1..20: mol_type = other DNA
tcatgcttgccctctttctc

SEQ ID NO: 288 - DNA, 20 bp, synthetic construct
  source 1..20: mol_type = other DNA
tgatggctgcacgggctcat

SEQ ID NO: 289 - DNA, 20 bp, synthetic construct
  source 1..20: mol_type = other DNA
cagcagagtggtgggctgat

SEQ ID NO: 290 - DNA, 20 bp, synthetic construct
  source 1..20: mol_type = other DNA
tgatgtctctgctttgcctg

SEQ ID NO: 291 - DNA, 20 bp, synthetic construct
  source 1..20: mol_type = other DNA
cagaagcttccggcacagcc

SEQ ID NO: 292 - DNA, 20 bp, synthetic construct
  source 1..20: mol_type = other DNA
gggtccccggtcacggatga

SEQ ID NO: 293 - DNA, 20 bp, synthetic construct
  source 1..20: mol_type = other DNA
gcttaagcttccgtgtccag

SEQ ID NO: 294 - DNA, 20 bp, synthetic construct
  source 1..20: mol_type = other DNA
atacttcccagcagagcggc

SEQ ID NO: 295 - DNA, 20 bp, synthetic construct
  source 1..20: mol_type = other DNA
agcaaccaaagagtcaggcc

SEQ ID NO: 296 - DNA, 20 bp, synthetic construct
  source 1..20: mol_type = other DNA
actttagagcgaaaggcttt

SEQ ID NO: 297 - DNA, 20 bp, synthetic construct
  source 1..20: mol_type = other DNA
ttttcgaagtacgcaatcaa

SEQ ID NO: 298 - DNA, 20 bp, synthetic construct
  source 1..20: mol_type = other DNA
ccagggatgtgtcgcctacc

SEQ ID NO: 299 - DNA, 20 bp, synthetic construct
  source 1..20: mol_type = other DNA
ccagttaccgtgaagtcaaa

SEQ ID NO: 300 - DNA, 20 bp, synthetic construct
  source 1..20: mol_type = other DNA
tgggcttcttaggtggtttc

SEQ ID NO: 301 - DNA, 20 bp, synthetic construct
  source 1..20: mol_type = other DNA
gtggtgccgctccctttggg

SEQ ID NO: 302 - DNA, 20 bp, synthetic construct
  source 1..20: mol_type = other DNA
tctccaggacccttttcacc

SEQ ID NO: 303 - DNA, 20 bp, synthetic construct
  source 1..20: mol_type = other DNA
gacaaggagcttcccaggac

SEQ ID NO: 304 - DNA, 20 bp, synthetic construct
  source 1..20: mol_type = other DNA
ccctggcgaagtttgaaaag

SEQ ID NO: 305 - DNA, 20 bp, synthetic construct
  source 1..20: mol_type = other DNA
cccctcagccttgccccctg

SEQ ID NO: 306 - DNA, 20 bp, synthetic construct
  source 1..20: mol_type = other DNA
tgggtggatgtggtggcccc

SEQ ID NO: 307 - DNA, 20 bp, synthetic construct
  source 1..20: mol_type = other DNA
cggcctcagcttttcgcttc

SEQ ID NO: 308 - DNA, 20 bp, synthetic construct
  source 1..20: mol_type = other DNA
tcggccccgtttcttgggaa

SEQ ID NO: 309 - DNA, 20 bp, synthetic construct
  source 1..20: mol_type = other DNA
gcggcagcggctgccaccac

SEQ ID NO: 310 - DNA, 20 bp, synthetic construct
  source 1..20: mol_type = other DNA
aagactccttcacggctttc

SEQ ID NO: 311 - DNA, 20 bp, synthetic construct
  source 1..20: mol_type = other DNA
ggtctcctgcacagatcgga

SEQ ID NO: 312 - DNA, 20 bp, synthetic construct
  source 1..20: mol_type = other DNA
gctgaccgtctcccgggtct

SEQ ID NO: 313 - DNA, 20 bp, synthetic construct
  source 1..20: mol_type = other DNA
cgagggtggacaccagcagg

SEQ ID NO: 314 - DNA, 20 bp, synthetic construct
  source 1..20: mol_type = other DNA
acaggtcttcagtcctttcc

SEQ ID NO: 315 - DNA, 20 bp, synthetic construct
  source 1..20: mol_type = other DNA
ctgctctccttgcttttccg

SEQ ID NO: 316 - DNA, 20 bp, synthetic construct
  source 1..20: mol_type = other DNA
ctgagtggtggtgatggtgg

SEQ ID NO: 317 - DNA, 20 bp, synthetic construct
  source 1..20: mol_type = other DNA
cttctcctctttgcagacgc

SEQ ID NO: 318 - DNA, 20 bp, synthetic construct
  source 1..20: mol_type = other DNA
ccgtcgctctccagtgagcc

SEQ ID NO: 319 - DNA, 20 bp, synthetic construct
  source 1..20: mol_type = other DNA
ggcaaccgcgggctgagtct

SEQ ID NO: 320 - DNA, 20 bp, synthetic construct
  source 1..20: mol_type = other DNA
ccgtggcggcggtggcaacc

SEQ ID NO: 321 - DNA, 20 bp, synthetic construct
  source 1..20: mol_type = other DNA
tacttttctgcggccgtggc

SEQ ID NO: 322 - DNA, 20 bp, synthetic construct
  source 1..20: mol_type = other DNA
tctttgcgctctccctcccc

SEQ ID NO: 323 - DNA, 20 bp, synthetic construct
  source 1..20: mol_type = other DNA
tgtttggccttggcatggag

SEQ ID NO: 324 - DNA, 20 bp, synthetic construct
  source 1..20: mol_type = other DNA
aactctctcggtcacgggcg

SEQ ID NO: 325 - DNA, 20 bp, synthetic construct
  source 1..20: mol_type = other DNA
tgctttgcaatccgctccgt

SEQ ID NO: 326 - DNA, 20 bp, synthetic construct
  source 1..20: mol_type = other DNA
agagacaacagctgccttta

SEQ ID NO: 327 - DNA, 20 bp, synthetic construct
  source 1..20: mol_type = other DNA
gaagctttgtcagagcccta

SEQ ID NO: 328 - DNA, 22 bp, synthetic construct
  source 1..22: mol_type = other DNA
ccttccctgaaggttccctccc

SEQ ID NO: 329 - DNA, 20 bp, synthetic construct
  source 1..20: mol_type = other DNA
  modified_base 1: mod_base = OTHER; note = 2'-O-methoxyethyl adenosine 3'-phosphorothioate
  modified_base 2: mod_base = OTHER; note = 2'-O-methoxyethyl thymidine 3'-phosphodiester
  modified_base 3: mod_base = OTHER; note = 2'-O-methoxyethyl guanosine 3'-phosphodiester
  modified_base 4: mod_base = OTHER; note = 2'-O-methoxyethyl thymidine 3'-phosphodiester
  modified_base 5: mod_base = OTHER; note = 2'-O-methoxyethyl 5-methylcytidine 3'-phosphodiester
  modified_base 6: mod_base = OTHER; note = 2'-deoxyadenosine 3'-phosphorothioate
  modified_base 7: mod_base = OTHER; note = 5-methyl-2'-deoxycytidine 3'-phosphorothioate
  modified_base 8: mod_base = OTHER; note = 2'-deoxyadenosine 3'-phosphorothioate
  modified_base 9: mod_base = OTHER; note = 2'-deoxythymidine 3'-phosphorothioate
  modified_base 10: mod_base = OTHER; note = 5-methyl-2'-deoxycytidine 3'-phosphorothioate
  modified_base 11..13: mod_base = OTHER; note = 2'-deoxyadenosine 3'-phosphorothioate
  modified_base 14: mod_base = OTHER; note = 2'-deoxyguanosine 3'-phosphorothioate
  modified_base 15: mod_base = OTHER; note = 5-methyl-2'-deoxycytidine 3'-phosphorothioate
  modified_base 16: mod_base = OTHER; note = 2'-O-methoxyethyl adenosine 3'-phosphodiester
  modified_base 17: mod_base = OTHER; note = 2'-O-methoxyethyl guanosine 3'-phosphodiester
  modified_base 18: mod_base = OTHER; note = 2'-O-methoxyethyl guanosine 3'-phosphorothioate
  modified_base 19: mod_base = OTHER; note = 2'-O-methoxyethyl adenosine 3'-phosphorothioate
  modified_base 20: mod_base = OTHER; note = 2'-O-methoxyethyl adenosine
atgtcacatcaaagcaggaa

SEQ ID NO: 330 - DNA, 20 bp, synthetic construct
  source 1..20: mol_type = other DNA
  modified_base 1: mod_base = OTHER; note = 2'-O-methoxyethyl thymidine 3'-phosphorothioate
  modified_base 2: mod_base = OTHER; note = 2'-O-methoxyethyl thymidine 3'-phosphodiester
  modified_base 3..4: mod_base = OTHER; note = 2'-O-methoxyethyl guanosine 3'-phosphodiester
  modified_base 5: mod_base = OTHER; note = 2'-O-methoxyethyl adenosine 3'-phosphodiester
  modified_base 6: mod_base = OTHER; note = 2'-deoxyguanosine 3'-phosphorothioate
  modified_base 7: mod_base = OTHER; note = 5-methyl-2'-deoxycytidine 3'-phosphorothioate
  modified_base 8: mod_base = OTHER; note = 2'-deoxythymidine 3'-phosphorothioate
  modified_base 9..10: mod_base = OTHER; note = 2'-deoxyguanosine 3'-phosphorothioate
  modified_base 11: mod_base = OTHER; note = 2'-deoxythymidine 3'-phosphorothioate
  modified_base 12: mod_base = OTHER; note = 5-methyl-2'-deoxycytidine 3'-phosphorothioate
  modified_base 13: mod_base = OTHER; note = 2'-deoxythymidine 3'-phosphorothioate
  modified_base 14: mod_base = OTHER; note = 2'-deoxyadenosine 3'-phosphorothioate
  modified_base 15: mod_base = OTHER; note = 5-methyl-2'-deoxycytidine 3'-phosphorothioate
  modified_base 16: mod_base = OTHER; note = 2'-O-methoxyethyl adenosine 3'-phosphodiester
  modified_base 17: mod_base = OTHER; note = 2'-O-methoxyethyl guanosine 3'-phosphodiester
  modified_base 18..19: mod_base = OTHER; note = 2'-O-methoxyethyl adenosine 3'-phosphorothioate
  modified_base 20: mod_base = OTHER; note = 2'-O-methoxyethyl guanosine
ttggagctggtctacagaag

SEQ ID NO: 331 - DNA, 20 bp, synthetic construct
  source 1..20: mol_type = other DNA
  modified_base 1: mod_base = OTHER; note = 2'-O-methoxyethyl thymidine 3'-phosphorothioate
  modified_base 2: mod_base = OTHER; note = 2'-O-methoxyethyl guanosine 3'-phosphodiester
  modified_base 3: mod_base = OTHER; note = 2'-O-methoxyethyl adenosine 3'-phosphodiester
  modified_base 4: mod_base = OTHER; note = 2'-O-methoxyethyl guanosine 3'-phosphodiester
  modified_base 5: mod_base = OTHER; note = 2'-O-methoxyethyl thymidine 3'-phosphodiester
  modified_base 6: mod_base = OTHER; note = 5-methyl-2'-deoxycytidine 3'-phosphorothioate
  modified_base 7: mod_base = OTHER; note = 2'-deoxythymidine 3'-phosphorothioate
  modified_base 8: mod_base = OTHER; note = 2'-deoxyguanosine 3'-phosphorothioate
  modified_base 9: mod_base = OTHER; note = 2'-deoxythymidine 3'-phosphorothioate
  modified_base 10: mod_base = OTHER; note = 2'-deoxyadenosine 3'-phosphorothioate
  modified_base 11..15: mod_base = OTHER; note = 2'-deoxythymidine 3'-phosphorothioate
  modified_base 16: mod_base = OTHER; note = 2'-O-methoxyethyl adenosine 3'-phosphodiester
  modified_base 17: mod_base = OTHER; note = 2'-O-methoxyethyl thymidine 3'-phosphodiester
  modified_base 18..19: mod_base = OTHER; note = 2'-O-methoxyethyl guanosine 3'-phosphorothioate
  modified_base 20: mod_base = OTHER; note = 2'-O-methoxyethyl adenosine
tgagtctgtatttttatgga

SEQ ID NO: 332 - DNA, 20 bp, synthetic construct
  source 1..20: mol_type = other DNA
  modified_base 1: mod_base = OTHER; note = 2'-O-methoxyethyl guanosine 3'-phosphorothioate
  modified_base 2: mod_base = OTHER; note = 2'-O-methoxyethyl guanosine 3'-phosphodiester
  modified_base 3: mod_base = OTHER; note = 2'-O-methoxyethyl adenosine 3'-phosphodiester
  modified_base 4: mod_base = OTHER; note = 2'-O-methoxyethyl guanosine 3'-phosphodiester
  modified_base 5: mod_base = OTHER; note = 2'-O-methoxyethyl thymidine 3'-phosphodiester
  modified_base 6: mod_base = OTHER; note = 5-methyl-2'-deoxycytidine 3'-phosphorothioate
  modified_base 7: mod_base = OTHER; note = 2'-deoxyadenosine 3'-phosphorothioate
  modified_base 8: mod_base = OTHER; note = 5-methyl-2'-deoxycytidine 3'-phosphorothioate
  modified_base 9: mod_base = OTHER; note = 2'-deoxyadenosine 3'-phosphorothioate
  modified_base 10: mod_base = OTHER; note = 2'-deoxythymidine 3'-phosphorothioate
  modified_base 11: mod_base = OTHER; note = 2'-deoxyguanosine 3'-phosphorothioate
  modified_base 12: mod_base = OTHER; note = 2'-deoxythymidine 3'-phosphorothioate
  modified_base 13: mod_base = OTHER; note = 5-methyl-2'-deoxycytidine 3'-phosphorothioate
  modified_base 14: mod_base = OTHER; note = 2'-deoxyadenosine 3'-phosphorothioate
  modified_base 15: mod_base = OTHER; note = 5-methyl-2'-deoxycytidine 3'-phosphorothioate
  modified_base 16: mod_base = OTHER; note = 2'-O-methoxyethyl adenosine 3'-phosphodiester
  modified_base 17: mod_base = OTHER; note = 2'-O-methoxyethyl thymidine 3'-phosphodiester
  modified_base 18: mod_base = OTHER; note = 2'-O-methoxyethyl 5-methylcytidine 3'-phosphorothioate
  modified_base 19: mod_base = OTHER; note = 2'-O-methoxyethyl adenosine 3'-phosphorothioate
  modified_base 20: mod_base = OTHER; note = 2'-O-methoxyethyl adenosine
ggagtcacatgtcacatcaa

SEQ ID NO: 333 - DNA, 20 bp, synthetic construct
  source 1..20: mol_type = other DNA
  modified_base 1: mod_base = OTHER; note = 2'-O-methoxyethyl guanosine 3'-phosphorothioate
  modified_base 2..3: mod_base = OTHER; note = 2'-O-methoxyethyl adenosine 3'-phosphodiester
  modified_base 4: mod_base = OTHER; note = 2'-O-methoxyethyl thymidine 3'-phosphodiester
  modified_base 5: mod_base = OTHER; note = 2'-O-methoxyethyl 5-methylcytidine 3'-phosphodiester
  modified_base 6: mod_base = OTHER; note = 5-methyl-2'-deoxycytidine 3'-phosphorothioate
  modified_base 7: mod_base = OTHER; note = 2'-deoxythymidine 3'-phosphorothioate
  modified_base 8: mod_base = OTHER; note = 2'-deoxyguanosine 3'-phosphorothioate
  modified_base 9..10: mod_base = OTHER; note = 2'-deoxythymidine 3'-phosphorothioate
  modified_base 11..12: mod_base = OTHER; note = 2'-deoxyguanosine 3'-phosphorothioate
  modified_base 13: mod_base = OTHER; note = 2'-deoxyadenosine 3'-phosphorothioate
  modified_base 14: mod_base = OTHER; note = 2'-deoxyguanosine 3'-phosphorothioate
  modified_base 15: mod_base = OTHER; note = 5-methyl-2'-deoxycytidine 3'-phosphorothioate
  modified_base 16: mod_base = OTHER; note = 2'-O-methoxyethyl thymidine 3'-phosphodiester
  modified_base 17: mod_base = OTHER; note = 2'-O-methoxyethyl guanosine 3'-phosphodiester
  modified_base 18: mod_base = OTHER; note = 2'-O-methoxyethyl guanosine 3'-phosphorothioate
  modified_base 19: mod_base = OTHER; note = 2'-O-methoxyethyl thymidine 3'-phosphorothioate
  modified_base 20: mod_base = OTHER; note = 2'-O-methoxyethyl 5-methylcytidine
gaatcctgttggagctggtc

SEQ ID NO: 334 - DNA, 20 bp, synthetic construct
  source 1..20: mol_type = other DNA
  modified_base 1: mod_base = OTHER; note = 2'-O-methoxyethyl thymidine 3'-phosphorothioate
  modified_base 2: mod_base = OTHER; note = 2'-O-methoxyethyl 5-methylcytidine 3'-phosphodiester
  modified_base 3..5: mod_base = OTHER; note = 2'-O-methoxyethyl adenosine 3'-phosphodiester
  modified_base 6: mod_base = OTHER; note = 2'-deoxyguanosine 3'-phosphorothioate
  modified_base 7: mod_base = OTHER; note = 5-methyl-2'-deoxycytidine 3'-phosphorothioate
  modified_base 8: mod_base = OTHER; note = 2'-deoxyadenosine 3'-phosphorothioate
  modified_base 9..10: mod_base = OTHER; note = 2'-deoxyguanosine 3'-phosphorothioate
  modified_base 11..12: mod_base = OTHER; note = 2'-deoxyadenosine 3'-phosphorothioate
  modified_base 13: mod_base = OTHER; note = 5-methyl-2'-deoxycytidine 3'-phosphorothioate
  modified_base 14: mod_base = OTHER; note = 2'-deoxythymidine 3'-phosphorothioate
  modified_base 15: mod_base = OTHER; note = 2'-deoxyguanosine 3'-phosphorothioate
  modified_base 16: mod_base = OTHER; note = 2'-O-methoxyethyl guanosine 3'-phosphodiester
  modified_base 17: mod_base = OTHER; note = 2'-O-methoxyethyl thymidine 3'-phosphodiester
  modified_base 18: mod_base = OTHER; note = 2'-O-methoxyethyl guanosine 3'-phosphorothioate
  modified_base 19: mod_base = OTHER; note = 2'-O-methoxyethyl adenosine 3'-phosphorothioate
  modified_base 20: mod_base = OTHER; note = 2'-O-methoxyethyl guanosine
tcaaagcaggaactggtgag

SEQ ID NO: 335 - DNA, 20 bp, synthetic construct
  source 1..20: mol_type = other DNA
  modified_base 1: mod_base = OTHER; note = 2'-O-methoxyethyl guanosine 3'-phosphorothioate
  modified_base 2: mod_base = OTHER; note = 2'-O-methoxyethyl guanosine 3'-phosphodiester
  modified_base 3: mod_base = OTHER; note = 2'-O-methoxyethyl thymidine 3'-phosphodiester
  modified_base 4: mod_base = OTHER; note = 2'-O-methoxyethyl 5-methylcytidine 3'-phosphodiester
  modified_base 5: mod_base = OTHER; note = 2'-O-methoxyethyl thymidine 3'-phosphodiester
  modified_base 6: mod_base = OTHER; note = 2'-deoxyadenosine 3'-phosphorothioate
  modified_base 7: mod_base = OTHER; note = 5-methyl-2'-deoxycytidine 3'-phosphorothioate
  modified_base 8: mod_base = OTHER; note = 2'-deoxyadenosine 3'-phosphorothioate
  modified_base 9: mod_base = OTHER; note = 2'-deoxyguanosine 3'-phosphorothioate
  modified_base 10..11: mod_base = OTHER; note = 2'-deoxyadenosine 3'-phosphorothioate
  modified_base 12: mod_base = OTHER; note = 2'-deoxyguanosine 3'-phosphorothioate
  modified_base 13: mod_base = OTHER; note = 5-methyl-2'-deoxycytidine 3'-phosphorothioate
  modified_base 14..15: mod_base = OTHER; note = 2'-deoxyadenosine 3'-phosphorothioate
  modified_base 16..17: mod_base = OTHER; note = 2'-O-methoxyethyl guanosine 3'-phosphodiester
  modified_base 18: mod_base = OTHER; note = 2'-O-methoxyethyl thymidine 3'-phosphorothioate
  modified_base 19: mod_base = OTHER; note = 2'-O-methoxyethyl guanosine 3'-phosphorothioate
  modified_base 20: mod_base = OTHER; note = 2'-O-methoxyethyl thymidine
ggtctacagaagcaaggtgt

SEQ ID NO: 336 - DNA, 20 bp, synthetic construct
  source 1..20: mol_type = other DNA
  modified_base 1: mod_base = OTHER; note = 2'-O-methoxyethyl 5-methylcytidine 3'-phosphorothioate
  modified_base 2: mod_base = OTHER; note = 2'-O-methoxyethyl adenosine 3'-phosphodiester
  modified_base 3: mod_base = OTHER; note = 2'-O-methoxyethyl thymidine 3'-phosphodiester
  modified_base 4..5: mod_base = OTHER; note = 2'-O-methoxyethyl 5-methylcytidine 3'-phosphodiester
  modified_base 6: mod_base = OTHER; note = 5-methyl-2'-deoxycytidine 3'-phosphorothioate
  modified_base 7: mod_base = OTHER; note = 2'-deoxyadenosine 3'-phosphorothioate
  modified_base 8: mod_base = OTHER; note = 2'-deoxyguanosine 3'-phosphorothioate
  modified_base 9: mod_base = OTHER; note = 5-methyl-2'-deoxycytidine 3'-phosphorothioate
  modified_base 10: mod_base = OTHER; note = 2'-deoxythymidine 3'-phosphorothioate
  modified_base 11: mod_base = OTHER; note = 2'-deoxyadenosine 3'-phosphorothioate
  modified_base 12..13: mod_base = OTHER; note = 5-methyl-2'-deoxycytidine 3'-phosphorothioate
  modified_base 14: mod_base = OTHER; note = 2'-deoxyadenosine 3'-phosphorothioate
  modified_base 15: mod_base = OTHER; note = 2'-deoxythymidine 3'-phosphorothioate
  modified_base 16..17: mod_base = OTHER; note = 2'-O-methoxyethyl guanosine 3'-phosphodiester
  modified_base 18..19: mod_base = OTHER; note = 2'-O-methoxyethyl adenosine 3'-phosphorothioate
  modified_base 20: mod_base = OTHER; note = 2'-O-methoxyethyl thymidine
catcccagctaccatggaat

SEQ ID NO: 337 - DNA, 20 bp, synthetic construct
  source 1..20: mol_type = other DNA
  modified_base 1: mod_base = OTHER; note = 2'-O-methoxyethyl 5-methylcytidine 3'-phosphorothioate
  modified_base 2..3: mod_base = OTHER; note = 2'-O-methoxyethyl thymidine 3'-phosphodiester
  modified_base 4..5: mod_base = OTHER; note = 2'-O-methoxyethyl 5-methylcytidine 3'-phosphodiester
  modified_base 6..7: mod_base = OTHER; note = 2'-deoxythymidine 3'-phosphorothioate
  modified_base 8: mod_base = OTHER; note = 2'-deoxyguanosine 3'-phosphorothioate
  modified_base 9: mod_base = OTHER; note = 2'-deoxyadenosine 3'-phosphorothioate
  modified_base 10..11: mod_base = OTHER; note = 5-methyl-2'-deoxycytidine 3'-phosphorothioate
  modified_base 12: mod_base = OTHER; note = 2'-deoxythymidine 3'-phosphorothioate
  modified_base 13: mod_base = OTHER; note = 5-methyl-2'-deoxycytidine 3'-phosphorothioate
  modified_base 14: mod_base = OTHER; note = 2'-deoxyguanosine 3'-phosphorothioate
  modified_base 15: mod_base = OTHER; note = 2'-deoxyadenosine 3'-phosphorothioate
  modified_base 16: mod_base = OTHER; note = 2'-O-methoxyethyl thymidine 3'-phosphodiester
  modified_base 17: mod_base = OTHER; note = 2'-O-methoxyethyl guanosine 3'-phosphodiester
  modified_base 18: mod_base = OTHER; note = 2'-O-methoxyethyl 5-methylcytidine 3'-phosphorothioate
  modified_base 19: mod_base = OTHER; note = 2'-O-methoxyethyl thymidine 3'-phosphorothioate
  modified_base 20: mod_base = OTHER; note = 2'-O-methoxyethyl guanosine
cttccttgacctcgatgctg

SEQ ID NO: 338 - DNA, 20 bp, synthetic construct
  source 1..20: mol_type = other DNA
  modified_base 1: mod_base = OTHER; note = 2'-O-methoxyethyl guanosine 3'-phosphorothioate
  modified_base 2: mod_base = OTHER; note = 2'-O-methoxyethyl guanosine 3'-phosphodiester
  modified_base 3: mod_base = OTHER; note = 2'-O-methoxyethyl thymidine 3'-phosphodiester
  modified_base 4: mod_base = OTHER; note = 2'-O-methoxyethyl 5-methylcytidine 3'-phosphodiester
  modified_base 5: mod_base = OTHER; note = 2'-O-methoxyethyl thymidine 3'-phosphodiester
  modified_base 6..7: mod_base = OTHER; note = 5-methyl-2'-deoxycytidine 3'-phosphorothioate
  modified_base 8: mod_base = OTHER; note = 2'-deoxythymidine 3'-phosphorothioate
  modified_base 9: mod_base = OTHER; note = 2'-deoxyguanosine 3'-phosphorothioate
  modified_base 10: mod_base = OTHER; note = 5-methyl-2'-deoxycytidine 3'-phosphorothioate
  modified_base 11: mod_base = OTHER; note = 2'-deoxyadenosine 3'-phosphorothioate
  modified_base 12: mod_base = OTHER; note = 5-methyl-2'-deoxycytidine 3'-phosphorothioate
  modified_base 13: mod_base = OTHER; note = 2'-deoxyadenosine 3'-phosphorothioate
  modified_base 14: mod_base = OTHER; note = 2'-deoxyguanosine 3'-phosphorothioate
  modified_base 15: mod_base = OTHER; note = 2'-deoxyadenosine 3'-phosphorothioate
  modified_base 16: mod_base = OTHER; note = 2'-O-methoxyethyl thymidine 3'-phosphodiester
  modified_base 17: mod_base = OTHER; note = 2'-O-methoxyethyl 5-methylcytidine 3'-phosphodiester
  modified_base 18..19: mod_base = OTHER; note = 2'-O-methoxyethyl guanosine 3'-phosphorothioate
  modified_base 20: mod_base = OTHER; note = 2'-O-methoxyethyl adenosine
ggtctcctgcacagatcgga

SEQ ID NO: 339 - DNA, 20 bp, synthetic construct
  source 1..20: mol_type = other DNA
  modified_base 1: mod_base = OTHER; note = 2'-MOE adenosine 3'-phosphorothioate
  modified_base 2: mod_base = OTHER; note = 2'-MOE guanosine 3'-phosphodiester
  modified_base 3..5: mod_base = OTHER; note = 2'-MOE 5-methylcytidine 3'-phosphodiester
  modified_base 6: mod_base = OTHER; note = 2'-deoxythymidine 3'-phosphorothioate
  modified_base 7..8: mod_base = OTHER; note = 2'-deoxyadenosine 3'-phosphorothioate
  modified_base 9: mod_base = OTHER; note = 5-methyl-2'-deoxycytidine 3'-phosphorothioate
  modified_base 10: mod_base = OTHER; note = 2'-deoxyadenosine 3'-phosphorothioate
  modified_base 11: mod_base = OTHER; note = 2'-deoxythymidine 3'-phosphorothioate
  modified_base 12..14: mod_base = OTHER; note = 5-methyl-2'-deoxycytidine 3'-phosphorothioate
  modified_base 15: mod_base = OTHER; note = 2'-deoxyadenosine 3'-phosphorothioate
  modified_base 16: mod_base = OTHER; note = 2'-MOE guanosine 3'-phosphodiester
  modified_base 17: mod_base = OTHER; note = 2'-MOE 5-methylcytidine 3'-phosphodiester
  modified_base 18: mod_base = OTHER; note = 2'-MOE thymidine 3'-phosphorothioate
  modified_base 19: mod_base = OTHER; note = 2'-MOE adenosine 3'-phosphorothioate
  modified_base 20: mod_base = OTHER; note = 2'-MOE 5-methylcytidine
agccctaacatcccagctac

(19) United States
(12) Patent Application Publication
(10) Pub. No.: US 2026/0102423 A1
(43) Pub. Date: Apr. 16, 2026

(54) COMPOSITIONS AND METHODS FOR MODULATING MECP2 EXPRESSION

(71) Applicant: Ionis Pharmaceuticals, Inc., Carlsbad, CA (US)

(72) Inventor: Susan M. Freier, San Diego, CA (US)

(73) Assignee: Ionis Pharmaceuticals, Inc., Carlsbad, CA (US)

(21) Appl. No.: 19/223,874

(22) Filed: May 30, 2025

Related U.S. Application Data
(62) Division of application No. 17/408,920, filed on Aug. 23, 2021, now Pat. No. 12,343,357.
Application No. 17/408,920 is a division of application No. 16/535,888, filed on Aug. 8, 2019, now Pat. No. 11,129,844.
Application No. 16/535,888 is a continuation-in-part of application No. 15/554,407, now abandoned.
Application No. 16/535,888 is a continuation-in-part of application No. 15/554,409, now abandoned.
(60) Provisional application No. 62/127,693, filed on Mar. 3, 2015.
(60) Provisional application No. 62/127,682, filed on Mar. 3, 2015.

(51) Int. Cl. A61K 31/712; A61P 25/22; C12N 15/113

(52) U.S. Cl. CPC A61K 31/712; A61P 25/22; C12N 15/113; C12N 2310/11

(57) ABSTRACT

Disclosed herein are compounds and methods for decreasing MECP2 mRNA and protein expression. Such compounds and methods are useful to treat, prevent, or ameliorate MECP2 associated disorders and syndromes. Such MECP2 associated disorders include MECP2 duplication syndrome.

### SEQUENCE LISTING

The present application is being filed along with a Sequence Listing in electronic format. The Sequence Listing is provided as a file entitled BIOL0263USD2SEQ.xml created May 15, 2025, which is 476 Kb in size. The information in the electronic format of the sequence listing is incorporated herein by reference in its entirety.

### FIELD

Provided are compositions and methods for modulating expression of methyl CpG binding protein 2 (MECP2) mRNA and protein in an animal. Such methods are useful to treat, prevent, or ameliorate neurological disorders, including MECP2 duplication syndrome, by reducing expression and amount of MECP2 mRNA and protein in an animal.

### BACKGROUND

Methyl CpG binding protein 2 (MECP2) is located on chromosome Xq28 and plays a fundamental role in epigenetics, controlling chromatin states, and expression of thousands of genes (Chahrour et al., Science, 2008, 320:1224-1229; Nan et al., Nature, 1998, 393:386-389; Jones et al., Nat. Genet., 1998, 19:187-191). MECP2 expression must be maintained within a fairly narrow range to assure proper gene expression and neuronal function (Nan et al., Nature, 1988, 393:386-389). MECP2 duplication syndrome caused by overexpression of MECP2 is characterized by autism, intellectual disability, motor dysfunction, anxiety, epilepsy, recurrent respiratory tract infections, and early death, typically in males (Ramocki et al., Am J Med Genet A, 2010, 152A:1079-1088). Underexpression of MECP2 is associated with Rett Syndrome, which is characterized by normal early growth and development followed by a slowing of development, loss of purposeful use of the hands, distinctive hand movements, slowed brain and head growth, problems with walking, seizures, and intellectual disability, typically in females (Weaving et al., J Med Genet, 2005, 42:1-7).

Currently there is a lack of acceptable options for treating such neurological disorders. It is therefore an object herein to provide compositions and methods for the treatment of such disorders.

### SUMMARY

Provided herein are compositions and methods for modulating expression and amount of methyl CpG binding protein 2 (MECP2) mRNA and protein. In certain embodiments, compounds useful for modulating expression and amount of MECP2 mRNA and protein are antisense compounds. In certain embodiments, the antisense compounds are modified antisense oligonucleotides. In certain embodiments, the antisense compounds are single-stranded antisense oligonucleotides. In certain embodiments, the antisense compounds are not siRNA compounds.

In certain embodiments, modulation can occur in a cell or tissue. In certain embodiments, the cell or tissue is in an animal. In certain embodiments, the animal is a human. In certain embodiments, MECP2 mRNA levels are reduced. In certain embodiments, MECP2 protein levels are reduced. Such reduction can occur in a time-dependent manner or in a dose-dependent manner.

Also provided are compositions and methods useful for preventing, treating, and ameliorating disorders and syndromes associated with MECP2 overexpression. In certain embodiments, a disorder associated with MECP2 overexpression is a neurological disorder. In certain embodiments, the neurological disorder is MECP2 duplication syndrome. In certain embodiments, MECP2 duplication syndrome is characterized by having additional copies of MECP2, which leads to overexpression of MECP2.

In certain embodiments, MECP2 duplication syndrome is characterized by autism, intellectual disability, motor dysfunction, anxiety, epilepsy, recurrent respiratory tract infections, and early death. In certain embodiments, MECP2 duplication syndrome is inherited in an X-linked pattern.

In certain embodiments, methods of treatment include administering a MECP2 antisense compound to an individual in need thereof. In certain embodiments, methods of treatment include administering a MECP2 modified antisense oligonucleotide to an individual in need thereof.

In certain embodiments, MECP2 levels are reduced sufficiently to prevent, treat, and ameliorate symptoms of MECP2 duplication syndrome, but not enough to cause symptoms of Rett Syndrome.

### BRIEF DESCRIPTION OF THE DRAWINGS

FIG. 1 displays representative EEG traces for WT mice, MECP2-TG1 mice without Isis No. 628785 treatment, and MECP2-TG1 mice that received treatment with Isis No. 628785.

### DETAILED DESCRIPTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Additionally, as used herein, the use of “and” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this disclosure, including, but not limited to, patents, patent applications, published patent applications, articles, books, treatises, and GENBANK Accession Numbers and associated sequence information obtainable through databases such as National Center for Biotechnology Information (NCBI) and other data referred to throughout in the disclosure herein are hereby expressly incorporated by reference for the portions of the document discussed herein, as well as in their entirety.

### Definitions

Unless specific definitions are provided, the nomenclature utilized in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis.

Unless otherwise indicated, the following terms have the following meanings:

“2′-O-methoxyethyl” (also 2′-MOE and 2′-OCH₂CH₂—OCH₃ and MOE) refers to an O-methoxy-ethyl modification of the 2′ position of a furanose ring. A 2′-O-methoxyethyl modified sugar is a modified sugar.

“2′-MOE nucleoside” (also 2′-O-methoxyethyl nucleoside) means a nucleoside comprising a 2′-MOE modified sugar moiety.

“2′-substituted nucleoside” means a nucleoside comprising a substituent at the 2′-position of the furanose ring other than H or OH. In certain embodiments, 2′ substituted nucleosides include nucleosides with bicyclic sugar modifications.

“5-methylcytosine” means a cytosine modified with a methyl group attached to the 5 position. A 5-methylcytosine is a modified nucleobase.

“Administered concomitantly” refers to the co-administration of two pharmaceutical agents in any manner in which the pharmacological effects of both are manifest in the patient at the same time. Concomitant administration does not require that both pharmaceutical agents be administered in a single pharmaceutical composition, in the same dosage form, or by the same route of administration. The effects of both pharmaceutical agents need not manifest themselves at the same time. The effects need only be overlapping for a period of time and need not be coextensive.

“Administering” means providing a pharmaceutical agent to an animal, and includes, but is not limited to administering by a medical professional and self-administering.

“Amelioration” refers to a lessening, slowing, stopping, or reversing of at least one indicator of the seventy of a syndrome or condition. The severity of indicators may be determined by subjective or objective measures, which are known to those skilled in the art.

“Animal” refers to a human or non-human animal, including, but not limited to, mice, rats, rabbits, dogs, cats, pigs, and non-human primates, including, but not limited to, monkeys and chimpanzees.

“Antibody” refers to a molecule characterized by reacting specifically with an antigen in some way, where the antibody and the antigen are each defined in terms of the other. Antibody may refer to a complete antibody molecule or any fragment or region thereof, such as the heavy chain, the light chain, Fab region, and Fc region.

“Antisense activity” means any detectable or measurable activity attributable to the hybridization of an antisense compound to its target nucleic acid. In certain embodiments, antisense activity is a decrease in the amount or expression of a target nucleic acid or protein encoded by such target nucleic acid.

“Antisense compound” means an oligomeric compound that is capable of undergoing hybridization to a target nucleic acid through hydrogen bonding. Examples of antisense compounds include single-stranded and double-stranded compounds, such as, antisense oligonucleotides, siRNAs, shRNAs, ssRNAs, and occupancy-based compounds.

“Antisense inhibition” or “inhibition” means reduction of target nucleic acid levels in the presence of an antisense compound complementary to a target nucleic acid compared to target nucleic acid levels or in the absence of the antisense compound.

“Antisense mechanisms” are all those mechanisms involving hybridization of a compound with a target nucleic acid, wherein the outcome or effect of the hybridization is either target degradation or target occupancy with concomitant stalling of the cellular machinery involving, for example, transcription or splicing.

“Antisense oligonucleotide” means a single-stranded oligonucleotide having a nucleobase sequence that permits hybridization to a corresponding segment of a target nucleic acid.

“Base complementarity” refers to the capacity for the precise base pairing of nucleobases of an antisense oligonucleotide with corresponding nucleobases in a target nucleic acid (i.e., hybridization), and is mediated by Watson-Crick, Hoogsteen or reversed Hoogsteen hydrogen binding between corresponding nucleobases.

“Bicyclic sugar” means a furanose ring modified by the bridging of two atoms. A bicyclic sugar is a modified sugar.

“Bicyclic nucleoside” (also BNA) means a nucleoside having a sugar moiety comprising a bridge connecting two carbon atoms of the sugar ring, thereby forming a bicyclic ring system. In certain embodiments, the bridge connects the 4′-carbon and the 2′-carbon of the sugar ring.

“Cap structure” or “terminal cap moiety” means chemical modifications, which have been incorporated at either terminus of an antisense compound.

“cEt” or “constrained ethyl” means a bicyclic nucleoside having a sugar moiety comprising a bridge connecting the 4′-carbon and the 2′-carbon, wherein the bridge has the formula: 4′-CH(CH₃)—O-2′.

“Constrained ethyl nucleoside” (also cEt nucleoside) means a nucleoside comprising a bicyclic sugar moiety comprising a 4′-CH(CH₃)—O-2′ bridge.

“Chemically distinct region” refers to a region of an antisense compound that is in some way chemically different than another region of the same antisense compound. For example, a region having 2′-O-methoxyethyl nucleosides is chemically distinct from a region having nucleosides without 2′-O-methoxyethyl modifications.

“Chimeric antisense compound” means an antisense compound that has at least two chemically distinct regions, each position having a plurality of subunits.

“Co-administration” means administration of two or more pharmaceutical agents to an individual. The two or more pharmaceutical agents may be in a single pharmaceutical composition, or may be in separate pharmaceutical compositions. Each of the two or more pharmaceutical agents may be administered through the same or different routes of administration. Co-administration encompasses parallel or sequential administration.

“Complementarity” means the capacity for pairing between nucleobases of a first nucleic acid and a second nucleic acid.

“Comprise,” “comprises,” and “comprising” will be understood to imply the inclusion of a stated step or element or group of steps or elements but not the exclusion of any other step or element or group of steps or elements.

“Contiguous nucleobases” means nucleobases immediately adjacent to each other.

“Designing” or “designed to” refer to the process of designing an oligomeric compound that specifically hybridizes with a selected nucleic acid molecule.

“Diluent” means an ingredient in a composition that lacks pharmacological activity, but is pharmaceutically necessary or desirable. For example, in drugs that are injected, the diluent may be a liquid, e.g. saline solution.

“Dose” means a specified quantity of a pharmaceutical agent provided in a single administration, or in a specified time period. In certain embodiments, a dose may be administered in one, two, or more boluses, tablets, or injections. For example, in certain embodiments where subcutaneous administration is desired, the desired dose requires a volume not easily accommodated by a single injection, therefore, two or more injections may be used to achieve the desired dose. In certain embodiments, the pharmaceutical agent is administered by infusion over an extended period of time or continuously. Doses may be stated as the amount of pharmaceutical agent per hour, day, week, or month.

“Effective amount” in the context of modulating an activity or of treating or preventing a condition means the administration of that amount of pharmaceutical agent to an individual in need of such modulation, treatment, or prophylaxis, either in a single dose or as part of a series, that is effective for modulation of that effect, or for treatment or prophylaxis or improvement of that condition. The effective amount may vary among individuals depending on the health and physical condition of the individual to be treated, the taxonomic group of the individuals to be treated, the formulation of the composition, assessment of the individual's medical condition, and other relevant factors.

“Efficacy” means the ability to produce a desired effect.

“Expression” includes all the functions by which a gene's coded information is converted into structures present and operating in a cell. Such structures include, but are not limited to the products of transcription and translation.

“Fully complementary” or “100% complementary” means each nucleobase of a first nucleic acid has a complementary nucleobase in a second nucleic acid. In certain embodiments, a first nucleic acid is an antisense compound and a target nucleic acid is a second nucleic acid.

“Gapmer” means a chimeric antisense compound in which an internal region having a plurality of nucleosides that support RNase H cleavage is positioned between external regions having one or more nucleosides, wherein the nucleosides comprising the internal region are chemically distinct from the nucleoside or nucleosides comprising the external regions. The internal region may be referred to as a “gap” and the external regions may be referred to as the “wings.”

“Hotspot region” is a range of nucleobases on a target nucleic acid amenable to antisense compounds for reducing the amount or activity of the target nucleic acid as demonstrated in the examples hereinbelow.

“Hybridization” means the annealing of complementary nucleic acid molecules. In certain embodiments, complementary nucleic acid molecules include, but are not limited to, an antisense compound and a target nucleic acid. In certain embodiments, complementary nucleic acid molecules include, but are not limited to, an antisense oligonucleotide and a nucleic acid target.

“Identifying an animal having a MECP2 associated disorder” means identifying an animal having been diagnosed with a MECP2 associated disorder or predisposed to develop a MECP2 associated disorder.

Individuals predisposed to develop a MECP2 associated disorder include those having one or more risk factors for developing a MECP2 associated disorder, including, having a personal or family history or genetic predisposition to one or more MECP2 associated disorders. Such identification may be accomplished by any method including evaluating an individual's medical history and standard clinical tests or assessments, such as genetic testing.

“Immediately adjacent” means there are no intervening elements between the immediately adjacent elements.

“Individual” means a human or non-human animal selected for treatment or therapy.

“Inhibiting MECP2” means reducing the level or expression of a MECP2 mRNA and/or protein. In certain embodiments, MECP2 mRNA and/or protein levels are inhibited in the presence of an antisense compound targeting MECP2, including an antisense oligonucleotide targeting MECP2, as compared to expression of MECP2 mRNA and/or protein levels in the absence of a MECP2 antisense compound, such as an antisense oligonucleotide.

“Inhibiting the expression or activity” refers to a reduction or blockade of the expression or activity and does not necessarily indicate a total elimination of expression or activity.

“Internucleoside linkage” refers to the chemical bond between nucleosides.

“Linked nucleosides” means adjacent nucleosides linked together by an internucleoside linkage.

“MECP2 antisense compound” means an antisense compound targeting MECP2.

“MECP2” means the mammalian gene methyl CpG binding protein 2 (MECP2), including the human gene methyl CpG binding protein 2 (MECP2). Human MECP2 has been mapped to human chromosome Xq28.

“MECP2 associated disorder” means any disorder or syndrome associated with any MECP2 nucleic acid or expression product thereof. Such disorders may include a neurological disorder. Such neurological disorders may include MECP2 duplication syndrome.

“MECP2 nucleic acid” means any nucleic acid encoding MECP2. For example, in certain embodiments, a MECP2 nucleic acid includes a DNA sequence encoding MECP2, an RNA sequence transcribed from DNA encoding MECP2 (including genomic DNA comprising introns and exons), and an mRNA sequence encoding MECP2.

“MECP2 mRNA” means any messenger RNA expression product of a DNA sequence encoding MECP2.

“MECP2 protein” means the polypeptide expression product of a MECP2 nucleic acid.

“Mismatch” or “non-complementary nucleobase” refers to the case when a nucleobase of a first nucleic acid is not capable of pairing with the corresponding nucleobase of a second or target nucleic acid.

“Modified internucleoside linkage” refers to a substitution or any change from a naturally occurring internucleoside bond (i.e., a phosphodiester internucleoside bond).

“Modified nucleobase” means any nucleobase other than adenine, cytosine, guanine, thymidine, or uracil. An “unmodified nucleobase” means the purine bases adenine (A) and guanine (G), and the pyrimidine bases thymine (T), cytosine (C), and uracil (U).

“Modified nucleoside” means a nucleoside having, independently, a modified sugar moiety and/or modified nucleobase.

“Modified nucleotide” means a nucleotide having, independently, a modified sugar moiety, modified internucleoside linkage, and/or modified nucleobase.

“Modified antisense oligonucleotide” means an oligonucleotide comprising at least one modified internucleoside linkage, modified sugar, and/or modified nucleobase.

“Modified sugar” means substitution and/or any change from a natural sugar moiety.

“Monomer” means a single unit of an oligomer. Monomers include, but are not limited to, nucleosides and nucleotides, whether naturally occurring or modified.

“Motif” means the pattern of unmodified and modified nucleosides in an antisense compound.

“Natural sugar moiety” means a sugar moiety found in DNA (2′-H) or RNA (2′-OH).

“Naturally occurring internucleoside linkage” means a 3′ to 5′ phosphodiester linkage.

“Non-complementary nucleobase” refers to a pair of nucleobases that do not form hydrogen bonds with one another or otherwise support hybridization.

“Nucleic acid” refers to molecules composed of monomeric nucleotides. A nucleic acid includes, but is not limited to, ribonucleic acids (RNA), deoxyribonucleic acids (DNA), single-stranded nucleic acids, double-stranded nucleic acids, small interfering ribonucleic acids (siRNA), and microRNAs (miRNA).

“Nucleobase” means a heterocyclic moiety capable of pairing with a base of another nucleic acid.

“Nucleobase complementarity” refers to a nucleobase that is capable of base pairing with another nucleobase. For example, in DNA, adenine (A) is complementary to thymine (T). For example, in RNA, adenine (A) is complementary to uracil (U). In certain embodiments, complementary nucleobase refers to a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair.

“Nucleobase sequence” means the order of contiguous nucleobases independent of any sugar, linkage, and/or nucleobase modification.

“Nucleoside” means a nucleobase linked to a sugar.

“Nucleoside mimetic” includes those structures used to replace the sugar or the sugar and the base and not necessarily the linkage at one or more positions of an oligomeric compound such as for example nucleoside mimetics having morpholino, cyclohexenyl, cyclohexyl, tetrahydropyranyl, bicyclo, or tricyclo sugar mimetics, e.g., non furanose sugar units. Nucleotide mimetic includes those structures used to replace the nucleoside and the linkage at one or more positions of an oligomeric compound such as for example peptide nucleic acids or morpholinos (morpholinos linked by —N(H)—C(═O)—O— or other non-phosphodiester linkage). Sugar surrogate overlaps with the slightly broader term nucleoside mimetic but is intended to indicate replacement of the sugar unit (furanose ring) only. The tetrahydropyranyl rings provided herein are illustrative of an example of a sugar surrogate wherein the furanose sugar group has been replaced with a tetrahydropyranyl ring system. “Mimetic” refers to groups that are substituted for a sugar, a nucleobase, and/or intemucleoside linkage. Generally, a mimetic is used in place of the sugar or sugar-internucleoside linkage combination, and the nucleobase is maintained for hybridization to a selected target.

“Nucleotide” means a nucleoside having a phosphate group covalently linked to the sugar portion of the nucleoside.

“Off-target effect” refers to an unwanted or deleterious biological effect associated with modulation of RNA or protein expression of a gene other than the intended target nucleic acid.

“Oligomeric compound” or “oligomer” means a polymer of linked monomeric subunits which is capable of hybridizing to at least a region of a nucleic acid molecule.

“Oligonucleotide” means a polymer of linked nucleosides each of which can be modified or unmodified, independent one from another.

“Parenteral administration” means administration through injection (e.g., bolus injection) or infusion. Parenteral administration includes subcutaneous administration, intravenous administration, intramuscular administration, intraarterial administration, intraperitoneal administration, or intracranial administration, e.g., intrathecal or intracerebroventricular administration.

“Peptide” means a molecule formed by linking at least two amino acids by amide bonds. Without limitation, as used herein, peptide refers to polypeptides and proteins.

“Pharmaceutical agent” means a substance that provides a therapeutic benefit when administered to an individual. For example, in certain embodiments, an antisense oligonucleotide targeted to MECP2 is a pharmaceutical agent.

“Pharmaceutical composition” means a mixture of substances suitable for administering to an individual. For example, a pharmaceutical composition may comprise an antisense oligonucleotide and a sterile aqueous solution.

“Pharmaceutically acceptable derivative” encompasses pharmaceutically acceptable salts, conjugates, prodrugs or isomers of the compounds described herein.

“Pharmaceutically acceptable salts” means physiologically and pharmaceutically acceptable salts of antisense compounds, i.e., salts that retain the desired biological activity of the parent oligonucleotide and do not impart undesired toxicological effects thereto.

“Phosphorothioate linkage” means a linkage between nucleosides where the phosphodiester bond is modified by replacing one of the non-bridging oxygen atoms with a sulfur atom. A phosphorothioate linkage is a modified internucleoside linkage.

“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an antisense compound.

“Prevent” or “preventing” refers to delaying or forestalling the onset or development of a disorder or syndrome for a period of time from minutes to days, weeks to months, or indefinitely.

“Prodrug” means a therapeutic agent that is prepared in an inactive form that is converted to an active form (i.e., drug) within the body or cells thereof by the action of endogenous enzymes or other chemicals and/or conditions.

“Prophylactically effective amount” refers to an amount of a pharmaceutical agent that provides a prophylactic or preventative benefit to an animal.

“Region” is defined as a portion of the target nucleic acid having at least one identifiable structure, function, or characteristic.

“Ribonucleotide” means a nucleotide having a hydroxy at the 2′ position of the sugar portion of the nucleotide. Ribonucleotides may be modified with any of a variety of substituents.

“Salt” means a physiologically and pharmaceutically acceptable salt(s) of antisense compounds, i.e., salts that retain the desired biological activity of the parent oligonucleotide and do not impart undesired toxicological effects thereto.

“Segments” are defined as smaller or sub-portions of regions within a target nucleic acid.

“Shortened” or “truncated” versions of antisense oligonucleotides taught herein have one, two or more nucleosides deleted.

“Side effects” means physiological responses attributable to a treatment other than desired effects. In certain embodiments, side effects include, without limitation, injection site reactions, liver function test abnormalities, renal function abnormalities, liver toxicity, renal toxicity, central nervous system abnormalities, and myopathies.

“Single-stranded antisense oligonucleotide” means an oligonucleotide which is not hybridized to a complementary strand. A single-stranded antisense oligonucleotide is not a siRNA.

“Sites” as used herein, are defined as unique nucleobase positions within a target nucleic acid.

“Slows progression” means decrease in the development of the disorder or syndrome.

“Specifically hybridizable” refers to an antisense compound having a sufficient degree of complementarity between an antisense oligonucleotide and a target nucleic acid to induce a desired effect, while exhibiting minimal or no effects on non-target nucleic acids under conditions in which specific binding is desired, i.e., under physiological conditions in the case of in vivo assays and therapeutic treatments.

“Standard cell assay” means the assay described in Example 1 and reasonable variations thereof.

“Stringent hybridization conditions” or “stringent conditions” refer to conditions under which an oligomeric compound will hybridize to its target sequence, but to a minimal number of other sequences.

“Targeting” or “targeted” means the process of design and selection of an antisense compound that will specifically hybridize to a target nucleic acid and induce a desired effect.

“Target nucleic acid,” “target RNA,” and “target RNA transcript” and “nucleic acid target” all mean a nucleic acid capable of being targeted by antisense compounds. In certain embodiments, the target nucleic acid is a MECP2 nucleic acid.

“Target region” means a portion of a target nucleic acid to which one or more antisense compounds is targeted.

“Target segment” means the sequence of nucleotides of a target nucleic acid to which an antisense compound is targeted. “5′ target site” refers to the 5′-most nucleotide of a target segment. “3′ target site” refers to the 3′-most nucleotide of a target segment.

“Therapeutically effective amount” means an amount of a pharmaceutical agent that provides a therapeutic benefit to an individual.

“Treat” or “treating” or “treatment” refers administering a composition to effect an alteration or improvement of the disorder or syndrome.

“Unmodified nucleobases” mean the purine bases adenine (A) and guanine (G), and the pyrimidine bases thymine (T), cytosine (C) and uracil (U).

“Unmodified nucleotide” means a nucleotide composed of naturally occurring nucleobases, sugar moieties, and internucleoside linkages. In certain embodiments, an unmodified nucleotide is an RNA nucleotide (i.e. β-D-ribonucleosides) or a DNA nucleotide (i.e. β-D-deoxyribonucleoside).

“Wing segment” means a plurality of nucleosides modified to impart to an oligonucleotide properties such as enhanced inhibitory activity, increased binding affinity for a target nucleic acid, or resistance to degradation by in vivo nucleases.

### Certain Embodiments

Certain embodiments provide methods, compounds, and compositions for inhibiting MECP2 mRNA and protein expression. Certain embodiments provide methods, compounds, and compositions for decreasing MECP2 mRNA and protein levels.

Certain embodiments provide antisense compounds targeted to a MECP2 nucleic acid. In certain embodiments, the MECP2 nucleic acid is the sequence set forth in GENBANK Accession No. NM_004992.3 (incorporated herein as SEQ ID NO: 2) and the complement of GENBANK Accession No. NT_167198.1 truncated from nucleotides 4203000 to U.S. Pat. No. 4,283,000 (incorporated herein as SEQ ID NO: 1).

Certain embodiments provide methods, compounds, and compositions for the treatment, prevention, or amelioration of disorders and syndromes associated with MECP2 in an individual in need thereof. Also contemplated are methods for the preparation of a medicament for the treatment, prevention, or amelioration of a disorder or syndrome associated with MECP2. MECP2 associated disorders and syndromes include neurological disorders. In certain embodiments, MECP2 associated disorders include MECP2 duplication syndrome.

The present disclosure provides the following non-limiting numbered embodiments:
Embodiment 1. A compound, comprising a modified antisense oligonucleotide consisting of 12 to 30 linked nucleosides and having a nucleobase sequence comprising at least 8, at least 9, at least 10, at least 11, at least 12, at least 13, at least 14, at least 15, at least 16, at least 17, at least 18, at least 19, or at least 20 consecutive nucleobases of any of the nucleobase sequences of SEQ ID NOs: 16-327.
Embodiment 2. The compound of embodiment 2, wherein the nucleobase sequence of the modified antisense oligonucleotide is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% complementary to SEQ ID NO: 1 or SEQ ID NO: 2.
Embodiment 3. The compound of any preceding embodiment, consisting of a single-stranded modified antisense oligonucleotide.
Embodiment 4. The compound of any preceding embodiment, wherein at least one internucleoside linkage is a modified internucleoside linkage.
Embodiment 5. The compound of embodiment 4, wherein at least one modified internucleoside linkage is a phosphorothioate internucleoside linkage.
Embodiment 6. The compound of embodiment 4, wherein each modified internucleoside linkage is a phosphorothioate internucleoside linkage.
Embodiment 7. The compound of any preceding embodiment, wherein at least one internucleoside linkage is a phosphodiester internucleoside linkage.
Embodiment 8. The compound of any preceding embodiment, wherein at least one internucleoside linkage is a phosphorothioate linkage and at least one internucleoside linkage is a phosphodiester linkage.
Embodiment 9. The compound of any preceding embodiment, wherein at least one nucleoside comprises a modified nucleobase.
Embodiment 10. The compound of embodiment 9, wherein the modified nucleobase is a 5-methylcytosine.
Embodiment 11. The compound of any preceding embodiment, wherein at least one nucleoside of the modified antisense oligonucleotide comprises a modified sugar.
Embodiment 12. The compound of embodiment 11, wherein the at least one modified sugar is a bicyclic sugar.
Embodiment 13. The compound of embodiment 12, wherein the bicyclic sugar comprises a 4′-CH(R)-0-2′ bridge wherein R is, independently, H, C₁-C₁₂ alkyl, or a protecting group.
Embodiment 14. The compound of embodiment 13, wherein R is methyl.
Embodiment 15. The compound of embodiment 13, wherein R is H.
Embodiment 16. The compound of embodiment 11, wherein the at least one modified sugar comprises a 2′-O-methoxyethyl group.
Embodiment 17. The compound of any preceding embodiment, wherein the modified antisense oligonucleotide comprises:
   a gap segment consisting of 10 linked deoxynucleosides;
   a 5′ wing segment consisting of 5 linked nucleosides; and
   a 3′ wing segment consisting of 5 linked nucleosides; wherein the gap segment is positioned between the 5′ wing segment and the 3′ wing segment and wherein each nucleoside of each wing segment comprises a modified sugar.
Embodiment 18. The compound of any preceding embodiment, wherein the modified antisense oligonucleotide consists of 20 linked nucleosides.
Embodiment 19. A composition comprising the compound of any preceding embodiment or salt thereof and at least one of a pharmaceutically acceptable carrier or diluent.
Embodiment 20. A method comprising administering to an animal the compound or composition of any preceding embodiment.
Embodiment 21. The method of embodiment 20, wherein the animal is a human.
Embodiment 22. The method of embodiment 20, wherein administering the compound prevents, treats, ameliorates, or slows progression of a MECP2 associated disorder or syndrome.
Embodiment 23. The method of embodiment 22, wherein the disease, disorder or condition is MECP2 duplication syndrome.
Embodiment 24. Use of the compound or composition of any preceding embodiment for the manufacture of a medicament for treating a neurological disorder.
Embodiment 25 A method comprising administering a MECP2 antisense compound to an animal for treating a MECP2 associated disorder.
Embodiment 26. A method comprising:
identifying an animal having a MECP2 associated disorder; and administering a MECP2 antisense compound.
Embodiment 27. The method of embodiment 25 or embodiment 26, wherein MECP2 associated disorder is a neurological disorder.
Embodiment 28. The method of any one of embodiments 25-27, wherein the MECP2 associated disorder is MECP2 duplication syndrome.
Embodiment 29. The method of any one of embodiments 25-28, wherein the animal is a human.
Embodiment 30. The method of any one of embodiments 25-29, wherein the administering is parenteral administration.
Embodiment 31. The method of embodiment 30, wherein the parenteral administration is any of intracerebroventricular administration or intrathecal administration.
Embodiment 32. The method of any one of embodiments 25-31, wherein the administering reduces MECP2 mRNA and or protein levels.
Embodiment 33. The method of embodiment 32, wherein the administering reduces MECP2 mRNA and or protein levels by 15, 20, 25, 30, 35, 40, 45, 50, 55, 60, or 65 percent.
Embodiment 34. The method of any one of embodiments 25-33, wherein the administering improves motor function.
Embodiment 35. The method of embodiment 34, wherein motor function is improved by 10, 15, 20, 25, 30, or 35 percent.
Embodiment 36. The method of any one of embodiments 25-35, wherein the administering improves anxiety.
Embodiment 37. The method of embodiment 36, wherein the administering improves anxiety by 5, 10, 15, 20, 25, 30, 35, 40, 45, or 50 percent.
Embodiment 38. The method of any one of embodiments 25-37, wherein the administering improves social interaction.
Embodiment 39. The method of embodiment 38, wherein the administering improves social interaction by 5, 10, 15, 20, 25, 30, 35, 40, 45, or 50 percent.
Embodiment 40. The method of any one of embodiments 25-39, wherein the administering improves activity.
Embodiment 41. The method of embodiment 40, wherein the administering improves activity by 5, 10, 15, 20, 25, 30, 35, 40, 45, 50, 55, 60, 65, 70, 75, or 80 percent.
Embodiment 42. The method of any one of embodiments 25-41, wherein the administering reduces seizures.
Embodiment 43. The method of any one of embodiments 25-42, wherein the administering normalizes EEG discharges.
Embodiment 44. The method of any one of embodiments 25-43, wherein at least one symptom of a MECP2 associated disorder is ameliorated, treated, prevented, or slowed.
Embodiment 45. The method of any one of embodiments 25-44, wherein the antisense compound is a modified antisense oligonucleotide.
Embodiment 46. The method of embodiment 45, wherein the modified antisense oligonucleotide has the nucleobase sequence of SEQ ID NO: 324, 103, 264, 31, or 112.

### Antisense Compounds

Oligomeric compounds include, but are not limited to, oligonucleotides, oligonucleosides, oligonucleotide analogs, oligonucleotide mimetics, antisense compounds, antisense oligonucleotides, and siRNAs. An oligomeric compound may be “antisense” to a target nucleic acid, meaning that is is capable of undergoing hybridization to a target nucleic acid through hydrogen bonding.

In certain embodiments, an antisense compound has a nucleobase sequence that, when written in the 5′ to 3′ direction, comprises the reverse complement of the target segment of a target nucleic acid to which it is targeted. In certain such embodiments, an antisense oligonucleotide has a nucleobase sequence that, when written in the 5′ to 3′ direction, comprises the reverse complement of the target segment of a target nucleic acid to which it is targeted.

In certain embodiments, an antisense compound targeted to a target nucleic acid is 12 to 30 subunits in length. In certain embodiments, an antisense compound targeted to a target nucleic acid is 12 to 25 subunits in length. In certain embodiments, an antisense compound targeted to a target nucleic acid is 12 to 22 subunits in length. In certain embodiments, an antisense compound targeted to a target nucleic acid is 14 to 20 subunits in length. In certain embodiments, an antisense compound targeted to a target nucleic acid is 15 to 25 subunits in length. In certain embodiments, an antisense compound targeted to a target nucleic acid is 18 to 22 subunits in length. In certain embodiments, an antisense compound targeted to a target nucleic acid is 19 to 21 subunits in length. In certain embodiments, the antisense compound is 8 to 80, 12 to 50, 13 to 30, 13 to50, 14to30, 14 to50, 15 to30, 15 to50, 16 to30, 16 to50, 17 to30, 17 to50, 18 to30, 18 to50, 19 to 30, 19 to 50, or 20 to 30 linked subunits in length.

In certain embodiments, an antisense compound targeted to a target nucleic acid is 12 subunits in length. In certain embodiments, an antisense compound targeted to a target nucleic acid is 13 subunits in length. In certain embodiments, an antisense compound targeted to a target nucleic acid is 14 subunits in length. In certain embodiments, an antisense compound targeted to a target nucleic acid is 15 subunits in length. In certain embodiments, an antisense compound targeted to a target nucleic acid is 16 subunits in length. In certain embodiments, an antisense compound targeted to a target nucleic acid is 17 subunits in length. In certain embodiments, an antisense compound targeted to a target nucleic acid is 18 subunits in length. In certain embodiments, an antisense compound targeted to a target nucleic acid is 19 subunits in length. In certain embodiments, an antisense compound targeted to a target nucleic acid is 20 subunits in length. In certain embodiments, an antisense compound targeted to a target nucleic acid is 21 subunits in length. In certain embodiments, an antisense compound targeted to a target nucleic acid is 22 subunits in length. In certain embodiments, an antisense compound targeted to a target nucleic acid is 23 subunits in length. In certain embodiments, an antisense compound targeted to a target nucleic acid is 24 subunits in length. In certain embodiments, an antisense compound targeted to a target nucleic acid is 25 subunits in length. In certain embodiments, an antisense compound targeted to a target nucleic acid is 26 subunits in length. In certain embodiments, an antisense compound targeted to a target nucleic acid is 27 subunits in length. In certain embodiments, an antisense compound targeted to a target nucleic acid is 28 subunits in length. In certain embodiments, an antisense compound targeted to a target nucleic acid is 29 subunits in length. In certain embodiments, an antisense compound targeted to a target nucleic acid is 30 subunits in length. In certain embodiments, the antisense compound targeted to a target nucleic acid is 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked subunits in length, or a range defined by any two of the above values. In certain embodiments the antisense compound is an antisense oligonucleotide, and the linked subunits are nucleosides.

In certain embodiments antisense oligonucleotides targeted to a target nucleic acid may be shortened or truncated. For example, a single subunit may be deleted from the 5′ end (5′ truncation), or alternatively from the 3′ end (3′ truncation). A shortened or truncated antisense compound targeted to a target nucleic acid may have two subunits deleted from the 5′ end, or alternatively may have two subunits deleted from the 3′ end, of the antisense compound. Alternatively, the deleted nucleosides may be dispersed throughout the antisense compound, for example, in an antisense compound having one nucleoside deleted from the 5′ end and one nucleoside deleted from the 3′ end.

When a single additional subunit is present in a lengthened antisense compound, the additional subunit may be located at the 5′ or 3′ end of the antisense compound. When two or more additional subunits are present, the added subunits may be adjacent to each other, for example, in an antisense compound having two subunits added to the 5′ end (5′ addition), or alternatively to the 3′ end (3′ addition), of the antisense compound. Alternatively, the added subunits may be dispersed throughout the antisense compound, for example, in an antisense compound having one subunit added to the 5′ end and one subunit added to the 3′ end.

It is possible to increase or decrease the length of an antisense compound, such as an antisense oligonucleotide, and/or introduce mismatch bases without eliminating activity. For example, in Woolf et al. (Proc. Natl. Acad. Sci. USA 89:7305-7309, 1992), a series of antisense oligonucleotides 13-25 nucleobases in length were tested for their ability to induce cleavage of a target RNA in an oocyte injection model. Antisense oligonucleotides 25 nucleobases in length with 8 or 11 mismatch bases near the ends of the antisense oligonucleotides were able to direct specific cleavage of the target mRNA, albeit to a lesser extent than the antisense oligonucleotides that contained no mismatches. Similarly, target specific cleavage was achieved using 13 nucleobase antisense oligonucleotides, including those with 1 or 3 mismatches.

Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo.

Maher and Dolnick (Nuc. Acid. Res. 16:3341-3358, 1988) tested a series of tandem 14 nucleobase antisense oligonucleotides, and a 28 and 42 nucleobase antisense oligonucleotides comprised of the sequence of two or three of the tandem antisense oligonucleotides, respectively, for their ability to arrest translation of human DHFR in a rabbit reticulocyte assay. Each of the three 14 nucleobase antisense oligonucleotides alone was able to inhibit translation, albeit at a more modest level than the 28 or 42 nucleobase antisense oligonucleotides.

### Antisense Compound Motifs

In certain embodiments, antisense compounds targeted to a target nucleic acid have chemically modified subunits arranged in patterns, or motifs, to confer to the antisense compounds properties such as enhanced inhibitory activity, increased binding affinity for a target nucleic acid, or resistance to degradation by in vivo nucleases.

Chimeric antisense compounds typically contain at least one region modified so as to confer increased resistance to nuclease degradation, increased cellular uptake, increased binding affinity for the target nucleic acid, and/or increased inhibitory activity. A second region of a chimeric antisense compound may optionally serve as a substrate for the cellular endonuclease RNase H, which cleaves the RNA strand of an RNA:DNA duplex.

Antisense compounds having a gapmer motif are considered chimeric antisense compounds. In a gapmer an internal region having a plurality of nucleotides that supports RNaseH cleavage is positioned between external regions having a plurality of nucleotides that are chemically distinct from the nucleosides of the internal region. In the case of an antisense oligonucleotide having a gapmer motif, the gap segment generally serves as the substrate for endonuclease cleavage, while the wing segments comprise modified nucleosides. In certain embodiments, the regions of a gapmer are differentiated by the types of sugar moieties comprising each distinct region. The types of sugar moieties that are used to differentiate the regions of a gapmer may in some embodiments include β-D-ribonucleosides, β-D-deoxyribonucleosides, 2′-modified nucleosides (such 2′-modified nucleosides may include 2′-MOE, and 2′-O—CH₃, among others), and bicyclic sugar modified nucleosides (such bicyclic sugar modified nucleosides may include those having a 4′-(CH₂)ₙ-O-2′ bridge, where n=1 or n=2 and 4′-CH₂—O—CH₂-2′). In certain embodiments, wings may include several modified sugar moieties, including, for example 2′-MOE. In certain embodiments, wings may include several modified and unmodified sugar moieties. In certain embodiments, wings may include various combinations of 2′-MOE nucleosides and 2′-deoxynucleosides.

Each distinct region may comprise uniform sugar moieties, variant, or alternating sugar moieties. The wing-gap-wing motif is frequently described as “X-Y-Z”, where “X” represents the length of the 5′ wing, “Y” represents the length of the gap, and “Z” represents the length of the 3′ wing. “X” and “Z” may comprise uniform, variant, or alternating sugar moieties. In certain embodiments, “X” and “Y” may include one or more 2′-deoxynucleosides. “Y” may comprise 2′-deoxynucleosides. As used herein, a gapmer described as “X-Y-Z” has a configuration such that the gap is positioned immediately adjacent to each of the 5′ wing and the 3′ wing. Thus, no intervening nucleotides exist between the 5′ wing and gap, or the gap and the 3′ wing. Any of the antisense compounds described herein can have a gapmer motif. In certain embodiments, “X” and “Z” are the same; in other embodiments they are different.

In certain embodiments, gapmers provided herein include, for example 20-mers having a motif of 5-10-5. In certain embodiments, gapmers provided herein include, for example 19-mers having a motif of 5-9-5. In certain embodiments, gapmers provided herein include, for example 18-mers having a motif of 5-8-5.

In certain embodiments, gapmers provided herein include, for example 18-mers having a motif of 4-8-6. In certain embodiments, gapmers provided herein include, for example 18-mers having a motif of 6-8-4. In certain embodiments, gapmers provided herein include, for example 18-mers having a motif of 5-7-6.

### Target Nucleic Acids, Target Regions and Nucleotide Sequences

Nucleotide sequences that encode MECP2 include, without limitation, the following: GENBANK Accession No. NM_004992.3 (incorporated herein as SEQ ID NO: 2) and the complement of GENBANK Accession No. NT_167198.1 truncated from nucleotides 4203000 to U.S. Pat. No. 4,283,000 (incorporated herein as SEQ ID NO: 1).

It is understood that the sequence set forth in each SEQ ID NO in the Examples contained herein is independent of any modification to a sugar moiety, an internucleoside linkage, or a nucleobase. As such, antisense compounds defined by a SEQ ID NO may comprise, independently, one or more modifications to a sugar moiety, an intemucleoside linkage, or a nucleobase. Antisense compounds described by Isis Number (Isis No) indicate a combination of nucleobase sequence and motif.

In certain embodiments, a target region is a structurally defined region of the target nucleic acid. For example, a target region may encompass a 3′ UTR, a 5′ UTR, an exon, an intron, an exon/intron junction, a coding region, a translation initiation region, translation termination region, or other defined nucleic acid region. The structurally defined regions for MECP2 can be obtained by accession number from sequence databases such as NCBI and such information is incorporated herein by reference. In certain embodiments, a target region may encompass the sequence from a 5′ target site of one target segment within the target region to a 3′ target site of another target segment within the same target region.

Targeting includes determination of at least one target segment to which an antisense compound hybridizes, such that a desired effect occurs. In certain embodiments, the desired effect is a reduction in mRNA target nucleic acid levels. In certain embodiments, the desired effect is reduction of levels of protein encoded by the target nucleic acid or a phenotypic change associated with the target nucleic acid.

A target region may contain one or more target segments. Multiple target segments within a target region may be overlapping. Alternatively, they may be non-overlapping. In certain embodiments, target segments within a target region are separated by no more than about 300 nucleotides. In certain embodiments, target segments within a target region are separated by a number of nucleotides that is, is about, is no more than, is no more than about, 250, 200, 150, 100, 90, 80, 70, 60, 50, 40, 30, 20, or 10 nucleotides on the target nucleic acid, or is a range defined by any two of the preceeding values. In certain embodiments, target segments within a target region are separated by no more than, or no more than about, 5 nucleotides on the target nucleic acid. In certain embodiments, target segments are contiguous. Contemplated are target regions defined by a range having a starting nucleic acid that is any of the 5′ target sites or 3′ target sites listed herein.

Suitable target segments may be found within a 5′ UTR, a coding region, a 3′ UTR, an intron, an exon, or an exon/intron junction. Target segments containing a start codon or a stop codon are also suitable target segments. A suitable target segment may specifically exclude a certain structurally defined region such as the start codon or stop codon.

The determination of suitable target segments may include a comparison of the sequence of a target nucleic acid to other sequences throughout the genome. For example, the BLAST algorithm may be used to identify regions of similarity amongst different nucleic acids. This comparison can prevent the selection of antisense compound sequences that may hybridize in a non-specific manner to sequences other than a selected target nucleic acid (i.e., non-target or off-target sequences).

There may be variation in activity (e.g., as defined by percent reduction of target nucleic acid levels) of the antisense compounds within an active target region. In certain embodiments, reductions in MECP2 mRNA levels are indicative of inhibition of MECP2 expression. Reductions in levels of an MECP2 protein are also indicative of inhibition of target mRNA expression. Phenotypic changes are indicative of inhibition of MECP2 expression. Improvement in neurological function is indicative of inhibition of MECP2 expression. Improved motor function, activity, social behavior, and memory are indicative of inhibition of MECP2 expression. Reduction of anxiety-like behaviors is indicative of inhibition of MECP2 expression.

### Hybridization

In some embodiments, hybridization occurs between an antisense compound disclosed herein and an MECP2 nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Stringent conditions are sequence-dependent and are determined by the nature and composition of the nucleic acid molecules to be hybridized.

Methods of determining whether a sequence is specifically hybridizable to a target nucleic acid are well known in the art. In certain embodiments, the antisense compounds provided herein are specifically hybridizable with a MECP2 nucleic acid.

### Complementarity

An antisense compound and a target nucleic acid are complementary to each other when a sufficient number of nucleobases of the antisense compound can hydrogen bond with the corresponding nucleobases of the target nucleic acid, such that a desired effect will occur (e.g., antisense inhibition of a target nucleic acid, such as a MECP2 nucleic acid).

Non-complementary nucleobases between an antisense compound and a target nucleic acid may be tolerated provided that the antisense compound remains able to specifically hybridize to a target nucleic acid.

Moreover, an antisense compound may hybridize over one or more segments of a target nucleic acid such that intervening or adjacent segments are not involved in the hybridization event (e.g., a loop structure, mismatch or hairpin structure).

In certain embodiments, the antisense compounds provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a MECP2 nucleic acid, a target region, target segment, or specified portion thereof. Percent complementarity of an antisense compound with a target nucleic acid can be determined using routine methods.

For example, an antisense compound in which 18 of 20 nucleobases of the antisense compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an antisense compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, the antisense compounds provided herein, or specified portions thereof, are fully complementary (i.e., 100% complementary) to a target nucleic acid, or specified portion thereof. For example, an antisense compound may be fully complementary to a MECP2 nucleic acid, or a target region, or a target segment or target sequence thereof. As used herein, “fully complementary” means each nucleobase of an antisense compound is capable of precise base pairing with the corresponding nucleobases of a target nucleic acid. For example, a 20 nucleobase antisense compound is fully complementary to a target sequence that is 400 nucleobases long, so long as there is a corresponding 20 nucleobase portion of the target nucleic acid that is fully complementary to the antisense compound. Fully complementary can also be used in reference to a specified portion of the first and/or the second nucleic acid. For example, a 20 nucleobase portion of a 30 nucleobase antisense compound can be “fully complementary” to a target sequence that is 400 nucleobases long. The 20 nucleobase portion of the 30 nucleobase oligonucleotide is fully complementary to the target sequence if the target sequence has a corresponding 20 nucleobase portion wherein each nucleobase is complementary to the 20 nucleobase portion of the antisense compound. At the same time, the entire 30 nucleobase antisense compound may or may not be fully complementary to the target sequence, depending on whether the remaining 10 nucleobases of the antisense compound are also complementary to the target sequence.

The location of a non-complementary nucleobase may be at the 5′ end or 3′ end of the antisense compound. Alternatively, the non-complementary nucleobase or nucleobases may be at an internal position of the antisense compound. When two or more non-complementary nucleobases are present, they may be contiguous (i.e., linked) or non-contiguous. In one embodiment, a non-complementary nucleobase is located in the wing segment of a gapmer antisense oligonucleotide.

In certain embodiments, antisense compounds that are, or are up to 11, 12, 13, 14, 15, 16, 17, 18, 19, or 20 nucleobases in length comprise no more than 4, no more than 3, no more than 2, or no more than 1 non-complementary nucleobase(s) relative to a target nucleic acid, or specified portion thereof.

In certain embodiments, antisense compounds that are, or are up to 11, 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, or specified portion thereof.

The antisense compounds provided herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an antisense compound. In certain embodiments, the antisense compounds, are complementary to at least an 8 nucleobase portion of a target segment. In certain embodiments, the antisense compounds are complementary to at least a 9 nucleobase portion of a target segment. In certain embodiments, the antisense compounds are complementary to at least a 10 nucleobase portion of a target segment. In certain embodiments, the antisense compounds, are complementary to at least an 11 nucleobase portion of a target segment. In certain embodiments, the antisense compounds, are complementary to at least a 12 nucleobase portion of a target segment. In certain embodiments, the antisense compounds, are complementary to at least a 13 nucleobase portion of a target segment. In certain embodiments, the antisense compounds, are complementary to at least a 14 nucleobase portion of a target segment. In certain embodiments, the antisense compounds, are complementary to at least a 15 nucleobase portion of a target segment. Also contemplated are antisense compounds that are complementary to at least a 9, 10, 11, 12, 13, 14, 15, 16, 17, 18, 19, 20, or more nucleobase portion of a target segment, or a range defined by any two of these values.

### Identity

The antisense compounds provided herein may also have a defined percent identity to a particular nucleotide sequence, SEQ ID NO, or compound represented by a specific Isis number, or portion thereof. As used herein, an antisense compound is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the antisense compounds described herein as well as compounds having non-identical bases relative to the antisense compounds provided herein also are contemplated. The non-identical bases may be adjacent to each other or dispersed throughout the antisense compound. Percent identity of an antisense compound is calculated according to the number of bases that have identical base pairing relative to the sequence to which it is being compared.

In certain embodiments, the antisense compounds, or portions thereof, are at least 70%, 75%, 80%, 85%, 90%, 95%, 96%, 97%, 98%, 99% or 100% identical to one or more of the antisense compounds or SEQ ID NOs, or a portion thereof, disclosed herein.

In certain embodiments, a portion of the antisense compound is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid. In certain embodiments, a portion of the antisense oligonucleotide is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

### Modifications

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar.

Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense compounds encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased inhibitory activity.

Chemically modified nucleosides may also be employed to increase the binding affinity of a shortened or truncated antisense oligonucleotide for its target nucleic acid. Consequently, comparable results can often be obtained with shorter antisense compounds that have such chemically modified nucleosides.

### Modified Internucleoside Linkages

The naturally occurring internucleoside linkage of RNA and DNA is a 3′ to 5′ phosphodiester linkage. Antisense compounds having one or more modified, i.e. non-naturally occurring, internucleoside linkages are often selected over antisense compounds having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, antisense compounds targeted to a MECP2 nucleic acid comprise one or more modified internucleoside linkages. In certain embodiments, the modified internucleoside linkages are interspersed throughout the antisense compound. In certain embodiments, the modified internucleoside linkages are phosphorothioate linkages. In certain embodiments, each internucleoside linkage of an antisense compound is a phosphorothioate internucleoside linkage.

### Modified Sugar Moieties

Antisense compounds can optionally contain one or more nucleosides wherein the sugar group has been modified. Such sugar modified nucleosides may impart enhanced nuclease stability, increased binding affinity, or some other beneficial biological property to the antisense compounds. In certain embodiments, nucleosides comprise chemically modified ribofuranose ring moieties. Examples of chemically modified ribofuranose rings include without limitation, addition of substitutent groups (including 5′ and 2′ substituent groups, bridging of non-geminal ring atoms to form bicyclic nucleic acids (BNA), replacement of the ribosyl ring oxygen atom with S, N(R), or C(R₁)(R₂) (R, R₁ and R₂ are each independently H, C₁-C₁₂ alkyl or a protecting group) and combinations thereof. Examples of chemically modified sugars include 2′-F-5′-methyl substituted nucleoside (see PCT International Application WO 2008/101157 Published on Aug. 21, 2008 for other disclosed 5′,2′-bis substituted nucleosides) or replacement of the ribosyl ring oxygen atom with S with further substitution at the 2′-position (see published U.S. Patent Application US2005-0130923, published on Jun. 16, 2005) or alternatively 5′-substitution of a BNA (see PCT International Application WO 2007/134181 Published on Nov. 22, 2007 wherein LNA is substituted with for example a 5′-methyl or a 5′-vinyl group).

Examples of nucleosides having modified sugar moieties include without limitation nucleosides comprising 5′-vinyl, 5′-methyl (R or S), 4-S, 2′-F, 2′-OCH₃, 2′-OCH₂CH₃, 2′-OCH₂CH₂F and 2′-O(CH₂)₂OCH₃ substituent groups. The substituent at the 2′ position can also be selected from allyl, amino, azido, thio, 0-allyl, 0-C₁-C₁₀ alkyl, OCF₃, OCH₂F, O(CH₂)₂SCH₃, O(CH₂)₂-O—N(Rₘ)(Rₙ), 0-CH₂—C(═O)—N(Rₘ)(Rₙ), and O—CH₂—C(═O)—N(R₁)—(CH₂)₂-N(Rₘ)(Rₙ), where each R₁, Rₘ and Rₙ is, independently, H or substituted or unsubstituted C₁-C₁₀ alkyl.

As used herein, “bicyclic nucleosides” refer to modified nucleosides comprising a bicyclic sugar moiety. Examples of bicyclic nucleosides include without limitation nucleosides comprising a bridge between the 4′ and the 2′ ribosyl ring atoms. In certain embodiments, antisense compounds provided herein include one or more bicyclic nucleosides comprising a 4′ to 2′ bridge. Examples of such 4′ to 2′ bridged bicyclic nucleosides, include but are not limited to one of the formulae: 4′-(CH₂)—O-2′ (LNA); 4′-(CH₂)—S-2′; 4′-(CH₂)₂-O-2′ (ENA); 4′-CH(CH₃)—O-2′ and 4′-CH(CH₂OCH₃)—O-2′ (and analogs thereof see U.S. Pat. No. 7,399,845, issued on Jul. 15, 2008); 4′-C(CH₃)(CH₃)—O-2′ (and analogs thereof see published International Application WO/2009/006478, published Jan. 8, 2009); 4′-CH₂—N(OCH₃)-2′ (and analogs thereof see published International Application WO/2008/150729, published Dec. 11, 2008); 4′-CH₂—O—N(CH₃)-2′ (see published U.S. Patent Application US2004-0171570, published Sep. 2, 2004); 4′-CH₂—N(R)—O-2′, wherein R is H, C₁-C₁₂ alkyl, or a protecting group (see U.S. Pat. No. 7,427,672, issued on Sep. 23, 2008); 4′-CH₂—C(H)(CH₃)-2′ (see Chattopadhyaya et al., J. Org. Chem., 2009, 74, 118-134); and 4′-CH₂—C—(═CH₂)-2′ (and analogs thereof see published International Application WO 2008/154401, published on Dec. 8, 2008).

Further reports related to bicyclic nucleosides can also be found in published literature (see for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129(26) 8362-8379; Elayadi et al., Curr. Opinion Invest. Drugs, 2001, 2, 558-561; Braasch et al., Chem. Biol., 2001, 8, 1-7; and Orum et al., Curr. Opinion Mol. Ther., 2001, 3, 239-243; U.S. Pat. Nos. 6,268,490; 6,525,191; 6,670,461; 6,770,748; 6,794,499; 7,034,133; 7,053,207; 7,399,845; 7,547,684; and 7,696,345; U.S. Patent Publication No. US2008-0039618; US2009-0012281; U.S. Patent Serial Nos. 60/989,574; 61/026,995; 61/026,998; 61/056,564; 61/086,231; 61/097,787; and 61/099,844; Published PCT International applications WO 1994/014226; WO 2004/106356; WO 2005/021570; WO 2007/134181; WO 2008/150729; WO 2008/154401; and WO 2009/006478. Each of the foregoing bicyclic nucleosides can be prepared having one or more stereochemical sugar configurations including for example α-L-ribofuranose and β-D-ribofuranose (see PCT international application PCT/DK98/00393, published on Mar. 25, 1999 as WO 99/14226).

In certain embodiments, bicyclic sugar moieties of BNA nucleosides include, but are not limited to, compounds having at least one bridge between the 4′ and the 2′ position of the pentofuranosyl sugar moiety wherein such bridges independently comprises 1 or from 2 to 4 linked groups independently selected from —[C(Rₐ)(R_{b})]ₙ—, —C(Rₐ)═C(R_{b})—, —C(Rₐ)═N—, —C(═O)—, —C(═NRₐ)—, —C(═S)—, —O—, —Si(Rₐ)₂—, —S(═O)ᵣ—, and —N(Rₐ)—;
wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

In certain embodiments, the bridge of a bicyclic sugar moiety is —[C(Rₐ)(R_{b})]ₙ—, —[C(Rₐ)(R_{b})]ₙ—O—C(RₐR_{b})—N(R)—O— or —C(RₐR_{b})—O—N(R)—. In certain embodiments, the bridge is 4′-CH₂-2′, 4′-(CH₂)₂-2′, 4′-(CH₂)₃-2′, 4′-CH₂—O-2′, 4′-(CH₂)₂-O-2′, 4′-CH₂—O—N(R)-2′ and 4′-CH₂—N(R)—O-2′- wherein each R is, independently, H, a protecting group or C₁-C₁₂ alkyl.

In certain embodiments, bicyclic nucleosides are further defined by isomeric configuration. For example, a nucleoside comprising a 4′-2′ methylene-oxy bridge, may be in the α-L configuration or in the β-D configuration. Previously, α-L-methyleneoxy (4′-CH₂—O-2′) BNA's have been incorporated into antisense oligonucleotides that showed antisense activity (Frieden et al., Nucleic Acids Research, 2003, 21, 6365-6372).

In certain embodiments, bicyclic nucleosides include, but are not limited to, (A) α-L-methyleneoxy (4′-CH₂—O-2′) BNA, (B) β-D-methyleneoxy (4′-CH₂—O-2′) BNA, (C) ethyleneoxy (4′-(CH₂)₂-O-2′) BNA, (D) aminooxy (4′-CH₂—O—N(R)-2′) BNA, (E) oxyamino (4′-CH₂—N(R)—O-2′) BNA, and (F) methyl(methyleneoxy) (4′-CH(CH₃)—O-2′) BNA, (G) methylene-thio (4′-CH₂—S-2′) BNA, (H) methylene-amino (4′-CH₂—N(R)-2′) BNA, (I) methyl carbocyclic (4′-CH₂—CH(CH₃)-2′) BNA, and (J) propylene carbocyclic (4′-(CH₂)₃-2′) BNA as depicted below.

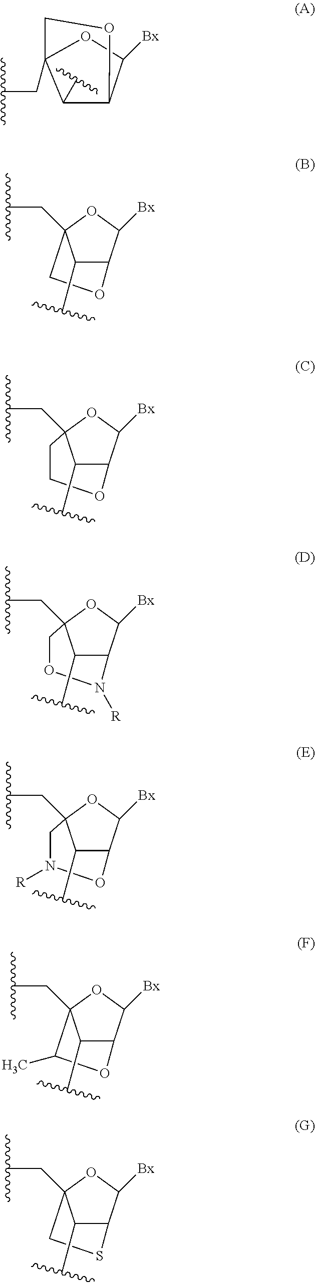

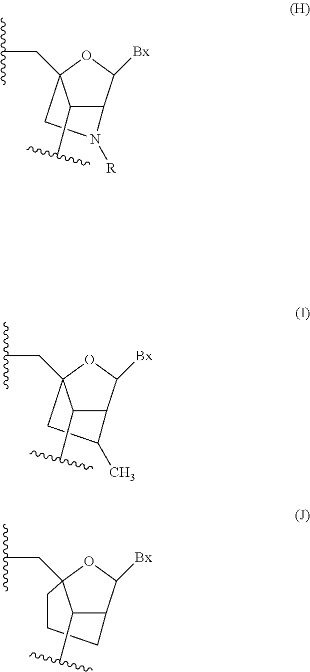

wherein Bx is the base moiety and R is independently H, a protecting group or C₁-C₁₂ alkyl.

In certain embodiments, bicyclic nucleosides are provided having Formula I:

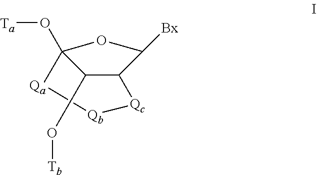

wherein:
Bx is a heterocyclic base moiety;
-Qₐ-Q_{b}-Q_{c}- is —CH₂—N(R_{c})—CH₂—, —C(═O)—N(R_{c})—CH₂—, —CH₂—O—N(R_{c})—, —CH₂—N(R_{c})—O— or —N(R_{c})—O—CH₂;
R_{c} is C₁-C₁₂ alkyl or an amino protecting group; and
Tₐ and T_{b} are each, independently H, a hydroxyl protecting group, a conjugate group, a reactive phosphorus group, a phosphorus moiety or a covalent attachment to a support medium.

In certain embodiments, bicyclic nucleosides are provided having Formula II:

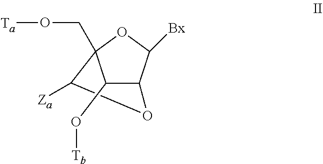

wherein:
Bx is a heterocyclic base moiety;
Tₐ and T_{b} are each, independently H, a hydroxyl protecting group, a conjugate group, a reactive phosphorus group, a phosphorus moiety or a covalent attachment to a support medium;
Zₐ is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, substituted amide, thiol or substituted thio.

In one embodiment, each of the substituted groups is, independently, mono or poly substituted with substituent groups independently selected from halogen, oxo, hydroxyl, OJ_{c}, NJ_{c}J_{d}, SJ_{c}, N₃, OC(═X)J_{c}, and NJ_{c}C(═X)NJ_{c}J_{d}, wherein each J_{c}, J_{d} and Jₑ is, independently, H, C₁-C₆ alkyl, or substituted C₁-C₆ alkyl and X is O or NJ_{c}.

In certain embodiments, bicyclic nucleosides are provided having Formula III:

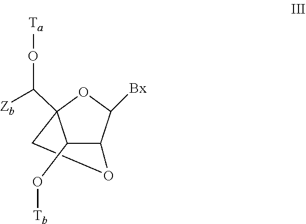

wherein:
Bx is a heterocyclic base moiety;
Tₐ and T_{b} are each, independently H, a hydroxyl protecting group, a conjugate group, a reactive phosphorus group, a phosphorus moiety or a covalent attachment to a support medium;
Z_{b} is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl or substituted acyl (C(═O)—).

In certain embodiments, bicyclic nucleosides are provided having Formula IV:

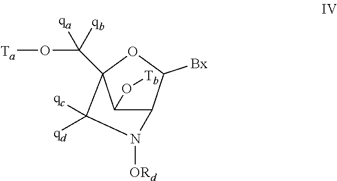

wherein:
Bx is a heterocyclic base moiety;
Tₐ and T_{b} are each, independently H, a hydroxyl protecting group, a conjugate group, a reactive phosphorus group, a phosphorus moiety or a covalent attachment to a support medium;
Rd is C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl;
each qₐ, q_{b}, q_{c} and q_{d} is, independently, H, halogen, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl, C₁-C₆ alkoxyl, substituted C₁-C₆ alkoxyl, acyl, substituted acyl, C₁-C₆ aminoalkyl or substituted C₁-C₆ aminoalkyl; In certain embodiments, bicyclic nucleosides are provided having Formula V:

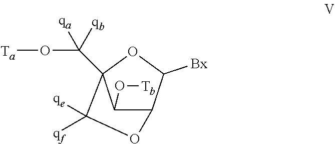

wherein:
Bx is a heterocyclic base moiety;
Tₐ and T_{b} are each, independently H, a hydroxyl protecting group, a conjugate group, a reactive phosphorus group, a phosphorus moiety or a covalent attachment to a support medium;
qₐ, q_{b}, qₑ and q_{f} are each, independently, hydrogen, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxy, substituted C₁-C₁₂ alkoxy, OJⱼ, SJⱼ, SO₂Jⱼ, SO₂Jⱼ, NⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ;
or qₑ and q_{f} together are ═C(q_{g})(qₕ);
q_{g} and qₕ are each, independently, H, halogen, C₁-C₁₂ alkyl or substituted C₁-C₁₂ alkyl.

The synthesis and preparation of the methyleneoxy (4′-CH₂—O-2′) BNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). BNAs and preparation thereof are also described in WO 98/39352 and WO 99/14226.

Analogs of methyleneoxy (4′-CH₂—O-2′) BNA and 2′-thio-BNAs, have also been prepared (Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (Wengel et al., WO 99/14226). Furthermore, synthesis of 2′-amino-BNA, a novel conformationally restricted high-affinity oligonucleotide analog has been described in the art (Singh et al., J. Org. Chem., 1998, 63, 10035-10039). In addition, 2′-amino- and 2′-methylamino-BNA's have been prepared and the thermal stability of their duplexes with complementary RNA and DNA strands has been previously reported.

In certain embodiments, bicyclic nucleosides are provided having Formula VI:

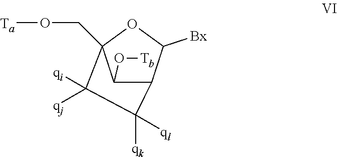

wherein:
Bx is a heterocyclic base moiety;
Tₐ and T_{b} are each, independently H, a hydroxyl protecting group, a conjugate group, a reactive phosphorus group, a phosphorus moiety or a covalent attachment to a support medium;
each qᵢ, qⱼ, qₖ and qₗ is, independently, H, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxyl, substituted C₁-C₁₂ alkoxyl, OJⱼ, SJⱼ, SOJⱼ, SO₂Jⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ; and
q₁ and qⱼ or qₗ and qₖ together are ═C(q_{g})(qₕ), wherein q_{g} and qₕ are each, independently, H, halogen, C₁-C₁₂ alkyl or substituted C₁-C₁₂ alkyl.

One carbocyclic bicyclic nucleoside having a 4′-(CH₂)₃-2′ bridge and the alkenyl analog bridge 4′-CH═CH—CH₂-2′ have been described (Freier et al., Nucleic Acids Research, 1997, 25(22), 4429-4443 and Albaek et al., J. Org. Chem., 2006, 71, 7731-7740). The synthesis and preparation of carbocyclic bicyclic nucleosides along with their oligomerization and biochemical studies have also been described (Srivastava et al., J. Am. Chem. Soc., 2007, 129(26), 8362-8379).

As used herein, “4′-2′ bicyclic nucleoside” or “4′ to 2′ bicyclic nucleoside” refers to a bicyclic nucleoside comprising a furanose ring comprising a bridge connecting two carbon atoms of the furanose ring connects the 2′ carbon atom and the 4′ carbon atom of the sugar ring.

As used herein, “monocylic nucleosides” refer to nucleosides comprising modified sugar moieties that are not bicyclic sugar moieties. In certain embodiments, the sugar moiety, or sugar moiety analogue, of a nucleoside may be modified or substituted at any position.

As used herein, “2′-modified sugar” means a furanosyl sugar modified at the 2′ position. In certain embodiments, such modifications include substituents selected from: a halide, including, but not limited to substituted and unsubstituted alkoxy, substituted and unsubstituted thioalkyl, substituted and unsubstituted amino alkyl, substituted and unsubstituted alkyl, substituted and unsubstituted allyl, and substituted and unsubstituted alkynyl. In certain embodiments, 2′ modifications are selected from substituents including, but not limited to: O[(CH₂)ₙO]ₘCH₃, O(CH₂)ₙNH₂, O(CH₂)ₙCH₃, O(CH₂)ₙF, O(CH₂)ₙONH₂, OCH₂C(═O)N(H)CH₃, and O(CH₂)ₙON[(CH₂)ₙCH₃]2, where n and m are from 1 to about 10. Other 2′-substituent groups can also be selected from: C₁-C₁₂ alkyl, substituted alkyl, alkenyl, alkynyl, alkaryl, aralkyl, O-alkaryl or O-aralkyl, SH, SCH₃, OCN, Cl, Br, CN, F, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving pharmacokinetic properties, or a group for improving the pharmacodynamic properties of an antisense compound, and other substituents having similar properties. In certain embodiments, modified nucleosides comprise a 2′-MOE side chain (Baker et al., J Biol. Chem., 1997, 272, 11944-12000). Such 2′-MOE substitution have been described as having improved binding affinity compared to unmodified nucleosides and to other modified nucleosides, such as 2′- O-methyl, O-propyl, and O-aminopropyl. Oligonucleotides having the 2′-MOE substituent also have been shown to be antisense inhibitors of gene expression with promising features for in vivo use (Martin, Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926).

As used herein, a “modified tetrahydropyran nucleoside” or “modified THP nucleoside” means a nucleoside having a six-membered tetrahydropyran “sugar” substituted in for the pentofuranosyl residue in normal nucleosides (a sugar surrogate). Modified THP nucleosides include, but are not limited to, what is referred to in the art as hexitol nucleic acid (HNA), anitol nucleic acid (ANA), manitol nucleic acid (MNA) (see Leumann, Bioorg. Med. Chem., 2002, 10, 841-854), fluoro HNA (F-HNA) or those compounds having Formula VII:

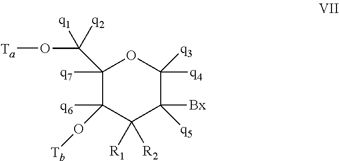

wherein independently for each of said at least one tetrahydropyran nucleoside analog of Formula VII:
Bx is a heterocyclic base moiety;
Tₐ and T_{b} are each, independently, an intemucleoside linking group linking the tetrahydropyran nucleoside analog to the antisense compound or one of Tₐ and T_{b} is an internucleoside linking group linking the tetrahydropyran nucleoside analog to the antisense compound and the other of Tₐ and T_{b} is H, a hydroxyl protecting group, a linked conjugate group or a 5′ or 3′-terminal group;
q₁, q₂, q₃, q₄, q₅, q₆ and q₇ are each independently, H, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl; and each of R₁ and R₂ is selected from hydrogen, hydroxyl, halogen, substituted or unsubstituted alkoxy, NJ₁J₂, SJ₁, N₃, OC(═X)J₁, OC(═X)NJ₁J₂, NJ₃C(═X)NJ₁J₂ and CN, wherein X is O, S or NJ₁ and each J₁, J₂ and J₃ is, independently, H or C₁-C₆ alkyl.

In certain embodiments, the modified THP nucleosides of Formula VII are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, THP nucleosides of Formula VII are provided wherein one of R₁ and R₂ is fluoro. In certain embodiments, R₁ is fluoro and R₂ is H; R₁ is methoxy and R₂ is H, and R₁ is H and R₂ is methoxyethoxy.

As used herein, “2′-modified” or “2′-substituted” refers to a nucleoside comprising a sugar comprising a substituent at the 2′ position other than H or OH. 2′-modified nucleosides, include, but are not limited to, bicyclic nucleosides wherein the bridge connecting two carbon atoms of the sugar ring connects the 2′ carbon and another carbon of the sugar ring; and nucleosides with non-bridging 2′substituents, such as allyl, amino, azido, thio, 0-allyl, 0-C₁-C₁₀ alkyl, —OCF₃, O—(CH₂)₂-O—CH₃, 2′-O(CH₂)₂SCH₃, O—(CH₂)₂-O—N(Rₘ)(Rₙ), or O—CH₂—C(═O)—N(Rₘ)(Rₙ), where each Rₘ and Rₙ is, independently, H or substituted or unsubstituted C₁-C₁₀ alkyl. 2′-modified nucleosides may further comprise other modifications, for example at other positions of the sugar and/or at the nucleobase.

As used herein, “2′-F” refers to a nucleoside comprising a sugar comprising a fluoro group at the 2′ position.

As used herein, “2′-OMe” or “2′-OCH₃” or “2′-O-methyl” each refers to a nucleoside comprising a sugar comprising an —OCH₃ group at the 2′ position of the sugar ring.

As used herein, “MOE” or “2′-MOE” or “2′-OCH₂CH₂OCH₃” or “2′-O-methoxyethyl” each refers to a nucleoside comprising a sugar comprising a —OCH₂CH₂OCH₃ group at the 2′ position of the sugar ring.

As used herein, “oligonucleotide” refers to a compound comprising a plurality of linked nucleosides.

In certain embodiments, one or more of the plurality of nucleosides is modified. In certain embodiments, an oligonucleotide comprises one or more ribonucleosides (RNA) and/or deoxyribonucleosides (DNA).

Many other bicyclo and tricyclo sugar surrogate ring systems are also known in the art that can be used to modify nucleosides for incorporation into antisense compounds (see for example review article: Leumann, Bioorg. Med. Chem., 2002, 10, 841-854).

Such ring systems can undergo various additional substitutions to enhance activity.

Methods for the preparations of modified sugars are well known to those skilled in the art.

In nucleotides having modified sugar moieties, the nucleobase moieties (natural, modified or a combination thereof) are maintained for hybridization with an appropriate nucleic acid target.

In certain embodiments, antisense compounds comprise one or more nucleosides having modified sugar moieties. In certain embodiments, the modified sugar moiety is 2′-MOE. In certain embodiments, the 2′-MOE modified nucleosides are arranged in a gapmer motif. In certain embodiments, the modified sugar moiety is a bicyclic nucleoside having a (4′-CH(CH₃)—O-2′) bridging group. In certain embodiments, the (4′-CH(CH₃)—O-2′) modified nucleosides are arranged throughout the wings of a gapmer motif.

### Compositions and Methods for Formulating Pharmaceutical Compositions

Antisense oligonucleotides may be admixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disorder, or dose to be administered.

An antisense compound targeted to a MECP2 nucleic acid can be utilized in pharmaceutical compositions by combining the antisense compound with a suitable pharmaceutically acceptable diluent or carrier. A pharmaceutically acceptable diluent includes phosphate-buffered saline (PBS). PBS is a diluent suitable for use in compositions to be delivered parenterally. Accordingly, in one embodiment, employed in the methods described herein is a pharmaceutical composition comprising an antisense compound targeted to a MECP2 nucleic acid and a pharmaceutically acceptable diluent. In certain embodiments, the pharmaceutically acceptable diluent is PBS. In certain embodiments, the antisense compound is an antisense oligonucleotide.

Pharmaceutical compositions comprising antisense compounds encompass any pharmaceutically acceptable salts, esters, or salts of such esters, or any other oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of an antisense compound which are cleaved by endogenous nucleases within the body, to form the active antisense compound.

### Conjugated Antisense Compounds

Antisense compounds may be covalently linked to one or more moieties or conjugates which enhance the activity, cellular distribution or cellular uptake of the resulting antisense oligonucleotides. Typical conjugate groups include cholesterol moieties and lipid moieties. Additional conjugate groups include carbohydrates, phospholipids, biotin, phenazine, folate, phenanthridine, anthraquinone, acridine, fluoresceins, rhodamines, coumarins, and dyes.

Antisense compounds can also be modified to have one or more stabilizing groups that are generally attached to one or both termini of antisense compounds to enhance properties such as, for example, nuclease stability. Included in stabilizing groups are cap structures. These terminal modifications protect the antisense compound having terminal nucleic acid from exonuclease degradation, and can help in delivery and/or localization within a cell. The cap can be present at the 5′-terminus (5′-cap), or at the 3′-terminus (3′-cap), or can be present on both termini. Cap structures are well known in the art and include, for example, inverted deoxy abasic caps. Further 3′ and 5′-stabilizing groups that can be used to cap one or both ends of an antisense compound to impart nuclease stability include those disclosed in WO 03/004602 published on Jan. 16, 2003.

### Cell Culture and Antisense Compounds Treatment

The effects of antisense compounds on the level, activity or expression of MECP2 nucleic acids can be tested in vitro in a variety of cell types. Cell types used for such analyses are available from commercial vendors (e.g. American Type Culture Collection, Manassas, VA; Zen-Bio, Inc., Research Triangle Park, NC; Clonetics Corporation, Walkersville, MD) and are cultured according to the vendor's instructions using commercially available reagents (e.g. Invitrogen Life Technologies, Carlsbad, CA). Illustrative cell types include, but are not limited to, HepG2 cells, Hep3B cells, and primary hepatocytes. In certain embodiments, cells are patient cells, such as B-lymphoblast cells.

### In Vitro Testing of Antisense Oligonucleotides

Described herein are methods for treatment of cells with antisense oligonucleotides, which can be modified appropriately for treatment with other antisense compounds.

Cells may be treated with antisense oligonucleotides when the cells reach approximately 60-80% confluency in culture.

One reagent commonly used to introduce antisense oligonucleotides into cultured cells includes the cationic lipid transfection reagent LIPOFECTIN (Invitrogen, Carlsbad, CA). Antisense oligonucleotides may be mixed with LIPOFECTIN in OPTI-MEM 1 (Invitrogen, Carlsbad, CA) to achieve the desired final concentration of antisense oligonucleotide and a LIPOFECTIN concentration that may range from 2 to 12 μg/mL per 100 nM antisense oligonucleotide.

Another reagent used to introduce antisense oligonucleotides into cultured cells includes LIPOFECTAMINE (Invitrogen, Carlsbad, CA). Antisense oligonucleotide is mixed with LIPOFECTAMINE in OPTI-MEM 1 reduced serum medium (Invitrogen, Carlsbad, CA) to achieve the desired concentration of antisense oligonucleotide and a LIPOFECTAMINE concentration that may range from 2 to 12 μg/mL per 100 nM antisense oligonucleotide.

Another reagent used to introduce antisense oligonucleotides into cultured cells includes TURBOFECT (Thermo Scientific, Carlsbad, CA).

Another technique used to introduce antisense oligonucleotides into cultured cells includes electroporation.

Cells are treated with antisense oligonucleotides by routine methods. Cells may be harvested 16-24 hours after antisense oligonucleotide treatment, at which time RNA or protein levels of target nucleic acids are measured by methods known in the art and described herein. In general, when treatments are performed in multiple replicates, the data are presented as the average of the replicate treatments.

The concentration of antisense oligonucleotide used varies from cell line to cell line. Methods to determine the optimal antisense oligonucleotide concentration for a particular cell line are well known in the art. Antisense oligonucleotides are typically used at concentrations ranging from 1 nM to 300 nM when transfected with LIPOFECTAMINE. Antisense oligonucleotides are used at higher concentrations ranging from 625 to 20,000 nM when transfected using electroporation.

### RNA Isolation

RNA analysis can be performed on total cellular RNA or poly(A)+mRNA. Methods of RNA isolation are well known in the art. RNA is prepared using methods well known in the art, for example, using the TRIZOL Reagent (Invitrogen, Carlsbad, CA) according to the manufacturer's recommended protocols.

### Analysis of Inhibition of Target Levels or Expression

Inhibition of levels or expression of a MECP2 nucleic acid can be assayed in a variety of ways known in the art. For example, target nucleic acid levels can be quantitated by, e.g., Northern blot analysis, competitive polymerase chain reaction (PCR), or quantitative real-time PCR. RNA analysis can be performed on total cellular RNA or poly(A)+mRNA. Methods of RNA isolation are well known in the art. Northern blot analysis is also routine in the art. Quantitative real-time PCR can be conveniently accomplished using the commercially available ABI PRISM 7600, 7700, or 7900 Sequence Detection System, available from PE-Applied Biosystems, Foster City, CA and used according to manufacturer's instructions.

### Quantitative Real-Time PCR Analysis of Target RNA Levels

Quantitation of target RNA levels may be accomplished by quantitative real-time PCR using the ABI PRISM 7600, 7700, or 7900 Sequence Detection System (PE-Applied Biosystems, Foster City, CA) according to manufacturer's instructions. Methods of quantitative real-time PCR are well known in the art.

Prior to real-time PCR, the isolated RNA is subjected to a reverse transcriptase (RT) reaction, which produces complementary DNA (cDNA) that is then used as the substrate for the real-time PCR amplification. The RT and real-time PCR reactions are performed sequentially in the same sample well. RT and real-time PCR reagents may be obtained from Invitrogen (Carlsbad, CA). RT real-time-PCR reactions are carried out by methods well known to those skilled in the art.

Gene (or RNA) target quantities obtained by real time PCR are normalized using either the expression level of a gene whose expression is constant, such as cyclophilin A, or by quantifying total RNA using RIBOGREEN (Invitrogen, Inc. Carlsbad, CA). Cyclophilin A expression is quantified by real time PCR, by being run simultaneously with the target, multiplexing, or separately. Total RNA is quantified using RIBOGREEN RNA quantification reagent (Invetrogen, Inc. Eugene, OR). Methods of RNA quantification by RIBOGREEN are taught in Jones, L. J., et al, (Analytical Biochemistry, 1998, 265, 368-374). A CYTOFLUOR 4000 instrument (PE Applied Biosystems) is used to measure RIBOGREEN fluorescence.

Probes and primers are designed to hybridize to a MECP2 nucleic acid. Methods for designing real-time PCR probes and primers are well known in the art, and may include the use of software such as PRIMER EXPRESS Software (Applied Biosystems, Foster City, CA).

Analysis of Protein Levels Antisense inhibition of MECP2 nucleic acids can be assessed by measuring MECP2 protein levels.

Protein levels of MECP2 can be evaluated or quantitated in a variety of ways well known in the art, such as immunoprecipitation, Western blot analysis (immunoblotting), enzyme-linked immunosorbent assay (ELISA), quantitative protein assays, protein activity assays (for example, caspase activity assays), immunohistochemistry, immunocytochemistry or fluorescence-activated cell sorting (FACS). Antibodies directed to a target can be identified and obtained from a variety of sources, such as the MSRS catalog of antibodies (Aerie Corporation, Birmingham, MI), or can be prepared via conventional monoclonal or polyclonal antibody generation methods well known in the art.

### In Vivo Testing of Antisense Compounds

Antisense compounds, for example, antisense oligonucleotides, are tested in animals to assess their ability to inhibit expression of MECP2 and produce phenotypic changes, such as, improved behavior, motor function, and cognition. In certain embodiments, motor function is measured by walking initiation analysis, rotarod, grip strength, pole climb, open field performance, balance beam, hindpaw footprint testing in the animal. In certain embodiments, behavior is measured by elevated plus maze and three-chamber social interaction. Testing may be performed in normal animals, or in experimental models. For administration to animals, antisense oligonucleotides are formulated in a pharmaceutically acceptable diluent, such as phosphate-buffered saline. Administration includes parenteral routes of administration, such as intraperitoneal, intravenous, and subcutaneous. Calculation of antisense oligonucleotide dosage and dosing frequency is within the abilities of those skilled in the art, and depends upon factors such as route of administration and animal body weight. Following a period of treatment with antisense oligonucleotides, RNA is isolated from CNS tissue or CSF and changes in MECP2 nucleic acid expression are measured.

### Certain Indications

In certain embodiments, provided herein are methods of treating an individual comprising administering one or more pharmaceutical compositions described herein. In certain embodiments, the individual has a neurological disorder. In certain embodiments, the individual is at risk for developing a neurological disorder, including, but not limited to, MECP2 duplication syndrome. In certain embodiments, the individual has been identified as having a MECP2 associated disorder. In certain embodiments, provided herein are methods for prophylactically reducing MECP2 expression in an individual. Certain embodiments include treating an individual in need thereof by administering to an individual a therapeutically effective amount of an antisense compound targeted to a MECP2 nucleic acid.

In one embodiment, administration of a therapeutically effective amount of an antisense compound targeted to a MECP2 nucleic acid is accompanied by monitoring of MECP2 levels in an individual, to determine an individual's response to administration of the antisense compound. An individual's response to administration of the antisense compound may be used by a physician to determine the amount and duration of therapeutic intervention.

In certain embodiments, administration of an antisense compound targeted to a MECP2 nucleic acid results in reduction of MECP2 mRNA and or protein expression by at least 15, 20, 25, 30, 35, 40, 45, 50, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or a range defined by any two of these values.

In certain embodiments, administration of an antisense compound targeted to a MECP2 nucleic acid results in improved motor function in an animal. In certain embodiments, administration of a MECP2 antisense compound improves motor function by at least 15, 20, 25, 30, 35, 40, 45, 50, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or a range defined by any two of these values.

In certain embodiments, administration of an antisense compound targeted to a MECP2 nucleic acid results in improved anxiety in an animal. In certain embodiments, administration of a MECP2 antisense compound improves anxiety by at least 15, 20, 25, 30, 35, 40, 45, 50, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or a range defined by any two of these values.

In certain embodiments, administration of an antisense compound targeted to a MECP2 nucleic acid results in improved social interaction in an animal. In certain embodiments, administration of a MECP2 antisense compound improves social interaction by at least 15, 20, 25, 30, 35, 40, 45, 50, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or a range defined by any two of these values.

In certain embodiments, administration of an antisense compound targeted to a MECP2 nucleic acid results in improved activity in an animal. In certain embodiments, administration of a MECP2 antisense compound improves activity by at least 15, 20, 25, 30, 35, 40, 45, 50, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or a range defined by any two of these values.

In certain embodiments, administration of an antisense compound targeted to a MECP2 nucleic acid results in reduction of seizures. In certain embodiments, administration of a MECP2 antisense compound reduces seizures by at least 15, 20, 25, 30, 35, 40, 45, 50, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or a range defined by any two of these values.

In certain embodiments, administration of an antisense compound targeted to a MECP2 nucleic acid results in normalized EEG discharges.

In certain embodiments, pharmaceutical compositions comprising an antisense compound targeted to MECP2 are used for the preparation of a medicament for treating a patient suffering or susceptible to a neurological disorder including MECP2 duplication syndrome.

### Certain Amplicon Regions

Certain antisense oligonucleotides described herein may target the amplicon region of the primer probe set. Additional assays may be used to measure the potency and efficacy of these compounds.

### Certain Hotspot Regions

### 1. Nucleobases 28-382, 386-437, 439-464, 478-513, 519-602, 606-716, 720-789, 797-973, 977-1126, 1130-1189, 1192-1275, 1310-1337, 1440-1509, and 1514-1793

In certain embodiments, modified antisense oligonucleotides are complementary to nucleobases 28-382, 386-437, 439-464, 478-513, 519-602, 606-716, 720-789, 797-973, 977-1126, 1130-1189, 1192-1275, 1310-1337, 1440-1509, and 1514-1793 of SEQ ID NO: 2. In certain embodiments, nucleobases 28-382, 386-437, 439-464, 478-513, 519-602, 606-716, 720-789, 797-973, 977-1126, 1130-1189, 1192-1275, 1310-1337, 1440-1509, and 1514-1793 of SEQ ID NO: 2 are hotspot regions. In certain embodiments, such modified antisense oligonucleotides are 20 nucleobases in length. In certain embodiments, such modified antisense oligonucleotides are gapmers. In certain such embodiments, the gapmers are 5-10-5 MOE gapmers. In certain embodiments, the nucleosides of the modified antisense oligonucleotides are linked by phosphorothioate and phosphodiester internucleoside linkages.

The nucleobase sequences of SEQ ID Nos: 17, 18, 22-24, 50-60, 62-84, 86-93, 95-96, 99-102, 129-131, 133, 135-158, 161-171, 173-174, 177-180, 207-213, 215-237, 239-244, 246-252, 256-258, 284-288, 290, 292, 293, 296-305, 307-315, and 317-327 are complementary to nucleobases 28-382, 386-437, 439-464, 478-513, 519-602, 606-716, 720-789, 797-973, 977-1126, 1130-1189, 1192-1275, 1310-1337, 1440-1509, and 1514-1793 of SEQ ID NO: 2.

In certain embodiments, modified antisense oligonucleotides complementary to nucleobases 28-382, 386-437, 439-464, 478-513, 519-602, 606-716, 720-789, 797-973, 977-1126, 1130-1189, 1192-1275, 1310-1337, 1440-1509, and 1514-1793 of SEQ ID NO: 2 achieve at least 25% reduction of MECP2 RNA in vitro in the standard cell assay.

### 2. Nucleobases 44-79, 87-126, 131-273, 321-376, 478-513, 535-570, 630-716, 834-928, 930-973, 977-1004, 1081-1126, 1130-1189, 1224-1275, 1440-1509, 1514-1745, and 1750-1785

In certain embodiments, modified antisense oligonucleotides are complementary to nucleobases 44-79, 87-126, 131-273, 321-376, 478-513, 535-570, 630-716, 834-928, 930-973, 977-1004, 1081-1126, 1130-1189, 1224-1275, 1440-1509, 1514-1745, and 1750-1785 of SEQ ID NO: 2. In certain embodiments, nucleobases 44-79, 87-126, 131-273, 321-376, 478-513, 535-570, 630-716, 834-928, 930-973, 977-1004, 1081-1126, 1130-1189, 1224-1275, 1440-1509, 1514-1745, and 1750-1785 of SEQ ID NO: 2 are hotspot regions. In certain embodiments, such modified antisense oligonucleotides are 20 nucleobases in length. In certain embodiments, such modified antisense oligonucleotides are gapmers. In certain such embodiments, the gapmers are 5-10-5 MOE gapmers. In certain embodiments, the nucleosides of the modified antisense oligonucleotides are linked by phosphorothioate and phosphodiester internucleoside linkages.

The nucleobase sequences of SEQ ID Nos: 17, 18, 22-24, 50, 52, 54, 58, 63-65, 68-73, 77-79, 81, 83, 88, 90, 91, 93, 100, 102, 133, 137, 141-143, 146, 147, 154-156, 158, 161-163, 165-169, 171, 173, 174, 177-179, 210, 216, 218-220, 223, 224, 226-228, 232-234, 236, 239-242, 244, 246, 247, 251, 257, 258, 284, 287, 288, 292, 293, 298, 303, 307, 310, 311, 314, 315, 317-319, and 321-327 are complementary to nucleobases 44-79, 87-126, 131-273, 321-376, 478-513, 535-570, 630-716, 834-928, 930-973, 977-1004, 1081-1126, 1130-1189, 1224-1275, 1440-1509, 1514-1745, and 1750-1785 of SEQ ID NO: 2.

In certain embodiments, modified antisense oligonucleotides complementary to nucleobases 44-79, 87-126, 131-273, 321-376, 478-513, 535-570, 630-716, 834-928, 930-973, 977-1004, 1081-1126, 1130-1189, 1224-1275, 1440-1509, 1514-1745, and 1750-1785 of SEQ ID NO: 2 achieve at least 50% reduction of MECP2 RNA in vitro in the standard cell assay.

### 3. Nucleobases 1902-2000, 7300-7418, 67188-67239, 67241-67266, 67280-67315, 67321-67404, 68164-68274, 68278-68347, 68355-68531, 68535-68684, 68688-68747, 68750-68833, 68868-68895, 68998-69067, and 69072-69351

In certain embodiments, modified antisense oligonucleotides are complementary to nucleobases 1902-2000, 7300-7418, 67188-67239, 67241-67266, 67280-67315, 67321-67404, 68164-68274, 68278-68347, 68355-68531, 68535-68684, 68688-68747, 68750-68833, 68868-68895, 68998-69067, and 69072-69351 of SEQ ID NO: 1. In certain embodiments, nucleobases 1902-2000, 7300-7418, 67188-67239, 67241-67266, 67280-67315, 67321-67404, 68164-68274, 68278-68347, 68355-68531, 68535-68684, 68688-68747, 68750-68833, 68868-68895, 68998-69067, and 69072-69351 of SEQ ID NO: 1 are hotspot regions. In certain embodiments, such modified antisense oligonucleotides are 20 nucleobases in length. In certain embodiments, such modified oligonucleotides are gapmers. In certain such embodiments, the gapmers are 5-10-5 MOE gapmers. In certain embodiments, the nucleosides of the modified oligonucleotides are linked by phosphorothioate and phosphodiester internucleoside linkages.

The nucleobase sequences of SEQ ID Nos: 17, 18, 22-24, 56-60, 62-84, 86-93, 95-96, 100-102, 135-156, 158, 161-171, 173-174, 177-179, 212-213, 215-237, 239-244, 246-251, 256-258, 290-293, 296-305, 307-315, and 317-327 are complementary to nucleobases 1902-2000, 7300-7418, 67188-67239, 67241-67266, 67280-67315, 67321-67404, 68164-68274, 68278-68347, 68355-68531, 68535-68684, 68688-68747, 68750-68833, 68868-68895, 68998-69067, and 69072-69351 of SEQ ID NO: 1.

In certain embodiments, modified oligonucleotides complementary to nucleobases 1902-2000, 7300-7418, 67188-67239, 67241-67266, 67280-67315, 67321-67404, 68164-68274, 68278-68347, 68355-68531, 68535-68684, 68688-68747, 68750-68833, 68868-68895, 68998-69067, and 69072-69351 of SEQ ID NO: 1 achieve at least 25% reduction of MECP2 RNA in vitro in the standard cell assay.

### 4. Nucleobases 1918-1953, 1961-2000, 7300-7418, 67123-67178, 67280-67315, 67337-67372, 68188-68274, 68392-68486, 68488-68531, 68535-68562, 68639-68684, 68688-68747, 68782-68833, 68998-69067, 69072-69303, and 69308-69343

In certain embodiments, modified antisense oligonucleotides are complementary to nucleobases 1918-1953, 1961-2000, 7300-7418, 67123-67178, 67280-67315, 67337-67372, 68188-68274, 68392-68486, 68488-68531, 68535-68562, 68639-68684, 68688-68747, 68782-68833, 68998-69067, 69072-69303, and 69308-69343 of SEQ ID NO: 1. In certain embodiments, nucleobases 1918-1953, 1961-2000, 7300-7418, 67123-67178, 67280-67315, 67337-67372, 68188-68274, 68392-68486, 68488-68531, 68535-68562, 68639-68684, 68688-68747, 68782-68833, 68998-69067, 69072-69303, and 69308-69343 of SEQ ID NO: 1 are hotspot regions. In certain embodiments, such modified oligonucleotides are 20 nucleobases in length. In certain embodiments, such modified oligonucleotides are gapmers. In certain such embodiments, the gapmers are 5-10-5 MOE gapmers. In certain embodiments, the nucleosides of the modified oligonucleotides are linked by phosphorothioate and phosphodiester internucleoside linkages.

The nucleobase sequences of SEQ ID Nos: 17, 18, 22-24, 52, 54, 58, 63-65, 68-73, 77-79, 81, 83, 88, 90, 91, 93, 100, 102, 133, 137, 141-143, 146, 147, 154-156, 158, 161-163, 165-169, 171, 173, 174, 177-179, 210, 216, 218-220, 223, 224, 226-228, 232-234, 236, 239, 240-242, 244, 246, 247, 251, 257, 258, 287, 288, 292, 293, 298, 303, 307, 310, 311, 314, 315, 317-319, and 321-327 are complementary to nucleobases 1918-1953, 1961-2000, 7300-7418, 67123-67178, 67280-67315, 67337-67372, 68188-68274, 68392-68486, 68488-68531, 68535-68562, 68639-68684, 68688-68747, 68782-68833, 68998-69067, 69072-69303, and 69308-69343 of SEQ ID NO: 1.

In certain embodiments, modified antisense oligonucleotides complementary to nucleobases 1918-1953, 1961-2000, 7300-7418, 67123-67178, 67280-67315, 67337-67372, 68188-68274, 68392-68486, 68488-68531, 68535-68562, 68639-68684, 68688-68747, 68782-68833, 68998-69067, 69072-69303, and 69308-69343 of SEQ ID NO: 1 achieve at least 50% reduction of MECP2 RNA in vitro in the standard cell assay.

### EXAMPLES

### Non-Limiting Disclosure and Incorporation by Reference

While certain methods described herein have been described with specificity in accordance with certain embodiments, the following examples serve only to illustrate the compounds described herein and are not intended to limit the same. Each of the references recited in the present application is incorporated herein by reference in its entirety.

### Example 1: Screening of Antisense Oligonucleotides Targeting MECP2

Antisense oligonucleotides (ASOs) that target human Methyl CpG Binding Protein 2 (MECP2), the complement of GENBANK accession number NT_167198.1 truncated from 4203000 to 4283000, SEQ ID NO: 1, were synthesized using standard solid phase oligonucleotide synthetic methods. They are chimeric oligonucleotides (“gapmers”), composed of a central “gap” region consisting of 2′-deoxynucleotides, which is flanked on both sides (5′ and 3′) by “wings” that are composed of modified nucleotides. The internucleoside (backbone) linkages are phosphorothioate or phosphodiester throughout the oligonucleotides. The sequences and structures of the antisense oligonucleotides and their start and stop sites along SEQ ID NO: 1 are shown in the tables below. ASOs were designed to target exons and introns along the MECP2 pre-mRNA and some ASOs also target the mRNA. Isis Numbers 628567 (Table 1), 628553 (Table 2), 628566 (Table 3), and 628552 (Table 4) have mismatches to SEQ ID NO: 1 but are 100% complementary to human MECP2 mRNA, GENBANK accession number NM_004992.3 (SEQ ID NO: 2), with start sites of 246, 123, 238, and 115, respectively, on SEQ ID NO: 2. Isis Number 18078 does not target MECP2 and was used as a negative control.

The antisense oligonucleotides were analyzed for their effects on target mRNA levels. HepG2 cells were plated at a density of 20,000 cells per well in 96-well plates and were electroporated with 4.00 μM oligonucleotide or with no oligonucleotide for untreated controls. After approximately 24 hours, RNA was isolated from the cells, and MECP2 mRNA levels were measured by quantitative real-time PCR using primer probe set RTS4253 (forward: 5′-TGAAGGAGTCTTCTATCCGATCTGT-3′, SEQ ID NO: 12; reverse: 5′-CACTTCCTTGACCTCGATGCT-3′, SEQ ID NO: 13; probe: 5′-AGACCGTACTCCCCATCAAGAAGCGC-3′, SEQ ID NO: 14). MECP2 mRNA levels were adjusted according to total RNA content, as measured by RIBOGREEN®. Results are presented as average percent inhibition of MECP2 mRNA expression level, relative to untreated control cells, in the tables below. The levels of MECP2 mRNA in untreated control cells (UTC) represents 0% inhibition, and an undetectable level of MECP2 mRNA represents 100% inhibition. A negative inhibition value means that the level of MECP2 mRNA detected was greater than that detected in untreated control cells. The results show that many of the antisense oligonucleotides inhibited MECP2 mRNA levels. The antisense oligonucleotides marked with an asterisk (*) target the region of the primer probe set. Additional assays may be used to measure the potency and efficacy of these antisense oligonucleotides.

TABLE 1
Inhibition of human MECP2 by antisense oligonucleotides in vitro

| | | | | % | SEQ |
|---|---|---|---|---|---|
| Isis | | Start | Stop | Inhi- | ID |
| No. | Sequence (5′ to 3′) | site | site | bition | NO: |
| 18078 | Gₑₛ Tₑₛ Gₑₛ ^{m}Cₑₛ Gₑₛ C_{ds} G_{ds} C_{ds} G_{ds} A_{ds} G_{ds} C_{ds} C_{ds} C_{ds} | n/a | n/a | 0.5 | 15 |
| | Gₑₛ Aₑₛ Aₑₛ Aₑₛ Tₑₛ ^{m}Cₑ | | | | |
| 628543 | ^{m}Cₑₛ Tₑₒ ^{m}Cₑₒ Tₑₒ ^{m}Cₑₒ ^{m}C_{ds} G_{ds} A_{ds} G_{ds} A_{ds} G_{ds} G_{ds} A_{ds} | 1894 | 1913 | −1.7 | 16 |
| | G_{ds} G_{ds} Gₑₒ Aₑₒ Gₑₛ ^{m}Cₑₛ Gₑ | | | | |
| 628547 | Gₑₛ ^{m}Cₑₒ ^{m}Cₑₒ Aₑₒ Tₑₒ T_{ds} T_{ds} T_{ds} ^{m}C_{ds} ^{m}C_{ds} G_{ds} G_{ds} A_{ds} | 1926 | 1945 | 61.8 | 17 |
| | ^{m}C_{ds} G_{ds} Gₑₒ ^{m}Cₑₒ Tₑₛ Tₑₛ Tₑ | | | | |
| 628551 | Tₑₛ ^{m}Cₑₒ Tₑₒ ^{m}Cₑₒ Tₑₒ ^{m}C_{ds} ^{m}C_{ds} T_{ds} ^{m}C_{ds} ^{m}C_{ds} T_{ds} ^{m}C_{ds} | 1981 | 2000 | 54.2 | 18 |
| | G_{ds} ^{m}C_{d} ^{m}C_{ds} Tₑₒ ^{m}Cₑₒ ^{m}Cₑₒ ^{m}Cₑₛ Tₑₛ ^{m}Cₑ | | | | |
| 628739 | Aₑₛ ^{m}Cₑₒ ^{m}Cₑₒ ^{m}Cₑₒ ^{m}Cₑₒ ^{m}C_{ds} G_{ds} ^{m}C_{ds} ^{m}C_{ds} ^{m}C_{ds} ^{m}C_{ds} | 2036 | 2055 | 33.6 | 19 |
| | ^{m}C_{ds} ^{m}C_{ds} G_{ds} G_{ds} ^{m}Cₑₒ Aₑₒ Aₑₛ Gₑₛ Gₑ | | | | |
| 628743 | Aₑₛ Gₑₒ Aₑₒ Gₑₒ Aₑₒ ^{m}C_{ds} ^{m}C_{ds} T_{ds} ^{m}C_{ds} A_{ds} A_{ds} ^{m}C_{ds} T_{ds} | 4053 | 4072 | 36.6 | 20 |
| | T_{ds} G_{ds} Tₑₒ ^{m}Cₑₒ Aₑₛ ^{m}Cₑₛ Gₑ | | | | |
| 628747 | ^{m}Cₑₛ Aₑₒ Tₑₒ Tₑₒ Aₑₒ A_{ds} G_{ds} A_{ds} T_{ds} A_{ds} A_{ds} ^{m}C_{ds} ^{m}C_{ds} | 6590 | 6609 | 43.4 | 21 |
| | A_{ds} T_{ds} ^{m}Cₑₒ Aₑₒ Tₑₛ Tₑₛ Tₑ | | | | |
| 628555 | Gₑₛ Gₑₒ Aₑₒ Aₑₒ ^{m}Cₑₒ T_{ds} G_{ds} G_{ds} T_{ds} G_{ds} A_{ds} G_{ds} T_{ds} | 7308 | 7327 | 60.7 | 22 |
| | ^{m}C_{ds} T_{ds} Gₑₒ Tₑₒ Aₑₛ Tₑₛ Tₑ | | | | |
| 628559 | Gₑₛ Aₑₒ Aₑₒ Gₑₒ ^{m}Cₑₒ A_{ds} A_{ds} G_{ds} G_{ds} T_{ds} G_{ds} T_{ds} A_{ds} T_{ds} | 7351 | 7370 | 62.7 | 23 |
| | T_{ds} ^{m}Cₑₒ Tₑₒ Gₑₛ Gₑₛ Gₑ | | | | |
| 628563 | ^{m}Cₑₛ Tₑₒ Aₑₒ ^{m}Cₑₒ ^{m}Cₑₒ A_{ds} T_{ds} G_{ds} G_{ds} A_{ds} A_{ds} T_{ds} ^{m}C_{ds} | 7383 | 7402 | 63.1 | 24 |
| | ^{m}C_{ds} T_{ds} Gₑₒ Tₑₒ Tₑₛ Gₑₛ Gₑ | | | | |
| 628751 | Tₑₛ Tₑₒ Tₑₒ Tₑₒ ^{m}Cₑₒ T_{ds} A_{ds} T_{ds} A_{ds} A_{ds} A_{ds} T_{ds} ^{m}C_{ds} | 9115 | 9134 | 78.3 | 25 |
| | ^{m}C_{ds} A_{ds} Tₑₒ Gₑₒ Tₑₛ Aₑₛ Tₑ | | | | |
| 628755 | Tₑₛ Aₑₒ Gₑₒ ^{m}Cₑₒ ^{m}C_{ds} ^{m}C_{ds} ^{m}C_{ds} A_{ds} ^{m}C_{ds} T_{ds} ^{m}C_{ds} ^{m}C_{ds} | 11509 | 11528 | 62.1 | 26 |
| | ^{m}C_{ds} G_{ds} G_{ds} Aₑₒ Tₑₒ Aₑₛ Aₑₛ Gₑ | | | | |
| 628759 | Aₑₛ ^{m}Cₑₒ Tₑₒ ^{m}Cₑₒ Aₑₒ A_{ds} G_{ds} ^{m}C_{ds} ^{m}C_{ds} ^{m}C_{ds} A_{ds} A_{ds} | 14390 | 14409 | 39.1 | 27 |
| | G_{ds} G_{ds} A_{ds} Gₑₒ Tₑₒ Tₑₛ ^{m}Cₑₛ Aₑ | | | | |
| 628763 | Gₑₛ Cₑₒ Tₑₒ Tₑₒ Tₑₒ A_{ds} A_{ds} T_{ds} G_{ds} C_{ds} T_{ds} T_{ds} T_{ds} A_{ds} | 17349 | 17368 | 89.3 | 28 |
| | T_{ds} Tₑₒ Tₑₒ Tₑₛ Tₑₛ Aₑ | | | | |
| 628767 | Tₑₛ Gₑₒ ^{m}Cₑₒ ^{m}Cₑₒ Aₑₒ A_{ds} ^{m}C_{ds} A_{ds} G_{ds} ^{m}C_{ds} A_{ds} G_{ds} G_{ds} | 19691 | 19710 | 82.2 | 29 |
| | ^{m}C_{ds} ^{m}C_{ds} ^{m}Cₑₒ Aₑₒ Gₑₛ ^{m}Cₑₛ Gₑ | | | | |
| 628771 | Gₑₛ Aₑₒ Tₑₒ Aₑₒ Tₑₒ ^{m}C_{ds} A_{ds} G_{ds} T_{ds} G_{ds} A_{ds} G_{ds} G_{ds} A_{ds} | 22318 | 22337 | 69.0 | 30 |
| | A_{ds} Gₑₒ Tₑₒ Tₑₛ Gₑₛ Tₑ | | | | |
| 628775 | ^{m}Cₑₛ Gₑₒ Tₑₒ Gₑₒ ^{m}Cₑₒ ^{m}C_{ds} A_{ds} T_{ds} G_{ds} G_{ds} A_{ds} A_{ds} G_{ds} | 24936 | 24955 | 82.8 | 31 |
| | T_{ds} ^{m}C_{ds} ^{m}Cₑₒ Tₑₒ Tₑₛ ^{m}Cₑₛ ^{m}Cₑ | | | | |
| 628779 | Gₑₛ Gₑₒ Tₑₒ Gₑₒ Aₑₒ G_{ds} ^{m}C_{ds} T_{ds} G_{ds} A_{ds} T_{ds} G_{ds} ^{m}C_{ds} | 27172 | 27191 | 75.8 | 32 |
| | T_{ds} A_{ds} Tₑₒ Aₑₒ Tₑₛ Gₑₛ Aₑ | | | | |
| 628783 | Aₑₛ Gₑₒ Gₑₒ ^{m}Cₑₒ Gₑₒ G_{ds} ^{m}C_{ds} A_{ds} G_{ds} T_{ds} G_{ds} G_{ds} ^{m}C_{ds} | 29717 | 29736 | 35.2 | 33 |
| | T_{ds} T_{ds} Aₑₒ ^{m}Cₑₒ Gₑₛ ^{m}Cₑₛ ^{m}Cₑ | | | | |
| 628787 | Aₑₛ Gₑₒ ^{m}Cₑₒ ^{m}Cₑₒ ^{m}Cₑₒ ^{m}C_{ds} T_{ds} T_{ds} A_{ds} A_{ds} T_{ds} T_{ds} T_{ds} | 31758 | 31777 | 81.2 | 34 |
| | T_{ds} G_{ds} Tₑₒ Tₑₒ ^{m}Cₑₛ Tₑₛ ^{m}Cₑ | | | | |
| 628791 | Tₑₛ Gₑₒ Gₑₒ ^{m}Cₑₒ Gₑₒ G_{ds} ^{m}C_{ds} T_{ds} ^{m}C_{ds} A_{ds} A_{ds} G_{ds} A_{ds} | 34273 | 34292 | 39.2 | 35 |
| | A_{ds} ^{m}C_{ds} ^{m}Cₑₒ Aₑₒ Gₑₛ ^{m}Cₑₛ ^{m}Cₑ | | | | |
| 628795 | ^{m}Cₑₛ Aₑₒ Aₑₒ Aₑₒ Tₑₒ A_{ds} T_{ds} T_{ds} A_{ds} G_{ds} A_{ds} A_{ds} T_{ds} A_{ds} | 36288 | 36307 | 62.8 | 36 |
| | G_{ds} Aₑₒ ^{m}Cₑₒ Tₑₛ ^{m}Cₑₛ Aₑ | | | | |
| 628799 | Tₑₛ Gₑₒ Gₑₒ Gₑₒ Aₑₒ ^{m}C_{ds} T_{ds} ^{m}C_{ds} A_{ds} G_{ds} A_{ds} T_{ds} T_{ds} | 39071 | 39090 | 67.2 | 37 |
| | ^{m}C_{ds} T_{ds} Aₑₒ Tₑₒ Aₑₛ Gₑₛ Gₑ | | | | |
| 628803 | Gₑₛ Tₑₒ ^{m}Cₑₒ ^{m}Cₑₒ Tₑₒ G_{ds} G_{ds} A_{ds} A_{ds} ^{m}C_{ds} G_{ds} A_{ds} ^{m}C_{ds} | 41073 | 41092 | 66.0 | 38 |
| | A_{ds} G_{ds} Gₑₒ ^{m}Cₑₒ Tₑₛ Tₑₛ Gₑ | | | | |
| 628807 | ^{m}Cₑₛ ^{m}Cₑₒ Aₑₒ Aₑₒ Aₑₒ T_{ds} T_{ds} T_{ds} A_{ds} T_{ds} A_{ds} A_{ds} ^{m}C_{ds} | 43580 | 43599 | 18.3 | 39 |
| | T_{ds} T_{ds} Aₑₒ Aₑₒ Gₑₛ Aₑₛ Aₑ | | | | |
| 628811 | Gₑₛ Gₑₒ Tₑₒ Gₑₒ Aₑₒ T_{ds} G_{ds} T_{ds} G_{ds} T_{ds} A_{ds} T_{ds} T_{ds} T_{ds} | 45768 | 45787 | 86.2 | 40 |
| | T_{ds} Aₑₒ ^{m}Cₑₒ Tₑₛ Aₑₛ ^{m}Cₑ | | | | |
| 628815 | Tₑₛ Gₑₒ Gₑₒ Tₑₒ Gₑₒ G_{ds} G_{ds} A_{ds} ^{m}C_{ds} A_{ds} A_{ds} A_{ds} A_{ds} A_{ds} | 47850 | 47869 | 64.6 | 41 |
| | T_{ds} Tₑₒ Gₑₒ Tₑₛ Gₑₛ Gₑ | | | | |
| 628819 | Aₑₛ Aₑₒ Aₑₒ Tₑₒ Aₑₒ A_{ds} G_{ds} ^{m}C_{ds} A_{ds} T_{ds} ^{m}C_{ds} T_{ds} G_{ds} | 49865 | 49884 | 52.8 | 42 |
| | G_{ds} ^{m}C_{ds} Aₑₒ Tₑₒ Tₑₛ Tₑₛ Gₑ | | | | |
| 628823 | Tₑₛ Aₑₒ ^{m}Cₑₒ Aₑₒ Tₑₒ T_{ds} G_{ds} A_{ds} A_{ds} A_{ds} A_{ds} A_{ds} ^{m}C_{ds} | 52552 | 52571 | 17.2 | 43 |
| | A_{ds} G_{ds} ^{m}Cₑₒ ^{m}Cₑₒ Aₑₛ Gₑₛ Aₑ | | | | |
| 628827 | Gₑₛ Gₑₒ Aₑₒ Tₑₒ ^{m}Cₑₒ ^{m}C_{ds} A_{ds} T_{ds} G_{ds} ^{m}C_{ds} G_{ds} A_{ds} G_{ds} | 54569 | 54588 | 29.1 | 44 |
| | A_{ds} G_{ds} Aₑₒ Aₑₒ Gₑₛ ^{m}Cₑₛ Aₑ | | | | |
| 628831 | Tₑₛ Aₑₒ Tₑₒ Aₑₒ Aₑₒ T_{ds} A_{ds} T_{ds} ^{m}C_{ds} A_{ds} T_{ds} T_{ds} ^{m}C_{ds} | 56608 | 56627 | 61.7 | 45 |
| | A_{ds} G_{ds} ^{m}Cₑₒ ^{m}Cₑₒ Tₑₛ ^{m}Cₑₛ Aₑ | | | | |
| 628835 | ^{m}Cₑₛ Aₑₒ Gₑₒ ^{m}Cₑₒ Aₑₒ G_{ds} G_{ds} A_{ds} A_{ds} G_{ds} A_{ds} G_{ds} T_{ds} | 59223 | 59242 | 15.9 | 46 |
| | ^{m}C_{ds} ^{m}C_{ds} Aₑₒ Gₑₒ Aₑₛ Gₑₛ Aₑ | | | | |
| 628839 | Aₑₛ Gₑₒ Aₑₒ Aₑₒ Aₑₒ ^{m}C_{ds} ^{m}C_{ds} T_{ds} G_{ds} ^{m}C_{ds} ^{m}C_{ds} A_{ds} G_{ds} | 61278 | 61297 | 64.4 | 47 |
| | G_{ds} T_{ds} Gₑₒ Tₑₒ Gₑₛ Gₑₛ Tₑ | | | | |
| 628843 | ^{m}Cₑₛ ^{m}Cₑₒ Aₑₒ Gₑₒ Gₑₒ T_{ds} G_{ds} T_{ds} G_{ds} G_{ds} ^{m}C_{ds} ^{m}C_{ds} | 63401 | 63420 | 20.5 | 48 |
| | ^{m}C_{ds} A_{ds} G_{ds} Gₑₒ Gₑₒ Tₑₛ Gₑₛ Gₑ | | | | |
| 628847 | Gₑₛ Gₑₒ ^{m}Cₑₒ Aₑₒ Tₑₒ ^{m}C_{ds} ^{m}C_{ds} T_{ds} A_{ds} ^{m}C_{ds} A_{ds} A_{ds} | 65432 | 65451 | 58.1 | 49 |
| | ^{m}C_{ds} ^{m}C_{ds} ^{m}C_{ds} Aₑₒ ^{m}Cₑₒ Aₑₛ Gₑₛ Aₑ | | | | |
| 628567 | Tₑₛ Gₑₒ Aₑₒ ^{m}Cₑₒ Tₑₒ T_{ds} T_{ds} T_{ds} ^{m}C_{ds} T_{ds} T_{ds} ^{m}C_{ds} ^{m}C_{ds} | 67048 | 67067 | 51.8 | 50 |
| | ^{m}C_{ds} T_{ds} G_{ds} Aₑₒ Gₑₛ ^{m}Cₑₛ ^{m}Cₑ | | | | |
| 628571 | Gₑₛ Gₑₒ Gₑₒ Tₑₒ Tₑₒ T_{ds} G_{ds} T_{ds} ^{m}C_{ds} ^{m}C_{ds} T_{ds} T_{ds} G_{ds} A_{ds} | 67084 | 67103 | 40.4 | 51 |
| | G_{ds} Gₑₒ ^{m}Cₑₒ ^{m}Cₑₛ ^{m}Cₑₛ Tₑ | | | | |
| 628575 | ^{m}Cₑₛ Tₑₒ ^{m}Cₑₒ Tₑₒ Tₑₒ ^{m}C_{ds} T_{ds} T_{ds} T_{ds} ^{m}C_{ds} T_{ds} T_{ds} A_{ds} | 67123 | 67142 | 55.4 | 52 |
| | T_{ds} ^{m}C_{ds} Tₑₒ Tₑₒ Tₑₛ ^{m}Cₑₛ Tₑ | | | | |
| 628579 | ^{m}Cₑₛ Tₑₒ Tₑₒ Gₑₒ ^{m}Cₑₒ ^{m}C_{ds} ^{m}C_{ds} T_{ds} ^{m}C_{ds} T_{ds} T_{ds} T_{ds} | 67135 | 67154 | 49.6 | 53 |
| | T_{ds} ^{m}C_{ds} T_{ds} ^{m}C_{ds} Tₑₒ Tₑₒ ^{m}Cₑₛ Tₑₛ Tₑ | | | | |
| 628583 | Tₑₛ Gₑₒ Gₑₒ ^{m}Cₑₒ Tₑₒ G_{ds} ^{m}C_{ds} A_{ds} ^{m}C_{ds} G_{ds} G_{ds} G_{ds} ^{m}C_{ds} | 67153 | 67172 | 57.4 | 54 |
| | T_{ds} ^{m}C_{ds} Aₑₒ Tₑₒ Gₑₛ ^{m}Cₑₛ Tₑ | | | | |
| 628587 | Gₑₛ Tₑₒ Gₑₒ Gₑₒ Tₑₒ G_{ds} G_{ds} G_{ds} ^{m}C_{ds} T_{ds} G_{ds} A_{ds} T_{ds} G_{ds} | 67165 | 67184 | 30.9 | 55 |
| | G_{ds} ^{m}Cₑₒ Tₑₒ Gₑₛ ^{m}Cₑₛ Aₑ | | | | |
| 628591 | ^{m}Cₑₛ Tₑₒ Gₑₒ ^{m}Cₑₒ Tₑₒ T_{ds} T_{ds} G_{ds} ^{m}C_{ds} ^{m}C_{ds} T_{ds} G_{ds} ^{m}C_{ds} | 67196 | 67215 | 50.0 | 56 |
| | ^{m}C_{ds} T_{ds} ^{m}Cₑₒ Tₑₒ Gₑₛ ^{m}Cₑₛ Gₑ | | | | |
| 628595 | Aₑₛ Aₑₒ Gₑₒ ^{m}Cₑₒ Tₑₒ T_{ds} ^{m}C_{ds} ^{m}C_{ds} G_{ds} G_{ds} ^{m}C_{ds} A_{ds} | 67241 | 67260 | 47.3 | 57 |
| | ^{m}C_{ds} A_{ds} G_{ds} ^{m}Cₑₒ ^{m}Cₑₒ Gₑₛ Gₑₛ Gₑ | | | | |
| 628599 | Gₑₛ Gₑₒ Tₑₒ ^{m}Cₑₒ Aₑₒ ^{m}C_{ds} G_{ds} G_{ds} A_{ds} T_{ds} G_{ds} A_{ds} T_{ds} | 67280 | 67299 | 50.6 | 58 |
| | G_{ds} G_{ds} Aₑₒ Gₑₒ ^{m}Cₑₛ Gₑₛ ^{m}Cₑ | | | | |
| 628603 | Tₑₛ Tₑₒ ^{m}Cₑₒ ^{m}Cₑₒ Gₑₒ T_{ds} G_{ds} T_{ds} ^{m}C_{ds} ^{m}C_{ds} A_{ds} G_{ds} ^{m}C_{ds} | 67329 | 67348 | 28.8 | 59 |
| | ^{m}C_{ds} T_{ds} Tₑₒ ^{m}Cₑₒ Aₑₛ Gₑₛ Gₑ | | | | |
| *628607 | ^{m}Cₑₛ Aₑₒ Gₑₒ ^{m}Cₑₒ Aₑₒ G_{ds} A_{ds} G_{ds} ^{m}C_{ds} G_{ds} G_{ds} ^{m}C_{ds} | 67361 | 67380 | 33.0 | 60 |
| | ^{m}C_{ds} A_{ds} G_{ds} Aₑₒ Tₑₒ Tₑₛ Tₑₛ Tₑ | | | | |
| 628851 | Tₑₛ Gₑₒ Tₑₒ ^{m}Cₑₒ ^{m}Cₑₒ ^{m}C_{ds} T_{ds} G_{ds} ^{m}C_{ds} ^{m}C_{ds} ^{m}C_{ds} T_{ds} | 67434 | 67453 | 20.0 | 61 |
| | ^{m}C_{ds} ^{m}C_{ds} ^{m}C_{ds} Tₑₒ Gₑₒ ^{m}Cₑₛ ^{m}Cₑₛ ^{m}Cₑ | | | | |
| 628611 | Gₑₛ ^{m}Cₑₒ Gₑₒ Aₑₒ Aₑₒ A_{ds} G_{ds} G_{ds} ^{m}C_{ds} T_{ds} T_{ds} T_{ds} T_{ds} | 68164 | 68183 | 71.0 | 62 |
| | ^{m}C_{ds} ^{m}C_{ds} ^{m}Cₑₒ Tₑₒ Gₑₛ Gₑₛ Gₑ | | | | |
| 628615 | Gₑₛ Aₑₒ Aₑₒ Gₑₒ Tₑₒ A_{ds} ^{m}C_{ds} G_{ds} ^{m}C_{ds} A_{ds} A_{ds} T_{ds} ^{m}C_{ds} | 68191 | 68210 | 65.9 | 63 |
| | A_{ds} A_{ds} ^{m}Cₑₒ Tₑₒ ^{m}Cₑₛ ^{m}Cₑₛ Aₑ | | | | |
| 628619 | Gₑₛ Gₑₒ Gₑₒ Aₑₒ Tₑₒ G_{ds} T_{ds} G_{ds} T_{ds} ^{m}C_{ds} G_{ds} ^{m}C_{ds} ^{m}C_{ds} | 68213 | 68232 | 62.3 | 64 |
| | T_{ds} A_{ds} ^{m}Cₑₒ ^{m}Cₑₒ Tₑₛ Tₑₛ Tₑ | | | | |
| 628623 | ^{m}Cₑₛ Gₑₒ Tₑₒ Gₑₒ Aₑₒ A_{ds} G_{ds} T_{ds} ^{m}C_{ds} A_{ds} A_{ds} A_{ds} A_{ds} | 68239 | 68258 | 68.7 | 65 |
| | T_{ds} ^{m}C_{ds} Aₑₒ Tₑₒ Tₑₛ Aₑₛ Gₑ | | | | |
| 628627 | Tₑₛ Tₑₒ Aₑₒ Gₑₒ Gₑₒ T_{ds} G_{ds} G_{ds} T_{ds} T_{ds} T_{ds} ^{m}C_{ds} T_{ds} G_{ds} | 68286 | 68305 | 43.0 | 66 |
| | ^{m}C_{ds} Tₑₒ ^{m}Cₑₒ Tₑₛ ^{m}Cₑₛ Gₑ | | | | |
| 628631 | ^{m}Cₑₛ Gₑₒ Gₑₒ ^{m}Cₑₒ ^{m}Cₑₒ T_{ds} ^{m}C_{ds} T_{ds} G_{ds} ^{m}C_{ds} ^{m}C_{ds} A_{ds} | 68328 | 68347 | 36.5 | 67 |
| | G_{ds} T_{ds} T_{ds} ^{m}Cₑₒ ^{m}Cₑₒ Tₑₛ Gₑₛ Gₑ | | | | |
| 628635 | Aₑₛ ^{m}Cₑₒ ^{m}Cₑₒ ^{m}Cₑₒ Tₑₒ T_{ds} T_{ds} T_{ds} ^{m}C_{ds} A_{ds} ^{m}C_{ds} ^{m}C_{ds} | 68400 | 68419 | 53.2 | 68 |
| | T_{ds} G_{ds} ^{m}C_{ds} Aₑₒ ^{m}Cₑₒ Aₑₛ ^{m}Cₑₛ ^{m}Cₑ | | | | |
| 628639 | Aₑₛ Aₑₒ Gₑₒ Gₑₒ Aₑₒ G_{ds} ^{m}C_{ds} T_{ds} T_{ds} ^{m}C_{ds} ^{m}C_{ds} ^{m}C_{ds} A_{ds} | 68428 | 68447 | 60.0 | 69 |
| | G_{ds} G_{ds} Aₑₒ ^{m}Cₑₒ Tₑₛ Tₑₛ Tₑ | | | | |
| 628643 | Tₑₛ Gₑₒ Gₑₒ ^{m}Cₑₒ Gₑₒ A_{ds} A_{ds} G_{ds} T_{ds} T_{ds} T_{ds} G_{ds} A_{ds} A_{ds} | 68455 | 68474 | 57.8 | 70 |
| | A_{ds} Aₑₒ Gₑₒ Gₑₛ ^{m}Cₑₛ Aₑ | | | | |
| 628647 | Aₑₛ Gₑₒ ^{m}Cₑₒ ^{m}Cₑₒ Tₑₒ T_{ds} G_{ds} ^{m}C_{ds} ^{m}C_{ds} ^{m}C_{ds} ^{m}C_{ds} ^{m}C_{ds} | 68467 | 68486 | 59.3 | 71 |
| | T_{ds} G_{ds} G_{ds} ^{m}Cₑₒ Gₑₒ Aₑₛ Aₑₛ Gₑ | | | | |
| 628651 | Tₑₛ Gₑₒ Gₑₒ Aₑₒ Tₑₒ G_{ds} T_{ds} G_{ds} G_{ds} T_{ds} G_{ds} G_{ds} ^{m}C_{ds} | 68492 | 68511 | 50.3 | 72 |
| | ^{m}C_{ds} ^{m}C_{ds} ^{m}Cₑₒ Aₑₒ ^{m}Cₑₛ ^{m}Cₑₛ ^{m}Cₑ | | | | |
| 628655 | Gₑₛ ^{m}Cₑₒ Tₑₒ Tₑₒ Tₑₒ T_{ds} ^{m}C_{ds} G_{ds} ^{m}C_{ds} T_{ds} T_{ds} ^{m}C_{ds} ^{m}C_{ds} | 68535 | 68554 | 67.2 | 73 |
| | T_{ds} G_{ds} ^{m}Cₑₒ ^{m}Cₑₒ Gₑₛ Gₑₛ Gₑ | | | | |
| 628659 | Gₑₛ Tₑₒ Tₑₒ Tₑₒ ^{m}Cₑₒ T_{ds} T_{ds} G_{ds} G_{ds} G_{ds} A_{ds} A_{ds} T_{ds} G_{ds} | 68567 | 68586 | 41.1 | 74 |
| | G_{ds} ^{m}Cₑₒ ^{m}Cₑₒ Tₑₛ Gₑₛ Aₑ | | | | |
| 628663 | Gₑₛ Gₑₒ ^{m}Cₑₒ Tₑₒ Gₑₒ ^{m}C_{ds} ^{m}C_{ds} A_{ds} ^{m}C_{ds} ^{m}C_{ds} A_{ds} ^{m}C_{ds} | 68599 | 68618 | 40.4 | 75 |
| | A_{ds} ^{m}C_{ds} T_{ds} ^{m}Cₑₒ ^{m}Cₑₒ ^{m}Cₑₛ ^{m}Cₑₛ Gₑ | | | | |
| *628667 | Tₑₛ Tₑₒ ^{m}Cₑₒ Aₑₒ ^{m}Cₑₒ G_{ds} G_{ds} ^{m}C_{ds} T_{ds} T_{ds} T_{ds} ^{m}C_{ds} T_{ds} | 68631 | 68650 | 38.2 | 76 |
| | T_{ds} T_{ds} Tₑₒ Tₑₒ Gₑₛ Gₑₛ ^{m}Cₑ | | | | |
| *628671 | ^{m}Cₑₛ Tₑₒ ^{m}Cₑₒ ^{m}Cₑₒ Tₑₒ G_{ds} ^{m}C_{ds} A_{ds} ^{m}C_{ds} A_{ds} G_{ds} A_{ds} T_{ds} | 68659 | 68678 | 76.9 | 77 |
| | ^{m}C_{ds} G_{ds} Gₑₒ Aₑₒ Tₑₛ Aₑₛ Gₑ | | | | |
| *628675 | Tₑₛ ^{m}Cₑₒ Tₑₒ ^{m}Cₑₒ ^{m}Cₑₒ ^{m}C_{ds} G_{ds} G_{ds} G_{ds} T_{ds} ^{m}C_{ds} T_{ds} T_{ds} | 68696 | 68715 | 85.3 | 78 |
| | G_{ds} ^{m}C_{ds} Gₑₒ ^{m}Cₑₒ Tₑₛ Tₑₛ ^{m}Cₑ | | | | |
| *628679 | ^{m}Cₑₛ Tₑₒ Tₑₒ ^{m}Cₑₒ Aₑₒ ^{m}C_{ds} ^{m}C_{ds} A_{ds} ^{m}C_{ds} T_{ds} T_{ds} ^{m}C_{ds} | 68728 | 68747 | 74.5 | 79 |
| | ^{m}C_{ds} T_{ds} T_{ds} Gₑₒ Aₑₒ ^{m}Cₑₛ ^{m}Cₑₛ Tₑ | | | | |
| 628683 | Tₑₛ ^{m}Cₑₒ Aₑₒ Gₑₒ Tₑₒ ^{m}C_{ds} ^{m}C_{ds} T_{ds} T_{ds} T_{ds} ^{m}C_{ds} ^{m}C_{ds} | 68774 | 68793 | 43.0 | 80 |
| | ^{m}C_{ds} G_{ds} ^{m}C_{ds} Tₑₒ ^{m}Cₑₒ Tₑₛ Tₑₛ ^{m}Cₑ | | | | |
| 628687 | ^{m}Cₑₛ Tₑₒ Tₑₒ Gₑₒ ^{m}Cₑₒ T_{ds} T_{ds} T_{ds} T_{ds} ^{m}C_{ds} ^{m}C_{ds} G_{ds} ^{m}C_{ds} | 68806 | 68825 | 69.2 | 81 |
| | ^{m}C_{ds}^{m}C_{ds} Aₑₒ Gₑₒ Gₑₛ Gₑₛ ^{m}Cₑ | | | | |
| 628691 | Gₑₛ Gₑₒ Tₑₒ Gₑₒ Aₑₒ T_{ds} G_{ds} G_{ds} T_{ds} G_{ds} G_{ds} T_{ds} G_{ds} G_{ds} | 68876 | 68895 | 49.4 | 82 |
| | T_{ds} Gₑₒ ^{m}Cₑₒ Tₑₛ ^{m}Cₑₛ ^{m}Cₑ | | | | |
| 628695 | Gₑₛ ^{m}Cₑₒ Tₑₒ Gₑₒ ^{m}Cₑₒ T_{ds} G_{ds} ^{m}C_{ds} T_{ds} ^{m}C_{ds} A_{ds} A_{ds} G_{ds} | 68998 | 69017 | 62.3 | 83 |
| | T_{ds} ^{m}C_{ds} ^{m}Cₑₒ Tₑₒ Gₑₛ Gₑₛ Gₑ | | | | |
| 628699 | ^{m}Cₑₛ Tₑₒ ^{m}Cₑₒ ^{m}Cₑₒ Aₑₒ G_{ds} T_{ds} G_{ds} A_{ds} G_{ds} ^{m}C_{ds} ^{m}C_{ds} T_{ds} | 69040 | 69059 | 45.7 | 84 |
| | ^{m}C_{ds} ^{m}C_{ds} Tₑₒ ^{m}Cₑₒ Tₑₛ Gₑₛ Gₑ | | | | |
| 628703 | Aₑₛ Aₑₒ ^{m}Cₑₒ ^{m}Cₑₒ Gₑₒ ^{m}C_{ds} G_{ds} G_{ds} G_{ds} ^{m}C_{ds} T_{ds} G_{ds} A_{ds} | 69085 | 69104 | 23.7 | 85 |
| | G_{ds} T_{ds} ^{m}Cₑₒ Tₑₒ Tₑₛ Aₑₛ Gₑ | | | | |
| 628707 | Tₑₛ Gₑₒ Gₑₒ ^{m}Cₑₒ Gₑₒ G_{ds} ^{m}C_{ds} G_{ds} G_{ds} T_{ds} G_{ds} G_{ds} ^{m}C_{ds} | 69098 | 69117 | 43.7 | 86 |
| | A_{ds} A_{ds} ^{m}Cₑₒ ^{m}Cₑₒ Gₑₛ ^{m}Cₑₛ Gₑ | | | | |
| 628711 | Tₑₛ Tₑₒ Tₑₒ Tₑₒ ^{m}Cₑₒ T_{ds} G_{ds} ^{m}C_{ds} G_{ds} G_{ds} ^{m}C_{ds} ^{m}C_{ds} G_{ds} | 69111 | 69130 | 44.4 | 87 |
| | T_{ds} G_{ds} Gₑₒ ^{m}Cₑₒ Gₑₛ Gₑₛ ^{m}Cₑ | | | | |
| 628715 | ^{m}Cₑₛ Tₑₒ ^{m}Cₑₒ Tₑₒ ^{m}Cₑₒ ^{m}C_{ds} ^{m}C_{ds} T_{ds} ^{m}C_{ds} ^{m}C_{ds} ^{m}C_{ds} | 69136 | 69155 | 54.7 | 88 |
| | ^{m}C_{ds} T_{ds} ^{m}C_{ds} G_{ds} Gₑₒ Tₑₒ Gₑₛ Tₑₛ Tₑ | | | | |
| 628719 | ^{m}Cₑₛ Tₑₒ Tₑₒ Gₑₒ Gₑₒ ^{m}C_{ds} A_{ds} T_{ds} G_{ds} G_{ds} A_{ds} G_{ds} G_{ds} | 69168 | 69187 | 41.5 | 89 |
| | A_{ds} T_{ds} Gₑₒ Aₑₒ Aₑₛ Aₑₛ ^{m}Cₑ | | | | |
| 628723 | Tₑₛ ^{m}Cₑₒ ^{m}Cₑₒ Gₑₒ Gₑₒ ^{m}C_{ds} T_{ds} G_{ds} T_{ds} ^{m}C_{ds} ^{m}C_{ds} A_{ds} | 69200 | 69219 | 59.6 | 90 |
| | ^{m}C_{ds} A_{ds} G_{ds} Gₑₒ ^{m}Cₑₒ Tₑₛ ^{m}Cₑₛ ^{m}Cₑ | | | | |
| 628727 | Aₑₛ Aₑₒ Tₑₒ ^{m}Cₑₒ ^{m}Cₑₒ G_{ds} ^{m}C_{ds} T_{ds} ^{m}C_{ds} ^{m}C_{ds} G_{ds} T_{ds} G_{ds} | 69244 | 69263 | 50.4 | 91 |
| | T_{ds} A_{ds} Aₑₒ Aₑₒ Gₑₛ Tₑₛ ^{m}Cₑ | | | | |
| 628731 | ^{m}Cₑₛ Aₑₒ Gₑₒ ^{m}Cₑₒ Tₑₒ G_{ds} ^{m}C_{ds} ^{m}C_{ds} T_{ds} T_{ds} T_{ds} A_{ds} T_{ds} | 69276 | 69295 | 43.3 | 92 |
| | T_{ds} ^{m}C_{ds} Tₑₒ Tₑₒ Gₑₛ Tₑₛ Tₑ | | | | |
| 628735 | Gₑₛ Tₑₒ ^{m}Cₑₒ Aₑₒ Gₑₒ A_{ds} G_{ds} ^{m}C_{ds} ^{m}C_{ds} ^{m}C_{ds} T_{ds} A_{ds} | 69308 | 69327 | 70.7 | 93 |
| | ^{m}C_{ds} ^{m}C_{ds} ^{m}C_{ds} Aₑₒ Tₑₒ Aₑₛ Aₑₛ Gₑ | | | | |

Superscript “m” indicates 5-methylcytosine. Subscripts: “o” indicates a phosphodiester internucleoside linkage, “s” indicates a phosphorothioate internucleoside linkage, “e” indicates a 2′-methoxyethyl modified nucleoside, and “d” indicates a 2′-deoxynucleoside.

TABLE 2
Inhibition of human MECP2 by antisense oligonucleotides in vitro

| | | Start | Stop | % | UNANNOTATED | ANNOTATED |
|---|---|---|---|---|---|---|
| Isis No. | Sequence (5′ to 3′) | site | site | Inhibition | SEQ ID NO: | SEQ ID NO |
| 18078 | Gₑₛ Tₑₛ Gₑₛ ^{m}Cₑₛ Gₑₛ C_{ds} G_{ds} C_{ds} G_{ds} A_{ds} G_{ds} C_{ds} C_{ds} C_{ds} | n/a | n/a | 3.8 | 15 | N/A |
| | Gₑₛ Aₑₛ Aₑₛ Aₑₛ Tₑₛ ^{m}Cₑ | | | | | |
| 628541 | Aₑₛ Gₑₒ ^{m}Cₑₒ Gₑₒ ^{m}Cₑₒ G_{ds} ^{m}C_{ds} G_{ds} ^{m}C_{ds} G_{ds} ^{m}C_{ds} ^{m}C_{ds} | 1878 | 1897 | 15.3 | 94 | N/A |
| | G_{ds} ^{m}C_{ds} ^{m}C_{ds} Gₑₒ Aₑₒ ^{m}Cₑₛ Gₑₛ ^{m}Cₑ | | | | | |
| 628545 | ^{m}Cₑₛ Tₑₒ Tₑₒ Tₑₒ Tₑₒ A_{ds} ^{m}C_{ds} ^{m}C_{ds} A_{ds} ^{m}C_{ds} A_{ds} G_{ds} ^{m}C_{ds} | 1910 | 1929 | 48.4 | 95 | N/A |
| | ^{m}C_{ds} ^{m}C_{ds} Tₑₒ ^{m}Cₑₒ Tₑₛ ^{m}Cₑₛ Tₑ | | | | | |
| 628549 | ^{m}Cₑₛ ^{m}Cₑₒ Gₑₒ ^{m}Cₑₒ Tₑₒ ^{m}C_{ds} G_{ds} G_{ds} ^{m}C_{ds} G_{ds} ^{m}C_{ds} G_{ds} | 1953 | 1972 | 32.9 | 96 | N/A |
| | G_{ds} ^{m}C_{ds} G_{ds} Gₑₒ ^{m}Cₑₒ Gₑₛ Gₑₛ ^{m}Cₑ | | | | | |
| 628741 | Tₑₛ ^{m}Cₑₒ Aₑₒ Gₑₒ Tₑₒ T_{ds} T_{ds} G_{ds} G_{ds} G_{ds} T_{ds} G_{ds} A_{ds} T_{ds} | 3047 | 3066 | 45.7 | 97 | N/A |
| | T_{ds} ^{m}Cₑₒ Gₑₒ Gₑₛ Tₑₛ ^{m}Cₑ | | | | | |
| 628745 | ^{m}Cₑₛ Aₑₒ Gₑₒ ^{m}Cₑₒ Aₑₒ ^{m}C_{ds} A_{ds} G_{ds} ^{m}C_{ds} G_{ds} G_{ds} G_{ds} A_{ds} | 5561 | 5580 | 42.2 | 98 | N/A |
| | A_{ds} ^{m}C_{ds} Aₑₒ ^{m}Cₑₒ Aₑₛ Tₑₛ Tₑ | | | | | |
| 628553 | Tₑₛ Aₑₒ Tₑₒ Tₑₒ Tₑₒ T_{ds} T_{ds} A_{ds} T_{ds} G_{ds} G_{ds} A_{ds} G_{ds} ^{m}C_{ds} | 7292 | 7311 | 36.6 | 99 | N/A |
| | A_{ds} Gₑₒ Tₑₒ ^{m}Cₑₛ Tₑₛ ^{m}Cₑ | | | | | |
| 628557 | Aₑₛ Tₑₒ Gₑₒ Tₑₒ ^{m}Cₑₒ A_{ds} ^{m}C_{ds} A_{ds} T_{ds} ^{m}C_{ds} A_{ds} A_{ds} A_{ds} | 7324 | 7343 | 70.2 | 100 | N/A |
| | G_{ds} ^{m}C_{ds} Aₑₒ Gₑₒ Gₑₛ Aₑₛ Aₑ | | | | | |
| 628561 | Tₑₛ Tₑₒ Gₑₒ Gₑₒ Aₑₒ G_{ds} ^{m}C_{ds} T_{ds} G_{ds} G_{ds} T_{ds} ^{m}C_{ds} T_{ds} A_{ds} | 7367 | 7386 | 43.4 | 101 | N/A |
| | ^{m}C_{ds} Aₑₒ Gₑₒ Aₑₛ Aₑₛ Gₑ | | | | | |
| 628565 | Aₑₛ Gₑₒ ^{m}Cₑₒ ^{m}Cₑₒ ^{m}Cₑₒ T_{ds} A_{ds} A_{ds} ^{m}C_{ds} A_{ds} T_{ds} ^{m}C_{ds} | 7399 | 7418 | 72.5 | 102 | N/A |
| | ^{m}C_{ds} ^{m}C_{ds} A_{ds} Gₑₒ ^{m}Cₑₒ Tₑₛ Aₑₛ ^{m}Cₑ | | | | | |
| 628749 | ^{m}Cₑₛ Aₑₒ ^{m}Cₑₒ Aₑₒ ^{m}Cₑₒ T_{ds} G_{ds} A_{ds} ^{m}C_{ds} ^{m}C_{ds} T_{ds} T_{ds} T_{ds} | 7615 | 7634 | 96.4 | 103 | N/A |
| | ^{m}C_{ds} A_{ds} Gₑₒ Gₑₒ Gₑₛ ^{m}Cₑₛ Tₑ | | | | | |
| 628753 | Tₑₛ Aₑₒ Aₑₒ Aₑₒ Aₑₒ A_{ds} A_{ds} G_{ds} G_{ds} A_{ds} T_{ds} T_{ds} T_{ds} ^{m}C_{ds} | 10408 | 10427 | 12.3 | 104 | N/A |
| | ^{m}C_{ds} Tₑₒ Aₑₒ Aₑₛ Gₑₛ Tₑ | | | | | |
| 628757 | Gₑₛ Tₑₒ Aₑₒ ^{m}Cₑₒ Aₑₒ ^{m}C_{ds} A_{ds} ^{m}C_{ds} A_{ds} ^{m}C_{ds} G_{ds} ^{m}C_{ds} | 13332 | 13351 | 85.5 | 105 | N/A |
| | T_{ds} T_{ds} T_{ds} Tₑₒ Tₑₒ Tₑₛ Tₑₛ Tₑ | | | | | |
| 628761 | Gₑₛ Aₑₒ Aₑₒ Aₑₒ Gₑₒ ^{m}C_{ds} ^{m}C_{ds} G_{ds} A_{ds} G_{ds} ^{m}C_{ds} ^{m}C_{ds} T_{ds} | 15686 | 15705 | 51.4 | 106 | N/A |
| | G_{ds} G_{ds} ^{m}Cₑₒ ^{m}Cₑₒ Gₑₛ Gₑₛ Gₑ | | | | | |
| 628765 | Gₑₛ Aₑₒ Aₑₒ Gₑₒ Aₑₒ A_{ds} A_{ds} A_{ds} T_{ds} G_{ds} T_{ds} G_{ds} G_{ds} A_{ds} | 18630 | 18649 | 78.3 | 107 | N/A |
| | T_{ds} Tₑₒ Tₑₒ Tₑₛ Tₑₛ Tₑ | | | | | |
| 628769 | ^{m}Cₑₛ Gₑₒ Aₑₒ Gₑₒ Aₑₒ A_{ds} T_{ds} G_{ds} A_{ds} G_{ds} A_{ds} ^{m}C_{ds} T_{ds} | 21317 | 21336 | 63.6 | 108 | N/A |
| | ^{m}C_{ds} ^{m}C_{ds} Gₑₒ Tₑₒ Aₑₛ Tₑₛ ^{m}Cₑ | | | | | |
| 628773 | Aₑₛ Aₑₒ Aₑₒ ^{m}Cₑₒ ^{m}Cₑₒ ^{m}C_{ds} A_{ds} A_{ds} A_{ds} ^{m}C_{ds} ^{m}C_{ds} A_{ds} | 23339 | 23358 | 48.9 | 109 | N/A |
| | ^{m}C_{ds} ^{m}C_{ds} T_{ds} Tₑₒ Aₑₒ ^{m}Cₑₛ ^{m}Cₑₛ ^{m}Cₑ | | | | | |
| 628777 | Aₑₛ Aₑₒ Aₑₒ Aₑₒ Tₑₒ A_{ds} A_{ds} A_{ds} G_{ds} T_{ds} ^{m}C_{ds} A_{ds} G_{ds} G_{ds} | 26037 | 26056 | 65.5 | 110 | N/A |
| | A_{ds} Gₑₒ Gₑₒ ^{m}Cₑₛ Tₑₛ Gₑ | | | | | |
| 628781 | Aₑₛ Aₑₒ Aₑₒ Aₑₒ Aₑₒ T_{ds} G_{ds} G_{ds} A_{ds} G_{ds} G_{ds} G_{ds} ^{m}C_{ds} A_{ds} | 28177 | 28196 | 12.2 | 111 | N/A |
| | ^{m}C_{ds} Aₑₒ Gₑₒ Tₑₛ Gₑₛ Gₑ | | | | | |
| 628785 | Gₑₛ Gₑₒ Tₑₒ Tₑₒ Tₑₒ T_{ds} T_{ds} ^{m}C_{ds} T_{ds} ^{m}C_{ds} ^{m}C_{ds} T_{ds} T_{ds} T_{ds} | 30744 | 30763 | 92.2 | 112 | N/A |
| | A_{ds} Tₑₒ Tₑₒ Aₑₛ Tₑₛ ^{m}Cₑ | | | | | |
| 628789 | Tₑₛ Aₑₒ Tₑₒ Gₑₒ Tₑₒ T_{ds} G_{ds} G_{ds} ^{m}C_{ds} ^{m}C_{ds} T_{ds} A_{ds} G_{ds} A_{ds} | 33273 | 33292 | 52.4 | 113 | N/A |
| | A_{ds} ^{m}Cₑₒ Tₑₒ ^{m}Cₑₛ Tₑ | | | | | |
| 628793 | Tₑₛ Gₑₒ ^{m}Cₑₒ Tₑₒ ^{m}Cₑₒ T_{ds} ^{m}C_{ds} A_{ds} T_{ds} A_{ds} T_{ds} T_{ds} ^{m}C_{ds} | 35287 | 35306 | 79.3 | 114 | N/A |
| | A_{ds} ^{m}C_{ds} ^{m}Cₑₒ ^{m}Cₑₒ Aₑₛ ^{m}Cₑₛ Gₑ | | | | | |
| 628797 | Gₑₛ Tₑₒ Gₑₒ ^{m}Cₑₒ Aₑₒ G_{ds} A_{ds} G_{ds} A_{ds} ^{m}C_{ds} T_{ds} ^{m}C_{ds} A_{ds} | 38049 | 38068 | 21.7 | 115 | N/A |
| | A_{ds} G_{ds} Gₑₒ Gₑₒ Aₑₛ Gₑₛ Gₑ | | | | | |
| 628801 | Gₑₛ ^{m}Cₑₒ Tₑₒ Aₑₒ Aₑₒ G_{ds} ^{m}C_{ds} ^{m}C_{ds} T_{ds} ^{m}C_{ds} ^{m}C_{ds} T_{ds} G_{ds} | 40072 | 40091 | 65.0 | 116 | N/A |
| | G_{ds} T_{ds} Gₑₒ Aₑₒ Aₑₛ ^{m}Cₑₛ ^{m}Cₑ | | | | | |
| 628805 | Gₑₛ Tₑₒ Aₑₒ Tₑₒ Gₑₒ A_{ds} A_{ds} ^{m}C_{ds} A_{ds} T_{ds} ^{m}C_{ds} A_{ds} G_{ds} | 42580 | 42599 | 69.6 | 117 | N/A |
| | ^{m}C_{ds} T_{ds} Gₑₒ Aₑₒ ^{m}Cₑₛ Gₑₛ ^{m}Cₑ | | | | | |
| 628809 | Aₑₛ Gₑₒ Gₑₒ ^{m}Cₑₒ Gₑₒ ^{m}C_{ds} G_{ds} ^{m}C_{ds} T_{ds} G_{ds} G_{ds} T_{ds} G_{ds} | 44735 | 44754 | 16.5 | 118 | N/A |
| | ^{m}C_{ds} A_{ds} Aₑₒ Gₑₒ ^{m}Cₑₛ ^{m}Cₑₛ Tₑ | | | | | |
| 628813 | ^{m}Cₑₛ Aₑₒ Gₑₒ ^{m}Cₑₒ ^{m}Cₑₒ A_{ds} ^{m}C_{ds} T_{ds} ^{m}C_{ds} T_{ds} T_{ds} T_{ds} T_{ds} | 46834 | 46853 | 59.1 | 119 | N/A |
| | T_{ds} T_{ds} Tₑₒ Tₑₒ Tₑₛ Gₑₛ Aₑ | | | | | |
| 628817 | Gₑₛ Tₑₒ Aₑₒ ^{m}Cₑₒ ^{m}Cₑₒ T_{ds} G_{ds} G_{ds} G_{ds} A_{ds} G_{ds} G_{ds} A_{ds} | 48863 | 48882 | 68.0 | 120 | N/A |
| | A_{ds} ^{m}C_{ds} Tₑₒ Aₑₒ ^{m}Cₑₛ Aₑₛ Aₑ | | | | | |
| 628821 | Aₑₛ Gₑₒ Gₑₒ Gₑₒ ^{m}Cₑₒ G_{ds} A_{ds} G_{ds} A_{ds} G_{ds} A_{ds} T_{ds} ^{m}C_{ds} | 50865 | 50884 | 80.7 | 121 | N/A |
| | ^{m}C_{ds} A_{ds} Gₑₒ Gₑₒ Aₑₛ ^{m}Cₑₛ Tₑ | | | | | |
| 628825 | Gₑₛ Gₑₒ Aₑₒ Tₑₒ Tₑₒ A_{ds} G_{ds} G_{ds} G_{ds} A_{ds} A_{ds} T_{ds} T_{ds} A_{ds} | 53552 | 53571 | 59.3 | 122 | N/A |
| | G_{ds} Aₑₒ Tₑₒ Gₑₛ ^{m}Cₑₛ Aₑ | | | | | |
| 628829 | Gₑₛ Gₑₒ Aₑₒ Aₑₒ Aₑₒ G_{ds} ^{m}C_{ds} ^{m}C_{ds} T_{ds} G_{ds} T_{ds} ^{m}C_{ds} T_{ds} | 55596 | 55615 | 54.0 | 123 | N/A |
| | T_{ds} T_{ds} Tₑₒ Aₑₒ Aₑₛ Aₑₛ Aₑ | | | | | |
| 628833 | ^{m}Cₑₛ ^{m}Cₑₒ Aₑₒ Gₑₒ Aₑₒ T_{ds} G_{ds} G_{ds} T_{ds} G_{ds} T_{ds} T_{ds} T_{ds} | 57622 | 57641 | 85.3 | 124 | N/A |
| | ^{m}C_{ds} ^{m}C_{ds} Aₑₒ Aₑₒ Tₑₛ Tₑₛ ^{m}Cₑ | | | | | |
| 628837 | Aₑₛ ^{m}Cₑₒ Tₑₒ Tₑₒ ^{m}Cₑₒ T_{ds} A_{ds} G_{ds} A_{ds} ^{m}C_{ds} ^{m}C_{ds} G_{ds} G_{ds} | 60266 | 60285 | 48.3 | 125 | N/A |
| | G_{ds} ^{m}C_{ds} Gₑₒ ^{m}Cₑₒ Aₑₛ Gₑₛ Tₑ | | | | | |
| 628841 | Gₑₛ Tₑₒ Aₑₒ ^{m}Cₑₒ Aₑₒ A_{ds} T_{ds} G_{ds} A_{ds} A_{ds} T_{ds} G_{ds} A_{ds} A_{ds} | 62361 | 62380 | 69.7 | 126 | N/A |
| | ^{m}C_{ds} Tₑₒ Tₑₒ Tₑₛ Tₑₛ Tₑ | | | | | |
| 628845 | ^{m}Cₑₛ Aₑₒ Aₑₒ Aₑₒ ^{m}Cₑₒ A_{ds} T_{ds} A_{ds} T_{ds} ^{m}C_{ds} T_{ds} A_{ds} ^{m}C_{ds} | 64407 | 64426 | 27.4 | 127 | N/A |
| | T_{ds} G_{ds} ^{m}Cₑₒ Aₑₒ Tₑₛ Tₑₛ ^{m}Cₑ | | | | | |
| 628849 | Aₑₛ ^{m}Cₑₒ Aₑₒ Gₑₒ Gₑₒ T_{ds} A_{ds} A_{ds} ^{m}C_{ds} ^{m}C_{ds} ^{m}C_{ds} ^{m}C_{ds} | 66432 | 66451 | 68.5 | 128 | N/A |
| | A_{ds} T_{ds} ^{m}C_{ds} Tₑₒ Aₑₒ Gₑₛ Gₑₛ ^{m}Cₑ | | | | | |
| 628569 | Gₑₛ Gₑₒ Aₑₒ Gₑₒ Gₑₒ T_{ds} ^{m}C_{ds} ^{m}C_{ds} T_{ds} G_{ds} G_{ds} T_{ds} ^{m}C_{ds} | 67064 | 67083 | 41.4 | 129 | N/A |
| | T_{ds} T_{ds} ^{m}Cₑₒ Tₑₒ Gₑₛ Aₑₛ ^{m}Cₑ | | | | | |
| 628573 | Tₑₛ Tₑₒ Aₑₒ Tₑₒ ^{m}Cₑₒ T_{ds} T_{ds} T_{ds} ^{m}C_{ds} T_{ds} T_{ds} ^{m}C_{ds} A_{ds} | 67113 | 67132 | 27.1 | 130 | N/A |
| | ^{m}C_{ds} ^{m}C_{ds} Tₑₒ Tₑₒ Tₑₛ Tₑₛ Tₑ | | | | | |
| 628577 | ^{m}Cₑₛ Tₑₒ ^{m}Cₑₒ Tₑₒ Tₑₒ T_{ds} ^{m}C_{ds} T_{ds} ^{m}C_{ds} T_{ds} T_{ds} ^{m}C_{ds} T_{ds} | 67129 | 67148 | 43.9 | 131 | N/A |
| | T_{ds} T_{ds} ^{m}Cₑₒ Tₑₒ Tₑₛ Aₑₛ Tₑ | | | | | |
| 628581 | ^{m}CₑₛAₑₒ ^{m}Cₑₒ Gₑₒ Gₑₒ G_{ds} ^{m}C_{ds} T_{ds} ^{m}C_{ds} A_{ds} T_{ds} G_{ds} | 67147 | 67166 | 15.0 | 132 | N/A |
| | ^{m}C_{ds} T_{ds} T_{ds} Gₑₒ ^{m}Cₑₒ ^{m}Cₑₛ ^{m}Cₑₛ Tₑ | | | | | |
| 628585 | Gₑₛ Gₑₒ ^{m}Cₑₒ Tₑₒ Gₑₒ A_{ds} T_{ds} G_{ds} G_{ds} ^{m}C_{ds} T_{ds} G_{ds} ^{m}C_{ds} | 67159 | 67178 | 73.7 | 133 | N/A |
| | A_{ds} ^{m}C_{ds} Gₑₒ Gₑₒ Gₑₛ ^{m}Cₑₛ Tₑ | | | | | |
| 628589 | Tₑₛ Gₑₒ ^{m}Cₑₒ Gₑₒ Gₑₒ G_{ds} ^{m}C_{ds} T_{ds} ^{m}C_{ds} A_{ds} G_{ds} ^{m}C_{ds} A_{ds} | 67180 | 67199 | 22.6 | 134 | N/A |
| | G_{ds} A_{ds} Gₑₒ Tₑₒ Gₑₛ Gₑₛ Tₑ | | | | | |
| 628593 | Gₑₛ Aₑₒ ^{m}Cₑₒ ^{m}Cₑₒ ^{m}Cₑₒ T_{ds} T_{ds} ^{m}C_{ds} T_{ds} G_{ds} A_{ds} T_{ds} G_{ds} | 67212 | 67231 | 47.4 | 135 | N/A |
| | T_{ds} ^{m}C_{ds} Tₑₒ ^{m}Cₑₒ Tₑₛ Gₑₛ ^{m}Cₑ | | | | | |
| 628597 | Aₑₛ Gₑₒ Gₑₒ ^{m}Cₑₒ Aₑₒ G_{ds} A_{ds} A_{ds} G_{ds} ^{m}C_{ds} T_{ds} T_{ds} ^{m}C_{ds} | 67247 | 67266 | 48.6 | 136 | N/A |
| | ^{m}C_{ds} G_{ds} Gₑₒ ^{m}Cₑₒ Aₑₛ ^{m}Cₑₛ Aₑ | | | | | |
| 628601 | Tₑₛ ^{m}Cₑₒ Aₑₒ Tₑₒ Aₑₒ ^{m}C_{ds} A_{ds} T_{ds} G_{ds} G_{ds} G_{ds} T_{ds} ^{m}C_{ds} | 67296 | 67315 | 53.7 | 137 | N/A |
| | ^{m}C_{ds} ^{m}C_{ds} ^{m}Cₑₒ Gₑₒ Gₑₛ Tₑₛ ^{m}Cₑ | | | | | |
| 628605 | Tₑₛ Tₑₒ Tₑₒ ^{m}Cₑₒ ^{m}Cₑₒ T_{ds} T_{ds} T_{ds} G_{ds} ^{m}C_{ds} T_{ds} T_{ds} A_{ds} | 67345 | 67364 | 46.5 | 138 | N/A |
| | A_{ds} G_{ds} ^{m}Cₑₒ Tₑₒ Tₑₛ ^{m}Cₑₛ ^{m}Cₑ | | | | | |
| 628609 | Tₑₛ Aₑₒ ^{m}Cₑₒ Aₑₒ ^{m}Cₑₒ A_{ds} T_{ds} ^{m}C_{ds} A_{ds} T_{ds} A_{ds} ^{m}C_{ds} T_{ds} | 67377 | 67396 | 40.1 | 139 | N/A |
| | T_{ds} ^{m}C_{ds} ^{m}Cₑₒ ^{m}Cₑₒ Aₑₛ Gₑₛ ^{m}Cₑ | | | | | |
| 628613 | Tₑₛ ^{m}Cₑₒ Aₑₒ Aₑₒ ^{m}Cₑₒ T_{ds} ^{m}C_{ds} ^{m}C_{ds} A_{ds} ^{m}C_{ds} T_{ds} T_{ds} T_{ds} | 68180 | 68199 | 42.9 | 140 | N/A |
| | A_{ds} G_{ds} Aₑₒ Gₑₒ ^{m}Cₑₛ Gₑₛ Aₑ | | | | | |
| 628617 | Gₑₛ ^{m}Cₑₒ ^{m}Cₑₒ Tₑₒ Aₑₒ ^{m}C_{ds} ^{m}C_{ds} T_{ds} T_{ds} T_{ds} T_{ds} ^{m}C_{ds} G_{ds} | 68203 | 68222 | 65.5 | 141 | N/A |
| | A_{ds} A_{ds} Gₑₒ Tₑₒ Aₑₛ ^{m}Cₑₛ Gₑ | | | | | |
| 628621 | Gₑₛ Gₑₒ Tₑₒ ^{m}Cₑₒ ^{m}Cₑₒ A_{ds} G_{ds} G_{ds} G_{ds} A_{ds} T_{ds} G_{ds} T_{ds} | 68219 | 68238 | 62.9 | 142 | N/A |
| | G_{ds} T_{ds} ^{m}Cₑₒ Gₑₒ ^{m}Cₑₛ ^{m}Cₑₛ Tₑ | | | | | |
| 628625 | Tₑₛ ^{m}Cₑₒ ^{m}Cₑₒ ^{m}Cₑₒ Tₑₒ ^{m}C_{ds} T_{ds} ^{m}C_{ds} ^{m}C_{ds} ^{m}C_{ds} A_{ds} G_{ds} | 68255 | 68274 | 65.6 | 143 | N/A |
| | T_{ds} T_{ds} A_{ds} ^{m}Cₑₒ ^{m}Cₑₒ Gₑₛ Tₑₛ Gₑ | | | | | |
| 628629 | Tₑₛ Gₑₒ Gₑₒ Aₑₒ Gₑₒ ^{m}C_{ds} T_{ds} T_{ds} T_{ds} G_{ds} G_{ds} G_{ds} A_{ds} G_{ds} | 68311 | 68330 | 28.3 | 144 | N/A |
| | A_{ds} Tₑₒ Tₑₒ Tₑₛ Gₑₛ Gₑ | | | | | |
| 628633 | ^{m}Cₑₛ Gₑₒ Tₑₒ Gₑₒ Gₑₒ ^{m}C_{ds} ^{m}C_{ds} G_{ds} ^{m}C_{ds} ^{m}C_{ds} T_{ds} T_{ds} G_{ds} | 68374 | 68393 | 25.5 | 145 | N/A |
| | G_{ds} G_{ds} Tₑₒ ^{m}Cₑₒ Tₑₛ ^{m}Cₑₛ Gₑ | | | | | |
| 628637 | Aₑₛ Gₑₒ Gₑₒ Aₑₒ ^{m}Cₑₒ T_{ds} T_{ds} T_{ds} T_{ds} ^{m}C_{ds} T_{ds} ^{m}C_{ds} ^{m}C_{ds} | 68416 | 68435 | 86.1 | 146 | N/A |
| | A_{ds} G_{ds} Gₑₒ Aₑₒ ^{m}Cₑₛ ^{m}Cₑₛ ^{m}Cₑ | | | | | |
| 628641 | Aₑₛ Gₑₒ Gₑₒ ^{m}Cₑₒ Aₑₒ T_{ds} ^{m}C_{ds} T_{ds} T_{ds} G_{ds} A_{ds} ^{m}C_{ds} A_{ds} | 68440 | 68459 | 81.0 | 147 | N/A |
| | A_{ds} G_{ds} Gₑₒ Aₑₒ Gₑₛ ^{m}Cₑₛ Tₑ | | | | | |
| 628645 | Gₑₛ ^{m}Cₑₒ ^{m}Cₑₒ ^{m}Cₑₒ ^{m}Cₑₒ ^{m}C_{ds} T_{ds} G_{ds} G_{ds} ^{m}C_{ds} G_{ds} A_{ds} | 68461 | 68480 | 29.6 | 148 | N/A |
| | A_{ds} G_{ds} T_{ds} Tₑₒ Tₑₒ Gₑₛ Aₑₛ Aₑ | | | | | |
| 628649 | ^{m}Cₑₛ ^{m}Cₑₒ ^{m}Cₑₒ ^{m}Cₑₒ Aₑₒ ^{m}C_{ds} ^{m}C_{ds} ^{m}C_{ds} ^{m}C_{ds} ^{m}C_{ds} ^{m}C_{ds} | 68480 | 68499 | 39.9 | 149 | N/A |
| | T_{ds} ^{m}C_{ds} A_{ds} G_{ds} ^{m}Cₑₒ ^{m}Cₑₒ Tₑₛ Tₑₛ Gₑ | | | | | |
| 628653 | ^{m}Cₑₛ ^{m}Cₑₒ Aₑₒ Tₑₒ Gₑₒ A_{ds} ^{m}C_{ds} ^{m}C_{ds} T_{ds} G_{ds} G_{ds} G_{ds} T_{ds} | 68504 | 68523 | 39.7 | 150 | N/A |
| | G_{ds} G_{ds} Aₑₒ Tₑₒ Gₑₛ Tₑₛ Gₑ | | | | | |
| 628657 | ^{m}Cₑₛ Tₑₒ Gₑₒ Aₑₒ Gₑₒ G_{ds} G_{ds} T_{ds} ^{m}C_{ds} G_{ds} G_{ds} ^{m}C_{ds} ^{m}C_{ds} | 68551 | 68570 | 28.6 | 151 | N/A |
| | T_{ds} ^{m}C_{ds} Aₑₒ Gₑₒ ^{m}Cₑₛ Tₑₛ Tₑ | | | | | |
| 628661 | ^{m}Cₑₛ ^{m}Cₑₒ ^{m}Cₑₒ Gₑₒ Gₑₒ ^{m}C_{ds} T_{ds} T_{ds} T_{ds} ^{m}C_{ds} G_{ds} G_{ds} | 68583 | 68602 | 37.3 | 152 | N/A |
| | ^{m}C_{ds} ^{m}C_{ds} ^{m}C_{ds} ^{m}Cₑₒ Gₑₒ Tₑₛ Tₑₛ Tₑ | | | | | |
| 628665 | Tₑₛ Gₑₒ Gₑₒ ^{m}Cₑₒ ^{m}Cₑₒ T_{ds} ^{m}C_{ds} G_{ds} G_{ds} ^{m}C_{ds} G_{ds} G_{ds} | 68615 | 68634 | 38.2 | 153 | N/A |
| | ^{m}C_{ds} A_{ds} G_{ds} ^{m}Cₑₒ Gₑₒ Gₑₛ ^{m}Cₑₛ Tₑ | | | | | |
| *628669 | Tₑₛ ^{m}Cₑₒ Gₑₒ Gₑₒ Aₑₒ T_{ds} A_{ds} G_{ds} A_{ds} A_{ds} G_{ds} A_{ds} ^{m}C_{ds} | 68647 | 68666 | 66.3 | 154 | N/A |
| | T_{ds} ^{m}C_{ds} ^{m}Cₑₒ Tₑₒ Tₑₛ ^{m}Cₑₛ Aₑ | | | | | |
| *628673 | Tₑₛ Aₑₒ ^{m}Cₑₒ Gₑₒ Gₑₒ T_{ds} ^{m}C_{ds} T_{ds} ^{m}C_{ds} ^{m}C_{ds} T_{ds} G_{ds} ^{m}C_{ds} | 68665 | 68684 | 83.3 | 155 | N/A |
| | A_{ds} ^{m}C_{ds} Aₑₒ Gₑₒ Aₑₛ Tₑₛ ^{m}Cₑ | | | | | |
| *628677 | Aₑₛ ^{m}Cₑₒ ^{m}Cₑₒ Tₑₒ ^{m}Cₑₒ G_{ds} A_{ds} T_{ds} G_{ds} ^{m}C_{ds} T_{ds} G_{ds} A_{ds} | 68712 | 68731 | 70.8 | 156 | N/A |
| | ^{m}C_{ds} ^{m}C_{ds} Gₑₒ Tₑₒ ^{m}Cₑₛ Tₑₛ ^{m}Cₑ | | | | | |
| 628681 | ^{m}Cₑₛ Tₑₒ Tₑₒ ^{m}Cₑₒ Tₑₒ ^{m}C_{ds} A_{ds} ^{m}C_{ds} ^{m}C_{ds} G_{ds} A_{ds} G_{ds} G_{ds} | 68758 | 68777 | 24.2 | 157 | N/A |
| | G_{ds} T_{ds} Gₑₒ Gₑₒ Aₑₛ ^{m}Cₑₛ Aₑ | | | | | |
| 628685 | Gₑₛ Gₑₒ Gₑₒ ^{m}Cₑₒ Tₑₒ ^{m}C_{ds} T_{ds} T_{ds} A_{ds} ^{m}C_{ds} A_{ds} G_{ds} G_{ds} | 68790 | 68809 | 62.3 | 158 | N/A |
| | T_{ds} ^{m}C_{ds} Tₑₒ Tₑₒ ^{m}Cₑₛ Aₑₛ Gₑ | | | | | |
| 628689 | ^{m}Cₑₛ Tₑₒ Gₑₒ ^{m}Cₑₒ Tₑₒ G_{ds} ^{m}C_{ds} T_{ds} G_{ds} ^{m}C_{ds} G_{ds} ^{m}C_{ds} | 68835 | 68854 | 73.7 | 159 | N/A |
| | ^{m}C_{ds} ^{m}C_{ds} ^{m}C_{ds} Tₑₒ Tₑₒ Gₑₛ Gₑₛ Gₑ | | | | | |
| 628693 | Tₑₛ ^{m}Cₑₒ Gₑₒ Gₑₒ Gₑₒ ^{m}C_{ds} T_{ds} ^{m}C_{ds} A_{ds} G_{ds} G_{ds} T_{ds} G_{ds} | 68946 | 68965 | 13.0 | 160 | N/A |
| | G_{ds} A_{ds} Gₑₒ Gₑₒ Tₑₛ Gₑₛ Gₑ | | | | | |
| 628697 | ^{m}Cₑₛ Tₑₒ Gₑₒ Gₑₒ Gₑₒ ^{m}C_{ds} A_{ds} T_{ds} ^{m}C_{ds} T_{ds} T_{ds} ^{m}C_{ds} T_{ds} | 69024 | 69043 | 59.0 | 161 | N/A |
| | ^{m}C_{ds} ^{m}C_{ds} Tₑₒ ^{m}Cₑₒ Tₑₛ Tₑₛ Tₑ | | | | | |
| 628701 | Gₑₛ Tₑₒ ^{m}Cₑₒ Tₑₒ Tₑₒ A_{ds} G_{ds} ^{m}C_{ds} T_{ds} G_{ds} G_{ds} ^{m}C_{ds} T_{ds} | 69072 | 69091 | 63.7 | 162 | N/A |
| | ^{m}C_{ds} ^{m}C_{ds} Tₑₒ Tₑₒ Gₑₛ Gₑₛ Gₑ | | | | | |
| 628705 | Gₑₛ Gₑₒ Tₑₒ Gₑₒ Gₑₒ ^{m}C_{ds} A_{ds} A_{ds} ^{m}C_{ds} ^{m}C_{ds} G_{ds} ^{m}C_{ds} G_{ds} | 69091 | 69110 | 59.5 | 163 | N/A |
| | G_{ds} G_{ds} ^{m}Cₑₒ Tₑₒ Gₑₛ Aₑₛ Gₑ | | | | | |
| 628709 | ^{m}Cₑₛ Gₑₒ Gₑₒ ^{m}Cₑₒ ^{m}Cₑₒ G_{ds} T_{ds} G_{ds} G_{ds} ^{m}C_{ds} G_{ds} G_{ds} | 69104 | 69123 | 39.5 | 164 | N/A |
| | ^{m}C_{ds} G_{ds} G_{ds} Tₑₒ Gₑₒ Gₑₛ ^{m}Cₑₛ Aₑ | | | | | |
| 628713 | Tₑₛ Gₑₒ Tₑₒ Tₑₒ Tₑₒ G_{ds} T_{ds} A_{ds} ^{m}C_{ds} T_{ds} T_{ds} T_{ds} T_{ds} ^{m}C_{ds} | 69120 | 69139 | 58.1 | 165 | N/A |
| | T_{ds} Gₑₒ ^{m}Cₑₒ Gₑₛ Gₑₛ ^{m}Cₑ | | | | | |
| 628717 | Aₑₛ Aₑₒ Aₑₒ ^{m}Cₑₒ Aₑₒ A_{ds} T_{ds} G_{ds} T_{ds} ^{m}C_{ds} T_{ds} T_{ds} T_{ds} G_{ds} | 69152 | 69171 | 59.3 | 166 | N/A |
| | ^{m}C_{ds} Gₑₒ ^{m}Cₑₒ Tₑₛ ^{m}Cₑₛ Tₑ | | | | | |
| 628721 | ^{m}Cₑₛ Tₑₒ ^{m}Cₑₒ ^{m}Cₑₒ Tₑₒ ^{m}C_{ds} T_{ds} ^{m}C_{ds} T_{ds} G_{ds} T_{ds} T_{ds} T_{ds} | 69184 | 69203 | 61.6 | 167 | N/A |
| | G_{ds} G_{ds} ^{m}Cₑₒ ^{m}Cₑₒ Tₑₛ Tₑₛ Gₑ | | | | | |
| 628725 | Aₑₛ Gₑₒ Tₑₒ ^{m}Cₑₒ Aₑₒ G_{ds} ^{m}C_{ds} T_{ds} A_{ds} A_{ds} ^{m}C_{ds} T_{ds} ^{m}C_{ds} | 69228 | 69247 | 66.3 | 168 | N/A |
| | T_{ds} ^{m}C_{ds} Tₑₒ ^{m}Cₑₒ Gₑₛ Gₑₛ Tₑ | | | | | |
| 628729 | Tₑₛ Gₑₒ Tₑₒ Tₑₒ Gₑₒ G_{ds} T_{ds} T_{ds} T_{ds} G_{ds} ^{m}C_{ds} T_{ds} T_{ds} T_{ds} | 69260 | 69279 | 79.7 | 169 | N/A |
| | G_{ds} ^{m}Cₑₒ Aₑₒ Aₑₛ Tₑₛ ^{m}Cₑ | | | | | |
| 628733 | Tₑₛ Aₑₒ Aₑₒ Gₑₒ Gₑₒ A_{ds} G_{ds} A_{ds} A_{ds} G_{ds} A_{ds} G_{ds} A_{ds} ^{m}C_{ds} | 69292 | 69311 | 35.1 | 170 | N/A |
| | A_{ds} Aₑₒ ^{m}Cₑₒ Aₑₛ Gₑₛ ^{m}Cₑ | | | | | |
| 628737 | Tₑₛ Tₑₒ Aₑₒ Aₑₒ Tₑₒ ^{m}C_{ds} G_{ds} G_{ds} G_{ds} A_{ds} A_{ds} G_{ds} ^{m}C_{ds} | 69324 | 69343 | 78.1 | 171 | N/A |
| | T_{ds} T_{ds} Tₑₒ Gₑₒ Tₑₛ ^{m}Cₑₛ Aₑ | | | | | |

Superscript “m” indicates 5-methylcytosine. Subscripts: “o” indicates a phosphodiester internucleoside linkage, “s” indicates a phosphorothioate internucleoside linkage, “e” indicates a 2′-methoxyethyl modified nucleoside, and “d” indicates a 2′-deoxynucleoside.

TABLE 3
Inhibition of human MECP2 by antisense oligonucleotides in vitro

| Isis | | Start | Stop | % | UNANNOTATED | ANNOTATED |
|---|---|---|---|---|---|---|
| No. | Sequence (5′ to 3′) | site | site | Inhibition | SEQ ID NO | SEQ ID NO |
| 18078 | Gₑₛ Tₑₛ Gₑₛ ^{m}Cₑₛ Gₑₛ C_{ds} G_{ds} C_{ds} G_{ds} A_{ds} G_{ds} C_{ds} C_{ds} C_{ds} | n/a | n/a | 0.6 | 15 | N/A |
| | Gₑₛ Aₑₛ Aₑₛ Aₑₛ Tₑₛ ^{m}Cₑ | | | | | |
| 628542 | Gₑₛ Aₑₒ Gₑₒ Gₑₒ Aₑₒ G_{ds} G_{ds} G_{ds} A_{ds} G_{ds} ^{m}C_{ds} G_{ds} ^{m}C_{ds} | 1886 | 1905 | 23.9 | 172 | N/A |
| | G_{ds} ^{m}C_{ds} Gₑₒ ^{m}Cₑₒ Gₑₛ ^{m}Cₑₛ ^{m}Cₑ | | | | | |
| 628546 | ^{m}Cₑₛ ^{m}Cₑₒ Gₑₒ Gₑₒ Aₑₒ ^{m}C_{ds} G_{ds} G_{ds} ^{m}C_{ds} T_{ds} T_{ds} T_{ds} T_{ds} | 1918 | 1937 | 60.4 | 173 | N/A |
| | A_{ds} ^{m}C_{ds} ^{m}Cₑₒ Aₑₒ ^{m}Cₑₛ Aₑₛ Gₑ | | | | | |
| 628550 | ^{m}Cₑₛ Tₑₒ ^{m}Cₑₒ ^{m}Cₑₒ Tₑₒ ^{m}C_{ds} ^{m}C_{ds} T_{ds} ^{m}C_{ds} ^{m}C_{ds} G_{ds} ^{m}C_{ds} | 1961 | 1980 | 67.0 | 174 | N/A |
| | T_{ds} ^{m}C_{ds} G_{ds} Gₑₒ ^{m}Cₑₒ Gₑₛ ^{m}Cₑₛ Gₑ | | | | | |
| 628742 | Aₑₛ Tₑₒ Gₑₒ ^{m}Cₑₒ Tₑₒ T_{ds} ^{m}C_{ds} A_{ds} T_{ds} T_{ds} T_{ds} T_{ds} T_{ds} A_{ds} | 3547 | 3566 | 82.9 | 175 | N/A |
| | ^{m}C_{ds} Aₑₒ Gₑₒ Tₑₛ Aₑₛ Tₑ | | | | | |
| 628746 | Gₑₛ Aₑₒ Gₑₒ ^{m}Cₑₒ ^{m}Cₑₒ A_{ds} G_{ds} A_{ds} G_{ds} G_{ds} ^{m}C_{ds} T_{ds} G_{ds} | 6078 | 6097 | 38.7 | 176 | N/A |
| | G_{ds} G_{ds} Tₑₒ Gₑₒ ^{m}Cₑₛ Gₑₛ Gₑ | | | | | |
| 628554 | Tₑₛ Gₑₒ Aₑₒ Gₑₒ Tₑₒ ^{m}C_{ds} T_{ds} G_{ds} T_{ds} A_{ds} T_{ds} T_{ds} T_{ds} T_{ds} | 7300 | 7319 | 57.7 | 177 | 331 |
| | T_{ds} Aₑₒ Tₑₒ Gₑₛ Gₑₛ Aₑ | | | | | |
| 628558 | Gₑₛ Gₑₒ Aₑₒ Gₑₒ Tₑₒ ^{m}C_{ds} A_{ds} ^{m}C_{ds} A_{ds} T_{ds} G_{ds} T_{ds} ^{m}C_{ds} | 7332 | 7351 | 82.8 | 178 | 332 |
| | A_{ds} ^{m}C_{ds} Aₑₒ Tₑₒ ^{m}Cₑₛ Aₑₛ Aₑ | | | | | |
| 628562 | Gₑₛ Aₑₒ Aₑₒ Tₑₒ ^{m}Cₑₒ ^{m}C_{ds} T_{ds} G_{ds} T_{ds} T_{ds} G_{ds} G_{ds} A_{ds} | 7375 | 7394 | 67.3 | 179 | 333 |
| | G_{ds} ^{m}C_{ds} Tₑₒ Gₑₒ Gₑₛ Tₑₛ ^{m}Cₑ | | | | | |
| 628566 | ^{m}Cₑₛ Tₑₒ Tₑₒ ^{m}Cₑₒ ^{m}Cₑₒ ^{m}C_{ds} T_{ds} G_{ds} A_{ds} G_{ds} ^{m}C_{ds} ^{m}C_{ds} | 7407 | 7426 | 32.1 | 180 | N/A |
| | ^{m}C_{ds} T_{ds} A_{ds} Aₑₒ ^{m}Cₑₒ Aₑₛ Tₑₛ ^{m}Cₑ | | | | | |
| 628750 | ^{m}Cₑₛ ^{m}Cₑₒ ^{m}Cₑₒ Aₑₒ ^{m}Cₑₒ A_{ds} G_{ds} ^{m}C_{ds} A_{ds} G_{ds} T_{ds} A_{ds} | 8615 | 8634 | 54.0 | 181 | N/A |
| | A_{ds} A_{ds} A_{ds} Gₑₒ Aₑₒ Gₑₛ Aₑₛ Aₑ | | | | | |
| 628754 | Aₑₛ ^{m}Cₑₒ ^{m}Cₑₒ ^{m}Cₑₒ ^{m}Cₑₒ A_{ds} G_{ds} T_{ds} A_{ds} G_{ds} T_{ds} T_{ds} G_{ds} | 11009 | 11028 | 40.8 | 182 | N/A |
| | A_{ds} G_{ds} Aₑₒ Tₑₒ Tₑₛ Aₑₛ ^{m}Cₑ | | | | | |
| 628758 | Aₑₛ Tₑₒ Aₑₒ Gₑₒ Tₑₒ A_{ds} G_{ds} T_{ds} T_{ds} G_{ds} ^{m}C_{ds} ^{m}C_{ds} A_{ds} | 13862 | 13881 | 38.7 | 183 | N/A |
| | G_{ds} A_{ds} Gₑₒ Gₑₒ Gₑₛ Tₑₛ Gₑ | | | | | |
| 628762 | Gₑₛ Gₑₒ ^{m}Cₑₒ Tₑₒ Tₑₒ ^{m}C_{ds} T_{ds} A_{ds} T_{ds} T_{ds} G_{ds} T_{ds} A_{ds} A_{ds} | 16687 | 16706 | 95.8 | 184 | N/A |
| | A_{ds} Aₑₒ ^{m}Cₑₒ Tₑₛ Aₑₛ Tₑ | | | | | |
| 628766 | Aₑₛ ^{m}Cₑₒ Tₑₒ Gₑₒ Gₑₒ T_{ds} T_{ds} T_{ds} T_{ds} T_{ds} A_{ds} A_{ds} G_{ds} A_{ds} | 19134 | 19153 | 83.1 | 185 | N/A |
| | G_{ds} Aₑₒ Tₑₒ Gₑₛ Gₑₛ Gₑ | | | | | |
| 628770 | Tₑₛ Aₑₒ Aₑₒ Aₑₒ Aₑₒ T_{ds} ^{m}C_{ds} T_{ds} A_{ds} T_{ds} G_{ds} G_{ds} G_{ds} A_{ds} | 21818 | 21837 | −7.4 | 186 | N/A |
| | A_{ds} Tₑₒ Aₑₒ Aₑₛ Aₑₛ Aₑ | | | | | |
| 628774 | Gₑₛ Aₑₒ Aₑₒ Aₑₒ Tₑₒ G_{ds} T_{ds} G_{ds} G_{ds} G_{ds} ^{m}C_{ds} T_{ds} T_{ds} G_{ds} | 23936 | 23955 | 62.6 | 187 | N/A |
| | G_{ds} ^{m}Cₑₒ Aₑₒ Tₑₛ Gₑₛ Gₑ | | | | | |
| 628778 | Aₑₛ Aₑₒ ^{m}Cₑₒ Aₑₒ Tₑₒ G_{ds} G_{ds} T_{ds} T_{ds} T_{ds} A_{ds} G_{ds} T_{ds} A_{ds} | 26672 | 26691 | 72.8 | 188 | N/A |
| | G_{ds} Aₑₒ Aₑₒ Aₑₛ ^{m}Cₑₛ ^{m}Cₑ | | | | | |
| 628782 | Gₑₛ Gₑₒ Tₑₒ Aₑₒ Tₑₒ T_{ds} A_{ds} T_{ds} A_{ds} A_{ds} T_{ds} T_{ds} T_{ds} T_{ds} | 28682 | 28701 | 49.9 | 189 | N/A |
| | G_{ds} Tₑₒ Aₑₒ Aₑₛ Tₑₛ Tₑ | | | | | |
| 628786 | ^{m}Cₑₛ Aₑₒ Aₑₒ ^{m}Cₑₒ Aₑₒ T_{ds} T_{ds} ^{m}C_{ds} ^{m}C_{ds} A_{ds} T_{ds} T_{ds} T_{ds} | 31258 | 31277 | 90.5 | 190 | N/A |
| | A_{ds} T_{ds} Tₑₒ Tₑₒ Aₑₛ Gₑₛ Gₑ | | | | | |
| 628790 | Aₑₛ Tₑₒ Tₑₒ Tₑₒ Tₑₒ ^{m}C_{ds} A_{ds} ^{m}C_{ds} ^{m}C_{ds} ^{m}C_{ds} T_{ds} T_{ds} T_{ds} | 33773 | 33792 | 63.0 | 191 | N/A |
| | A_{ds} A_{ds} Aₑₒ Aₑₒ Aₑₛ Tₑₛ ^{m}Cₑ | | | | | |
| 628794 | Tₑₛ Aₑₒ Aₑₒ Tₑₒ Aₑₒ ^{m}C_{ds} A_{ds} G_{ds} T_{ds} G_{ds} A_{ds} ^{m}C_{d}s A_{ds} | 35787 | 35806 | 68.0 | 192 | N/A |
| | A_{ds} G_{ds} ^{m}Cₑₒ Aₑₒ Tₑₛ ^{m}Cₑₛ ^{m}Cₑ | | | | | |
| 628798 | Tₑₛ ^{m}Cₑₒ ^{m}Cₑₒ Aₑₒ Tₑₒ ^{m}C_{ds} T_{ds} T_{ds} G_{ds} ^{m}C_{ds} A_{ds} G_{ds} G_{ds} | 38549 | 38568 | 77.6 | 193 | N/A |
| | T_{ds} G_{ds} Gₑₒ Aₑₒ Gₑₛ Tₑₛ Aₑ | | | | | |
| 628802 | Gₑₛ Aₑₒ Aₑₒ Gₑₒ ^{m}Cₑₒ ^{m}C_{ds} A_{ds} A_{ds} A_{ds} A_{ds} A_{ds} A_{ds} G_{ds} | 40573 | 40592 | 60.5 | 194 | N/A |
| | ^{m}C_{ds} A_{ds} Aₑₒ ^{m}Cₑₒ Aₑₛ Aₑₛ Aₑ | | | | | |
| 628806 | ^{m}Cₑₛ ^{m}Cₑₒ Aₑₒ Aₑₒ Gₑₒ A_{ds} ^{m}C_{ds} A_{ds} A_{ds} G_{ds} G_{ds} A_{ds} A_{ds} | 43080 | 43099 | 57.5 | 195 | N/A |
| | A_{ds} A_{ds} Aₑₒ ^{m}Cₑₒ Gₑₛ Gₑₛ Gₑ | | | | | |
| 628810 | ^{m}Cₑₛ Tₑₒ Aₑₒ Gₑₒ ^{m}Cₑₒ T_{ds} A_{ds} T_{ds} ^{m}C_{ds} A_{ds} G_{ds} ^{m}C_{ds} T_{ds} | 45258 | 45277 | 66.7 | 196 | N/A |
| | G_{ds} G_{ds} Gₑₒ ^{m}Cₑₒ Aₑₛ Tₑₛ Gₑ | | | | | |
| 628814 | Tₑₛ Gₑₒ ^{m}Cₑₒ ^{m}Cₑₒ Tₑₒ T_{ds} G_{ds} T_{ds} T_{ds} G_{ds} G_{ds} G_{ds} T_{ds} A_{ds} | 47334 | 47353 | 72.0 | 197 | N/A |
| | G_{ds} Tₑₒ Aₑₒ ^{m}Cₑₛ Aₑₛ Gₑ | | | | | |
| 628818 | Gₑₛ ^{m}Cₑₒ Tₑₒ Aₑₒ Aₑₒ G_{ds} T_{ds} T_{ds} A_{ds} G_{ds} A_{ds} A_{ds} ^{m}C_{ds} | 49363 | 49382 | 49.4 | 198 | N/A |
| | T_{ds} ^{m}C_{ds} ^{m}Cₑₒ Gₑₒ Tₑₛ Gₑₛ Gₑ | | | | | |
| 628822 | Aₑₛ ^{m}Cₑₒ Aₑₒ ^{m}Cₑₒ Gₑₒ ^{m}C_{ds} ^{m}C_{ds} T_{ds} G_{ds} T_{ds} A_{ds} A_{ds} T_{ds} | 51552 | 51571 | 81.5 | 199 | N/A |
| | ^{m}C_{ds} ^{m}C_{ds} Tₑₒ Gₑₒ ^{m}Cₑₛ Aₑₛ Tₑ | | | | | |
| 628826 | ^{m}Cₑₛ Aₑₒ Aₑₒ ^{m}Cₑₒ Tₑₒ G_{ds} G_{ds} A_{ds} G_{ds} G_{ds} ^{m}C_{ds} ^{m}C_{ds} G_{ds} | 54069 | 54088 | 9.7 | 200 | N/A |
| | G_{ds} G_{ds} ^{m}Cₑₒ Gₑₒ ^{m}Cₑₛ Gₑₛ Aₑ | | | | | |
| 628830 | Aₑₛ Gₑₒ ^{m}Cₑₒ ^{m}Cₑₒ ^{m}Cₑₒ A_{ds} ^{m}C_{ds} A_{ds} ^{m}C_{ds} A_{ds} G_{ds} ^{m}C_{ds} | 56096 | 56115 | 41.4 | 201 | N/A |
| | T_{ds} G_{ds} T_{ds} ^{m}Cₑₒ Tₑₒ ^{m}Cₑₛ Aₑₛ Gₑ | | | | | |
| 628834 | Tₑₛ Tₑₒ ^{m}Cₑₒ ^{m}Cₑₒ Tₑₒ ^{m}C_{ds} A_{ds} T_{ds} G_{ds} A_{ds} A_{ds} T_{ds} G_{ds} | 58122 | 58141 | 47.6 | 202 | N/A |
| | T_{ds} G_{ds} Aₑₒ ^{m}Cₑₒ ^{m}Cₑₛ Tₑₛ Gₑ | | | | | |
| 628838 | Gₑₛ Aₑₒ Gₑₒ Gₑₒ Aₑₒ A_{ds} ^{m}C_{ds} T_{ds} T_{ds} G_{ds} T_{ds} ^{m}C_{ds} T_{ds} | 60766 | 60785 | 68.3 | 203 | N/A |
| | G_{ds} A_{ds} Gₑₒ Aₑₒ Tₑₛ ^{m}Cₑₛ Aₑ | | | | | |
| 628842 | ^{m}Cₑₛ Aₑₒ Gₑₒ ^{m}Cₑₒ Tₑₒ A_{ds} ^{m}C_{ds} T_{ds} ^{m}C_{ds} G_{ds} ^{m}C_{ds} T_{ds} A_{ds} | 62880 | 62899 | 70.8 | 204 | N/A |
| | G_{ds} A_{ds} Aₑₒ Aₑₒ Gₑₛ Gₑₛ Gₑ | | | | | |
| 628846 | ^{m}Cₑₛ Tₑₒ ^{m}Cₑₒ ^{m}Cₑₒ ^{m}Cₑₒ ^{m}C_{ds} A_{ds} T_{ds} A_{ds} A_{ds} A_{ds} G_{ds} | 64930 | 64949 | 1.6 | 205 | N/A |
| | G_{ds} A_{ds} G_{ds} Gₑₒ Gₑₒ Aₑₛ Gₑₛ Gₑ | | | | | |
| 628850 | ^{m}Cₑₛ ^{m}Cₑₒ Aₑₒ Tₑₒ ^{m}Cₑₒ A_{ds} T_{ds} A_{ds} ^{m}C_{ds} A_{ds} ^{m}C_{ds} T_{ds} | 66932 | 66951 | 62.6 | 206 | N/A |
| | ^{m}C_{ds} A_{ds} G_{ds} Aₑₒ Tₑₒ ^{m}Cₑₛ Tₑₛ Tₑ | | | | | |
| 628570 | Tₑₛ Tₑₒ Gₑₒ Aₑₒ Gₑₒ G_{ds} ^{m}C_{ds} ^{m}C_{ds} ^{m}C_{ds} T_{ds} G_{ds} G_{ds} Aₐₛ | 67074 | 67093 | 31.1 | 207 | N/A |
| | G_{ds} G_{ds} Tₑₒ ^{m}Cₑₒ ^{m}Cₑₛ Tₑₛ Gₑ | | | | | |
| 628574 | Tₑₛ Tₑₒ ^{m}Cₑₒ Tₑₒ Tₑₒ T_{ds} ^{m}C_{ds} T_{ds} T_{ds} A_{ds} T_{ds} ^{m}C_{ds} T_{ds} T_{ds} | 67120 | 67139 | 39.8 | 208 | N/A |
| | T_{ds} ^{m}Cₑₒ Tₑₒ Tₑₛ ^{m}Cₑₛ Aₑ | | | | | |
| 628578 | Gₑₛ ^{m}Cₑₒ ^{m}Cₑₒ ^{m}Cₑₒ Tₑₒ ^{m}C_{ds} T_{ds} T_{ds} T_{ds} ^{m}C_{ds} T_{ds} ^{m}C_{ds} | 67132 | 67151 | 46.7 | 209 | N/A |
| | T_{ds} T_{ds} ^{m}C_{ds} Tₑₒ Tₑₒ Tₑₛ ^{m}Cₑₛ Tₑ | | | | | |
| 628582 | ^{m}Cₑₛ Tₑₒ Gₑₒ ^{m}Cₑₒ Aₑₒ ^{m}C_{ds} G_{ds} G_{ds} G_{ds} ^{m}C_{ds} T_{ds} ^{m}C_{ds} | 67150 | 67169 | 69.0 | 210 | N/A |
| | A_{ds} T_{ds} G_{ds} ^{m}Cₑₒ Tₑₒ Tₑₛ Gₑₛ ^{m}Cₑ | | | | | |
| 628586 | Gₑₛ Tₑₒ Gₑₒ Gₑₒ Gₑₒ ^{m}C_{ds} T_{ds} G_{ds} A_{ds} T_{ds} G_{ds} G_{ds} ^{m}C_{ds} | 67162 | 67181 | 31.7 | 211 | N/A |
| | T_{ds} G_{ds} ^{m}Cₑₒ Aₑₒ ^{m}Cₑₛ Gₑₛ Gₑ | | | | | |
| 628590 | ^{m}Cₑₛ ^{m}Cₑₒ Tₑₒ Gₑₒ ^{m}Cₑₒ ^{m}C_{ds} T_{ds} ^{m}C_{ds} T_{ds} G_{ds} ^{m}C_{ds} G_{ds} | 67188 | 67207 | 39.4 | 212 | N/A |
| | G_{ds} G_{ds} ^{m}C_{ds} Tₑₒ ^{m}Cₑₒ Aₑₛ Gₑₛ ^{m}Cₑ | | | | | |
| 628594 | ^{m}Cₑₛ Gₑₒ Gₑₒ Aₑₒ Gₑₒ ^{m}C_{ds} ^{m}C_{ds} T_{ds} G_{ds} A_{ds} ^{m}C_{ds} ^{m}C_{ds} | 67220 | 67239 | 50.9 | 213 | N/A |
| | ^{m}C_{ds} T_{ds} T_{ds} ^{m}Cₑₒ Tₑₒ Gₑₛ Aₑₛ Tₑ | | | | | |
| 628598 | Gₑₛ Gₑₒ Gₑₒ Aₑₒ Gₑₒ G_{ds} ^{m}C_{ds} A_{ds} G_{ds} A_{ds} A_{ds} G_{ds} ^{m}C_{ds} | 67250 | 67269 | 15.8 | 214 | N/A |
| | T_{ds} T_{ds} ^{m}Cₑₒ ^{m}Cₑₒ Gₑₛ Gₑₛ ^{m}Cₑ | | | | | |
| 628602 | ^{m}Cₑₛ ^{m}Cₑₒ Aₑₒ Gₑₒ ^{m}Cₑₒ ^{m}C_{ds} T_{ds} T_{ds} ^{m}C_{ds} A_{ds} G_{ds} G_{ds} | 67321 | 67340 | 37.1 | 215 | N/A |
| | ^{m}C_{ds} A_{ds} G_{ds} Gₑₒ Gₑₒ Tₑₛ Gₑₛ Gₑ | | | | | |
| 628606 | ^{m}Cₑₛ Gₑₒ Gₑₒ ^{m}Cₑₒ ^{m}Cₑₒ A_{ds} G_{ds} A_{ds} T_{ds} T_{ds} T_{ds} ^{m}C_{ds} ^{m}C_{ds} | 67353 | 67372 | 55.6 | 216 | N/A |
| | T_{ds} T_{ds} Tₑₒ Gₑₒ ^{m}Cₑₛ Tₑₛ Tₑ | | | | | |
| 628610 | Tₑₛ Gₑₒ Aₑₒ Tₑₒ ^{m}Cₑₒ A_{ds} A_{ds} A_{ds} T_{ds} A_{ds} ^{m}C_{ds} A_{ds} ^{m}C_{ds} | 67385 | 67404 | 53.0 | 217 | N/A |
| | A_{ds} T_{ds} ^{m}Cₑₒ Aₑₒ Tₑₛ Aₑₛ ^{m}Cₑ | | | | | |
| 628614 | Gₑₛ Tₑₒ Aₑₒ ^{m}Cₑₒ Gₑₒ ^{m}C_{ds} A_{ds} A_{ds} T_{ds} ^{m}C_{ds} A_{ds} A_{ds} ^{m}C_{ds} | 68188 | 68207 | 83.9 | 218 | N/A |
| | T_{ds} ^{m}C_{ds} ^{m}Cₑₒ Aₑₒ ^{m}Cₑₛ Tₑₛ Tₑ | | | | | |
| 628618 | Tₑₛ Gₑₒ Tₑₒ Gₑₒ Tₑₒ ^{m}C_{ds} G_{ds} ^{m}C_{ds} ^{m}C_{ds} T_{ds} A_{ds} ^{m}C_{ds} ^{m}C_{ds} | 68209 | 68228 | 50.1 | 219 | N/A |
| | T_{ds} T_{ds} Tₑₒ Tₑₒ ^{m}Cₑₛ Gₑₛ Aₑ | | | | | |
| 628622 | ^{m}Cₑₛ Aₑₒ Aₑₒ Aₑₒ Aₑₒ T_{ds} ^{m}C_{ds} A_{ds} T_{ds} T_{ds} A_{ds} G_{ds} G_{ds} | 68231 | 68250 | 58.6 | 220 | N/A |
| | G_{ds} T_{ds} ^{m}Cₑₒ ^{m}Cₑₒ Aₑₛ Gₑₛ Gₑ | | | | | |
| 628626 | Tₑₛ Tₑₒ Tₑₒ ^{m}Cₑₒ Tₑₒ G_{ds} ^{m}C_{ds} T_{ds} ^{m}C_{ds} T_{ds} ^{m}C_{ds} G_{ds} ^{m}C_{ds} | 68278 | 68297 | 35.9 | 221 | N/A |
| | ^{m}C_{ds} G_{ds} Gₑₒ Gₑₒ Aₑₛ Gₑₛ Gₑ | | | | | |
| 628630 | ^{m}Cₑₛ ^{m}Cₑₒ Aₑₒ Gₑₒ Tₑₒ T_{ds} ^{m}C_{ds} ^{m}C_{ds} T_{ds} G_{ds} G_{ds} A_{ds} G_{ds} | 68319 | 68338 | 51.3 | 222 | N/A |
| | ^{m}C_{ds} T_{ds} Tₑₒ Tₑₒ Gₑₛ Gₑₛ Gₑ | | | | | |
| 628634 | ^{m}Cₑₛ Aₑₒ ^{m}Cₑₒ ^{m}Cₑₒ Tₑₒ G_{ds} ^{m}C_{ds} A_{ds} ^{m}C_{ds} A_{ds} ^{m}C_{ds} ^{m}C_{ds} | 68392 | 68411 | 53.6 | 223 | N/A |
| | ^{m}C_{ds} T_{ds} ^{m}C_{ds} Tₑₒ Gₑₒ Aₑₛ ^{m}Cₑₛ Gₑ | | | | | |
| 628638 | Gₑₛ Aₑₒ Gₑₒ ^{m}Cₑₒ Tₑₒ T_{ds} ^{m}C_{ds} ^{m}C_{ds} ^{m}C_{ds} A_{ds} G_{ds} G_{ds} A_{ds} | 68425 | 68444 | 57.1 | 224 | N/A |
| | ^{m}C_{ds} T_{ds} Tₑₒ Tₑₒ Tₑₛ ^{m}Cₑₛ Tₑ | | | | | |
| 628642 | Gₑₛ Tₑₒ Tₑₒ Tₑₒ Gₑₒ A_{ds} A_{ds} A_{ds} A_{ds} G_{ds} G_{ds} ^{m}C_{ds} A_{ds} T_{ds} | 68448 | 68467 | 40.8 | 225 | N/A |
| | ^{m}C_{ds} Tₑₒ Tₑₒ Gₑₛ Aₑₛ ^{m}Cₑ | | | | | |
| 628646 | ^{m}Cₑₛ Tₑₒ Tₑₒ Gₑₒ ^{m}Cₑₒ ^{m}C_{ds} ^{m}C_{ds} ^{m}C_{ds} ^{m}C_{ds} T_{ds} G_{ds} G_{ds} | 68464 | 68483 | 58.5 | 226 | N/A |
| | ^{m}C_{ds} G_{ds} A_{ds} Aₑₒ Gₑₒ Tₑₛ Tₑₛ Tₑ | | | | | |
| 628650 | Tₑₛ Gₑₒ Tₑₒ Gₑₒ Gₑₒ T_{ds} G_{ds} G_{ds} ^{m}C_{ds} ^{m}C_{ds} ^{m}C_{ds} ^{m}C_{ds} A_{ds} | 68488 | 68507 | 57.7 | 227 | N/A |
| | ^{m}C_{ds} ^{m}C_{ds} ^{m}Cₑₒ ^{m}Cₑₒ ^{m}Cₑₛ ^{m}Cₑₛ Tₑ | | | | | |
| 628654 | Tₑₛ Tₑₒ Tₑₒ Gₑₒ Aₑₒ T_{ds} ^{m}C_{ds} A_{ds} ^{m}C_{ds} ^{m}C_{ds} A_{ds} T_{ds} G_{ds} | 68512 | 68531 | 69.1 | 228 | N/A |
| | A_{ds} ^{m}C_{ds} ^{m}Cₑₒ Tₑₒ Gₑₛ Gₑₛ Gₑ | | | | | |
| 628658 | Gₑₛ Gₑₒ Aₑₒ Aₑₒ Tₑₒ G_{ds} G_{ds} ^{m}C_{ds} ^{m}C_{ds} T_{ds} G_{ds} A_{ds} G_{ds} | 68559 | 68578 | 35.4 | 229 | N/A |
| | G_{ds} G_{ds} Tₑₒ ^{m}Cₑₒ Gₑₛ Gₑₛ ^{m}Cₑ | | | | | |
| 628662 | ^{m}Cₑₛ ^{m}Cₑₒ Aₑₒ ^{m}Cₑₒ Aₑₒ ^{m}C_{ds} T_{ds} ^{m}C_{ds} ^{m}C_{ds} ^{m}C_{ds} ^{m}C_{ds} G_{ds} | 68591 | 68610 | 58.1 | 230 | N/A |
| | G_{ds} ^{m}C_{ds} T_{ds} Tₑₒ Tₑₒ ^{m}Cₑₛ Gₑₛ Gₑ | | | | | |
| 628666 | Tₑₛ Tₑₒ Tₑₒ ^{m}Cₑₒ Tₑₒ T_{ds} T_{ds} T_{ds} T_{ds} G_{ds} G_{ds} ^{m}C_{ds} ^{m}C_{ds} T_{ds} | 68623 | 68642 | 47.3 | 231 | N/A |
| | ^{m}C_{ds} Gₑₒ Gₑₒ ^{m}Cₑₛ Gₑₛ Gₑ | | | | | |
| 628670 | ^{m}Cₑₛ Tₑₒ Gₑₒ ^{m}Cₑₒ Aₑₒ ^{m}C_{ds} A_{ds} G_{ds} A_{ds} T_{ds} ^{m}C_{ds} G_{ds} G_{ds} | 68656 | 68675 | 81.0 | 232 | N/A |
| | A_{ds} T_{ds} Aₑₒ Gₑₒ Aₑₛ Aₑₛ Gₑ | | | | | |
| *628674 | Gₑₛ Tₑₒ ^{m}Cₑₒ Tₑₒ Tₑₒ G_{ds} ^{m}C_{ds} G_{ds} ^{m}C_{ds} T_{ds} T_{ds} ^{m}C_{ds} T_{ds} | 68688 | 68707 | 69.4 | 233 | N/A |
| | T_{ds} G_{ds} Aₑₒ Tₑₒ Gₑₛ Gₑₛ Gₑ | | | | | |
| *628678 | ^{m}Cₑₛ Tₑₒ Tₑₒ ^{m}Cₑₒ ^{m}Cₑₒ T_{ds} T_{ds} G_{ds} A_{ds} ^{m}C_{ds} ^{m}C_{ds} T_{ds} | 68720 | 68739 | 95.8 | 234 | N/A |
| | ^{m}C_{ds} G_{ds} A_{ds} Tₑₒ Gₑₒ ^{m}Cₑₛ Tₑₛ Gₑ | | | | | |
| 628682 | Tₑₛ Tₑₒ ^{m}Cₑₒ ^{m}Cₑₒ ^{m}Cₑₒ G_{ds} ^{m}C_{ds} T_{ds} ^{m}C_{ds} T_{ds} T_{ds} ^{m}C_{ds} | 68766 | 68785 | 45.1 | 235 | N/A |
| | T_{ds} ^{m}C_{ds} A_{ds} ^{m}Cₑₒ ^{m}Cₑₒ Gₑₛ Aₑₛ Gₑ | | | | | |
| 628686 | Tₑₛ ^{m}Cₑₒ ^{m}Cₑₒ Gₑₒ ^{m}Cₑₒ ^{m}C_{ds} ^{m}C_{ds} A_{ds} G_{ds} G_{ds} G_{ds} ^{m}C_{ds} | 68798 | 68817 | 53.0 | 236 | N/A |
| | T_{ds} ^{m}C_{ds} T_{ds} Tₑₒ Aₑₒ ^{m}Cₑₛ Aₑₛ Gₑ | | | | | |
| 628690 | Tₑₛ Gₑₒ Gₑₒ Tₑₒ Gₑₒ G_{ds} T_{ds} G_{ds} ^{m}C_{ds} T_{ds} ^{m}C_{ds} ^{m}C_{ds} T_{ds} | 68868 | 68887 | 87.2 | 237 | N/A |
| | T_{ds} ^{m}C_{ds} Tₑₒ Tₑₒ Gₑₛ Gₑₛ Gₑ | | | | | |
| 628694 | ^{m}Cₑₛ Gₑₒ Gₑₒ Aₑₒ Gₑₒ ^{m}C_{ds} T_{ds} ^{m}C_{ds} T_{ds} ^{m}C_{ds} G_{ds} G_{ds} G_{ds} | 68954 | 68973 | 79.7 | 238 | N/A |
| | ^{m}C_{ds} T_{ds} ^{m}Cₑₒ Aₑₒ Gₑₛ Gₑₛ Tₑ | | | | | |
| 628698 | Aₑₛ Gₑₒ ^{m}Cₑₒ ^{m}Cₑₒ Tₑₒ ^{m}C_{ds} ^{m}C_{ds} T_{ds} ^{m}C_{ds} T_{ds} G_{ds} G_{ds} G_{ds} | 69032 | 69051 | 51.1 | 239 | N/A |
| | ^{m}C_{ds} A_{ds} Tₑₒ ^{m}Cₑₒ Tₑₛ Tₑₛ ^{m}Cₑ | | | | | |
| 628702 | ^{m}Cₑₛ Gₑₒ Gₑₒ Gₑₒ ^{m}Cₑₒ T_{ds} G_{ds} A_{ds} G_{ds} T_{ds} ^{m}C_{ds} T_{ds} T_{ds} | 69080 | 69099 | 69.6 | 240 | N/A |
| | A_{ds} G_{ds} ^{m}Cₑₒ Tₑₒ Gₑₛ Gₑₛ ^{m}Cₑ | | | | | |
| 628706 | Gₑₛ Gₑₒ ^{m}Cₑₒ Gₑₒ Gₑₒ T_{ds} G_{ds} G_{ds} ^{m}C_{ds} A_{ds} A_{ds} ^{m}C_{ds} ^{m}C_{ds} | 69094 | 69113 | 62.7 | 241 | N/A |
| | G_{ds} ^{m}C_{ds} Gₑₒ Gₑₒ Gₑₛ ^{m}Cₑₛ Tₑ | | | | | |
| 628710 | Tₑₛ ^{m}Cₑₒ Tₑₒ Gₑₒ ^{m}Cₑₒ G_{ds} G_{ds} ^{m}C_{ds} ^{m}C_{ds} G_{ds} T_{ds} G_{ds} G_{ds} | 69108 | 69127 | 54.2 | 242 | N/A |
| | ^{m}C_{ds} G_{ds} Gₑₒ ^{m}Cₑₒ Gₑₛ Gₑₛ Tₑ | | | | | |
| 628714 | ^{m}Cₑₛ ^{m}Cₑₒ ^{m}Cₑₒ ^{m}Cₑₒ Tₑₒ ^{m}C_{ds} G_{ds} G_{ds} T_{ds} G_{ds} T_{ds} T_{ds} T_{ds} | 69128 | 69147 | 47.5 | 243 | N/A |
| | G_{ds} T_{ds} Aₑₒ ^{m}Cₑₒ Tₑₛ Tₑₛ Tₑ | | | | | |
| 628718 | Gₑₛ Gₑₒ Aₑₒ Gₑₒ Gₑₒ A_{ds} T_{ds} G_{ds} A_{ds} A_{ds} A_{ds} ^{m}C_{ds} A_{ds} A_{ds} | 69160 | 69179 | 53.6 | 244 | N/A |
| | T_{ds} Gₑₒ Tₑₒ ^{m}Cₑₛ Tₑₛ Tₑ | | | | | |
| 628722 | Tₑₛ ^{m}Cₑₒ ^{m}Cₑₒ Aₑₒ ^{m}Cₑₒ A_{ds} G_{ds} G_{ds} ^{m}C_{ds} T_{ds} ^{m}C_{ds} ^{m}C_{ds} | 69192 | 69211 | 24.4 | 245 | N/A |
| | T_{ds} ^{m}C_{ds} T_{ds} ^{m}Cₑₒ Tₑₒ Gₑₛ Tₑₛ Tₑ | | | | | |
| 628726 | ^{m}Cₑₛ ^{m}Cₑₒ Gₑₒ Tₑₒ Gₑₒ T_{ds} A_{ds} A_{ds} A_{ds} G_{ds} T_{ds} ^{m}C_{ds} A_{ds} | 69236 | 69255 | 65.9 | 246 | N/A |
| | G_{ds} ^{m}C_{ds} Tₑₒ Aₑₒ Aₑₛ ^{m}Cₑₛ Tₑ | | | | | |
| 628730 | Tₑₛ Tₑₒ Tₑₒ Aₑₒ Tₑₒ T_{ds} ^{m}C_{ds} T_{ds} T_{ds} G_{ds} T_{ds} T_{ds} G_{ds} G_{ds} | 69268 | 69287 | 65.8 | 247 | N/A |
| | T_{ds} Tₑₒ Tₑₒ Gₑₛ ^{m}Cₑₛ Tₑ | | | | | |
| 628734 | ^{m}Cₑₛ ^{m}Cₑₒ Tₑₒ Aₑₒ ^{m}Cₑₒ ^{m}C_{ds} ^{m}C_{ds} A_{ds} T_{ds} A_{ds} A_{ds} G_{ds} | 69300 | 69319 | 48.9 | 248 | N/A |
| | G_{ds} A_{ds} G_{ds} Aₑₒ Aₑₒ Gₑₛ Aₑₛ Gₑ | | | | | |
| 628738 | Tₑₛ Aₑₒ Tₑₒ Tₑₒ Tₑₒ ^{m}C_{ds} A_{ds} G_{ds} T_{ds} T_{ds} A_{ds} A_{ds} T_{ds} ^{m}C_{ds} | 69332 | 69351 | 47.8 | 249 | N/A |
| | G_{ds} Gₑₒ Gₑₒ Aₑₛ Aₑₛ Gₑ | | | | | |

Superscript “m” indicates 5-methylcytosine. Subscripts: “o” indicates a phosphodiester internucleoside linkage, “s” indicates a phosphorothioate internucleoside linkage, “e” indicates a 2′-methoxyethyl modified nucleoside, and “d” indicates a 2′-deoxynucleoside.

TABLE 4
Inhibition or human MECP2 by antisense oligonucleotides in vitro

| | | | | % | | |
|---|---|---|---|---|---|---|
| Isis | | Start | Stop | Inhibi- | UNANNOTATED | ANNOTATED |
| No. | Sequence (5′ to 3′) | site | site | tion | SEQ ID NO: | SEQ ID NO |
| 18078 | Gₑₛ Tₑₛ Gₑₛ ^{m}Cₑₛ Gₑₛ C_{ds} G_{ds} C_{ds} G_{ds} A_{ds} G_{ds} C_{ds} C_{ds} C_{ds} | n/a | n/a | −9.9 | 15 | N/A |
| | Gₑₛ Aₑₛ Aₑₛ Aₑₛ Tₑₛ ^{m}Cₑ | | | | | |
| 628544 | Aₑₛ ^{m}Cₑₒ Aₑₒ Gₑₒ ^{m}Cₑₒ ^{m}C_{ds} T_{ds} ^{m}C_{ds} T_{ds} ^{m}C_{ds} T_{ds} | 1902 | 1921 | 45.6 | 250 | N/A |
| | ^{m}C_{ds} ^{m}C_{ds} G_{ds} Aₑₒ Gₑₒ Aₑₛ Gₑₛ Gₑ | | | | | |
| 628548 | ^{m}Cₑₛ Gₑₒ Gₑₒ ^{m}Cₑₒ Gₑₒ G_{ds} ^{m}C_{ds} G_{ds} G_{ds} ^{m}C_{ds} ^{m}C_{ds} A_{ds} | 1934 | 1953 | 69.6 | 251 | N/A |
| | T_{ds} T_{ds} T_{ds} Tₑₒ ^{m}Cₑₒ ^{m}Cₑₛ Gₑₛ Gₑ | | | | | |
| 628552 | Tₑₛ Gₑₒ Gₑₒ Aₑₒ Gₑₒ ^{m}C_{ds} A_{ds} G_{ds} T_{ds} ^{m}C_{ds} T_{ds} ^{m}C_{ds} T_{ds} | 1989 | 2008 | 40.3 | 252 | N/A |
| | ^{m}C_{ds} ^{m}C_{ds} Tₑₒ ^{m}Cₑₒ ^{m}Cₑₛ Tₑₛ ^{m}Cₑ | | | | | |
| 628740 | Tₑₛ Tₑₒ ^{m}Cₑₒ Aₑₒ Tₑₒ G_{ds} G_{ds} A_{ds} A_{ds} T_{ds} G_{ds} G_{ds} G_{ds} | 2547 | 2566 | 23.4 | 253 | N/A |
| | ^{m}C_{ds} G_{ds} Aₑₒ Gₑₒ Aₑₛ Aₑₛ Gₑ | | | | | |
| 628744 | Aₑₛ ^{m}Cₑₒ Aₑₒ Gₑₒ Aₑₒ G_{ds} G_{ds} ^{m}C_{ds} A_{ds} G_{ds} G_{ds} G_{ds} ^{m}C_{ds} | 4561 | 4580 | 62.6 | 254 | N/A |
| | A_{ds} G_{ds} Gₑₒ ^{m}Cₑₒ Aₑₛ ^{m}Cₑₛ Gₑ | | | | | |
| 628748 | Aₑₛ Aₑₒ Gₑₒ Aₑₒ Tₑₒ T_{ds} ^{m}C_{ds} A_{ds} T_{ds} G_{ds} ^{m}C_{ds} T_{ds} T_{ds} G_{ds} | 7090 | 7109 | 39.7 | 255 | N/A |
| | T_{ds} Tₑₒ Aₑₒ Gₑₛ Aₑₛ Aₑ | | | | | |
| 628556 | Tₑₛ ^{m}Cₑₒ Aₑₒ Aₑₒ Aₑₒ G_{ds} ^{m}C_{ds} A_{ds} G_{ds} G_{ds} A_{ds} A_{ds} ^{m}C_{ds} | 7316 | 7335 | 40.7 | 256 | 334 |
| | T_{ds} G_{ds} Gₑₒ Tₑₒ Gₑₛ Aₑₛ Gₑ | | | | | |
| 628560 | Gₑₛ Gₑₒ Tₑₒ ^{m}Cₑₒ Tₑₒ A_{ds} ^{m}C_{ds} A_{ds} G_{ds} A_{ds} A_{ds} G_{ds} ^{m}C_{ds} | 7359 | 7378 | 76.3 | 257 | 335 |
| | A_{ds} A_{ds} Gₑₒ Gₑₒ Tₑₛ Gₑₛ Tₑ | | | | | |
| 628564 | ^{m}Cₑₛ Aₑₒ Tₑₒ ^{m}Cₑₒ Tₑₒ ^{m}C_{ds} A_{ds} G_{ds} ^{m}C_{ds} T_{ds} A_{ds} ^{m}C_{ds} | 7391 | 7410 | 63.3 | 258 | 336 |
| | C_{ds} A_{ds} T_{ds} Gₑₒ Gₑₒ Aₑₛ Aₑₛ Tₑ | | | | | |
| 628752 | ^{m}Cₑₛ Aₑₒ ^{m}Cₑₒ ^{m}Cₑₒ Aₑₒ T_{ds} ^{m}C_{ds} ^{m}C_{ds} T_{ds} G_{ds} A_{ds} G_{ds} | 9908 | 9927 | 86.2 | 259 | N/A |
| | G_{ds} ^{m}C_{ds} ^{m}C_{ds} Aₑₒ Gₑₒ Gₑₛ ^{m}Cₑₛ Aₑ | | | | | |
| 628756 | Tₑₛ Aₑₒ Aₑₒ ^{m}Cₑₒ Tₑₒ T_{ds} T_{ds} T_{ds} T_{ds} T_{ds} ^{m}C_{ds} T_{ds} A_{ds} T_{ds} | 12623 | 12642 | 14 8 | 260 | N/A |
| | T_{ds} Aₑₒ Tₑₒ T_{cs} Aₑₛ Tₑ | | | | | |
| 628760 | Aₑₛ ^{m}Cₑₒ Aₑₒ G_{eO} Tₑₒ ^{m}C_{ds} A_{ds} ^{m}C_{ds} A_{ds} G_{ds} A_{ds} A_{ds} ^{m}C_{ds} | 14890 | 14909 | 80.6 | 261 | N/A |
| | A_{ds} A_{ds} ^{m}Cₑₒ Aₑₒ Aₑₛ Aₑₛ Gₑ | | | | | |
| 628764 | Gₑₛ Gₑₒ ^{m}Cₑₒ ^{m}Cₑₒ Tₑₒ A_{ds} A_{ds} T_{ds} T_{ds} T_{ds} T_{ds} T_{ds} T_{ds} A_{ds} | 17865 | 17884 | 80.6 | 262 | N/A |
| | T_{ds} ^{m}Cₑₒ Tₑₒ Tₑₛ Tₑₛ Gₑ | | | | | |
| 628768 | Aₑₛ ^{m}Cₑₒ Aₑₒ Gₑₒ Gₑₒ G_{ds} T_{ds} T_{ds} G_{ds} T_{ds} A_{ds} G_{ds} ^{m}C_{ds} | 20758 | 20777 | 91.0 | 263 | N/A |
| | ^{m}C_{ds} A_{ds} Tₑₒ ^{m}Cₑₒ Aₑₛ Gₑₛ Tₑ | | | | | |
| 628772 | Gₑₛ Aₑₒ Tₑₒ ^{m}Cₑₒ Aₑₒ ^{m}C_{ds} T_{ds} G_{ds} G_{ds} A_{ds} A_{ds} ^{m}C_{ds} A_{ds} | 22839 | 22858 | 91.6 | 264 | N/A |
| | ^{m}C_{ds} A_{ds} Aₑₒ Tₑₒ Gₑₛ Gₑₛ ^{m}Cₑ | | | | | |
| 628776 | Gₑₛ Gₑₒ Aₑₒ Aₑₒ Gₑₒ A_{ds} G_{ds} A_{ds} A_{ds} A_{ds} A_{ds} G_{ds} A_{ds} A_{ds} | 25437 | 25456 | 43.3 | 265 | N/A |
| | G_{ds} Gₑₒ Gₑₒ ^{m}Cₑₛ Aₑₛ ^{m}Cₑ | | | | | |
| 628780 | ^{m}Cₑₛ Aₑₒ Tₑₒ Tₑ Tₑₒ A_{ds} A_{ds} T_{ds} A_{ds} A_{ds} A_{ds} T_{ds} A_{ds} A_{ds} | 27672 | 27691 | 21.9 | 266 | N/A |
| | A_{ds} Tₑₒ ^{m}Cₑₒ ^{m}Cₑₛ ^{m}Cₑₛ Tₑ | | | | | |
| 628784 | Tₑₛ Tₑₒ Tₑₒ Aₑₒ ^{m}Cₑₒ ^{m}C_{ds} A_{ds} G_{ds} T_{ds} G_{ds} ^{m}C_{ds} ^{m}C_{ds} A_{ds} | 30227 | 30246 | 58.2 | 267 | N/A |
| | T_{ds} T_{ds} Tₑₒ Tₑₒ Tₑₛ ^{m}Cₑₛ ^{m}Cₑ | | | | | |
| 628788 | ^{m}Cₑₛ Aₑₒ Gₑₒ ^{m}Cₑₒ Aₑₒ A_{ds} A_{ds} T_{ds} T_{ds} T_{ds} ^{m}C_{ds} T_{ds} G_{ds} | 32258 | 32277 | 94.0 | 268 | N/A |
| | T_{ds} G_{ds} Gₑₒ Tₑₒ Tₑₛ Tₑₛ Tₑ | | | | | |
| 628792 | Gₑₛ ^{m}Cₑₒ Tₑₒ ^{m}Cₑₒ Tₑₒ ^{m}C_{ds} A_{ds} G_{ds} A_{ds} ^{m}C_{ds} ^{m}C_{ds} A_{ds} | 34773 | 34792 | 78.3 | 269 | N/A |
| | G_{ds} A_{ds} ^{m}C_{ds} ^{m}Cₑₒ Aₑₒ Gₑₛ Aₑₛ ^{m}Cₑ | | | | | |
| 628796 | Aₑₛ ^{m}Cₑₒ Aₑₒ Gₑₒ ^{m}Cₑₒ T_{ds} G_{ds} A_{ds} T_{ds} G_{ds} A_{ds} G_{ds} G_{ds} | 37542 | 37561 | 11.6 | 270 | N/A |
| | A_{ds} G_{ds} Gₑₒ Gₑₒ Tₑₛ Gₑₛ Gₑ | | | | | |
| 628800 | Tₑₛ Aₑₒ ^{m}Cₑₒ Aₑₒ ^{m}Cₑₒ A_{ds} A_{ds} A_{ds} T_{ds} A_{ds} ^{m}C_{ds} T_{ds} A_{ds} | 39572 | 39591 | 76.1 | 271 | N/A |
| | A_{ds} G_{ds} ^{m}Cₑₒ ^{m}Cₑₒ Aₑₛ ^{m}Cₑₛ Aₑ | | | | | |
| 628804 | Aₑₛ ^{m}Cₑₒ Tₑₒ Gₑₒ ^{m}Cₑₒ ^{m}C_{ds} A_{ds} ^{m}C_{ds} ^{m}C_{ds} A_{ds} ^{m}C_{ds} ^{m}C_{ds} | 41573 | 41592 | 63.7 | 272 | N/A |
| | A_{ds} T_{ds} G_{ds} Aₑₒ ^{m}Cₑₒ Tₑₛ Aₑₛ Aₑ | | | | | |
| 628808 | Gₑₛ Tₑₒ Tₑₒ Aₑₒ Gₑₒ A_{ds} A_{ds} G_{ds} T_{ds} T_{ds} G_{ds} A_{ds} T_{ds} T_{ds} | 44142 | 44161 | 80.7 | 273 | N/A |
| | T_{ds} Tₑₒ Tₑₒ Tₑₛ ^{m}Cₑₛ Tₑ | | | | | |
| 628812 | Aₑₛ Tₑₒ Aₑₒ ^{m}Cₑₒ Tₑₒ ^{m}C_{ds} A_{ds} ^{m}C_{ds} A_{ds} T_{ds} G_{ds} G_{ds} T_{ds} | 46268 | 46287 | 52.5 | 274 | N/A |
| | G_{ds} G_{ds} Aₑₒ Gₑₒ Aₑₛ Aₑₛ Aₑ | | | | | |
| 628816 | G_{qs} Aₑₒ Gₑₒ Aₑₒ Aₑₒ G_{ds} A_{ds} A_{ds} T_{ds} G_{ds} G_{ds} A_{ds} A_{ds} G_{ds} | 48363 | 48382 | 21.0 | 275 | N/A |
| | G_{ds} Gₑₒ Aₑₒ Gₑₛ Aₑₛ Aₑ | | | | | |
| 628820 | Tₑₛ Aₑₒ Gₑₒ Aₑₒ Gₑₒ G_{ds} G_{ds} T_{ds} T_{ds} G_{ds} G_{ds} A_{ds} G_{ds} G_{ds} | 50365 | 50384 | 25.7 | 276 | N/A |
| | A_{ds} Aₑₒ ^{m}Cₑₒ Aₑₛ Gₑₛ Gₑ | | | | | |
| 628824 | ^{m}Cₑₛ Tₑₒ Tₑₒ Aₑₒ Gₑₒ A_{ds} A_{ds} ^{m}C_{ds} A_{ds} A_{ds} A_{ds} G_{ds} A_{ds} | 53052 | 53071 | -5.2 | 277 | N/A |
| | G_{ds} A_{ds} Aₑₒ Gₑₒ Aₑₛ Aₑₛ Tₑ | | | | | |
| 628828 | Gₑₛ Aₑₒ ^{m}Cₑₒ Aₑₒ ^{m}Cₑₒ T_{ds} G_{ds} A_{ds} ^{m}C_{ds} A_{ds} ^{m}C_{ds} T_{ds} G_{ds} | 55069 | 55088 | 67.5 | 278 | N/A |
| | T_{ds} G_{ds} ^{m}Cₑₒ Aₑₒ Tₑₛ Gₑₛ Aₑ | | | | | |
| 628832 | Gₑₛ Gₑₒ Aₑₒ Gₑₒ Tₑₒ T_{ds} A_{ds} ^{m}C_{ds} ^{m}C_{ds} A_{ds} T_{ds} A_{ds} T_{ds} | 57122 | 57141 | 68.6 | 279 | N/A |
| | G_{ds} A_{ds} ^{m}Cₑₒ ^{m}Cₑₒ Tₑₛ Gₑₛ Gₑ | | | | | |
| 628836 | ^{m}Cₑₛ Gₑₒ Tₑₒ Aₑₒ Aₑₒ G_{ds} ^{m}C_{ds} T_{ds} T_{ds} ^{m}C_{ds} T_{ds} A_{ds} G_{ds} | 59723 | 59742 | 70.0 | 280 | N/A |
| | ^{m}C_{ds} A_{ds} Aₑₒ Gₑₒ Gₑₛ Aₑₛ Gₑ | | | | | |
| 628840 | Gₑₛ Gₑₒ Tₑₒ Aₑₒ Aₑₒ A_{ds} A_{ds} A_{ds} T_{ds} G_{ds} A_{ds} T_{ds} A_{ds} A_{ds} | 61802 | 61821 | −9.7 | 281 | N/A |
| | A_{ds} Aₑₒ Aₑₒ Aₑₛ ^{m}Cₑₛ Gₑ | | | | | |
| 628844 | Aₑₛ Gₑₒ ^{m}Cₑₒ ^{m}Cₑₒ Tₑₒ T_{ds} ^{m}C_{ds} T_{ds} ^{m}C_{ds} ^{m}C_{ds} T_{ds} G_{ds} | 63907 | 63926 | 82.8 | 282 | N/A |
| | ^{m}C_{ds} ^{m}C_{ds} T_{ds} ^{m}Cₑₒ Aₑₒ Gₑₛ ^{m}Cₑₛ Tₑ | | | | | |
| 628848 | Aₑₛ Gₑₒ Aₑₒ Aₑₒ Gₑₒ ^{m}C_{ds} A_{ds} G_{ds} ^{m}C_{ds} A_{ds} G_{ds} ^{m}C_{ds} ^{m}C_{ds} | 65932 | 65951 | 14.3 | 283 | N/A |
| | A_{ds} ^{m}C_{ds} ^{m}Cₑₒ Tₑₒ Gₑₛ ^{m}Cₑₛ Gₑ | | | | | |
| 628568 | Tₑₛ Gₑₒ Gₑₒ Tₑₒ ^{m}Cₑₒ T_{ds} T_{ds} ^{m}C_{ds} T_{ds} G_{ds} A_{ds} ^{m}C_{ds} T_{ds} | 67056 | 67075 | 56.8 | 284 | N/A |
| | T_{ds} T_{ds} Tₑₒ ^{m}Cₑₒ Tₑₛ Tₑₛ ^{m}Cₑ | | | | | |
| 628572 | ^{m}Cₑₛ Aₑₒ ^{m}Cₑₒ ^{m}Cₑₒ Tₑₒ T_{ds} T_{ds} T_{ds} T_{ds} A_{ds} A_{ds} A_{ds} ^{m}C_{ds} | 67102 | 67121 | 74.9 | 285 | N/A |
| | T_{ds} T_{ds} Gₑₒ Aₑₒ Gₑₛ Gₑₛ Gₑ | | | | | |
| 628576 | Tₑₒ Tₑₒ Tₑₒ ^{m}Cₑₒ Tₑₒ ^{m}C_{ds} T_{ds} T_{ds} ^{m}C_{ds} T_{ds} T_{ds} T_{ds} ^{m}C_{ds} | 67126 | 67145 | 47.3 | 286 | N/A |
| | T_{ds} T_{ds} Aₑₒ Tₑₒ ^{m}Cₑₛ Tₑₛ Tₑ | | | | | |
| 628580 | Tₑₛ ^{m}Cₑₒ Aₑₒ Tₑₒ Gₑₒ ^{m}C_{ds} T_{ds} T_{ds} G_{ds} ^{m}C_{ds} ^{m}C_{ds} ^{m}C_{ds} T_{ds} | 67140 | 67159 | 57.4 | 287 | N/A |
| | ^{m}C_{ds} T_{ds} Tₑₒ Tₑₒ ^{m}Cₑₛ Tₑₛ ^{m}Cₑ | | | | | |
| 628584 | Tₑₛ Gₑₒ Aₑₒ Tₑₒ Gₑₒ G_{ds} ^{m}C_{ds} T_{ds} G_{ds} ^{m}C_{ds} A_{ds} ^{m}C_{ds} G_{ds} | 67156 | 67175 | 58.1 | 288 | N/A |
| | G_{ds} G_{ds} ^{m}Cₑₒ Tₑₒ ^{m}Cₑₛ Aₑₛ Tₑ | | | | | |
| 628588 | ^{m}Cₑₛ Aₑₒ Gₑₒ ^{m}Cₑₒ Aₑₒ G_{ds} A_{ds} G_{ds} T_{ds} G_{ds} G_{ds} T_{ds} G_{ds} | 67172 | 67191 | 10.9 | 289 | N/A |
| | G_{ds} G_{ds} ^{m}Cₑₒ Tₑₒ Gₑₛ Aₑₛ Tₑ | | | | | |
| 628592 | Tₑₛ Gₑₒ Aₑₒ Tₑₒ Gₑₒ T_{ds} ^{m}C_{ds} T_{ds} ^{m}C_{ds} T_{ds} G_{ds} ^{m}C_{ds} T_{ds} | 67204 | 67223 | 37.9 | 290 | N/A |
| | T_{ds} T_{ds} Gₑₒ ^{m}Cₑₒ ^{m}Cₑₛ Tₑₛ Gₑ | | | | | |
| 628596 | ^{m}Cₑₛ Aₑₒ Gₑₒ Aₑₒ Aₑₒ G_{ds} ^{m}C_{ds} T_{ds} T_{ds} ^{m}C_{ds} ^{m}C_{ds} G_{ds} G_{ds} | 67244 | 67263 | 16.0 | 291 | N/A |
| | ^{m}C_{ds} A_{ds} ^{m}Cₑₒ Aₑₒ Gₑₛ ^{m}Cₑₛ ^{m}Cₑ | | | | | |
| 628600 | Gₑₛ Gₑₒ Gₑₒ Tₑₒ ^{m}Cₑₒ ^{m}C_{ds} ^{m}C_{ds} ^{m}C_{ds} G_{ds} G_{ds} T_{ds} ^{m}C_{ds} | 67288 | 67307 | 50.5 | 292 | N/A |
| | A_{ds} ^{m}C_{ds} G_{ds} Gₑₒ Aₑₒ Tₑₛ Gₑₛ Aₑ | | | | | |
| 628604 | Gₑₛ ^{m}Cₑₒ Tₑₒ Tₑₒ Aₑₒ A_{ds} G_{ds} ^{m}C_{ds} T_{ds} T_{ds} ^{m}C_{ds} ^{m}C_{ds} G_{ds} | 67337 | 67356 | 59.6 | 293 | N/A |
| | T_{ds} G_{ds} Tₑₒ ^{m}Cₑₒ ^{m}Cₑₛ Aₑₛ Gₑ | | | | | |
| 628608 | Aₑₛ Tₑₒ Aₑₒ Tₑₒ ^{m}Cₑₒ T_{ds} T_{ds} ^{m}C_{ds} ^{m}C_{ds} A_{ds} G_{ds} ^{m}C_{ds} A_{ds} | 67369 | 67388 | 19.9 | 294 | N/A |
| | G_{ds} A_{ds} Gₑₒ ^{m}Cₑₒ Gₑₛ Gₑₛ ^{m}Cₑ | | | | | |
| 628852 | Aₑₛ Gₑₒ ^{m}Cₑₒ Aₑₒ Aₑₒ ^{m}C_{ds} ^{m}C_{ds} A_{ds} A_{ds} A_{ds} G_{ds} A_{ds} G_{ds} | 67934 | 67953 | 76.1 | 295 | N/A |
| | T_{ds} ^{m}C_{ds} Aₑₒ Gₑₒ Gₑₛ ^{m}Cₑₛ ^{m}Cₑ | | | | | |
| 628612 | Aₑₛ ^{m}Cₑₒ Tₑₒ Tₑₒ Tₑₒ A_{ds} G_{ds} A_{ds} G_{ds} ^{m}C_{ds} G_{ds} A_{ds} A_{ds} | 68172 | 68191 | 36.2 | 296 | N/A |
| | A_{ds} G_{ds} Gₑₒ ^{m}Cₑₒ Tₑₛ Tₑₛ Tₑ | | | | | |
| 628616 | Tₑₛ Tₑₒ Tₑₒ Tₑₒ ^{m}Cₑₒ G_{ds} A_{ds} A_{ds} G_{ds} T_{ds} A_{ds} ^{m}C_{ds} G_{ds} | 68196 | 68215 | 41.9 | 297 | N/A |
| | ^{m}C_{ds} A_{ds} Aₑₒ Tₑₒ ^{m}Cₑₛ Aₑₛ Aₑ | | | | | |
| 628620 | ^{m}Cₑₛ ^{m}Cₑₒ Aₑₒ Gₑₒ Gₑₒ G_{ds} A_{ds} T_{ds} G_{ds} T_{ds} G_{ds} T_{ds} ^{m}C_{ds} | 68216 | 68235 | 62.3 | 298 | N/A |
| | G_{ds} ^{m}C_{ds} ^{m}Cₑₒ Tₑₒ Aₑₛ ^{m}Cₑₛ ^{m}Cₑ | | | | | |
| 628624 | ^{m}Cₑₛ ^{m}Cₑₒ Aₑₒ Gₑₒ Tₑₒ T_{ds} A_{ds} ^{m}C_{ds} ^{m}C_{ds} G_{ds} T_{ds} G_{ds} A_{ds} | 68247 | 68266 | 40.4 | 299 | N/A |
| | A_{ds} G_{ds} Tₑₒ ^{m}Cₑₒ Aₑₛ Aₑₛ Aₑ | | | | | |
| 628628 | Tₑₛ Gₑₒ Gₑₒ Gₑₒ ^{m}Cₑₒ T_{ds} T_{ds} ^{m}C_{ds} T_{ds} T_{ds} A_{ds} G_{ds} G_{ds} T_{ds} | 68294 | 68313 | 53.5 | 300 | N/A |
| | G_{ds} Gₑₒ Tₑₒ Tₑₛ Tₑₛ ^{m}Cₑ | | | | | |
| 628632 | Gₑₛ Tₑₒ Gₑₒ Gₑₒ Tₑₒ G_{ds} ^{m}C_{ds} ^{m}C_{ds} G_{ds} ^{m}C_{ds} T_{ds} ^{m}C_{ds} ^{m}C_{ds} | 68355 | 68374 | 65.8 | 301 | N/A |
| | ^{m}C_{ds} T_{ds} Tₑₒ Tₑₒ Gₑₛ Gₑₛ Gₑ | | | | | |
| 628636 | Tₑₛ ^{m}Cₑₒ Tₑₒ ^{m}Cₑₒ ^{m}Cₑₒ A_{ds} G_{ds} G_{ds} A_{ds} ^{m}C_{ds} ^{m}C_{ds} ^{m}C_{ds} | 68408 | 68427 | 38.7 | 302 | N/A |
| | T_{ds} T_{ds} T_{ds} Tₑₒ ^{m}Cₑₒ Aₑₛ ^{m}Cₑₛ ^{m}Cₑ | | | | | |
| 628640 | Gₑₛ Aₑₒ ^{m}Cₑₒ Aₑₒ Aₑₒ G_{ds} G_{ds} A_{ds} G_{ds} ^{m}C_{ds} T_{ds} T_{ds} ^{m}C_{ds} | 68431 | 68450 | 57.4 | 303 | N/A |
| | ^{m}C_{ds} ^{m}C_{ds} Aₑₒ Gₑₒ Gₑₛ Aₑₛ ^{m}Cₑ | | | | | |
| 628644 | ^{m}Cₑₛ ^{m}Cₑₒ ^{m}Cₑₒ Tₑₒ Gₑₒ G_{ds} ^{m}C_{ds} G_{ds} A_{ds} A_{ds} G_{ds} T_{ds} T_{ds} | 68458 | 68477 | 37.8 | 304 | N/A |
| | T_{ds} G_{ds} Aₑₒ Aₑₒ Aₑₛ Aₑₛ Gₑ | | | | | |
| 628648 | ^{m}Cₑₛ ^{m}Cₑₒ ^{m}Cₑₒ ^{m}Cₑₒ Tₑₒ ^{m}C_{ds} A_{ds} G_{ds} ^{m}C_{ds} ^{m}C_{ds} T_{ds} T_{ds} | 68473 | 68492 | 25.0 | 305 | N/A |
| | G_{ds} ^{m}C_{ds} ^{m}C_{ds} ^{m}Cₑₒ ^{m}Cₑₒ ^{m}Cₑₛ Tₑₛ Gₑ | | | | | |
| 628652 | Tₑₛ Gₑₒ Gₑₒ Gₑₒ Tₑₒ G_{ds} G_{ds} A_{ds} T_{ds} G_{ds} T_{ds} G_{ds} G_{ds} T_{ds} | 68496 | 68515 | 13.6 | 306 | N/A |
| | G_{ds} Gₑₒ ^{m}Cₑₒ ^{m}Cₑₛ ^{m}Cₑₛ ^{m}Cₑ | | | | | |
| 628656 | ^{m}Cₑₛ Gₑₒ Gₑₒ ^{m}Cₑₒ ^{m}Cₑₒ T_{ds} ^{m}C_{ds} A_{ds} G_{ds} ^{m}C_{ds} T_{ds} T_{ds} T_{ds} | 68543 | 68562 | 59.4 | 307 | N/A |
| | T_{ds} ^{m}C_{ds} Gₑₒ ^{m}Cₑₒ Tₑₛ Tₑₛ ^{m}Cₑ | | | | | |
| 628660 | Tₑₛ ^{m}Cₑₒ Gₑₒ Gₑₒ ^{m}Cₑₒ ^{m}C_{ds} ^{m}C_{ds} ^{m}C_{ds} G_{ds} T_{ds} T_{ds} T_{ds} | 68575 | 68594 | 33.8 | 308 | N/A |
| | ^{m}C_{ds} T_{ds} T_{ds} Gₑₒ Gₑₒ Gₑₛ Aₑₛ Aₑ | | | | | |
| 628664 | Gₑₛ ^{m}Cₑₒ Gₑₒ Gₑₒ ^{m}Cₑₒ A_{ds} G_{ds} ^{m}C_{ds} G_{ds} G_{ds} ^{m}C_{ds} T_{ds} G_{ds} | 68607 | 68626 | 31.0 | 309 | N/A |
| | ^{m}C_{ds} ^{m}C_{ds} Aₑₒ ^{m}Cₑₒ ^{m}Cₑₛ Aₑₛ ^{m}Cₑ | | | | | |
| *628668 | Aₑₛ Aₑₒ Gₑₒ Aₑₒ ^{m}Cₑₒ T_{ds} ^{m}C_{ds} ^{m}C_{ds} T_{ds} T_{ds} ^{m}C_{ds} A_{ds} ^{m}C_{ds} | 68639 | 68658 | 69.2 | 310 | N/A |
| | G_{ds} G_{ds} ^{m}Cₑₒ Tₑₒ Tₑₛ Tₑₛ ^{m}Cₑ | | | | | |
| *628672 | Gₑₛ Gₑₒ Tₑₒ ^{m}Cₑₒ Tₑₒ ^{m}C_{ds} ^{m}C_{ds} T_{ds} G_{ds} ^{m}C_{ds} A_{ds} ^{m}C_{ds} A_{ds} | 68662 | 68681 | 95.2 | 311 | N/A |
| | G_{ds} A_{ds} Tₑₒ ^{m}Cₑₒ Gₑₛ Gₑₛ Aₑ | | | | | |
| *628676 | Gₑₛ ^{m}Cₑₒ Tₑₒ Gₑₒ Aₑₒ ^{m}C_{ds} ^{m}C_{ds} G_{ds} T_{ds} ^{m}C_{ds} T_{ds} ^{m}C_{ds} | 68704 | 68723 | 40.4 | 312 | N/A |
| | ^{m}C_{ds} ^{m}C_{ds} G_{ds} Gₑₒ Gₑₒ Tₑₛ ^{m}Cₑₛ Tₑ | | | | | |
| 628680 | ^{m}Cₑₛ Gₑₒ Aₑₒ Gₑₒ Gₑₒ G_{ds} T_{ds} G_{ds} G_{ds} A_{ds} ^{m}C_{ds} A_{ds} ^{m}C_{ds} | 68750 | 68769 | 37.2 | 313 | N/A |
| | ^{m}C_{ds} A_{ds} Gₑₒ ^{m}Cₑₒ Aₑₛ Gₑₛ Gₑ | | | | | |
| 628684 | Aₑₛ ^{m}Cₑₒ Aₑₒ Gₑₒ Gₑₒ T_{ds} ^{m}C_{ds} T_{ds} T_{ds} ^{m}C_{ds} A_{ds} G_{ds} T_{ds} | 68782 | 68801 | 63.5 | 314 | N/A |
| | ^{m}C_{ds} ^{m}C_{ds} Tₑₒ Tₑₒ Tₑₛ ^{m}Cₑₛ ^{m}Cₑ | | | | | |
| 628688 | ^{m}Cₑₛ Tₑₒ Gₑₒ ^{m}Cₑₒ Tₑₒ ^{m}C_{ds} T_{ds} ^{m}C_{ds} ^{m}C_{ds} T_{ds} T_{ds} G_{ds} | 68814 | 68833 | 84.1 | 315 | N/A |
| | ^{m}C_{ds} T_{ds} T_{ds} Tₑₒ Tₑₒ ^{m}Cₑₛ ^{m}Cₑₛ Gₑ | | | | | |
| 628692 | ^{m}Cₑₛ Tₑₒ Gₑₒ Aₑₒ Gₑₒ T_{ds} G_{ds} G_{ds} T_{ds} G_{ds} G_{ds} T_{ds} G_{ds} A_{ds} | 68885 | 68904 | 11.9 | 316 | N/A |
| | T_{ds} Gₑₒ Gₑₒ Tₑₛ Gₑₛ Gₑ | | | | | |
| 628696 | ^{m}Cₑₛ Tₑₒ Tₑₒ ^{m}Cₑₒ Tₑₒ ^{m}C_{ds} ^{m}C_{ds} T_{ds} ^{m}C_{ds} T_{ds} T_{ds} T_{ds} G_{ds} | 69016 | 69035 | 70.9 | 317 | N/A |
| | ^{m}C_{ds} A_{ds} Gₑₒ Aₑₒ ^{m}Cₑₛ Gₑₛ ^{m}Cₑ | | | | | |
| 628700 | ^{m}Cₑₛ ^{m}Cₑₒ Gₑₒ Tₑₒ ^{m}Cₑₒ G_{ds} ^{m}C_{ds} T_{ds} ^{m}C_{ds} T_{ds} ^{m}C_{ds} ^{m}C_{ds} | 69048 | 69067 | 71.6 | 318 | N/A |
| | A_{ds} G_{ds} T_{ds} Gₑₒ Aₑₒ Gₑₛ ^{m}Cₑₛ ^{m}Cₑ | | | | | |
| 628704 | Gₑₛ Gₑₒ ^{m}Cₑₒ Aₑₒ Aₑₒ ^{m}C_{ds} ^{m}C_{ds} G_{ds} ^{m}C_{ds} G_{ds} G_{ds} G_{ds} | 69088 | 69107 | 72.3 | 319 | N/A |
| | ^{m}C_{ds} T_{ds} G_{ds} Aₑₒ Gₑₒ Tₑₛ ^{m}Cₑₛ Tₑ | | | | | |
| 628708 | ^{m}Cₑₛ ^{m}Cₑₒ Gₑₒ Tₑₒ Gₑₒ G_{ds} ^{m}C_{ds} G_{ds} G_{ds} ^{m}C_{ds} G_{ds} G_{ds} T_{ds} | 69101 | 69120 | 32.3 | 320 | N/A |
| | G_{ds} G_{ds} ^{m}Cₑₒ Aₑₒ Aₑₛ ^{m}Cₑₛ ^{m}Cₑ | | | | | |
| 628712 | Tₑₛ Aₑₒ ^{m}Cₑₒ Tₑₒ Tₑₒ T_{ds} T_{ds} ^{m}C_{ds} T_{ds} G_{ds} ^{m}C_{ds} G_{ds} G_{ds} | 69114 | 69133 | 55.9 | 321 | N/A |
| | ^{m}C_{ds} ^{m}C_{ds} Gₑₒ Tₑₒ Gₑₛ Gₑₛ ^{m}Cₑ | | | | | |
| 628716 | Tₑₛ ^{m}Cₑₒ Tₑₒ Tₑₒ Tₑₒ G_{ds} ^{m}C_{ds} G_{ds} ^{m}C_{ds} T_{ds} ^{m}C_{ds} T_{ds} ^{m}C_{ds} | 69144 | 69163 | 52.4 | 322 | N/A |
| | ^{m}C_{ds} ^{m}C_{ds} Tₑₒ ^{m}Cₑₒ ^{m}Cₑₛ ^{m}Cₑₛ ^{m}Cₑ | | | | | |
| 628720 | Tₑₛ Gₑₒ Tₑₒ Tₑₒ Tₑₒ G_{ds} G_{ds} ^{m}C_{ds} ^{m}C_{ds} T_{ds} T_{ds} G_{ds} G_{ds} | 69176 | 69195 | 55.1 | 323 | N/A |
| | ^{m}C_{ds} A_{ds} Tₑₒ Gₑₒ Gₑₛ Aₑₛ Gₑ | | | | | |
| 628724 | Aₑₛ Aₑₒ ^{m}Cₑₒ Tₑₒ ^{m}Cₑₒ T_{ds} ^{m}C_{ds} T_{ds} ^{m}C_{ds} G_{ds} G_{ds} T_{ds} ^{m}C_{ds} | 69220 | 69239 | 82.2 | 324 | N/A |
| | A_{ds} ^{m}C_{ds} Gₑₒ Gₑₒ Gₑₛ ^{m}Cₑₛ Gₑ | | | | | |
| 628728 | Tₑₛ Gₑₒ ^{m}Cₑₒ Tₑₒ Tₑₒ T_{ds} G_{ds} ^{m}C_{ds} A_{ds} A_{ds} T_{ds} ^{m}C_{ds} ^{m}C_{ds} | 69252 | 69271 | 77.9 | 325 | N/A |
| | G_{ds} ^{m}C_{ds} Tₑₒ ^{m}Cₑₒ ^{m}Cₑₛ Gₑₛ Tₑ | | | | | |
| 628732 | Aₑₛ Gₑₒ Aₑₒ Gₑₒ Aₑₒ ^{m}C_{ds} A_{ds} A_{ds} ^{m}C_{ds} A_{ds} G_{ds} ^{m}C_{ds} T_{ds} | 69284 | 69303 | 57.7 | 326 | N/A |
| | G_{ds} ^{m}C_{ds} ^{m}Cₑₒ Tₑₒ Tₑₛ Tₑₛ Aₑ | | | | | |
| 628736 | Gₑₛ Aₑₒ Aₑₒ Gₑₒ ^{m}Cₑₒ T_{ds} T_{ds} T_{ds} G_{ds} T_{ds} ^{m}C_{ds} A_{ds} G_{ds} | 69316 | 69335 | 90.1 | 327 | N/A |
| | A_{ds} G_{ds} ^{m}Cₑₒ ^{m}Cₑₒ ^{m}Cₑₛ Tₑₛ Aₑ | | | | | |

Superscript “m” indicates 5-methylcytosine. Subscripts: “o” indicates a phosphodiester internucleoside linkage, “s” indicates a phosphorothioate internucleoside linkage, “e” indicates a 2′-methoxyethyl modified nucleoside, and “d” indicates a 2′-deoxynucleoside.

### Example 2: Dose Response of Antisense Oligonucleotides Targeting MECP2 In Vitro

MECP2 targeting antisense oligonucleotides selected from Tables 1-4 were tested for dose response analysis in HepG2 cells. Isis Number 141923 does not target MECP2 and was used as a negative control. Cells were electroporated with 0, 0.111, 0.333, 1.00, 3.00, or 9.00 μM antisense oligonucleotide, and MECP2 mRNA was analyzed as described in Example 1. Results are presented in Tables 5 and 6 below. Isis Numbers 141923 and 628749 were included in both data sets as references for comparison. The results show that the antisense oligonucleotides targeting MECP2 inhibited MECP2 mRNA expression in a dose dependent manner.

TABLE 5
Dose repsonse in vitro

| | % Inhibition | | | | | |
|---|---|---|---|---|---|---|
| | 0.111 | | | | | SEQ ID |
| Isis No. | μM | 0.333 μM | 1.00 μM | 3.00 μM | 9.00 μM | NO: |
| 141923 | 101.7 | 124.0 | 97.3 | 105.3 | 70.6 | 328 |
| 628688 | 89.1 | 73.1 | 47.8 | 24.3 | 17.0 | 315 |
| 628724 | 83.6 | 76.8 | 41.2 | 17.4 | 14.1 | 324 |
| 628736 | 84.6 | 68.9 | 36.3 | 21.4 | 9.1 | 327 |
| 628749 | 63.8 | 36.4 | 19.3 | 7.7 | 3.7 | 103 |
| 628751 | 102.3 | 77.4 | 39.2 | 19.5 | 8.3 | 25 |
| 628752 | 76.0 | 77.0 | 47.4 | 28.0 | 18.4 | 259 |
| 628763 | 63.5 | 37.1 | 11.5 | 8.1 | 6.7 | 28 |
| 628767 | 82.2 | 56.7 | 33.0 | 16.0 | 12.0 | 29 |
| 628768 | 98.4 | 68.4 | 43.7 | 21.7 | 11.0 | 263 |
| 628772 | 84.3 | 60.6 | 34.4 | 13.7 | 5.4 | 264 |
| 628775 | 84.3 | 62.4 | 37.0 | 15.9 | 6.3 | 31 |
| 628787 | 81.8 | 60.5 | 38.6 | 26.6 | 10.0 | 34 |
| 628788 | 79.8 | 65.1 | 35.9 | 10.5 | 4.9 | 268 |
| 628811 | 69.1 | 46.8 | 20.6 | 22.1 | 4.2 | 40 |
| 628844 | 82.6 | 76.4 | 49.6 | 38.1 | 16.2 | 282 |

TABLE 6
Dose repsonse in vitro

| | % Inhibition | | | | | |
|---|---|---|---|---|---|---|
| | 0.111 | | | | | SEQ ID |
| Isis No. | μM | 0.333 μM | 1.00 μM | 3.00 μM | 9.00 μM | NO: |
| 141923 | 116.6 | 123.1 | 120.7 | 119.6 | 119.0 | 328 |
| 628558 | 94.5 | 65.4 | 49.2 | 19.9 | 9.2 | 178 |
| 628614 | 85.8 | 84.4 | 60.5 | 28.9 | 15.3 | 218 |
| 628637 | 93.1 | 80.1 | 63.4 | 25.3 | 8.3 | 146 |
| 628641 | 100.4 | 81.8 | 55.3 | 24.3 | 11.2 | 147 |
| 628690 | 101.7 | 77.9 | 51.5 | 28.0 | 16.4 | 237 |
| 628694 | 101.6 | 86.5 | 50.6 | 25.1 | 13.9 | 238 |
| 628742 | 103.3 | 73.6 | 48.8 | 20.1 | 14.7 | 175 |
| 628749 | 78.3 | 45.6 | 15.8 | 9.1 | 9.9 | 103 |
| 628757 | 88.6 | 70.5 | 39.0 | 21.7 | 13.5 | 105 |
| 628762 | 67.5 | 47.3 | 22.8 | 8.1 | 18.2 | 184 |
| 628766 | 119.8 | 77.5 | 65.6 | 31.5 | 18.0 | 185 |
| 628785 | 72.5 | 45.8 | 25.8 | 15.1 | 18.9 | 112 |
| 628786 | 85.6 | 55.5 | 36.0 | 17.3 | 10.6 | 190 |
| 628822 | 88.4 | 84.3 | 45.8 | 36.6 | 11.5 | 199 |
| 628833 | 90.6 | 70.1 | 55.2 | 32.1 | 10.8 | 124 |

### Example 3: Effect of Antisense Oligonucleotides Targeting MECP2 In Vivo

Antisense oligonucleotides (ASOs) that target human Methyl CpG Binding Protein 2 (MECP2), the complement of GENBANK accession number NT_167198.1 truncated from 4203000 to 4283000, SEQ ID NO: 2, were synthesized using standard solid phase oligonucleotide synthetic methods. They are chimeric oligonucleotides (“gapmers”), composed of a central “gap” region consisting of 2′-deoxynucleotides, which is flanked on both sides (5′ and 3′) by “wings” that are composed of modified nucleotides. The internucleoside (backbone) linkages are phosphorothioate or phosphodiester throughout the oligonucleotides. The sequences and structures of the antisense oligonucleotides and their start and stop sites along SEQ ID NO: 2 are shown in the table below.

TABLE 7
Antisense oligonucleotides targeted to human MECP2

| | | | | SEQ |
|---|---|---|---|---|
| Isis | | Start | Stop | ID |
| No. | Sequence (5′ to 3′) | site | site | NO. |
| 628724 | Aₑₛ Aₑₒ ^{m}Cₑₒ Tₑₒ ^{m}Cₑₒ T_{ds} ^{m}C_{ds} | 69220 | 69239 | 324 |
| | T_{ds} ^{m}C_{ds} G_{ds} G_{ds} T_{ds} ^{m}C_{ds} A_{ds} | | | |
| | ^{m}C_{ds} Gₑₒ Gₑₒ Gₑₛ ^{m}Cₑₛ Gₑ | | | |
| 628749 | ^{m}Cₑₛ Aₑₒ ^{m}Cₑₒ Aₑₒ ^{m}Cₑₒ T_{ds} G_{ds} | 7615 | 7634 | 103 |
| | A_{ds} ^{m}C_{ds} ^{m}C_{ds} T_{ds} T_{ds} T_{ds} ^{m}C_{ds} | | | |
| | A_{ds} Gₑₒ Gₑₒ Gₑₛ ^{m}Cₑₛ Tₑ | | | |
| 628772 | Gₑₛ Aₑₒ Tₑₒ ^{m}Cₑₒ Aₑₒ ^{m}C_{ds} T_{ds} | 22839 | 22858 | 264 |
| | G_{ds} G_{ds} A_{ds} A_{ds} ^{m}C_{ds} A_{ds} ^{m}C_{ds} | | | |
| | A_{ds} Aₑₒ Tₑₒ Gₑₛ Gₑₛ Tₑ | | | |
| 628775 | ^{m}Cₑₛ Gₑₒ Tₑₒ Gₑₒ ^{m}Cₑₒ ^{m}C_{ds} A_{ds} | 24936 | 24955 | 31 |
| | T_{ds} G_{ds} G_{ds} A_{ds} A_{ds} G_{ds} T_{ds} ^{m}C_{ds} | | | |
| | ^{m}Cₑₒ Tₑₒ Tₑₛ ^{m}Cₑₛ ^{m}Cₑₒ | | | |
| 628785 | Gₑₛ Gₑₒ Tₑₒ Tₑₒ Tₑₒ T_{ds} T_{ds} ^{m}C_{ds} | 30744 | 30763 | 112 |
| | T_{ds} ^{m}C_{ds} ^{m}C_{ds} T_{ds} T_{ds} T_{ds} A_{ds} Tₑₒ | | | |
| | Tₑₒ Aₑₛ Tₑₛ ^{m}Cₑₒ | | | |

Superscript “m” indicates 5-methylcytosine. Subscripts: “o” indicates a phosphodiester internucleoside linkage, “s” indicates a phosphorothioate internucleoside linkage, “e” indicates a 2′-methoxyethyl modified nucleoside, and “d” indicates a 2′-deoxynucleoside.

The antisense oligonucleotides were analyzed for their effects on MECP2 mRNA and protein levels in transgenic MECP2 duplication mice that overexpress wild type human MECP2 (F1 hybrid MECP2-TG1 mice(FVB/N×129)(Samaco et al., Nat Genet, 2012). At 8 weeks of age, FVB/N×129 mice display hypoactivity in the open field test, increased anxiety in the open field and elevated plus maze tests, abnormal social behavior in the 3-chamber test, and increased motor coordination in the rotarod test. Seven week old MECP2-TG mice were given stereotactic intracerebral injection of 500 μg of an antisense oligonucleotide listed in Table 7 or saline into the right ventricle of the brain. Wild type mice were given stereotactic intracerebral injection of saline into the right ventricle of the brain as a control. Each group consisted of two or three mice. Two weeks following the injection, the mice were sacrificed, and cortical brain samples were collected for analysis of MECP2 mRNA and protein levels. MECP2 and GAPDH protein levels were analyzed by western blot performed on the cortical sample lysates. Rabbit antiserum raised against the N-terminus of MECP2 and mouse anti-GAPDH 6C₅ (Advanced Immunochemicals, Long Beach, CA) were used as the primary antibodies. Western blot images were quantified using Image J software, and the MECP2 protein levels normalized to GAPDH levels are shown in Table 8 below.

Total MECP2 mRNA, human MECP2 mRNA (both the e1 and e2 isoforms), and mouse MECP2 mRNA (both the e 1 and e2 isoforms) were separately analyzed by RT-qPCR. The primers common to human and mouse used for total MECP2 mRNA were: 5′-TATTTGATCAATCCCCAGGG-3′, SEQ ID NO: 3, and 5′-CTCCCTCTCCCAGTTACCGT-3′, SEQ ID NO: 4. The human specific primers used for MECP2-e1 were 5′-AGGAGAGACTGGAAGAAAAGTC-3′, SEQ ID NO: 5, and 5′-CTTGAGGGGTTTGTCCTTGA-3′, SEQ ID NO: 6. The human specific primers used for MECP2-e2 were 5′-CTCACCAGTTCCTGCTTTGATGT-3′, SEQ ID NO: 7, and 5′-CTTGAGGGGTTTGTCCTTGA-3′, SEQ ID NO: 6. The mouse specific primers used for MECP2-e1 were 5′-AGGAGAGACTGGAGGAAAAGTC-3′, SEQ ID NO: 8, and 5′-CTTAAACTTCAGTGGCTTGTCTCTG-3′, SEQ ID NO: 9. The mouse specific primers used for MECP2-e2 were 5′-CTCACCAGTTCCTGCTTTGATGT-3′, SEQ ID NO: 7, and 5′-CTTAAACTTCAGTGGCTTGTCTCTG-3′, SEQ ID NO: 9. MECP2 mRNA levels were normalized to Hprt mRNA levels, which were analyzed using primer 5′-CGGGGGACATAAAAGTTATTG-3′, SEQ ID NO: 10, and 5′-TGCATTGTTTTACCAGTGTCAA-3′, SEQ ID NO: 11. Results are presented in Table 8 below as average normlized MECP2 mRNA levels relative to saline treated wild type (WT) mice. The results show that all of the antisense oligonucleotides tested inhibited MECP2 mRNA and protein levels in the transgenic mice, and human MECP2 mRNA levels were specifically inhibited, whereas mouse MECP2 mRNA levels were not inhibited. Isis Number 628785 was the most potent in the first experiments and was carried forward. Entries listed as “n/a” indicate that the corresponding experiment was not performed.

TABLE 8
MECP2 mRNA and protein levels in transgenic mice following ASO administration

| | MECP2 | Total | Human mRNA | | Mouse mRNA | | SEQ |
|---|---|---|---|---|---|---|---|
| Mouse/Isis | protein | MECP2 | MECP2-e1 | MECP2-e2 | MECP2-e1 | MECP2-e2 | ID |
| No. | level | mRNA | isoform | isoform | isoform | isoform | NO. |
| WT/PBS | 1.0 | 1.0 | 0.0 | 0.0 | 1.0 | 1.3 | |
| TG/PBS | 2.0 | 3.3 | 0.9 | 8.3 | 1.2 | 1.4 | |
| TG/628724 | 1.5 | 2.0 | n/a | n/a | n/a | n/a | 324 |
| TG/628749 | 1.6 | 2.5 | n/a | n/a | n/a | n/a | 103 |
| TG/628772 | 1.7 | 2.7 | n/a | n/a | n/a | n/a | 264 |
| TG/628775 | 1.4 | 2.1 | n/a | n/a | n/a | n/a | 31 |
| TG/628785 | 1.3 | 1.6 | 0.3 | 2.3 | 1.0 | 1.3 | 112 |

### Example 4: Effect of Gradual Infusion of Antisense Oligonucleotide Targeting MECP2 In Vivo

In order to gradually infuse antisense oligonucleotide into the right ventricle of the brain, micro-osmotic pumps (Alzet model 1004, Durect, Cupertino, CA) were filled with 500 μg of Isis No. 628785 or a control oligonucleotide that is not targeted to MECP2, dissolved in 100 μl saline. The pump was then connected through a plastic catheter to a cannula (Alzet Brain Infusion Kit 3, Durect, Cupertino, CA). The pump was designed to deliver the drug at a rate of 0.11 μl per hour for 28 days. The cannula and pump assembly was primed in sterile saline for two days at 37° C. Mice were anesthetized with isoflurane and placed on a computer-guided stereotaxic instrument (Angle Two Stereotaxic Instrument, Leica Microsystems, Bannockburn, IL). Anesthesia (isoflurane 3%) was continuously delivered via a small face mask. Ketoprofen 5 mg/kg was administered subcutaneously at the initiation of the surgery. After sterilizing the surgical site with betadine and 70% alcohol, a midline incision was made over the skull and a subcutaneous pocket was generated on the back of the animal. Next, the pump was inserted into the pocket and the cannula was stereotactically implanted to deliver the drug in the right ventricle using the following coordinates: AP=−0.2 mm, ML=1 mm, DV=−3 mm. The incision was sutured shut. Carprofen-containing food pellets were provided for 5 days after the surgery. 28 days after the initiation of the treatment the pump was disconnected from the cannula and removed. Two additional weeks were given to the animals to recover.

Isis No. 628785 was gradually infused into the right ventricles of the brains of 7-week old WT or TG mice using the micro-osmotic pumps. Each treatment group consisted of 4 or 5 animals. At the end of the four-week treatment period, western blot was performed as described in Example 3 to analyze MECP2 protein levels at 4, 8, and 12 weeks following the initiation of antisense oligonucleotide treatment. The results are shown in Table 9 below.

TABLE 9
MECP2 protein levels following antisense oligonucleotide infusion

| | MECP2 protein level (relative to WT/Control) | | |
|---|---|---|---|
| Mouse/Isis No. | 4 weeks | 8 weeks | 12 weeks |
| WT/Control | 1.0 | 1.0 | 1.0 |
| TG/Control | 2.9 | 2.7 | 2.3 |
| TG/628785 | 1.6 | 1.8 | 2.2 |

### Example 5: Behavioral Effects of Antisense Oligonucleotide Targeting Human MECP2 In Vivo

Following infusion of antisense oligonucleotide as described in Example 4, a battery of behavioral assays were performed to assess phenotypic effects of oligonucleotide treatment in TG mice treated with Isis No. 628785 or a control oligonucleotide and WT mice treated with a control oligonucleotide. Each treatment group contained at least 15 animals.

An open field test was performed two weeks and six weeks after the completion of the 4 week infusion by placing mice into the center of an open arena after habituation in the test room (40×40×30 cm). Their behavior was tracked by laser photobeam breaks for 30 min. Horizontal locomotor activity, rearing activity, time spent in the center of the arena, and entries to the center were analyzed using AccuScan Fusion software (Omnitech, Columbus, OH). The results are reported in table 10 below. The results show that the TG mice displayed hypoactivity in the open field test relative to WT mice at both time points, and treatment of TG mice with Isis No. 628785 restored activity close to WT levels.

TABLE 10
Open field test

| | Horizontal | | | | | | | |
|---|---|---|---|---|---|---|---|---|
| | activity | | | | | | | |
| | (activity | | Rearing | | Time in | | Entries | |
| Mouse/Isis | counts) | | episodes | | center (s) | | to center | |
| No. | 2 weeks | 6 weeks | 2 weeks | 6 weeks | 2 weeks | 6 weeks | 2 weeks | 6 weeks |
| WT/Control | 7134 | 5632 | 277 | 236 | 179 | n/a | 147 | 103 |
| TG/Control | 4116 | 3493 | 156 | 106 | 105 | n/a | 65 | 45 |
| TG/628785 | 5550 | 6114 | 170 | 205 | 93 | n/a | 75 | 99 |

Mice were tested in an elevated plus maze two weeks and six weeks after the completion of the 4 week infusion. After habituation in the test room, mice were placed in the center part of the maze facing one of the two open arms. Mouse behavior was video-tracked for 10 minutes, and the time the mice spent in the open arms and the entries to the open arms were recorded and analyzed using ANY-maze system (Stoelting, Wood Dale, IL). The results are shown in Table 11 below. The results show that the TG mice displayed increased anxiety in the elevated plus maze test relative to WT mice at both time points, and treatment of TG mice with Isis No. 628785 restored anxiety levels close to WT levels.

TABLE 11
Elevated plus maze

| Mouse/Isis | Time in open arms (s) | | Entries into open arms | |
|---|---|---|---|---|
| No. | 2 weeks | 6 weeks | 2 weeks | 6 weeks |
| WT/Control | 139 | 81 | 21 | 12 |
| TG/Control | 76 | 13 | 12 | 2 |
| TG/628785 | 91 | 55 | 11 | 7 |

Mice were assessed in a three-chamber social interaction test three weeks and seven weeks after the completion of the 4 week infusion. The apparatus comprised a clear Plexiglas box with removable partitions that separated the box into three chambers: left, central, and right. In the left and right chambers a cylindrical wire cup was placed with the open side down. Age and gender-matched mice were used as novel partners. Two days before the test, the novel partner mice were habituated to the wire cups (3 inches diameter by 4 inches in height) for 1 hour per day. After habituation in the test room, each mouse was placed in the central chamber and allowed to explore the three chambers for 10 minutes (habituation phase). The time spent in each chamber during the habituation phase was recorded automatically and analyzed using ANY-maze system (Stoelting, Wood Dale, IL). Next, a novel partner mouse was placed under a wire cup in either the left or the right chamber. An inanimate object was placed as a control under the wire cup of the opposite chamber. The location of the novel mouse was randomized between the left and right chambers for each test mouse to control for side preference. The mouse tested was allowed to explore again for an additional 10 minutes. The time spent investigating the novel partner (defined by rearing, sniffing or pawing at the wire cup) and the time spent investigating the inanimate object were measured manually. The results are shown in Table 12 below. The results show that the TG mice displayed hypoactivity and decreased social interaction in the three-chamber social interaction test relative to WT mice at both time points, and treatment of TG mice with Isis No. 628785 restored social interaction with a novel partner to WT levels at the 6 week time point.

TABLE 12
Three-chamber social interaction test

| | Time spent investigating | | | | Time spent investigating | | | |
|---|---|---|---|---|---|---|---|---|
| | chambers during | | | | novel partner or | | | |
| | habituation phase (s) | | | | inanimate object (s) | | | |
| Mouse/Isis | Left | | Right | | Novel partner | | Inanimate object | |
| No. | 2 weeks | 6 weeks | 2 weeks | 6 weeks | 2 weeks | 6 weeks | 2 weeks | 6 weeks |
| WT/Control | 52.4 | n/a | 44.1 | n/a | 141 | 107 | 38 | 37 |
| TG/Control | 35.5 | n/a | 29.9 | n/a | 106 | 59 | 33 | 28 |
| TG/628785 | 37.7 | n/a | 23.0 | n/a | 94 | 106 | 27 | 28 |

Mice were assessed in an accelerating rotarod test three weeks after the completion of the 4 week infusion. After habituation in the test room, motor coordination was measured using an accelerating rotarod apparatus (Ugo Basile, Varese, Italy). Mice were tested 2 consecutive days, 4 trials each, with an interval of 60 minutes between trials to rest. Each trial lasted for a maximum of 10 minutes; mice that never fell were given a measurement of 600 seconds. The rod accelerated from 4 to 40 r.p.m. in the first 5 minutes. The time that it took for each mouse to fall from the rod (latency to fall) was recorded. Results are shown in Table 13 below. The results show that the TG mice displayed increased performance in the rotarod test relative to WT mice, and treatment of TG mice with Isis No. 628785 restored performance to WT levels.

TABLE 13
Accelerating rotarod test

| Mouse/Isis | Latency to fall (s) | |
|---|---|---|
| No. | Day 1 | Day 2 |
| WT/Control | 174 | 300 |
| TG/Control | 275 | 400 |
| TG/628785 | 183 | 282 |

The results in tables 10-13 above show that treatment with Isis No. 628785 targeting MECP2 reversed behavioral phenotypes of the TG mice. The TG mice treated with Isis No. 628785 performed similarly to WT mice in the rotarod test 3-4 weeks after completion of the infusion. By 6-7 weeks after completion of the infusion, the hypoactivity, anxiety-like behaviors and social behavior of the TG mice were reversed, as evidenced by the open field, elevated plus maze and three-chamber tests, respectively.

### Example 6: Dose Response of Antisense Oligonucleotide Targeting Human MECP2 in Patient Cells

In order to test for a dose dependent effect of Isis No. 628785 on human cells, B-lymphoblast cells from two individuals affected with MECP2-duplication syndrome and age-matched control cells were cultured in suspension in RPMI 1640 medium with L-glutamine, penicillin-streptomycin, and 10% (v/v) fetal bovice serum. A day before transfection, cells were seeded in triplicate for each treatment in 6-well plates at 10⁶ cells per well in a total volume of 2 mL medium. Cells were transfected with Isis No. 628785 or control oligonucleotide at a concentration listed in Table 14 below with TurboFect transfection reagent (Thermo Scientific, Carlsbad, CA). Cells were harvested and RNA was extracted 48 hours after transfection, and MECP2 mRNA levels were analyzed as described in Example 1. Results are presented in Table 14 below as average normlized MECP2 mRNA levels for both patients' cells relative to untreated control cells. The results show that Isis No. 628785 inhibited MECP2 expression in human MECP2 duplication patient cells.

TABLE 14
Antisense oligonucleotide treatment of patient lymphoblasts

| | Concentration | Total relative |
|---|---|---|
| Cell type/Isis No. | (nM) | MECP2 mRNA |
| Control/Control | 600 | 1.0 |
| Patient/Control | 600 | 3.1 |
| Patient/628785 | 150 | 2.2 |
| Patient/628785 | 300 | 1.6 |
| Patient/628785 | 600 | 1.3 |

### Example 7: Reduction of Seizure Activity with an Antisense Oligonucleotide Targeting Human MECP2 In Vivo

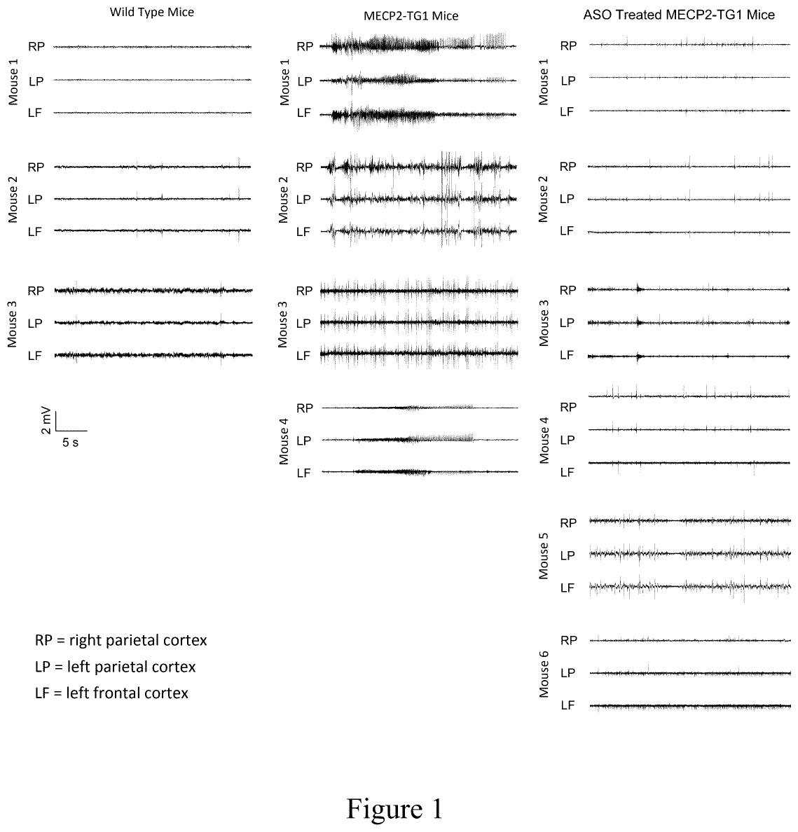

Without treatment, seizures and accompanying abnormal electrographic discharges occur in MECP2-TG1 mice as they age. In order to test the effect of antisense oligonucleotide treatment on seizure activity in MECP2-TG1 mice, electrocephalography recordings were performed and behavioral seizure activity was observed. 25-35 week old MECP2-TG1 mice that had been treated as described in Example 4 were anaesthetized with isoflurane and mounted in a stereotaxic frame for the surgical implantation of three recording electrodes (Teflon-coated silver wire, 125 μm in diameter) in the subdural space of the left frontal cortex, the left parietal cortex, and the right parietal cortex, with a reference electrode placed in the occipital region of the skull. After 3-5 days of surgical recovery, cortical EEG activity and behavior were recorded for 2h per day over 3-5 days. Strong electrographic seizure events were typically accompanied by behavioral seizures. FIG. 1 displays representative EEG traces for WT mice, MECP2-TG1 mice without Isis No. 628785 treatment, and MECP2-TG1 mice that received treatment with Isis No. 628785. Treatment of MECP2-TG1 mice with Isis No. 628785 eliminated both behavioral seizures and abnormal EEG discharges.

1. A compound comprising a modified antisense oligonucleotide consisting of 12 to 30 linked nucleosides and having a nucleobase sequence comprising at least 8, at least 9, at least 10, at least 11, at least 12, at least 13, at least 14, at least 15, at least 16, at least 17, at least 18, at least 19, or 20 consecutive nucleobases of any of the nucleobase sequences of SEQ ID NOs: 16-327.

2. The compound of claim 1, wherein the nucleobase sequence of the modified antisense oligonucleotide is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% complementary to SEQ ID NO: 1 or SEQ ID NO: 2.

3. The compound of claim 1, consisting of a single-stranded modified antisense oligonucleotide.

4. The compound of claim 1, wherein at least one internucleoside linkage is a modified internucleoside linkage.

5. The compound of claim 4, wherein at least one modified internucleoside linkage is a phosphorothioate internucleoside linkage.

6. The compound of claim 4, wherein each modified internucleoside linkage is a phosphorothioate internucleoside linkage.

7. The compound of claim 1, wherein at least one internucleoside linkage is a phosphodiester internucleoside linkage.

8. The compound of claim 1, wherein at least one internucleoside linkage is a phosphorothioate linkage and at least one internucleoside linkage is a phosphodiester linkage.

9. The compound of claim 1, wherein at least one nucleoside comprises a modified nucleobase.

10. The compound of claim 9, wherein the modified nucleobase is a 5-methylcytosine.

11. The compound of claim 1, wherein at least one nucleoside of the modified antisense oligonucleotide comprises a modified sugar moiety.

12. The compound of claim 11, wherein the at-least-ene-modified sugar moiety is a bicyclic sugar moiety.

13. The compound of claim 12, wherein the bicyclic sugar moiety comprises a 4′-CH(R)—O-2′ bridge wherein R is, independently, H, C₁-C₁₂ alkyl, or a protecting group.

14. The compound of claim 13, wherein R is methyl.

15. The compound of claim 13, wherein R is H.

16. The compound of claim 11, wherein the modified sugar moiety is a 2′-MOE modified sugar moiety.

17. The compound of claim 1, wherein the modified antisense oligonucleotide comprises:
a gap segment consisting of 10 linked deoxynucleosides;
a 5′ wing segment consisting of 5 linked nucleosides; and
a 3′ wing segment consisting of 5 linked nucleosides;
wherein the gap segment is positioned between the 5′ wing segment and the 3′ wing segment and wherein each nucleoside of each wing segment comprises a modified sugar.

18. The compound of claim 1, wherein the modified antisense oligonucleotide consists of 20 linked nucleosides.

19. A composition comprising the compound of claim 1 or a salt thereof and a pharmaceutically acceptable carrier or diluent.
